SEQUENCE LISTING (141 sequences)

SEQ ID NO: 1 - protein, 310 aa, Homo sapience
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHLASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 2 - protein, 16 aa, Artificial Sequence
  CDR
KSSQSLLASDGKTYLN

SEQ ID NO: 3 - protein, 11 aa, Artificial Sequence
  CDR
QSLLASDGKTY

SEQ ID NO: 4 - protein, 11 aa, Artificial Sequence
  CDR
KASENVGNYVS

SEQ ID NO: 5 - protein, 6 aa, Artificial Sequence
  CDR
ENVGNY

SEQ ID NO: 6 - protein, 11 aa, Artificial Sequence
  CDR
KASENVGTYVS

SEQ ID NO: 7 - protein, 8 aa, Artificial Sequence
  CDR
ASENVGTY

SEQ ID NO: 8 - protein, 10 aa, Artificial Sequence
  CDR
RASSSVNYMH

SEQ ID NO: 9 - protein, 7 aa, Artificial Sequence
  CDR
ASSSVNY

SEQ ID NO: 10 - protein, 11 aa, Artificial Sequence
  CDR
KASENVGSYVS

SEQ ID NO: 11 - protein, 8 aa, Artificial Sequence
  CDR
ASENVGSY

SEQ ID NO: 12 - protein, 16 aa, Artificial Sequence
  CDR
KSGQSLLYSNGKTYLT

SEQ ID NO: 13 - protein, 11 aa, Artificial Sequence
  CDR
KSGQSLLYSNG

SEQ ID NO: 14 - protein, 16 aa, Artificial Sequence
  CDR
KSSQSLLYSNGKTYLN

SEQ ID NO: 15 - protein, 11 aa, Artificial Sequence
  CDR
KSSQSLLYSNG

SEQ ID NO: 16 - protein, 8 aa, Artificial Sequence
  CDR
YLVSKLDS

SEQ ID NO: 17 - protein, 7 aa, Artificial Sequence
  CDR
LVSKLDS

SEQ ID NO: 18 - protein, 6 aa, Artificial Sequence
  CDR
LVSKLD

SEQ ID NO: 19 - protein, 8 aa, Artificial Sequence
  CDR
YGASYRYT

SEQ ID NO: 20 - protein, 7 aa, Artificial Sequence
  CDR
GASYRYT

SEQ ID NO: 21 - protein, 6 aa, Artificial Sequence
  CDR
GASYRY

SEQ ID NO: 22 - protein, 7 aa, Artificial Sequence
  CDR
GASNRYT

SEQ ID NO: 23 - protein, 7 aa, Artificial Sequence
  CDR
ATSYLAS

SEQ ID NO: 24 - protein, 7 aa, Artificial Sequence
  CDR
GASNRNT

SEQ ID NO: 25 - protein, 7 aa, Artificial Sequence
  CDR
QVSKLDP

SEQ ID NO: 26 - protein, 7 aa, Artificial Sequence
  CDR
LVSKVDS

SEQ ID NO: 27 - protein, 9 aa, Artificial Sequence
  CDR
WQGAHFPFT

SEQ ID NO: 28 - protein, 7 aa, Artificial Sequence
  CDR
QGAHFPF

SEQ ID NO: 29 - protein, 9 aa, Artificial Sequence
  CDR
GQSSRYPLT

SEQ ID NO: 30 - protein, 7 aa, Artificial Sequence
  CDR
QSSRYPL

SEQ ID NO: 31 - protein, 9 aa, Artificial Sequence
  CDR
QQWSSDPLT

SEQ ID NO: 32 - protein, 9 aa, Artificial Sequence
  CDR
LQNTYYPHT

SEQ ID NO: 33 - protein, 9 aa, Artificial Sequence
  CDR
VQGTHFPLT

SEQ ID NO: 34 - protein, 113 aa, Mus musculus
DVVMTQTPLTLSVTIGQPASISCKSSQSLLASDGKTYLNWLLQRPGQSPKRLIYLVSKLDSGVPDRFTGSGSGTGFTLQISRVEAEDLGIYYCWQGAHFPFTFGSGTKLEIKR

SEQ ID NO: 35 - protein, 108 aa, Mus musculus
NIIMTQSPKSMAMSVGERVTLSCKASENVGNYVSWYQQKPEQSPKLLIYGASYRYTGVPDRFTGSGSGTDFTLTISSVQAEDLADYHCGQSSRYPLTFGAGTKLELKR

SEQ ID NO: 36 - protein, 108 aa, Mus musculus
NIVMTQSPKSMSMSVGERVTLSCKASENVGTYVSWYQQKSDQSPKLLIYGASNRYTGVPDRFTGSGSATDFTLTITSVQSEDLADYHCGQSSRYPLTFGAGTKLELKR

SEQ ID NO: 37 - protein, 107 aa, Mus musculus
QIVLSQSPAILSASPGEKVTMTCRASSSVNYMHWYQQKPGSSPKHWIYATSYLASGVPARFSGSGSGTSYSLTISRVEAEDAATYYCQQWSSDPLTFGAGTKLELKR

SEQ ID NO: 38 - protein, 108 aa, Mus musculus
NIVMTQSPKSMSMSVGQRVTLSCKASENVGSYVSWYQQKPEQSPKLLIYGASNRNTGVPDRFTGSGSATDFTLTISSVQAEDLADYHCGQSSRYPLTFGGGTKLELKR

SEQ ID NO: 39 - protein, 113 aa, Mus musculus
DVVMTQTPLSLSVTIGQPASISCKSGQSLLYSNGKTYLTWLQQRPGQAPKLLMYQVSKLDPGIPDRFSGSGSETDFTLKISRVEAEDLGVYYCLQNTYYPHTFGAGTKLELKR

SEQ ID NO: 40 - protein, 113 aa, Mus musculus
DVVMTQTPLTLSVTIGQSASISCKSSQSLLYSNGKTYLNWLLQRPGQSPKRLIYLVSKVDSGVPDRFTGSGSGTDFTLSISRVEAEDLGVYYCVQGTHFPLTFGVGTKLELKR

SEQ ID NO: 41 - protein, 113 aa, Artificial Sequence
  Humanized Light chain variable region
DVVMTQTPLSLSVTPGQPASISCKSSQSLLASDGKTYLNWLLQRPGQSPKRLIYLVSKLDSGVPNRFSGSGSGTDFTLQISRVEAEDVGLYYCWQGAHFPFTFGSGTKLEIKR

SEQ ID NO: 42 - protein, 113 aa, Artificial Sequence
  Humanized Light chain variable region
DVVMTQTPLSLSVTPGQPASISCKSSQSLLASDGKTYLNWLLQRPGQSPKRLIYLVSKLDSGVPNRFSGSGSGTDFTLQISRVEAEDVGLYYCMQGAHFPFTFGGGTKVEIKR

SEQ ID NO: 43 - protein, 113 aa, Artificial Sequence
  Humanized Light chain variable region
DVVMTQSPLSLSVTLGQPASISCKSSQSLLASDGKTYLNWLQQRPGQSPRRLIYLVSKLDSGVPNRFSGSGSGTDFTLQISRVEAEDVGLYYCWQGAHFPFTFGSGTKLEIKR

SEQ ID NO: 44 - protein, 219 aa, Artificial Sequence
  Humanized Light chain variable region
DVVMTQTPLSLSVTPGQPASISCKSSQSLLASDGKTYLNWLLQRPGQSPKRLIYLVSKLDSGVPNRFSGSGSGTDFTLQISRVEAEDVGLYYCWQGAHFPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 13 aa, Artificial Sequence
  CDR
KASGYTFTSYYLY

SEQ ID NO: 46 - protein, 10 aa, Artificial Sequence
  CDR
GYTFTSYYLY

SEQ ID NO: 47 - protein, 5 aa, Artificial Sequence
  CDR
SYYLY

SEQ ID NO: 48 - protein, 13 aa, Artificial Sequence
  CDR
AASGFTFNTYAMN

SEQ ID NO: 49 - protein, 11 aa, Artificial Sequence
  CDR
ASGFTFNTYAM

SEQ ID NO: 50 - protein, 5 aa, Artificial Sequence
  CDR
TYAMN

SEQ ID NO: 51 - protein, 10 aa, Artificial Sequence
  CDR
GFAFNTYAMN

SEQ ID NO: 52 - protein, 10 aa, Artificial Sequence
  CDR
GYTFSSHWMQ

SEQ ID NO: 53 - protein, 5 aa, Artificial Sequence
  CDR
SHWMQ

SEQ ID NO: 54 - protein, 10 aa, Artificial Sequence
  CDR
GFTFNTYAMN

SEQ ID NO: 55 - protein, 13 aa, Artificial Sequence
  CDR
KASGYTFTNYYMY

SEQ ID NO: 56 - protein, 5 aa, Artificial Sequence
  CDR
NYYMY

SEQ ID NO: 57 - protein, 5 aa, Artificial Sequence
  CDR
YTFAD

SEQ ID NO: 58 - protein, 9 aa, Artificial Sequence
  CDR
YTFADYYMK

SEQ ID NO: 59 - protein, 5 aa, Artificial Sequence
  CDR
DYYMK

SEQ ID NO: 60 - protein, 10 aa, Artificial Sequence
  CDR
EIYPRSGGTN

SEQ ID NO: 61 - protein, 17 aa, Artificial Sequence
  CDR
EIYPRSGGTNINEKFLS

SEQ ID NO: 62 - protein, 12 aa, Artificial Sequence
  CDR
RIRSKSNNYATY

SEQ ID NO: 63 - protein, 19 aa, Artificial Sequence
  CDR
RIRSKSNNYATYYADSVKD

SEQ ID NO: 64 - protein, 11 aa, Artificial Sequence
  CDR
IRSKSNNYATY

SEQ ID NO: 65 - protein, 17 aa, Artificial Sequence
  CDR
AIYPGDGDTRFTQKFKG

SEQ ID NO: 66 - protein, 12 aa, Artificial Sequence
  CDR
YPGDGDTRFTQK

SEQ ID NO: 67 - protein, 19 aa, Artificial Sequence
  CDR
RIRSKSNNYATYYVDSVKD

SEQ ID NO: 68 - protein, 17 aa, Artificial Sequence
  CDR
EINPGNGGTNFNEKFKN

SEQ ID NO: 69 - protein, 13 aa, Artificial Sequence
  CDR
NPGNGGTNFNEKF

SEQ ID NO: 70 - protein, 10 aa, Artificial Sequence
  CDR
DINPNSGDTF

SEQ ID NO: 71 - protein, 17 aa, Artificial Sequence
  CDR
DINPNSGDTFYNHKFKG

SEQ ID NO: 72 - protein, 6 aa, Artificial Sequence
  CDR
TRSLLY

SEQ ID NO: 73 - protein, 4 aa, Artificial Sequence
  CDR
SLLY

SEQ ID NO: 74 - protein, 11 aa, Artificial Sequence
  CDR
VTDYGYVYFDA

SEQ ID NO: 75 - protein, 9 aa, Artificial Sequence
  CDR
DYGYVYFDA

SEQ ID NO: 76 - protein, 9 aa, Artificial Sequence
  CDR
DYYYVYFDV

SEQ ID NO: 77 - protein, 12 aa, Artificial Sequence
  CDR
GIYYDRSRAMDY

SEQ ID NO: 78 - protein, 11 aa, Artificial Sequence
  CDR
VTDYGHVYFDV

SEQ ID NO: 79 - protein, 9 aa, Artificial Sequence
  CDR
DYGHVYFDV

SEQ ID NO: 80 - protein, 4 aa, Artificial Sequence
  CDR
RFAY

SEQ ID NO: 81 - protein, 5 aa, Artificial Sequence
  CDR
TYYDY

SEQ ID NO: 82 - protein, 113 aa, Mus musculus
QVQLQQSGAEVVKPGASVKLSCKASGYTFTSYYLYWVKKGPGQGLDWIGEIYPRSGGTNINEKFLSKATLTADESSSTAYLQLSSLTSEDSAVYYCTRSLLYWGQGTTLIVSS

SEQ ID NO: 83 - protein, 120 aa, Mus musculus
EVQLVESGGGLVQPKGSLKLSCAASGFTFNTYAMNWVRQAPGKGLEWIARIRSKSNNYATYYADSVKDRFTISRDDSQSLLYLQMNNLKTEDTAIFYCVTDYGYVYFDAWGAGTTVTVSS

SEQ ID NO: 84 - protein, 120 aa, Mus musculus
EVQLLESGGGLVQPEGSLKLSCAASGFAFNTYAMNWVRQAPGKGLEWLARIRSKSNNYATYYADSVKDRFTISRDDSQGMLYLQMNNLKTEDTAMYYCVTDYYYVYFDVWGAGTTVTVSS

SEQ ID NO: 85 - protein, 121 aa, Mus musculus
QVQLQQSGAELARPGASVKLSCKASGYTFSSHWMQWVKQRPGQGLEWIGAIYPGDGDTRFTQKFKGKATLTADKSSNTAYMQLSSLASEDSAVYYCARGIYYDRSRAMDYWGQGTSVTVSS

SEQ ID NO: 86 - protein, 120 aa, Mus musculus
EVQLVESGGGLVQPKGSLKLSCATSGFTFNTYAMNWVRQAPGKGLEWVARIRSKSNNYATYYVDSVKDRFTISRDDSQSTVHLQMNNLKTEDTAIYYCVTDYGHVYFDVWGAGTTVTVSS

SEQ ID NO: 87 - protein, 113 aa, Mus musculus
QVQLQQSGAELVKPGASVKLSCKASGYTFTNYYMYWVKQRPGQGLEWIGEINPGNGGTNFNEKFKNKATLTVDKSSSTAYMQLSSLTSEDSAVYYCTTRFAYWGQGTLVIVSA

SEQ ID NO: 88 - protein, 114 aa, Mus musculus
EVQLQQSGPELVKPGASVKMSCKASGYTFADYYMKWVKQSHGKSLEWIGDINPNSGDTFYNHKFKGKATLTVDKSSSTAYMQLNSLTSEDSAVYYCARTYYDYWGQGTTLTVSS

SEQ ID NO: 89 - protein, 113 aa, Artificial Sequence
  Humanized Heavy chain variable region
QVQLVQSGAEVVKPGASVKLSCKASGYTFTSYYLYWVKKAPGQGLDWIGEIYPRSGGTNYAEKFQGRVTLTADTSTSTAYLELSSLTSEDTAVYYCTRSLLYWGQGTTLTVSS

SEQ ID NO: 90 - protein, 113 aa, Artificial Sequence
  Humanized Heavy chain variable region
QVQLVQSGAEVVKPGASVKLSCKASGYTFTSYYLYWVKKAPGQGLDWIGEIYPRSGGTNINEKFLSRVTLTADTSTSTAYLELSSLTSEDTAVYYCTRSLLYWGQGTTLTVSS

SEQ ID NO: 91 - protein, 113 aa, Artificial Sequence
  Humanized Heavy chain variable region
QVQLQQSGAEVVKPGASVKLSCKASGYTFTSYYLYWVKKAPGQGLDWIGEIYPRSGGTNINEKFLSRVTLTADTSTSTAYMDLSSLTSEDTAVYYCTRSLLYWGQGTTLTVSS

SEQ ID NO: 92 - protein, 113 aa, Artificial Sequence
  Humanized Heavy chain variable region
QVQLQQSGAEVVKPGASVKLSCKASGYTFTSYYLYWVKKAPGQGLDWIGEIYPRSGGTNINEKFLSRVTITADESTSTVYMQLSSLTSEDSAVYYCTRSLLYWGQGTTLTVSS

SEQ ID NO: 93 - protein, 443 aa, Artificial Sequence
  Humanized Heavy chain
QVQLVQSGAEVVKPGASVKLSCKASGYTFTSYYLYWVKKAPGQGLDWIGEIYPRSGGTNINEKFLSRVTLTADTSTSTAYLELSSLTSEDTAVYYCTRSLLYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 14 aa, Homo sapience
AGEGPKEGEAVVLP

SEQ ID NO: 95 - protein, 11 aa, Homo sapience
GPKEGEAVVLP

SEQ ID NO: 96 - protein, 11 aa, Homo sapience
  misc_feature (2)..(2): X represents Pro, Ala, Cys, Gly, Ser, Thr, or Val
  misc_feature (5)..(5): X represents Pro, Ala, Cys, Gly, Ser, Thr, or Val.
  misc_feature (8)..(8): X represents Pro, Ala, Cys, Gly, Ser, Thr, Val, Met, Leu, Ile or Phe.
GXKEXEAXVLP

SEQ ID NO: 97 - protein, 357 aa, Homo sapience
ISESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKSWISESVEADVRLRLANVSERDGGEYLCRATNFIGVAEKAFWLSVHGPRAAEEELVEADEAGSVYAG

SEQ ID NO: 98 - protein, 355 aa, Homo sapience
ISESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAG

SEQ ID NO: 99 - protein, 64 aa, Artificial Sequence
  Fynomer
  misc_feature (32)..(32): X represents any amino acid
  misc_feature (33)..(33): X represents any amino acid
  misc_feature (34)..(34): X represents any amino acid
  misc_feature (35)..(35): X represents any amino acid
  misc_feature (45)..(45): X represents any amino acid
  misc_feature (51)..(51): X represents any amino acid
GVTLFVALYDYEVYGPTPMLSFHKGEKFQILXXXXGPYWEARSLXTGETGXIPSNYVAPVDSIQ

SEQ ID NO: 100 - protein, 8 aa, Artificial Sequence
  RT-loop sequence
EVYGPTPM

SEQ ID NO: 101 - protein, 64 aa, Artificial Sequence
  Fynomer
GVTLFVALYDYEVYGPTPMLSFHKGEKFQILNSSEGPYWEARSLTTGETGLIPSNYVAPVDSIQ

SEQ ID NO: 102 - protein, 7 aa, Artificial Sequence
  conserved sequence
NSSEGPY

SEQ ID NO: 103 - protein, 64 aa, Artificial Sequence
  Fynomer
GVTLFVALYDYEVYGPTPMLSFHKGEKFQILRGGQGPYWEARSLTTGETGLIPSNYVAPVDSIQ

SEQ ID NO: 104 - protein, 7 aa, Artificial Sequence
  conserved sequence
RGGQGPY

SEQ ID NO: 105 - protein, 64 aa, Artificial Sequence
  Fynomer
GVTLFVALYDYEVYGPTPMLSFHKGEKFQILRGGDGPYWEARSLTTGETGLIPSNYVAPVDSIQ

SEQ ID NO: 106 - protein, 7 aa, Artificial Sequence
  conserved sequence
RGGDGPY

SEQ ID NO: 107 - protein, 64 aa, Artificial Sequence
  Fynomer
GVTLFVALYDYEVYGPTPMLSFHKGEKFQILKGGSGPYWEARSLTTGETGLIPSNYVAPVDSIQ

SEQ ID NO: 108 - protein, 7 aa, Artificial Sequence
  conserved sequence
KGGSGPY

SEQ ID NO: 109 - protein, 64 aa, Artificial Sequence
  Fynomer
GVTLFVALYDYEVYGPTPMLSFHKGEKFQILRKGKGPYWEARSLATGETGLIPSNYVAPVDSIQ

SEQ ID NO: 110 - protein, 7 aa, Artificial Sequence
  conserved sequence
RKGKGPY

SEQ ID NO: 111 - protein, 64 aa, Artificial Sequence
  Fynomer
GVTLFVALYDYEVYGPTPMLSFHKGEKFQILRRGSGPYWEARSLTTGETGLIPSNYVAPVDSIQ

SEQ ID NO: 112 - protein, 7 aa, Artificial Sequence
  conserved sequence
RRGSGPY

SEQ ID NO: 113 - protein, 64 aa, Artificial Sequence
  Fynomer
  misc_feature (51)..(51): X represents any amino acid.
GVTLFVALYDYEVMSTTALSFHKGEKFQILSQSPHGQYWEARSLTTGETGXIPSNYVAPVDSIQ

SEQ ID NO: 114 - protein, 7 aa, Artificial Sequence
  conserved sequence
EVMSTTA

SEQ ID NO: 115 - protein, 5 aa, Artificial Sequence
  SRC loop
SQSPH

SEQ ID NO: 116 - protein, 64 aa, Artificial Sequence
  Fynomer
GVTLFVALYDYEVMSTTALSFHKGEKFQILSQSPHGQYWEARSLTTGETGWIPSNYVAPVDSIQ

SEQ ID NO: 117 - protein, 8 aa, Artificial Sequence
  conserved sequence
SQSPHGQY

SEQ ID NO: 118 - protein, 19 aa, Homo sapience
AGEGPKEGEAVVLPEVEPG

SEQ ID NO: 119 - protein, 19 aa, Homo sapience
KEGEAVVLPEVEPGLTARE

SEQ ID NO: 120 - protein, 19 aa, Homo sapience
VVLPEVEPGLTAREQEATP

SEQ ID NO: 121 - protein, 17 aa, Homo sapience
PEPTGLEATTASTSTLP

SEQ ID NO: 122 - protein, 11 aa, Homo sapience
ETTQLPTTHQA

SEQ ID NO: 123 - protein, 24 aa, Homo sapience
ATTAQEPATSHPHRDMQPGHHETS

SEQ ID NO: 124 - protein, 21 aa, Artificial Sequence
  negative control peptide
QAAVTSHPHGGMQPGLHETSA

SEQ ID NO: 125 - protein, 541 aa, Artificial Sequence
  bi-specific binding agent hF6-HN-G7
  misc_feature (1)..(19): signaling sequence
  misc_feature (20)..(83): Fynomer portion
  misc_feature (84)..(98): linker
MEWSWVFLFFLSVTTGVHSGVTLFVALYDYEVYGPTPMLSFHKGEKFQILKGGSGPYWEARSLTTGETGLIPSNYVAPVDSIQGGGGSGGGGSGGGGSQVQLVQSGAEVVKPGASVKLSCKASGYTFTSYYLYWVKKAPGQGLDWIGEIYPRSGGTNINEKFLSRVTLTADTSTSTAYLELSSLTSEDTAVYYCTRSLLYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 311 aa, Mus musculus
MRRAALWLWLCALALRLQPALPQIVAVNVPPEDQDGSGDDSDNFSGSGTGALPDTLSRQTPSTWKDVWLLTATPTAPEPTSSNTETAFTSVLPAGEKPEEGEPVLHVEAEPGFTARDKEKEVTTRPRETVQLPITQRASTVRVTTAQAAVTSHPHGGMQPGLHETSAPTAPGQPDHQPPRVEGGGTSVIKEVVEDGTANQLPAGEGSGEQDFTFETSGENTAVAAVEPGLRNQPPVDEGATGASQSLLDRKEVLGGVIAGGLVGLIFAVCLVAFMLYRMKKKDEGSYSLEEPKQANGGAYQKPTKQEEFYA

SEQ ID NO: 127 - protein, 313 aa, Rattus norvegicus
MRRAALWLWLCALALRLQPALPQIVTANVPPEDQDGSGDDSDNFSGSGTGALPDMTLSRQTPSTWKDVWLLTATPTAPEPTSRDTEATLTSILPAGEKPEEGEPVAHVEAEPDFTARDKEKEATTRPRETTQLPVTQQASTAARATTAQASVTSHPHGDVQPGLHETLAPTAPGQPDHQPPSVEDGGTSVIKEVVEDETTNQLPAGEGSGEQDFTFETSGENTAVAGVEPDLRNQSPVDEGATGASQGLLDRKEVLGGVIAGGLVGLIFAVCLVAFMLYRMKKKDEGSYSLEEPKQANGGAYQKPTKQEEFYA

SEQ ID NO: 128 - protein, 338 aa, Macaca mulatta
MGATAYIPNSNSLSALLRGLELPHQTELLRVRALPTLLCPCALCRAPGCVQIVATNLPPEDQDGSGDDSDNFSGSGAGALQDITLSQQTPSTWKDTWLLTATPMSPEPTGLEATAASTSTLPAGEGPKEGEAVVLLEVEPDLTAREQEATPQPTETTQLPTTHQAPTARATTAQEPATSHPHRDMQPGHHETSAPAGPGQADLHTPRTEDGGPSATERAAEDGASSQLPAAEGSGEQDFTFETSGENTAIVAVEPDHRNQSPVDPGATGASQGLLDRKEVLGGIIAGGLVGLIFAVCLVGFMLYRMKKKDEGSYSLEEPKQANGGAYQKPTKQEEFYA

SEQ ID NO: 129 - protein, 310 aa, Canis lupus familiaris
MRRAALWLWLCALALRLQPALPQIVATNVPPEDQDGSGDDSDNFSGSGAGALQDITLSQQTPSTWKDMALLTAMPTAQEPTGADDIDSSTSILLTREGPEGGEAVLVAEAEPGFTDREKETAHPPSETTPHPTTHRASTARATTAQGPATLHPHRDAQPDHHQISVLAEPSQLDPHTPRVEDGGPSATERAAEDGVSTQLPAGEGSGEQDFTFDVSGENTAGTAVEPDQRNQPPVDRGATGASQGLLDRKEVLGGVIAGGLVGLIFAVCLVGFMLYRMKKKDEGSYSLEEPKQANGGAYQKPSKQEEFYA

SEQ ID NO: 130 - protein, 310 aa, Macaca fascicularis
MRRAALWLWLCALALSLQPAMPQIVATNLPPEDQDGSGDDSDNFSGSGAGALQDITLSQQTPSTWKDTWLVRATPMSPEPTGLEATAASTSTIQAGEGPKEGEAVVLLEVEPDLTAREQEATPQPTETTQLPTTHQAPTARATTAQEPATSHPHRDMQPGHHETSAPAGPGQADLHTPRTEDGGPSATERAAEDGASSQLPAAEGSGEQDFTFETSGENTAIVAVEPDHRNQSPVDPGATGASQGLLDRKEVLGGIIAGGLVGLIFAVCLVGFMLYRMKKKDEGSYSLEEPKQANGGAYQKPTKQEEFYA

SEQ ID NO: 131 - protein, 7 aa, Artificial Sequence
  RT loop sequence
EVYGPTP

SEQ ID NO: 132 - DNA, 1629 bp, Artificial Sequence
  Coding sequence for bi-specific binding agent hF6-HN-G7
  CDS (1)..(1629)
atggagtggtcctgggtgtttctgttcttcctcagcgtgaccacgggagtgcatagcggcgtgaccctgtttgtggccctgtacgactacgaggtgtacggccccacacccatgctgtccttccacaagggcgagaagttccagatcctgaagggcggctccggcccctactgggaggccagatccctgaccacaggcgagacaggcctgatcccctccaactacgtggcccccgtggactccattcagggcggcggaggatccggcggaggaggaagtggcggaggaggaagtcaagtccaactggtccaatcaggtgctgaggtcgtgaagccaggcgcgtcagttaagctctcctgcaaggcttccggatacaccttcacaagttattatctttactgggtcaaaaaagcacccggtcagggcctcgattggatcggtgaaatttacccccgctccggagggactaatattaacgaaaagttcctgagtcgagtgacacttactgctgatacttccacctccaccgcatacctcgagttgtcctccctcacatccgaggataccgccgtgtactactgcacaaggagcctgttgtattggggccaaggcactacactgacagtgtcttcagcttccaccaaaggtccatccgtgtttccactggcaccctcatcaaaaagcactagcggcggcaccgctgctctggggtgtctggtcaaggactattttcctgagcctgtgacagttagctggaacagcggcgcccttactagcggcgtccacacctttcccgccgtgcttcaatcctctggcctctactccctttcaagcgtggtcacagtccccagctcttcactcggtacccagacttatatatgtaatgttaatcacaagcctagcaacactaaggttgataaaaaggtggagcccaaaagctgtgacaagacgcatacatgccctccttgccccgcccccgagctcctgggcggcccttccgtctttctgttcccacccaaacccaaagataccctcatgatctccagaacgccagaagttacctgcgttgttgtagatgtgtctcacgaggaccccgaagtgaagtttaactggtatgtggatggagtggaggttcataacgccaagacaaaaccccgcgaagagcagtacaatagcacatatagggtcgtgagcgtcctcactgtcctccaccaggactggttgaacggtaaggaatataaatgtaaagtctccaacaaggctctgcccgcgccaattgaaaaaacaatctcaaaggcaaagggccagccacgggaacctcaggtctacacactgccacccagccgagaggagatgactaagaatcaggtctctctgacatgtctggtgaaggggttttatccatctgacattgccgttgaatgggaatcaaacgggcagcctgaaaataattacaagactactcctcccgtactggactccgacgggtcatttttcctctactctaaacttactgtcgataagtcaagatggcaacagggcaacgtcttcagttgcagcgtgatgcatgaagccctccataaccattatacgcagaaatctctcagtctgtctcccgggaagtaatga

SEQ ID NO: 133 - protein, 541 aa, Artificial Sequence; identical to SEQ ID NO: 125
  Synthetic Construct

SEQ ID NO: 134 - DNA, 1629 bp, Artificial Sequence
  Non coding sequence for bi-specific binding agent hF6-HN-G7
tacctcaccaggacccacaaagacaagaaggagtcgcactggtgccctcacgtatcgccgcactgggacaaacaccgggacatgctgatgctccacatgccggggtgtgggtacgacaggaaggtgttcccgctcttcaaggtctaggacttcccgccgaggccggggatgaccctccggtctagggactggtgtccgctctgtccggactaggggaggttgatgcaccgggggcacctgaggtaagtcccgccgcctcctaggccgcctcctccttcaccgcctcctccttcagttcaggttgaccaggttagtccacgactccagcacttcggtccgcgcagtcaattcgagaggacgttccgaaggcctatgtggaagtgttcaataatagaaatgacccagttttttcgtgggccagtcccggagctaacctagccactttaaatgggggcgaggcctccctgattataattgcttttcaaggactcagctcactgtgaatgacgactatgaaggtggaggtggcgtatggagctcaacaggagggagtgtaggctcctatggcggcacatgatgacgtgttcctcggacaacataaccccggttccgtgatgtgactgtcacagaagtcgaaggtggtttccaggtaggcacaaaggtgaccgtgggagtagtttttcgtgatcgccgccgtggcgacgagaccccacagaccagttcctgataaaaggactcggacactgtcaatcgaccttgtcgccgcgggaatgatcgccgcaggtgtggaaagggcggcacgaagttaggagaccggagatgagggaaagttcgcaccagtgtcaggggtcgagaagtgagccatgggtctgaatatatacattacaattagtgttcggatcgttgtgattccaactatttttccacctcgggttttcgacactgttctgcgtatgtacgggaggaacggggcgggggctcgaggacccgccgggaaggcagaaagacaagggtgggtttgggtttctatgggagtactagaggtcttgcggtcttcaatggacgcaacaacatctacacagagtgctcctggggcttcacttcaaattgaccatacacctacctcacctccaagtattgcggttctgttttggggcgcttctcgtcatgttatcgtgtatatcccagcactcgcaggagtgacaggaggtggtcctgaccaacttgccattccttatatttacatttcagaggttgttccgagacgggcgcggttaacttttttgttagagtttccgtttcccggtcggtgcccttggagtccagatgtgtgacggtgggtcggctctcctctactgattcttagtccagagagactgtacagaccacttccccaaaataggtagactgtaacggcaacttacccttagtttgcccgtcggacttttattaatgttctgatgaggagggcatgacctgaggctgcccagtaaaaaggagatgagatttgaatgacagctattcagttctaccgttgtcccgttgcagaagtcaacgtcgcactacgtacttcgggaggtattggtaatatgcgtctttagagagtcagacagagggcccttcattact

SEQ ID NO: 135 - protein, 541 aa, Artificial Sequence
  bi-specific binding agent hF6-A118C-HN-G7
MEWSWVFLFFLSVTTGVHSGVTLFVALYDYEVYGPTPMLSFHKGEKFQILKGGSGPYWEARSLTTGETGLIPSNYVAPVDSIQGGGGSGGGGSGGGGSQVQLVQSGAEVVKPGASVKLSCKASGYTFTSYYLYWVKKAPGQGLDWIGEIYPRSGGTNINEKFLSRVTLTADTSTSTAYLELSSLTSEDTAVYYCTRSLLYWGQGTTLTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 541 aa, Artificial Sequence
  bispecific binding agent hF6-S119C-HN-G7
MEWSWVFLFFLSVTTGVHSGVTLFVALYDYEVYGPTPMLSFHKGEKFQILKGGSGPYWEARSLTTGETGLIPSNYVAPVDSIQGGGGSGGGGSGGGGSQVQLVQSGAEVVKPGASVKLSCKASGYTFTSYYLYWVKKAPGQGLDWIGEIYPRSGGTNINEKFLSRVTLTADTSTSTAYLELSSLTSEDTAVYYCTRSLLYWGQGTTLTVSSACTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 541 aa, Artificial Sequence
  bispecific binding agent hF6-S239C-HN-G7
MEWSWVFLFFLSVTTGVHSGVTLFVALYDYEVYGPTPMLSFHKGEKFQILKGGSGPYWEARSLTTGETGLIPSNYVAPVDSIQGGGGSGGGGSGGGGSQVQLVQSGAEVVKPGASVKLSCKASGYTFTSYYLYWVKKAPGQGLDWIGEIYPRSGGTNINEKFLSRVTLTADTSTSTAYLELSSLTSEDTAVYYCTRSLLYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 541 aa, Artificial Sequence
  bispecific binding agent hF6-V282C-HN-G7
MEWSWVFLFFLSVTTGVHSGVTLFVALYDYEVYGPTPMLSFHKGEKFQILKGGSGPYWEARSLTTGETGLIPSNYVAPVDSIQGGGGSGGGGSGGGGSQVQLVQSGAEVVKPGASVKLSCKASGYTFTSYYLYWVKKAPGQGLDWIGEIYPRSGGTNINEKFLSRVTLTADTSTSTAYLELSSLTSEDTAVYYCTRSLLYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGCEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 541 aa, Artificial Sequence
  bispecific binding agent hF6-T289C-G7
MEWSWVFLFFLSVTTGVHSGVTLFVALYDYEVYGPTPMLSFHKGEKFQILKGGSGPYWEARSLTTGETGLIPSNYVAPVDSIQGGGGSGGGGSGGGGSQVQLVQSGAEVVKPGASVKLSCKASGYTFTSYYLYWVKKAPGQGLDWIGEIYPRSGGTNINEKFLSRVTLTADTSTSTAYLELSSLTSEDTAVYYCTRSLLYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKCKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 541 aa, Artificial Sequence
  bispecific agent hF6-N361C-HN-G7
MEWSWVFLFFLSVTTGVHSGVTLFVALYDYEVYGPTPMLSFHKGEKFQILKGGSGPYWEARSLTTGETGLIPSNYVAPVDSIQGGGGSGGGGSGGGGSQVQLVQSGAEVVKPGASVKLSCKASGYTFTSYYLYWVKKAPGQGLDWIGEIYPRSGGTNINEKFLSRVTLTADTSTSTAYLELSSLTSEDTAVYYCTRSLLYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKCQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 541 aa, Artificial Sequence
  bispecific binding agent hF6-V422C-HN-G7
MEWSWVFLFFLSVTTGVHSGVTLFVALYDYEVYGPTPMLSFHKGEKFQILKGGSGPYWEARSLTTGETGLIPSNYVAPVDSIQGGGGSGGGGSGGGGSQVQLVQSGAEVVKPGASVKLSCKASGYTFTSYYLYWVKKAPGQGLDWIGEIYPRSGGTNINEKFLSRVTLTADTSTSTAYLELSSLTSEDTAVYYCTRSLLYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNCFSCSVMHEALHNHYTQKSLSLSPGK

(19) United States
(12) Patent Application Publication
(10) Pub. No.: US 2022/0249679 A1
(43) Pub. Date: Aug. 11, 2022

(54) BI-SPECIFIC BINDING AGENTS TARGETING SYNDECAN-1 AND FIBROBLAST GROWTH FACTOR RECEPTOR

(71) Applicant: Mitsubishi Tanabe Pharma Corporation, Osaka-Shi, Osaka (JP)

(72) Inventors: Julia CORONELLA, Carlsbad, CA (US); Robyn RICHARDSON, San Diego, CA (US); Anjuli TIMMER, San Diego, CA (US); Roland NEWMAN, San Diego, CA (US); Marco GYMNOPOULOS, San Diego, CA (US)

(21) Appl. No.: 17/282,368

(22) PCT Filed: Oct. 1, 2019
(86) PCT No.: PCT/JP2019/038750

§ 371 Date: Apr. 1, 2021

Related U.S. Application Data
(60) Provisional application No. 62/740,337, filed on Oct. 2, 2018.

(51) Int. Cl. A61K 47/68; C07K 16/28

(52) U.S. Cl. CPC A61K 47/6803; A61K 47/6849; A61K 47/6889; C07K 16/2863; C07K 2317/24; C07K 2317/92; C07K 2317/33; C07K 2317/55; C07K 2317/565; C07K 2317/77; C07K 2317/31

(57) ABSTRACT

Presented herein, in certain embodiments, are bi-specific binding agents comprising an antibody portion that binds specifically to syndecan-1 and a Fynomer portion that binds specifically to a Fibroblast Growth Factor Receptor 3 (FGFR3), compositions thereof and uses thereof for treating a neoplasm.

### RELATED APPLICATIONS

This patent application is a national phase filing of, and claims the benefit of, International Patent Application No. based on PCT/JP2019/038750 filed on Oct. 1, 2019, entitled BI-SPECIFIC BINDING AGENTS TARGETING SYNDECAN-1 AND FIBROBLAST GROWTH FACTOR RECEPTOR, and naming Julia CORONELLA, Robyn RICHARDSON, Anjuli TIMMER, Roland NEWMAN and Marco GYMNOPOULOS as an inventors, which claims the benefit of U.S. Provisional Patent Application No. 62/740,337 filed on Oct. 2, 2018, entitled BI-SPECIFIC BINDING AGENTS TARGETING SYNDECAN-1 AND FIBROBLAST GROWTH FACTOR RECEPTOR, naming Julia CORONELLA, Robyn RICHARDSON, Anjuli TIMMER, Roland NEWMAN and Marco GYMNOPOULOS as inventors, and designated by attorney docket no. 057774-0459481. The entire content of the foregoing patent application is incorporated herein by reference, including all text, tables and drawings.

### SEQUENCE LISTING

The present application is being filed with a Sequence Listing. The Sequence Listing is submitted electronically in ASCII format via EFS-Web in the form of a text file. Said ASCII copy, created on Mar. 31, 2021, is named 057774-0559395-US.txt and is 125 KB in size, the contents of which are incorporated herein by reference in their entirety.

### FIELD OF THE INVENTION

Embodiments of the invention relate to a bi-specific binding agent comprising an antibody portion that binds specifically to CD138 (Syndecan-1), and a Fynomer portion that binds specifically to a Fibroblast Growth Factor Receptor 3 (FGFR3), as well as drug conjugates thereof, compositions thereof and uses thereof. The bi-specific binding agents disclosed herein can be used alone or in combination with other agents to effectively treat a neoplasm.

### BACKGROUND

The syndecan family includes four transmembrane heparan sulfate proteoglycans (HSPGs) mainly present on the cell surface. The structures of these different syndecans show high homology in vertebrates and invertebrates. All four syndecans are built up of a core protein decorated with varying number of glycosaminoglycan (GAG) side chains. Syndecans exert their function mainly through these GAG chains, but the different domains of the core protein have distinct roles as well. Syndecan-1 and syndecan-3 carry both heparan sulfate (HS) and chondroitin sulfate (CS) chains, whereas syndecan-2 and syndecan-4 carry only HS chains. Syndecans are involved in a wide range of biological processes including growth and differentiation, cell spreading, cell adhesion, cell migration, cytoskeletal organization, infiltration, and angiogenesis.

Syndecan-1 is a transmembrane (Type 1) heparan sulfate proteoglycan comprising an N-terminal extracellular domain, a transmembrane domain and a C-terminal intracellular signaling domain. In humans syndecan-1 (CD138) comprises a core protein of 310 amino acids in length and is encoded by the SDC1 gene. The SDC1 gene consists of five exons and is located on human chromosome 2. The first exon encodes a signal peptide, the second exon encodes attachment sites for heparan sulfate, the third and fourth exons encode a site for chondroitin sulfate binding and the fifth exon encodes the transmembrane and cytoplasmic domains.

Syndecan-1 is expressed on the basolateral surface of epithelial cells in adult tissues, on mesenchymal cells during development, and on lymphoid cells during distinct stages of differentiation. Syndecan-1 can bind hepatocyte growth factor (HGF), can interact with various growth factors and act as a coreceptor resulting in the activation of multiple signaling pathways effecting cell migration, cell-matrix interactions, growth, proliferation and survival. Several studies have implicated syndecan-1, and/or its dysfunctional signaling activity or over-expression in the pathogenesis of various neoplasms.

Fibroblast Growth Factor Receptors (FGFRs) are a family of highly conserved transmembrane tyrosine kinase receptors that are involved in various intracellular signaling pathways. FGFRs are composed of an extracellular ligand binding region comprising two to three immunoglobulin-like domains (D1, D2 and D3), a single-pass transmembrane region, and a cytoplasmic region having tyrosine kinase activity. There are 4 major FGF receptors (FGFR1-4), having multiple splice variants, most of which occur in exon III of the receptors (corresponding to domain D3) (e.g., see Holzmann et al. (2012) J. of Nucleic Acids 2012:950508). The D3 domain contains two parts encoded by 3 exons: (IIIa, IIIb, and IIIc) and gene splicing events lead to a D3 domain transcribed from the invariant IIIa portion of the gene combined with either the IIIb or IIIc portions. These splice variations generate seven highly homologous human FGFRs: FGFR1b, FGFR1c, FGFR2b, FGFR2c, FGFR3b, FGFR3c, and FGFR4, which have distinct tissue distribution and ligand specificity.

In humans, FGFRs can be activated by overexpression, or by binding one or more of the 22 known fibroblast growth factor (FGF) ligands. FGFR3 activation plays a critical role in embryogenesis, development, cell proliferation, cell survival, migration, differentiation and growth arrest. Activation of FGFR3 can lead to activation of several key pathways implicated in oncogenic signaling, including the mitogen-activated protein kinase (MAPK) and PI3K-AKT pathways.

Presented herein are bi-specific binding agents comprising an antibody portion that binds to syndecan-1 and a Fynomer portion that binds to an FGFR3, and uses thereof for the treatment of a neoplasm.

### SUMMARY OF THE INVENTION

In some aspects, presented herein is a bi-specific binding agent comprising an antibody portion (e.g., an antibody or antigen binding portion thereof) and one or more Fynomers, where the antibody portion binds specifically to syndecan-1 (CD138) and the one or more Fynomers bind specifically to a fibroblast growth factor receptor 3 (FGFR3). Accordingly, in certain embodiments, presented herein is a bi-specific binding agent that binds specifically to syndecan-1 and binds specifically to FGFR3, or one or more isoforms of FGFR3. In some embodiments, a Fynomer portion of a bi-specific binding agent binds specifically to FGFR3b and/or FGFR3c. In certain aspects, a bi-specific binding agent comprising an antibody portion (e.g., an antibody or antigen binding portion thereof) that binds specifically to syndecan-1 (CD138), a Fynomer that bind specifically to a fibroblast growth factor receptor 3 (FGFR3) and an antineoplastic agent or toxin.

In some embodiments, an antibody portion of a bi-specific binding agent comprises one or more complementarity determining regions (CDRs) selected from (i) a CDR-L1 (light chain CDR1) comprising an amino acid sequence having at least 90% identity to an amino acid sequence selected from SEQ ID NOs:2-15, (ii) a CDR-L2 (light chain CDR2) comprising an amino acid sequence having at least 90% identity to an amino acid sequence selected from SEQ ID NOs:16-26, (iii) a CDR-L3 (light chain CDR3) comprising an amino acid sequence having at least 90% identity to an amino acid sequence selected from SEQ ID NOs:27-33, (iv) a CDR-H1 (heavy chain CDR1) comprising an amino acid sequence having at least 90% identity to an amino acid sequence selected from SEQ ID NOs:45-59, (v) a CDR-H2 (heavy chain CDR2) comprising an amino acid sequence having at least 90% identity to an amino acid sequence selected from SEQ ID NOs:60-71, and (vi) a CDR-H3 (heavy chain CDR3) comprising an amino acid sequence having at least 90% identity to an amino acid sequence selected from SEQ ID NOs:72-81, wherein the antibody, or binding portion thereof, binds specifically to human syndecan-1. An antibody portion of a bi-specific binding agent may comprise any suitable CDR-L1 selected from Table 1, any suitable CDR-L2 selected from Table 2, any suitable CDR-L3 selected from Table 3, any suitable CDR-H1 selected from Table 6, any suitable CDR-H2 selected from Table 7 and any suitable CDR-H3 selected from Table 8, wherein the antibody, or binding portion thereof, binds specifically to human syndecan-1. In some embodiments, an antibody portion of a bi-specific binding agent comprises a CDR-L1 comprising the amino acid sequence of SEQ ID NO:2, a CDR-L2 comprising the amino acid sequence of SEQ ID NO:18, a CDR-L3 comprising the amino acid sequence of SEQ ID NO:28, a CDR-H1 comprising the amino acid sequence of SEQ ID NO:47, a CDR-H2 comprising the amino acid sequence of SEQ ID NO:60, and a CDR-H3 comprising the amino acid sequence of SEQ ID NO:73. In some embodiments, an antibody, or binding portion thereof, comprises a CDR-L1, CDR-L2, CDR-L3, CDR-H1, CDR-H2 and CDR-H3 comprising the amino acid sequences of SEQ ID NOs:2, 17, 27, 47, 61 and 73, respectively. In some embodiments, an antibody, or binding portion thereof, comprises a CDR-L1, CDR-L2, CDR-L3, CDR-H1, CDR-H2 and CDR-H3 comprising the amino acid sequences of SEQ ID NOs:2, 16, 27, 45, 60 and 72, respectively. In some embodiments, an antibody, or binding portion thereof, comprises a CDR-L1, CDR-L2, CDR-L3, CDR-H1, CDR-H2 and CDR-H3 comprising the amino acid sequences of SEQ ID NOs:2, 16, 27, 45, 61 and 72, respectively. In some embodiments, an antibody, or binding portion thereof, comprises a CDR-L1, CDR-L2, CDR-L3, CDR-H1, CDR-H2 and CDR-H3 comprising the amino acid sequences of SEQ ID NOs:2, 17, 27, 47, 61 and 73, respectively. In some embodiments, an antibody, or binding portion thereof, comprises a CDR-L1, CDR-L2, CDR-L3, CDR-H1, CDR-H2 and CDR-H3 comprising the amino acid sequences of SEQ ID NOs:5, 21, 30, 50, 64 and 75 respectively. In some embodiments, an antibody, or binding portion thereof, comprises a CDR-L1, CDR-L2, CDR-L3, CDR-H1, CDR-H2 and CDR-H3 comprising the amino acid sequences of SEQ ID NOs:4, 20, 29, 50, 63 and 75, respectively. In some embodiments, an antibody, or binding portion thereof, comprises a CDR-L1, CDR-L2, CDR-L3, CDR-H1, CDR-H2 and CDR-H3 comprising the amino acid sequences of SEQ ID NOs:4, 19, 29, 48, 63 and 74, respectively. In some embodiments, an antibody portion of a bi-specific binding agent comprises one or more constant regions of a human antibody (e.g., an IgG). In some embodiments, an antibody portion of a bi-specific binding agent comprises a humanized monoclonal antibody, or humanized antigen binding portion thereof, where the variable region sequences are humanized.

In some embodiments, a Fynomer of a bi-specific binding agent comprises a polypeptide selected from a polypeptide having an amino acid sequence at least 80% identical to the amino acid sequence of (i)

(SEQ ID NO: 99)
GVTLFVALYDYEVYGPTPMLSFHKGEKFQIL(X₁)(X₂)(X₃)(X₄)GPYWEARSL(X₅)TGETG
(X6)IPSNYVAPVDSIQ, wherein the amino acids (X₁), (X₂), (X₃), (X₄), (X₅),
and (X6) are any amino acid,
(ii)
(SEQ ID NO: 113)
GVTLFVALYDYEVMSTTALSFHKGEKFQILSQSPHGQYWEARSLTTGETG(X₇)IPSNYVA
PVDSIQ, where (X₇) is any amino acid,
(iii)
(SEQ ID NO: 101; FF2L4C3)
GVTLFVALYDYEVYGPTPMLSFHKGEKFQILNSSEGPYWEARSLTTGETGLIPSNYVAPV
DSIQ,
(iv)
(SEQ ID NO: 103; FF44L65G12)
GVTLFVALYDYEVYGPTPMLSFHKGEKFQILRGGQGPYWEARSLTTGETGLIPSNYVAP
VDSIQ,
(v)
(SEQ ID NO: 105; FF44L65G7)
GVTLFVALYDYEVYGPTPMLSFHKGEKFQILRGGDGPYWEARSLTTGETGLIPSNYVAP
VDSIQ,
(vi)
(SEQ ID NO: 107; FF48L66G7; “G7”)
GVTLFVALYDYEVYGPTPMLSFHKGEKFQILKGGSGPYWEARSLTTGETGLIPSNYVAP
VDSIQ,
(vii)
(SEQ ID NO: 109; FF43L65D5)
GVTLFVALYDYEVYGPTPMLSFHKGEKFQILRKGKGPYWEARSLATGETGLIPSNYVAP
VDSIQ,
(viii)
(SEQ ID NO: 111; FF44L65B7)
GVTLFVALYDYEVYGPTPMLSFHKGEKFQILRRGSGPYWEARSLTTGETGLIPSNYVAP
VDSIQ,
and
(ix)
(SEQ ID NO: 116; FF40L54A5)
GVTLFVALYDYEVMSTTALSFHKGEKFQILSQSPHGQYWEARSLTTGETGWIPSNYVAP
VDSIQ.

In some embodiments, a Fynomer of a bi-specific binding agent comprises or consists of a polypeptide having an amino acid sequence at least 80%, at least 85%, at least 90%, at least 95% or 100% identical to the amino acid sequence of SEQ ID NO:107. In some embodiments, a Fynomer of a bi-specific binding agent comprises or consists of a polypeptide having the amino acid sequence of SEQ ID NO:107.

In some embodiments, a bi-specific agent comprises an anti-neoplastic agent selected from the group consisting of a dolastatin, an auristatin, a maytansine, a tubulysin, a calicheamicin, a pyrrolobenzodiazepine (PBD), a duocarmycin, a doxorubicin, a pseudomonas exotoxin-A (PE38), an irinotecan and derivatives thereof. In certain embodiments, a bi-specific binding agent comprises a pyrrolobenzodiazepine toxin comprising the structure of chemical formula I:

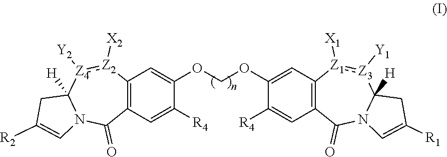

wherein
Z₁ and Z₂ are both N;
Z₃ and Z₄ are both C;

double-dash lines [symbol] represent a single bond or a double bond;

n is 1 to 10;

each of R₃ and R₄ are independently H, or a C₁₋₄ alkoxyl; and
each of R₁ and R₂ are independently selected from the group consisting of H, C₁₋₅ alkyl, C₃₋₆ cycloalkyl, C₂₋₅ alkenyl, and a phenyl optionally substituted with R₅,
wherein
   R₅ is selected from the group consisting of —NH₂, —NHR₆, and a piperazinyl substituted with R₇ having the structure

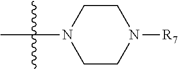

wherein R₆ comprises the linking group, and
R₇ is H, or a C₁₋₅ alkyl;
X₁ is null, a protecting group, or comprises the linking group;
X₂ is null, a protecting group, or comprises the linking group;

only one of X₁, X₂, R₁, and R₂ comprises the linking group; and

each of Y₁ and Y₂ are independently either null, OH, or SO₃H;
provided that:
(i) when X₁ comprises the linking group, Z₁ [symbol] Z₃ is N—C,
(ii) when X₂ comprises the linking group, Z₂ [symbol] Z₄ is N—C,
(iii) when X₁ comprises the protecting group, Z₁ [symbol] Z₃ is N—C, and
(iv) when X₂ comprises the protecting group, Z₂ [symbol] Z₄ is N—C
   Wherein null means the absence of the moiety or the presence of one or more hydrogens to complete a required valence.

In certain embodiments, an anti-neoplastic agent comprises the pyrrolobenzodiazepine toxin of formula I and a linking group, where the pyrrolobenzodiazepine toxin is attached to the linking group, and the linking group is attached to a bi-specific agent described herein. In certain embodiments, the linking group comprises a structure selected from the structure of chemical formula (A):

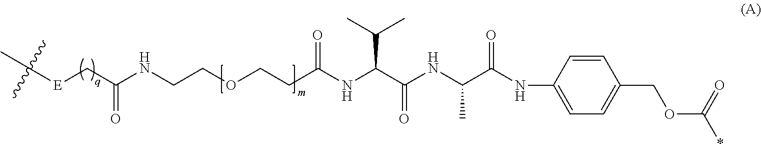

wherein
the asterisk indicates the point of attachment to a pyrrolobenzodiazepine toxin;
the wavy line indicates the point of attachment to the binding agent;
m is 1 to 20;
q is 1 to 10; and
E is a connecting group, and the structure of chemical formula (B):

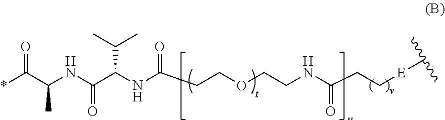

wherein
the asterisk indicates the point of attachment to the pyrrolobenzodiazepine toxin;
the wavy line indicates the point of attachment to the binding agent;
E comprises a connecting group;
v is 0 to 10; and
u is 0 or 1; wherein when u is 1, t is 1 to 10.

In some embodiments, an anti-neoplastic agent comprises a structure selected from the group consisting of formula II below;

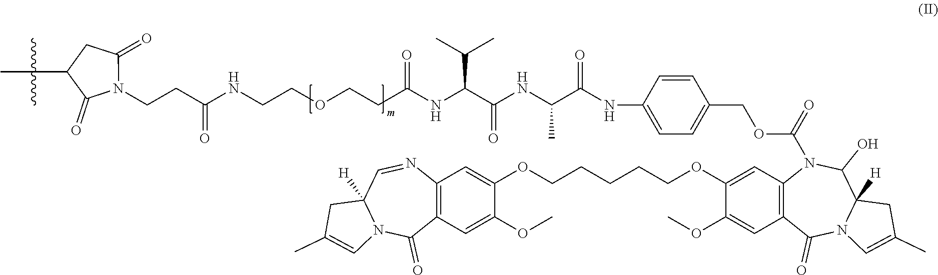

wherein m is 8; formula III below:

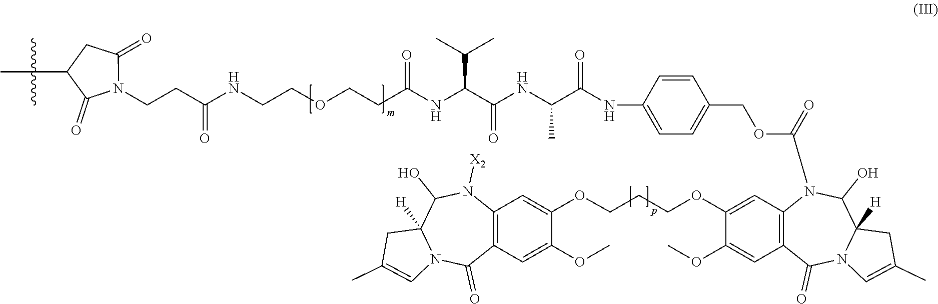

wherein m is 8, p is 3, and X₂ is a protecting group; formula V below:

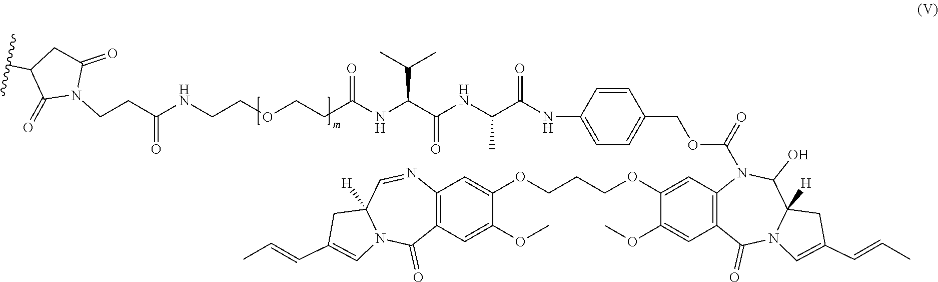

wherein m is 8, formula VI below:

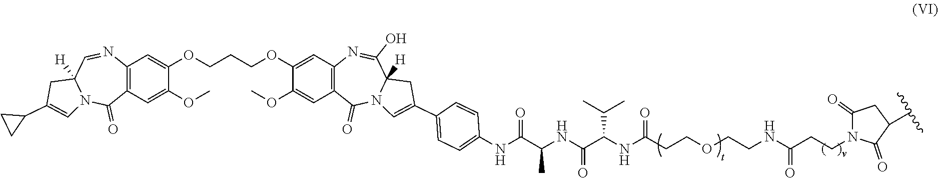

wherein t is 8, and v is 1; and formula VII below:

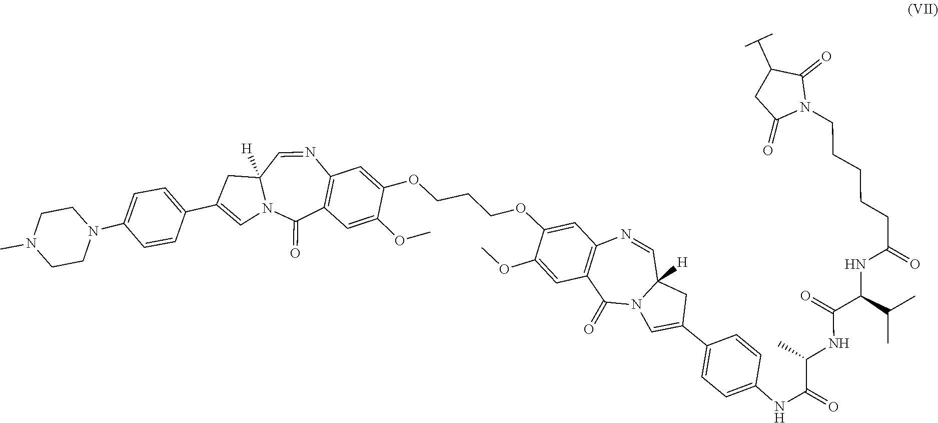

wherein the wavy line indicates the point of attachment to the binding agent.

In some embodiments, presented herein is a pharmaceutical composition comprising a bi-specific binding agent described herein and a pharmaceutically acceptable excipient, diluent, additive or carrier.

In some aspects, presented herein is a method of treating a subject having, or suspected of having, a neoplasm comprising administering to the subject a therapeutically effective amount of the bi-specific binding agent or pharmaceutical composition described herein. In certain embodiments, the neoplasm is selected from a carcinoma, sarcoma, neuroblastoma, glioblastoma, myeloma, lymphoma, melanoma or a solid or soft tissue tumor, where the neoplasm expresses syndecan-1 and/or an FGFR3. In some embodiments, a subject having, or suspected of having a neoplasm, is a human having or harboring a neoplastic cell that expresses human syndecan-1 and/or human FGFR3 on the cell surface of the neoplastic cell.

Certain aspects of the technology are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 shows an alignment of syndecan-1 proteins derived from human, cynomolgus monkey (cyno) and mouse. Peptides used for immunization were derived from the boxed areas.

FIG. 2 shows Fluorescence-activated cell sorting (FACS) histograms for F12P16F6 (FIG. 2A) and another representative positive hybridoma clone F12P16G3 (FIG. 2B) binding to CD138 on H929 cells.

FIG. 3 shows the results of kinetic binding analysis (i.e., a SPR sensorgram) for a representative hybridoma F12P16F6 (indicated in the figure as “12P16F6”) for binding to human CD138 at 167 nM (L1A1) to 10.4 nM (L1A4).

FIG. 4 shows binding of representative chimeric antibodies 12P16F6 hIgG1 (“chF6”, FIG. 4A) and 13P30A7 hIgG1 (“chP30A7”, FIG. 4B) to human CD138 expressing cells (human) and cynomolgus CD138 expressing cells (cyno). Control antibody (“Sec”, i.e., Secukinumab) showed little or no specific binding to CD138. Y-axis represents the concentration of the antibodies chF6, 13P30A7 or Secukinumab

FIG. 5A shows the humanized heavy chains compared to those of the parent mouse F12P16F6 (“P16F6 VL”). The designation cdr indicates the CDR grafting approach, the designation abb indicates the Grafting of abbreviated CDRs approach, the designation sdr indicates the SDR-transfer approach, the designation fra indicates The Frankenstein approach, and the designation ven indicates the Veneering approach. The designation repair indicates that the variable regions were subjected to a second round of humanization. FIG. 5B shows the humanized light chains compared to those of the parent mouse F12P16F6 (“P16F6 VL”).

FIG. 6 shows a picture of an SDS-PAGE gel ran under reducing conditions illustrating the molecular weight (kilodaltons, kDa) and purity of 11 representative humanized antibodies. Lane 1=12P16F6 hIgG1 (chF6), Lane 2=F6 aka-rep, Lane 3=F6 aks-rep, Lane 4=F6 akf-rep, Lane 5=F6 cka-rep, Lane 6=F6 ckf-rep (hF6), Lane 7=F6 f2ka-rep, Lane 8=F6 f2ks-rep, Lane 9=F6 f2kf-rep, Lane 10=F6 f1ka-rep, Lane 11=F6 f1ks-rep, Lane 12=F6 f1kf-rep, and MW=molecular weight marker. Molecular weight markers are labeled to the right of the gel.

FIG. 7 shows FACS analysis of cell-surface binding of eleven representative humanized antibodies to human CD138 on the surface of multiple myeloma cell line KMS-11 (FIG. 7B) and bladder cancer line RT112/84 (FIG. 7A). Secukinumab was used as a negative control.

FIG. 8 shows an illustration of an X-ray crystal structure derived from a human syndecan-1 peptide in complex with an antibody Fab fragment that was solved at 1.95 Å resolution. There is one copy each of the syndecan-1 peptide and Fab per asymmetric unit. FIG. 8 illustrates the syndecan-1-Fab binding interface. The Fab Heavy chain is shown in the form of ribbon side chain carbon atoms to the left of the figure. The Fab light chain is shown in the form of ribbon side chain carbon atoms to the right of the figure. The syndecan-1 peptide carbon atoms are shown sandwiched between the Fab heavy and light chains. Certain amino acids of the syndecan-1 peptide and certain side chains of the Fab fragment are labeled with their corresponding 3-letter amino acid abbreviation and positions.

FIG. 9 shows an alignment of the anti-FGFR3 Fynomer of SEQ ID NOs:101, 109, 103, 105, 107 and 111.

FIG. 10 shows internalization properties of anti-FGFR3 Fynomers designated as FF2L4C3 (SEQ ID NO:101); FF2L4D4; FF3L6G2; FF5L7D3; FF5L7D4; FF15L31B1 and FynSH3 (negative control).

FIG. 11 shows a FACS binding profile of Fynomer polypeptides specifically binding to FGFR3 on the surface of FGFR3 positive KMS-11 cells. FIG. 11A shows specific binding of FF2L4C3 (SEQ ID NO:101); FF43L65D5 (SEQ ID NO:109); FF44L65B7 (SEQ ID NO:111); FF44L65G7 (SEQ ID NO:105); FF44L65G12 (SEQ ID NO:103); FF48L66G7 (SEQ ID NO:107) and an anti-FGFR3 monoclonal antibody (positive control) to FGFR3-positive KMS-11 cells, while FIG. 11B shows no binding of the indicated Fynomers to an FGFR3 negative control cell line N87.

FIG. 12A shows an ELISA of FGFR3-Fynomers FF2L4C3-SEQ ID NO:101 and FF43L65D5-SEQ ID NO:109 (FIG. 12B) binding to plated coated human FGFR3b (huFGFR3b), human FGFR3c (huFGFR3c), cynomolgus monkey FGFR3c (cyFGFR3c), murine FGFR3c (muFGFR3c), human FGFR3 D1 domain (huFGFR3-D1), human FGFR3c D2 domain (huFGFR3-D2), human FGFR3 D1 and D2 domain (huFGFR3-D1D2), a negative control polyclonal antibody (IgG) and uncoated plates (PBS), as indicated on the x-axis. FIG. 12C shows an ELISA of FF44L65B7-SEQ ID NO:111 (FIG. 12C) and FF44L65G7-SEQ ID NO:105 (FIG. 12D) binding to plated coated human FGFR3b (huFGFR3b), human FGFR3c (huFGFR3c), cynomolgus monkey FGFR3c (cyFGFR3c), murine FGFR3c (muFGFR3c), human FGFR3 D1 domain (huFGFR3-D1), human FGFR3c D2 domain (huFGFR3-D2), human FGFR3 D1 and D2 domain (huFGFR3-D1D2), a negative control polyclonal antibody (IgG) and uncoated plates (PBS), as indicated on the x-axis. FIG. 12E shows an ELISA of FF44L65G12-SEQ ID NO:103 (FIG. 12E), and FF48L66G7-SEQ ID NO:107 (FIG. 12F) binding to plated coated human FGFR3b (huFGFR3b), human FGFR3c (huFGFR3c), cynomolgus monkey FGFR3c (cyFGFR3c), murine FGFR3c (muFGFR3c), human FGFR3 D1 domain (huFGFR3-D1), human FGFR3c D2 domain (huFGFR3-D2), human FGFR3 D1 and D2 domain (huFGFR3-D1D2), a negative control polyclonal antibody (IgG) and uncoated plates (PBS), as indicated on the x-axis.

FIG. 13 shows an internalization assay demonstrating the cytotoxic effect of toxin conjugated Fynomers FF2L4C3 (FIGS. 13A, B and C), FF43L65D5 (FIG. 13B), FF44L65G7 (FIG. 13B), FF44L65G12 (FIG. 13B), FF48L66G7 (FIG. 13B) and FF44L65B7 (FIG. 13C). The mouse monoclonal antibody 9E10, which binds a cytosolic myc protein, FynSH3, and no agent (cells only) were included as negative controls.

FIG. 14 shows an illustration of four embodiments of a bi-specific binding agent, each comprising an antibody portion having two Ig heavy chain and two Ig light chain polypeptides, and a Fynomer portion, where the Fynomer (represented as a sphere) is attached to different parts of the antibody. FIG. 14A shows a Fynomer attached to the C-terminal end of the heavy chain of the antibody. FIG. 14B shows a Fynomer attached to the N-terminal end of the heavy chain of the antibody. FIG. 14C shows a Fynomer attached to the C-terminal end of the light chain of the antibody. FIG. 14D shows a Fynomer attached to the N-terminal end of the light chain of the antibody.

FIG. 15 shows four representative bi-specific binding agents that were made and subjected to SDS PAGE under reducing conditions. Lane 7 was loaded with the full length anti-CD138 monoclonal antibody P16F6, which binds specifically to syndecan-1. Lane 7 provides a reference for the molecular weight of the heavy chain (upper band) and light chain (lower band) in the absence of an attached Fynomer. Lanes 2-6 show an embodiment of a bi-specific binding agent comprising the P16F6 antibody and the FGFR3-binding Fynomer G7 (SEQ ID NO:107) attached to the N-terminus of the heavy chain of the antibody (Lanes 2 and 6), attached to the C-terminus of the heavy chain of the antibody (Lane 4), attached to the N-terminus of the light chain of the antibody (Lane 5) and attached to the C-terminus of the light chain of the antibody (Lane 3). The increase in molecular weight of the heavy chains in lanes 2, 4 and 6 is evidence of the presence of the attached Fynomer (compare to heavy chain in control lane 7). The increase in molecular weight of the light chains in lanes 3 and 5 is evidence of the presence of the attached Fynomer (compare to light chain in control lane 7).

FIG. 16 shows a FACs histogram showing cell surface binding of the representative bi-specific binding agent “hF6-HN-G7” and humanized monoclonal antibody “hF6” to FGFR3c (FIG. 16A) and FGFR3b (FIG. 16B) expressed on the surface of transfected CHO cells. hF6 comprises the light chain of SEQ ID NO:44 and the heavy chain of SEQ ID NO:93. hF6-HN-G7 comprises the light chain of SEQ ID NO:44 and a fusion protein that includes the Fynomer of SEQ ID NO:107 (G7) and the heavy chain of SEQ ID NO:93, where the C-terminus of G7 is connected to the N-terminus of the heavy chain by a peptide bond.

FIG. 17 shows an in vitro cytotoxicity assay conducted on untransfected CHO cells (FIG. 17A), CHO cells expressing FGFR3b (FIG. 17B) and CHO cell expressing FGFR3c (FIG. 17C). Cell viability is shown on the y-axis and concentration of the binding agents is shown on the x-axis. The agents tested include Secukinumab (a monoclonal antibody that specifically binds to interleukin-17A) that was stochastically conjugated to the PBD toxin of formula II (Secukinumab-II), a Secukinumab fusion expressed with the G7 Fynomer at the heavy chain N-terminus and site specifically conjugated to the PBD toxin of formula II (Secukinumab-S119C-HN-G7-II), and the representative bi-specific binding agent hF6-S119C-HN-G7 that was site specifically conjugated to the PBD toxin of formula II (hF6-S119C-HN-G7-II). The hF6 antibody portion of the bi-specific binding agent hF6-S119C-HN-G7 comprises the light chain of SEQ ID NO:44 and the heavy chain of SEQ ID NO:93. Accordingly, hF6-S119C-HN-G7 is an hF6 antibody where the Fynomer of SEQ ID NO:107 (G7) and the heavy chain of SEQ ID NO:93 are expressed as a fusion protein where the C-terminus of G7 is connected to the N-terminus of the heavy chain by a peptide bond. S119C indicates that Serine at position 119 of the heavy chain constant region of hF6 was mutated to a Cysteine for covalent attachment of the PBD toxin (i.e., the anti-neoplastic agent). The representative PBD toxin used in this experiment was the toxin of formula II. The toxin was covalently attached to the sulfhydryl group of the cysteine at position 119 using maleimide chemistry.

FIG. 18 shows an exemplary anti-neoplastic agent of formula II.

FIG. 19 shows an in vitro cytotoxicity assay conducted on cell lines KMS-11 (FIG. 19A), OPM-2 (FIG. 19B) and ARH-77 (FIG. 19C) with a full length hF6 anti-CD138 monoclonal antibody conjugated stochastically to the anti-neoplastic agent of formula II (“hF6-II”), hF6-HN-G7 conjugated stochastically to the anti-neoplastic agent of formula II (hF6-HN-G7-II), and hF6-LN-G7 conjugated stochastically to the anti-neoplastic agent of formula II (hF6-LN-G7-II).

FIG. 20A shows an in vitro cytotoxicity assay conducted on the cell lines KMS-11 (FIG. 20A) and RT-112 (FIG. 20B) with hF6-T289C-HN-G7 site-specifically conjugated to the anti-neoplastic agent of formula II (hF6-T289C-HN-G7-II), hF6-S119C-HN-G7 site-specifically conjugated to the anti-neoplastic agent of formula II (hF6-S119C-HN-G7-II), hF6-V282C-HN-G7 site-specifically conjugated to the anti-neoplastic agent of formula II (hF6-V282C-HN-G7-II, FIGS. 20C and 20D only), hF6-II (FIGS. 20A and 20B only) and control antibody Secukinumab that was conjugated to the PBD toxin of formula II (Secukinumab-II). FIG. 20C shows an in vitro cytotoxicity assay conducted on the cell lines AN3CA (FIG. 20C) and HCC1806 (FIG. 20D) with hF6-T289C-HN-G7 site-specifically conjugated to the anti-neoplastic agent of formula II (hF6-T289C-HN-G7-II), hF6-S119C-HN-G7 site-specifically conjugated to the anti-neoplastic agent of formula II (hF6-S119C-HN-G7-II), hF6-V282C-HN-G7 site-specifically conjugated to the anti-neoplastic agent of formula II (hF6-V282C-HN-G7-II, FIGS. 20C and 20D only), hF6-II (FIGS. 20A and 20B only) and control antibody Secukinumab that was conjugated to the PBD toxin of formula II (Secukinumab-II).

FIG. 21A shows the graphical results of an in vivo xenograft study testing the ability of the indicated bi-specific binding agents to kill or inhibit the growth of AN3CA cells (FIG. 21A) implanted in a mouse. FIG. 21B shows the graphical results of an in vivo xenograft study testing the ability of the indicated bi-specific binding agents to kill or inhibit the growth of HCC1807 cells implanted in a mouse.

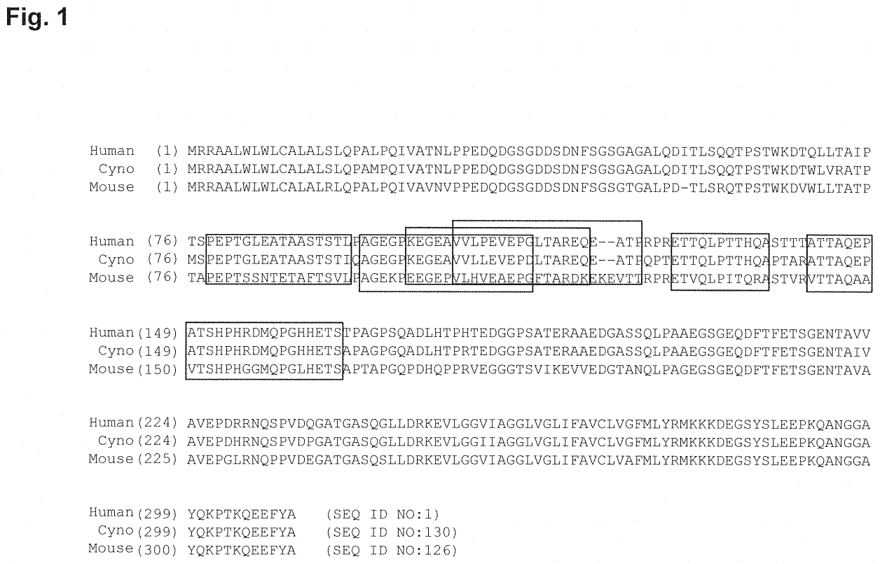

### DETAILED DESCRIPTION

Many neoplastic cells express a Fibroblast Growth Factor Receptor 3 (FGFR3, including one or more isoforms thereof) and syndecan-1 (CD138) on their cells surface. Presented herein are bi-specific binding agents that bind to syndecan-1 and an FGFR3 with high affinity. It was determined that these bi-specific binding agents can bind with high affinity and high selectivity to neoplastic cells that express both of the target receptors. In some embodiments, the bi-specific binding agents described herein induce internalization of the binding agent upon binding to one or both of the target receptors (i.e., CD138 and FGFR3). Further, by conjugating the bi-specific binding agents to a toxic payload, the bi-specific binding agents can specifically kill neoplastic cells that express one or both of the target receptors. Also, off-target cytotoxicity can be significantly reduced by incorporating a protease cleavable linking group between the toxic payload and the bi-specific binding agent, such that the toxin is release from the binding agent only after internalization and cleavage of the linking group by a cytosolic protease. The bi-specific binding agents herein represent a next generation of biologics that offer more efficient and selective killing of neoplastic cells while reducing adverse events.

In some embodiments, a bi-specific binding agent presented herein comprises an antibody portion (e.g., an antibody, or antigen binding portion thereof) and a Fynomer portion (i.e., a Fynomer), where the Fynomer is attached to the antibody portion. In some embodiments, the Fynomer is attached to the antibody portion by a covalent bond. In some embodiments, the antibody portion comprises an antibody, or antigen binding portion thereof, that binds specifically to syndecan-1 (i.e., a syndecan-1 polypeptide; e.g., CD138), or an extracellular portion thereof. In certain embodiments, the Fynomer portion comprises a Fynomer that binds specifically to an FGFR3 (e.g., FGFR3, or one or more isoforms thereof). In some embodiments, the bi-specific binding agent comprises a cytotoxic payload.

In some embodiments the bi-specific binding agents presented herein are used for the treatment, prevention and/or diagnosis of a neoplasm in a subject.

### Syndecans

Human syndecan-1 (e.g., SEQ ID NO:1) generally comprises an immature polypeptide sequence of 310 amino acids which includes an N-terminal single sequence from amino acids 1-22, an extracellular domain from about amino acid 23-254, a transmembrane domain from about amino acid 255 to 275 and a cytoplasmic domain from about amino acid 276 to 310, numbered from the N-terminus to the C-terminus. Methods of identifying leader sequences, extracellular domains, transmembrane domains, and cytoplasmic domains of a syndecan-1 receptor are known and any suitable method can be used to identify such domains or regions within a syndecan-1 polypeptide sequence derived from a suitable mammalian species.

In some embodiments syndecan-1 is a mammalian syndecan-1. A syndecan-1 may be derived from any mammalian species. In some embodiments, a syndecan-1 polypeptide is a human syndecan-1. In certain embodiments, an extracellular domain of syndecan-1 comprises an N-terminal portion of a syndecan-1 polypeptide that is typically expressed on the cell surface of an intact mammalian cell. In certain embodiments an extracellular domain of syndecan-1 is expressed in a soluble or a non-membrane bound form that lacks a cytoplasmic and/or transmembrane domain. In certain embodiments syndecan-1 and/or the extracellular domain of syndecan-1 comprises one or more amino acid additions, deletions or substitutions. A syndecan-1 polypeptide may comprise an amino acid sequence at least 80%, at least 85%, at least 90%, at least 95%, at least 98% or 100% identical to the syndecan-1 polypeptide of SEQ ID NO:1. In certain embodiments, a syndecan-1 polypeptide comprises a portion of (e.g., a sub-sequence of) a syndecan-1 protein. In some embodiments a portion of a syndecan-1 comprises an extracellular domain of syndecan-1, or a portion thereof.

### Antibodies & Ag Binding Portions Thereof

In some embodiments a bi-specific binding agent comprises an antibody, or a portion thereof (e.g., a binding portion thereof, or an antigen binding portion thereof) that binds specifically to syndecan-1 (CD138). An antibody, or a binding portion thereof, that binds specifically to syndecan-1 (CD138) is sometime referred to herein as an anti-CD138 antibody. Anti-CD138 antibodies and binding portions thereof that are contemplated for use in a bi-specific binding agent described herein are described in detail in International Patent Application No. PCT/JP2018/016847, which is incorporated herein by reference in its entirety.

In some embodiments an antibody is a monoclonal antibody, or binding portion thereof. Certain non-limiting examples of an antibody include monoclonal antibodies, chimeric antibodies, CDR-grafted antibodies, humanized antibodies, and human antibodies. Human antibodies can be obtained by a suitable method. For example, human antibodies can be obtained from trans-chromosomal animals engineered to produce fully human antibodies. In certain embodiments, an antibody is not polyclonal, and/or is not a polyclonal antibody.

An antibody, or binding portion thereof, can be generated, manufactured or produced by a suitable method. In some embodiments an antibody, or binding portion thereof, is derived, produced, obtained, isolated, and/or purified from a suitable species. In some embodiments an antibody, or binding portion thereof, is derived, produced, obtained, isolated, and/or purified from a rabbit, goat, horse, cow, rat, mouse, fish, bird, or llama, for example. In some embodiments an antibody is derived, produced, obtained, isolated, and/or purified from a bird (e.g., a chicken, or a bird egg). In some embodiments an antibody, or binding portion thereof, is derived, produced, obtained, isolated, and/or purified from a plant (e.g., a recombinant antibody, or binding portion thereof, produced by a genetically engineered plant). In some embodiments an antibody, or binding portion thereof, is derived, produced, obtained, isolated, and/or purified from a suitable mammal. In certain embodiments a suitable mammal is a genetically altered mammal (e.g., a trans-chromosomal or transgenic mammal) engineered to produce antibodies comprising human heavy chains and/or human light chains or portions thereof. In some embodiments an antibody, or binding portion thereof, is produced, obtained, isolated, or purified from a prokaryotic or eukaryotic cell (e.g., a recombinant antibody, or binding portion thereof, produced by a genetically engineered cell). In some embodiments an antibody, or binding portion thereof, is produced, obtained, isolated, or purified from a virus (e.g., a recombinant antibody, or binding portion thereof, produced by a genetically engineered virus).

An antibody, or binding portion thereof, or bi-specific agent can be expressed, isolated from and/or purified from a suitable expression system non-limiting examples of which include a suitable bacteria, phage, insect, virus, plant or mammalian expression system. For example, a nucleic acid encoding an antibody can be introduced into a suitable mammalian cell line that expresses and secretes the antibody into the cell culture media. Any suitable mammalian cell line can be used to generate an antibody or bi-specific binding agent. A method of producing an antibody or bi-specific binding agent, or a portion thereof, may comprise one or more of (i) introducing one or more nucleic acids into a suitable cell line wherein the nucleic acid directs the expression of the antibody, bi-specific binding agent or portion thereof; (ii) culturing the cell line using a suitable culturing method for a period of time that allows expression of the antibody or bi-specific binding agent; (iii) harvesting the cell line (e.g., by way of generating a lysate) or harvesting conditioned media produced from the cell line (e.g., where the antibody or bi-specific binding agent is secreted into the media); and (iv) isolating and/or purifying the antibody or bi-specific binding agent using a suitable method.

The modifier “monoclonal” is not to be construed as requiring production of an antibody, or binding portion thereof, by any particular method. A monoclonal antibody, or binding portion thereof, can be produced by any suitable method. For example, in certain embodiments, a monoclonal antibody is made by a hybridoma method (e.g., as described by Kohler et al., Nature, 256:495 (1975)), or a variation thereof. In some embodiments a monoclonal antibody, or binding portion thereof, is made by a recombinant DNA method. For example, a monoclonal antibody, or binding portion thereof, can be made by screening a recombinant library using a suitable expression system (e.g., a phage display expression system). In some embodiments a monoclonal antibody, or binding portion thereof, is isolated from a phage library, for example by using a technique described in Clackson et al., Nature, 352:624-628 (1991) and/or Marks et al., J.Mol.Biol., 222:581-597 (1991), or a variation thereof.

In certain embodiments, an antibody, or binding portion thereof, comprises one or more structural portions of a mammalian antibody. In certain embodiments an antibody, or binding portion thereof comprises one or more constant regions (e.g., constant regions derived from an antibody, e.g., a mammalian antibody). An antibody, or binding portion thereof, may comprise any suitable constant region of an antibody, or one or more portions thereof. In certain embodiments an antibody, or binding portion thereof, comprises a constant region of an antibody light chain and/or a constant region of an antibody heavy chain. In some embodiments an antibody, or binding portion thereof, comprises a lambda (k) light chain constant region, or a portion thereof. In some embodiments an antibody, or binding portion thereof, comprises a kappa (u) light chain constant region, or a portion thereof. In some embodiments an antibody, or binding portion thereof, comprises a polypeptide that is at least 75%, at least 80%, at least 85%, at least 90%, at least 95% or at least 99% identical to a polypeptide sequence of a light chain constant region of a mammalian antibody, or portion thereof. In some embodiments an antibody, or binding portion thereof, comprises a polypeptide that is at least 75%, at least 80%, at least 85%, at least 90%, at least 95% or at least 99% identical to a polypeptide sequence of a light chain constant region of a human antibody. In some embodiments a binding portion of an antibody does not include a light chain constant region. In some embodiments an antibody, or binding portion thereof, does not include one or more portions of a light chain constant region.

An antibody, or binding portion thereof, can include any suitable heavy chain constant region, or portion thereof. In mammals, an antibody can have at least five types/classes of Ig heavy chains denoted as IgA, IgD, IgE, IgG, and IgM, which are determined by the presence of distinct heavy chain constant regions, or portion thereof (e.g., CH1, CL, CH2, CH3 domains). In some embodiments an antibody, or binding portion thereof, comprises one or more heavy chain constant regions of an IgM, IgD, IgA, or IgE isotype, or a portion thereof. In some embodiments an antibody, or binding portion thereof, comprises a heavy chain constant region of an IgG₁, IgG₂, IgG₃ or IgG₄, or one or more portions thereof. In some embodiments an antibody, or binding portion thereof, comprises a polypeptide that is at least 75%, at least 80%, at least 85%, at least 90%, at least 95%, at least 99% identical, or 100% identical to a polypeptide sequence of a heavy chain constant region of a mammalian antibody. In some embodiments an antibody, or binding portion thereof, comprises a polypeptide that is at least 75%, at least 80%, at least 85%, at least 90%, at least 95%, at least 99% identical or 100% identical to a polypeptide sequence of a heavy chain constant region of a human antibody. In some embodiments an antibody, or binding portion thereof, comprises one or more additions, deletions and/or modification to a constant region. An antibody, or binding portion thereof, is sometimes modified to change the antibody class, or isotype of the antibody. In some embodiments an antibody, or binding portion thereof, comprises one or more additions, deletions and/or modification (one or more amino acid substitutions, deletions or additions) to modify one or more functions of the antibody, or binding portion thereof, for example to abolish, enhance or decrease serum half-life, Fc receptor binding, complement binding (e.g., C1q binding), glycosylation, sialylation, cellular toxicity, antibody-dependent cell-mediated phagocytosis (ADCP), antibody dependent cellular cytotoxicity (ADCC), and the like. In some embodiments a binding portion of an antibody does not include a heavy chain constant region. In some embodiments an antibody, or binding portion thereof, does not include one or more portions of a heavy chain constant region.

In some embodiments an antibody, or binding portion thereof, comprises or consists of one or more variable regions of an antibody, or a portion thereof. In some embodiments an antibody, or binding portion thereof, comprises one or more light chain variable regions, or a portion thereof. In some embodiments an antibody, or binding portion thereof, comprises one or more heavy chain variable regions, or a portion thereof. In certain embodiments an antibody, or binding portion thereof, comprises at least one light chain variable region and at least one heavy chain variable region. A light chain variable region and heavy chain variable region can be on the same or different polypeptides.

In mammals, the heavy chain variable region and light chain variable region of an antibody each contribute three CDRs (complementarity determining regions) commonly referred to as CDR1, CDR2 and CDR3, that are separated and/or flanked by framework regions (e.g., FR1, FR2, FR3 and FR4). The term “CDR” as used herein refers to an amino acid sequence of a polypeptide identified as a complementarity determining region. In certain embodiments, definitive delineation of a CDR polypeptide sequence and identification of residues comprising the binding site of antibody, or binding portion thereof, is accomplished by solving the structure of antibody, or binding portion thereof, and/or solving the structure of an antibody-antigen complex or the like. In certain embodiments, this is accomplished by any suitable method, such as X-ray crystallography and/or computer modeling. In certain embodiments, various methods of analysis are employed to identify or approximate the CDR sequences of an antibody or binding portion thereof. For example, the amino acid sequence and/or location of CDRs in a polypeptide sequence of an antibody, a binding portion thereof or variable region thereof, is identified using a suitable method, non-limiting examples of which include the Kabat or EU system (e.g., see Kabat, E. A., et al., 1991; Sequences of Proteins of Immunological Interest, Fifth Edition, NIH Publication No. 91-3242, as well as Johnson, G. and Wu, T. T. 2000, Nucleic Acids Research), and/or the Chothia Numbering Scheme (e.g., Chothia & Lesk, (1987) J.Mol.Biol., 196:901-917; Chothia et al., Nature, (1989) 342:878-883; and A1-Lazikani et al., (1997) JMB 273, 927-948). In some embodiments the amino sequence and/or location of CDRs of an antibody are identified using the AbM method and/or contact method. The “AbM” definition uses an integrated suite of computer programs produced by Oxford Molecular Group that model antibody structure (see e.g., Martin et al., Proc. Natl. Acad. Sci. (USA), 86:9268-9272 (1989); “AbM™, A Computer Program for Modeling Variable Regions of Antibodies,” Oxford, UK; Oxford Molecular, Ltd.). The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198 (1999). In certain embodiments, a contact definition is based on an analysis of the available complex crystal structures (see e.g., MacCallum et al., J.Mol.Biol, 5:732-45 (1996)).

In some embodiments an antibody comprises at least 6 distinct CDRs (e.g., 3 distinct heavy chain CDRs and 3 distinct light chain CDRs). In certain embodiments, a binding portion of an antibody comprises at least 2, at least 3, at least 4, at least 5 or at least 6 distinct CDRs. In some embodiments a binding portion of an antibody comprises 3 to 6 distinct CDRs.

In certain embodiments, an antibody or binding portion thereof, comprises one, two or three CDRs of a light chain variable region. In some embodiments a light chain variable region of an antibody, or binding portion thereof, comprises one or more CDRs (e.g., one, two, three, or more CDRs). The amino acid sequences representing a CDR in a light chain variable region of an antibody is referred to as CDR-L1 (light chain CDR1), CDR-L2 (light chain CDR2), and CDR-L3 (light chain CDR3), which are numbered sequentially (i.e., L1, L2 and L3) in the direction from the amino terminus (N-terminus) to the carboxy terminus (C-terminus) of a light chain variable region. For example, in a polypeptide representing a light chain variable region of an antibody, or binding portion thereof, CDR-L1, when present, is the most N-terminal light chain CDR; CDR-L3, when present, is the most C-terminal light chain CDR; and CDR-L2, when present, is located (i) between CDR-L1 and CDR-L3, (ii) on the N-terminal side of CDR-L3 or (iii) on the C-terminal side of CDR-L1, of a light chain variable region or binding portion of an antibody. The terms “CDR-L1”, “CDR-L2” and “CDR-L3” refer to, in part, an amino acid sequence of a polypeptide identified as, or disclosed herein as, a complementarity determining region of an antibody (e.g., a CDR of a light chain variable region). Non-limiting examples of amino acid sequences of a CDR-L1, CDR-L2 and CDR-L3 are provided in Tables 1-3, respectively. A light chain variable region or antigen binding portion of an antibody may comprise any combination of a CDR-L1, a CDR-L2, and a CDR-L3 disclosed herein, wherein the binding portion of the antibody retains specific binding to syndecan-1, or a portion thereof. In certain embodiments, a light chain variable region or antigen binding portion of an antibody comprises a single light chain CDR comprising an amino acid sequence at least 70% identical to a CDR-L3 selected from Table 3. In certain embodiments, a light chain variable region or antigen binding portion of an antibody comprises an amino acid sequence at least 70% identical to a CDR-L3 selected from Table 3, and/or another suitable CDR-L2 and/or CDR-L1 polypeptide sequence, where the antibody, or binding portion thereof, retains specific binding to syndecan-1, or a portion thereof. In certain embodiments, the light chain CDRs of a light chain variable region or antigen binding portion of an antibody consists of a CDR-L3 and a CDR-L2, where the CDR-L3 comprises an amino acid sequence at least 70% identical to a CDR-L3 selected from Table 3 and the CDR-L2 comprises an amino acid sequence at least 70% identical to a CDR-L2 selected from Table 2. In certain embodiments, a light chain variable region or antigen binding portion of an antibody comprises an amino acid sequence at least 70% identical to a CDR-L3 selected from Table 3 and an amino acid sequence at least 70% identical to a CDR-L2 selected from Table 2, and any other suitable CDR-L1 polypeptide sequence, where the antibody, or binding portion thereof, retains specific binding to syndecan-1, or a portion thereof. In certain embodiments, a light chain variable region or antigen binding portion of an antibody comprises three light chain CDRs consisting of an amino acid sequence at least 70% identical to a CDR-L3 selected from Table 3, an amino acid sequence at least 70% identical to a CDR-L2 selected from Table 2 and an amino acid sequence selected at least 70% identical to a CDR-L1 of Table 1. In certain embodiments, a light chain variable region or antigen binding portion of an antibody comprises an amino acid sequence at least 70% identical to a CDR-L3 selected from Table 3, an amino acid sequence at least 70% identical to a CDR-L2 selected from Table 2 and an amino acid sequence at least 70% identical to a CDR-L1 selected from Table 1, where the antibody, or binding portion thereof, retains specific binding to syndecan-1, or a portion thereof.

In some embodiments an antibody, or binding portion thereof, comprises one or more light chain CDRs that are at least 70%, 75%, 85%, 86%, 87%, 88%, 89%, 90%, 91%, 92%, 93%, 94%, 95%, 96%, 97%, 98%, or at least 99% identical to any one of the CDR sequences listed in Tables 1, 2 or 3. In some embodiments an antibody, or binding portion thereof, comprises a CDR-L1 that is at least 70%, 75%, 85%, 86%, 87%, 88%, 89%, 90%, 91%, 92%, 93%, 94%, 95%, 96%, 97%, 98%, or at least 99% identical to any one of the sequences shown in Table 1. In some embodiments an antibody, or binding portion thereof, comprises a CDR-L1 of any one of the sequences shown in Table 1.

TABLE 1
CDR-L1 Sequences

| | Hybridoma Clone/ | Amino |
|---|---|---|
| SEQ ID | Antibody Name | Acid Sequence |
| SEQ ID NO: 2 | F12P16F6 | *KSSQSLLASDGKTYLN |
| SEQ ID NO: 3 | F12P16F6 | QSLLASDGKTY |
| SEQ ID NO: 4 | F13P30A7 | *KASENVGNYVS |
| SEQ ID NO: 5 | F13P30A7 | ENVGNY |
| SEQ ID NO: 6 | F13P18D8 | *KASENVGTYVS |
| SEQ ID NO: 7 | F13P18D8 | ASENVGTY |
| SEQ ID NO: 8 | Fl2P7G11 | *RASSSVNYMH |
| SEQ ID NO: 9 | F12P7G11 | ASSSVNY |
| SEQ ID NO: 10 | F13P14D3 | *KASENVGSYVS |
| SEQ ID NO: 11 | F13P14D3 | ASENVGSY |
| SEQ ID NO: 12 | F11AP11E5 | *KSGQSLLYSNGKTYLT |
| SEQ ID NO: 13 | F11AP11E5 | KSGQSLLYSNG |
| SEQ ID NO: 14 | F12P18D4.a | *KSSQSLLYSNGKTYLN |
| SEQ ID NO: 15 | F12P18D4.a | KSSQSLLYSNG |

*Indicates that the CDR was defined by the Kabat Method.

In some embodiments a syndecan-1 antibody, or binding portion thereof, comprises a CDR-L2 that is at least 70%, 75%, 85%, 86%, 87%, 88%, 89%, 90%, 91%, 92%, 93%, 94%, 95%, 96%, 97%, 98%, or at least 99% identical to any one of the sequences shown in Table 2. In some embodiments a syndecan-1 antibody, or binding portion thereof, comprises a CDR-L2 of any one of the sequences shown in Table 2.

TABLE 2
CDR-L2 Sequences

| | Hybridoma Clone/ | Amino |
|---|---|---|
| SEQ ID | Antibody Name | Acid Sequence |
| SEQ ID NO: 16 | F12P16F6 | YLVSKLDS |
| SEQ ID NO: 17 | F12P16F6 | *LVSKLDS |
| SEQ ID NO: 18 | F12P16F6 | LVSKLD |
| SEQ ID NO: 19 | F13P30A7 | YGASYRYT |
| SEQ ID NO: 20 | F13P30A7 | *GASYRYT |
| SEQ ID NO: 21 | F13P30A7 | GASYRY |
| SEQ ID NO: 22 | F13P18D8 | *GASNRYT |
| SEQ ID NO: 23 | Fl2P7G11 | *ATSYLAS |
| SEQ ID NO: 24 | F13P14D3 | *GASNRNT |
| SEQ ID NO: 25 | F11AP11E5 | *QVSKLDP |
| SEQ ID NO: 26 | F12P18D4.a | *LVSKVDS |

*Indicates that the CDR was defined by the Kabat Method.

In some embodiments a syndecan-1 antibody, or binding portion thereof, comprises a CDR-L3 that is at least 70%, 75%, 85%, 86%, 87%, 88%, 89%, 90%, 91%, 92%, 93%, 94%, 95%, 96%, 97%, 98%, or at least 99% identical to any one of the sequences shown in Table 3. In some embodiments a syndecan-1 antibody, or binding portion thereof, comprises a CDR-L3 of any one of the sequences shown in Table 3.

TABLE 3
CDR-L3 Sequences

| | Hybridoma Clone/ | Amino |
|---|---|---|
| SEQ ID | Antibody Name | Acid Sequence |
| SEQ ID NO: 27 | F12P16F6 | *WQGAHFPFT |
| SEQ ID NO: 28 | F12P16F6 | QGAHFPF |
| SEQ ID NO: 29 | F13P30A7/F13P14D3/ | *GQSSRYPLT |
| | F13P18D8 | |
| SEQ ID NO: 30 | F13P30A7/F13P14D3/ | QSSRYPL |
| | Fl3P18D8 | |
| SEQ ID NO: 31 | F12P7G11 | *QQWSSDPLT |
| SEQ ID NO: 32 | F11AP11E5 | *LQNTYYPHT |
| SEQ ID NO: 33 | F12P18D4.a | *VQGTHFPLT |

*Indicates that the CDR was defined by the Kabat Method.

In some embodiments a syndecan-1 antibody, or binding portion thereof, comprises a light chain variable region having at least 70%, 75%, 85%, 86%, 87%, 88%, 89%, 90%, 91%, 92%, 93%, 94%, 95%, 96%, 97%, 98%, or at least 99% identity to an amino acid sequence of Table 4. In some embodiments a syndecan-1 antibody, or binding portion thereof, comprises a light chain variable region sequence of Table 4.

TABLE 4
MOUSE VARIABLE LIGHT CHAIN SEQUENCES

| | Hybridoma | |
|---|---|---|
| | Clone/ | Mouse |
| SEQ ID | Antibody Name | Variable Light Chain Amino Acid Sequence |
| SEQ ID | F12P16F6 | DVVMTQTPLTLSVTIGQPASISCKSSQSLLASDGKTYLN |
| NO: 34 | | WLLQRPGQSPKRLIYLVSKLDSGVPDRFTGSGSGTGFTL |
| | | QISRVEAEDLGIYYCWQGAHFPFTFGSGTKLEIKR |
| SEQ ID | F13P30A7 | NIIMTQSPKSMAMSVGERVTLSCKASENVGNYVSWYQ |
| NO: 35 | | QKPEQSPKLLIYGASYRYTGVPDRFTGSGSGTDFTLTISS |
| | | VQAEDLADYHCGQSSRYPLTFGAGTKLELKR |
| SEQ ID | F13P18D8 | NIVMTQSPKSMSMSVGERVTLSCKASENVGTYVSWYQ |
| NO: 36 | | QKSDQSPKLLIYGASNRYTGVPDRFTGSGSATDFTLTITS |
| | | VQSEDLADYHCGQSSRYPLTFGAGTKLELKR |
| SEQ ID | F12P7G11 | QIVLSQSPAILSASPGEKVTMTCRASSSVNYMHWYQQK |
| NO: 37 | | PGSSPKHWIYATSYLASGVPARFSGSGSGTSYSLTISRVE |
| | | AEDAATYYCQQWSSDPLTFGAGTKLELKR |
| SEQ ID | F13P14D3 | NIVMTQSPKSMSMSVGQRVTLSCKASENVGSYVSWYQ |
| NO: 38 | | QKPEQSPKLLIYGASNRNTGVPDRFTGSGSATDFTLTISS |
| | | VQAEDLADYHCGQSSRYPLTFGGGTKLELKR |
| SEQ ID | F11AP11E5 | DVVMTQTPLSLSVTIGQPASISCKSGQSLLYSNGKTYLT |
| NO: 39 | | WLQQRPGQAPKLLMYQVSKLDPGIPDRFSGSGSETDFTL |
| | | KISRVEAEDLGVYYCLQNTYYPHTFGAGTKLELKR |
| SEQ ID | F12P18D4.a | DVVMTQTPLTLSVTIGQSASISCKSSQSLLYSNGKTYLN |
| NO: 40 | | WLLQRPGQSPKRLIYLVSKVDSGVPDRFTGSGSGTDFTL |
| | | SISRVEAEDLGVYYCVQGTHFPLTFGVGTKLELKR |

In some embodiments a syndecan-1 antibody, or binding portion thereof, comprises a humanized light chain variable region having at least 70%, 75%, 85%, 86%, 87%, 88%, 89%, 90%, 91%, 92%, 93%, 94%, 95%, 96%, 97%, 98%, or at least 99% identity to a sequence of Table 5. In some embodiments a syndecan-1 antibody, or binding portion thereof, comprises a humanized light chain variable region sequence of Table 5.

TABLE 5
Humanized Light Chains Variable Regions

| | Humanized | Humanized Light Chain Variable Region Amino Acid |
|---|---|---|
| | Light Chain | Sequences 41-43 & Complete Humanized Light Chain |
| SEQ ID | Name | Sequence 44 |
| SEQ ID | hF6 | DVVMTQTPLSLSVTPGQPASISCKSSQSLLASDGKTYL |
| NO: 41 | (sdr/cdr/ven- | NWLLQRPGQSPKRLIYLVSKLDSGVPNRFSGSGSGTDF |
| | rep) | TLQISRVEAEDVGLYYCWQGAHFPFTFGSGTKLEIKR |
| SEQ ID | P16F6 abb- | DVVMTQTPLSLSVTPGQPASISCKSSQSLLASDGKTYL |
| NO: 42 | rep | NWLLQRPGQSPKRLIYLVSKLDSGVPNRFSGSGSGTDF |
| | | TLQISRVEAEDVGLYYCMQGAHFPFTFGGGTKVEIKR |
| SEQ ID | P16F6 fra- | DVVMTQSPLSLSVTLGQPASISCKSSQSLLASDGKTYL |
| NO: 43 | rep | NWLQQRPGQSPRRLIYLVSKLDSGVPNRFSGSGSGTDF |
| | | TLQISRVEAEDVGLYYCWQGAHFPFTFGSGTKLEIKR |
| SEQ ID | hF6 | DVVMTQTPLSLSVTPGQPASISCKSSQSLLASDGKTYL |
| NO: 44 | (sdr/cdr/ven- | NWLLQRPGQSPKRLIYLVSKLDSGVPNRFSGSGSGTDF |
| | rep) | TLQISRVEAEDVGLYYCWQGAHFPFTFGSGTKLEIKRT |
| | (Complete | VAAPSVFIFPPSDEQLKSGTASVVCLLNNFYPREAKVQ |
| | Light Chain | WKVDNALQSGNSQESVTEQDSKDSTYSLSSTLTLSKA |
| | sequence) | DYEKHKVYACEVTHQGLSSPVTKSFNRGEC |

In certain embodiments, a syndecan-1 antibody, or binding portion thereof, comprises one, two or three CDRs of a heavy chain variable region. In some embodiments a heavy chain variable region comprises one or more distinct CDRs (e.g., one, two, three, or more distinct CDRs). The amino acid sequences representing a CDR in a heavy chain variable region of an antibody is referred to as CDR-H1 (heavy chain CDR1), CDR-H2 (heavy chain CDR2), and CDR-H3 (heavy chain CDR3), which are numbered sequentially (i.e., H1, H2 and H3) in the direction from the amino terminus (N-terminus) to the carboxy terminus (C-terminus) of a heavy chain variable region. For example, in a polypeptide representing a heavy chain variable region of a syndecan-1 antibody, or a binding portion thereof, CDR-H1, when present, is the most N-terminal CDR; CDR-H3, when present, is the most C-terminal CDR; and CDR-H2, when present, is located (i) between CDR-H1 and CDR-H3, (ii) on the N-terminal side of CDR-H3 or (iii) on the C-terminal side of CDR-H1, of a heavy chain variable region. The terms “CDR-H1”, “CDR-H2” and “CDR-H3” refer to, in part, an amino acid sequence of a polypeptide identified as, or disclosed herein as, a complementarity determining region of a syndecan-1 antibody, or binding portion thereof (e.g., a CDR of a heavy chain variable region of a syndecan-1 antibody). Non-limiting examples of amino acid sequences of a CDR-H1, CDR-H2 and CDR-H3 are provided in Tables 6-8, respectively. A heavy chain variable region or antigen binding portion of a syndecan-1 antibody may comprise any combination of a CDR-H1, a CDR-H2, and a CDR-H3, where the syndecan-1 antibody, or binding portion thereof, retains specific binding to syndecan-1, or a portion thereof. In certain embodiments, a heavy chain variable region or antigen binding portion of a syndecan-1 antibody, or binding portion thereof, comprises a single heavy chain CDR consisting of an amino acid sequence at least 70% identical to a CDR-H3 selected from Table 8. In certain embodiments, a heavy chain variable region or antigen binding portion of a syndecan-1 antibody comprises an amino acid sequence at least 70% identical to a CDR-H3 selected from Table 8, and any other suitable CDR-H2 and/or CDR-H1 polypeptide sequence, where the syndecan-1 antibody, or binding portion thereof, retains specific binding to syndecan-1, or a portion thereof. In certain embodiments, the heavy chain CDRs of a heavy chain variable region or antigen binding portion of a syndecan-1 antibody consists of a CDR-H3 and a CDR-H2, where the CDR-H3 comprises an amino acid sequence at least 70% identical to a CDR-H3 selected from Table 8 and the CDR-H2 comprises an amino acid sequence at least 70% identical to a CDR-H2 selected from Table 7. In certain embodiments, a heavy chain variable region or antigen binding portion of a syndecan-1 antibody comprises an amino acid sequence at least 70% identical to a CDR-H3 selected from Table 8 and an amino acid sequence at least 70% identical to a CDR-H2 selected from Table 7, and any other suitable CDR-H1 polypeptide sequence, where the syndecan-1 antibody, or binding portion thereof, retains specific binding to syndecan-1 or a portion thereof. In certain embodiments, a heavy chain variable region or antigen binding portion of a syndecan-1 antibody comprises three heavy chain CDRs consisting of an amino acid sequence at least 70% identical to a CDR-H3 selected from Table 8, an amino acid sequence at least 70% identical to a CDR-H2 selected from Table 7 and an amino acid sequence selected at least 70% identical to a CDR-H1 of Table 6. In certain embodiments, a heavy chain variable region or antigen binding portion of a syndecan-1 antibody comprises an amino acid sequence at least 70% identical to a CDR-H3 selected from Table 8, an amino acid sequence at least 70% identical to a CDR-H2 selected from Table 7 and an amino acid sequence at least 70% identical to a CDR-H1 selected from Table 6, where the syndecan-1 antibody retains specific binding to syndecan-1, or a portion thereof.

In some embodiments a syndecan-1 antibody, or binding portion thereof, comprises one or more heavy chain CDRs with at least 70%, 75%, 85%, 86%, 87%, 88%, 89%, 90%, 91%, 92%, 93%, 94%, 95%, 96%, 97%, 98%, or at least 99% identity to any one of the CDRs of Tables 6, 7 or 8. In some embodiments a syndecan-1 antibody, or binding portion thereof, comprises a CDR-H1 that is at least 70%, 75%, 85%, 86%, 87%, 88%, 89%, 90%, 91%, 92%, 93%, 94%, 95%, 96%, 97%, 98%, or at least 99% identical to any one of the sequences shown in Table 6. In some embodiments a syndecan-1 antibody, or binding portion thereof, comprises a CDR-H1 of any one of the sequences shown in Table 6.

TABLE 6
CDR-H1 Sequences

| | Hybridoma Clone/ | |
|---|---|---|
| SEQ ID | Antibody Name | Amino Acid Sequence |
| SEQ ID NO: 45 | F12P16F6 | KASGYTFTSYYLY |
| SEQ ID NO: 46 | F12P16F6 | GYTFTSYYLY |
| SEQ ID NO: 47 | F12P16F6 | *SYYLY |
| SEQ ID NO: 48 | F13P30A7 | AASGFTFNTYAMN |
| SEQ ID NO: 49 | F13P30A7 | ASGFTFNTYAM |
| SEQ ID NO: 50 | F13P30A7/F13P18D8/ | *TYAMN |
| | F13P14D3 | |
| SEQ ID NO: 51 | F13P18D8 | GFAFNTYAMN |
| SEQ ID NO: 52 | F12P7G11 | GYTFSSHWMQ |
| SEQ ID NO: 53 | F12P7G11 | *SHWMQ |
| SEQ ID NO: 54 | F13P14D3 | GFTFNTYAMN |
| SEQ ID NO: 55 | F11AP11E5 | KASGYTFTNYYMY |
| SEQ ID NO: 56 | F11AP11E5 | *NYYMY |
| SEQ ID NO: 57 | F12P18D4.a | YTFAD |
| SEQ ID NO: 58 | F12P18D4.a | YTFADYYMK |
| SEQ ID NO: 59 | F12P18D4.a | *DYYMK |

*Indicates that the CDR was defined by the Kabat Method.

In some embodiments a syndecan-1 antibody, or binding portion thereof, comprises a CDR-H2 that is at least 70%, 75%, 85%, 86%, 87%, 88%, 89%, 90%, 91%, 92%, 93%, 94%, 95%, 96%, 97%, 98%, or at least 99% identical to any one of the sequences shown in Table 7. In some embodiments a syndecan-1 antibody, or binding portion thereof, comprises a CDR-H2 of any one of the sequences shown in Table 7.

TABLE 7
CDR-H2 Sequences

| | Hybridoma Clone/ | |
|---|---|---|
| SEQ ID | Antibody Name | Amino Acid Sequence |
| SEQ ID NO: 60 | F12P16F6 | EIYPRSGGTN |
| SEQ ID NO: 61 | F12P16F6 | *EIYPRSGGTNINEKFLS |
| SEQ ID NO: 62 | F13P30A7, | RIRSKSNNYATY |
| | F13P18D8, | |
| | F13P14D3 | |
| SEQ ID NO: 63 | F13P30A7, | *RIRSKSNNYATYYADSVKD |
| | F13P18D8 | |
| SEQ ID NO: 64 | F13P30A7, F13P18D8, | IRSKSNNYATY |
| | F13P14D3 | |
| SEQ ID NO: 65 | F12P7G11 | *AIYPGDGDTRFTQKFKG |
| SEQ ID NO: 66 | F12P7G11 | YPGDGDTRFTQK |
| SEQ ID NO: 67 | F13P14D3 | *RIRSKSNNYATYYVDSVKD |
| SEQ ID NO: 68 | F11AP11E5 | *EINPGNGGTNFNEKFKN |
| SEQ ID NO: 69 | F11AP11E5 | NPGNGGTNFNEKF |
| SEQ ID NO: 70 | F12P18D4.a | DINPNSGDTF |
| SEQ ID NO: 71 | F12P18D4.a | *DINPNSGDTFYNHKFKG |

*Indicates that the CDR was defined by the Kabat Method.

In some embodiments a syndecan-1 antibody, or binding portion thereof, comprises a CDR-H3 that is at least 70%, 75%, 85%, 86%, 87%, 88%, 89%, 90%, 91%, 92%, 93%, 94%, 95%, 96%, 97%, 98%, or at least 99% identical to any one of the sequences shown in Table 8. In some embodiments a syndecan-1 antibody, or binding portion thereof, comprises a CDR-H3 of any one of the sequences shown in Table 8.

TABLE 8
CDR-H3 Sequences

| | Hybridoma Clone/ | Amino |
|---|---|---|
| SEQ ID | Antibody Name | Acid Sequence |
| SEQ ID NO: 72 | F12P16F6 | TRSLLY |
| SEQ ID NO: 73 | F12P16F6 | *SLLY |
| SEQ ID NO: 74 | F13P30A7 | VTDYGYVYFDA |
| SEQ ID NO: 75 | F13P30A7 | *DYGYVYFDA |
| SEQ ID NO: 76 | F13P18D8 | *DYYYVYFDV |
| SEQ ID NO: 77 | F12P7G11 | *GIYYDRSRAMDY |
| SEQ ID NO: 78 | F13P14D3 | VTDYGHVYFDV |
| SEQ ID NO: 79 | F13P14D3 | *DYGHVYFDV |
| SEQ ID NO: 80 | F11AP11E5 | *RFAY |
| SEQ ID NO: 81 | F12P18D4.a | *TYYDY |

*Indicates that the CDR was defined by the Kabat Method.

In some embodiments, an antibody, or binding portion thereof, comprises, or consists of, a CDR-L1 comprising an amino acid sequence at least 90%, at least 95%, or 100% identical to any one of the amino acid sequences of Table 1, a CDR-L2 comprising an amino acid sequence at least 90%, at least 95%, or 100% identical to any one of the amino acid sequences of Table 2, a CDR-L3 comprising an amino acid sequence at least 90%, at least 95%, or 100% identical to any one of the amino acid sequences of Table 3, a CDR-H1 comprising an amino acid sequence at least 90%, at least 95%, or 100% identical to any one of the amino acid sequences of Table 6, a CDR-H2 comprising an amino acid sequence at least 90%, at least 95%, or 100% identical to any one of the amino acid sequences of Table 7, and a CDR-H3 comprising an amino acid sequence at least 90%, at least 95%, or 100% identical to any one of the amino acid sequences of Table 8.

In some embodiments an antibody, or binding portion thereof, comprises, or consists of, a CDR-L1 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:2 or 3, a CDR-L2 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:16, 17 or 18, a CDR-L3 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:27 or 28, a CDR-H1 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:45, 46 or 47, a CDR-H2 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:60 or 61, and a CDR-H3 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:72 or 73.

In some embodiments an antibody, or binding portion thereof, comprises, or consists of, a CDR-L1 comprising an amino acid sequence at least 80%, at least 85%, at least 90%, or 100% identical to the amino acid sequence of SEQ ID NO:3, a CDR-L2 comprising an amino acid sequence at least 80%, at least 85%, at least 90%, or 100% identical to the amino acid sequence of SEQ ID NO:18, a CDR-L3 comprising an amino acid sequence at least 80%, at least 85%, at least 90%, or 100% identical to the amino acid sequence of SEQ ID NO:28, a CDR-H1 comprising an amino acid sequence at least 80%, at least 85%, at least 90%, or 100% identical to the amino acid sequence of SEQ ID NO:47, a CDR-H2 comprising an amino acid sequence at least 80%, at least 85%, at least 90%, or 100% identical to the amino acid sequence of SEQ ID NO:60, and a CDR-H3 comprising an amino acid sequence at least 80%, at least 85%, at least 90%, or 100% identical to the amino acid sequence of SEQ ID NO:73.

In some embodiments an antibody, or binding portion thereof, comprises a CDR-L1 comprising an amino acid sequence at least 80%, at least 85%, at least 90%, or 100% identical to the amino acid sequence of SEQ ID NO:2, a CDR-L2 comprising an amino acid sequence at least 80%, at least 85%, at least 90%, or 100% identical to the amino acid sequence of SEQ ID NO:17, a CDR-L3 comprising an amino acid sequence at least 80%, at least 85%, at least 90%, or 100% identical to the amino acid sequence of SEQ ID NO:27, a CDR-H1 comprising an amino acid sequence at least 80%, at least 85%, at least 90%, or 100% identical to the amino acid sequence of SEQ ID NO:47, a CDR-H2 comprising an amino acid sequence at least 80%, at least 85%, at least 90%, or 100% identical to the amino acid sequence of SEQ ID NO:61, and a CDR-H3 comprising an amino acid sequence at least 80%, at least 85%, at least 90%, or 100% identical to the amino acid sequence of SEQ ID NO:73. In some embodiments an antibody, or binding portion thereof, comprises a CDR-L1 comprising an amino acid sequence at least 80%, at least 85%, at least 90%, or 100% identical to the amino acid sequence of SEQ ID NO:2, a CDR-L2 comprising an amino acid sequence at least 80%, at least 85%, at least 90%, or 100% identical to the amino acid sequence of SEQ ID NO:16, a CDR-L3 comprising an amino acid sequence at least 80%, at least 85%, at least 90%, or 100% identical to the amino acid sequence of SEQ ID NO:27, a CDR-H1 comprising an amino acid sequence at least 80%, at least 85%, at least 90%, or 100% identical to the amino acid sequence of SEQ ID NO:45, a CDR-H2 comprising an amino acid sequence at least 80%, at least 85%, at least 90%, or 100% identical to the amino acid sequence of SEQ ID NO:60, and a CDR-H3 comprising an amino acid sequence at least 80%, at least 85%, at least 90%, or 100% identical to the amino acid sequence of SEQ ID NO:72. In some embodiments, an antibody, or binding portion thereof, comprises a CDR-L1, CDR-L2 and a CDR-L3 comprising the amino acid sequences of SEQ ID NOs:2, 16 and 27, respectively, and a CDR-H1, CDR-H2 and a CDR-H3 comprising the amino acid sequences of SEQ ID NOs:45, 60 and 72, respectively. In some embodiments, an antibody, or binding portion thereof, comprises a CDR-L1, CDR-L2 and a CDR-L3 comprising the amino acid sequences of SEQ ID NOs:2, 16 and 27, respectively, and a CDR-H1, CDR-H2 and a CDR-H3 comprising the amino acid sequences of SEQ ID NOs:46, 61 and 72, respectively. In some embodiments, an antibody, or binding portion thereof, comprises a CDR-L1, CDR-L2 and a CDR-L3 comprising the amino acid sequences of SEQ ID NOs:2, 16 and 27, respectively, and a CDR-H1, CDR-H2 and a CDR-H3 comprising the amino acid sequences of SEQ ID NOs:45, 61 and 72, respectively.

In some embodiments an antibody, or binding portion thereof, comprises, or consists of, a CDR-L1 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:4 or 5, a CDR-L2 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:19, 20 or 21, a CDR-L3 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:29 or 30, a CDR-H1 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:48, 49 or 50, a CDR-H2 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:62, 63 or 64, and a CDR-H3 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:74 or 75.

In some embodiments an antibody, or binding portion thereof, comprises, or consists of, a CDR-L1 comprising an amino acid sequence at least 80%, at least 90%, or 100% identical to the amino acid sequence of SEQ ID NO:5, a CDR-L2 comprising an amino acid sequence at least 80%, at least 90%, or 100% identical to the amino acid sequence of SEQ ID NO:21, a CDR-L3 comprising an amino acid sequence at least 80%, at least 90%, or 100% identical to the amino acid sequence of SEQ ID NO:30, a CDR-H1 comprising an amino acid sequence at least 80%, at least 90%, or 100% identical to the amino acid sequence of SEQ ID NO:50, a CDR-H2 comprising an amino acid sequence at least 80%, at least 90%, or 100% identical to the amino acid sequence of SEQ ID NO:62 or 64, and a CDR-H3 comprising an amino acid sequence at least 80%, at least 90%, or 100% identical to the amino acid sequence of SEQ ID NO:75.

In some embodiments an antibody, or binding portion thereof, comprises a CDR-L1 comprising an amino acid sequence at least 80%, at least 90%, or 100% identical to the amino acid sequence of SEQ ID NO:4, a CDR-L2 comprising an amino acid sequence at least 80%, at least 90%, or 100% identical to the amino acid sequence of SEQ ID NO:20, a CDR-L3 comprising an amino acid sequence at least 80%, at least 90%, or 100% identical to the amino acid sequence of SEQ ID NO:29, a CDR-H1 comprising an amino acid sequence at least 80%, at least 90%, or 100% identical to the amino acid sequence of SEQ ID NO:50, a CDR-H2 comprising an amino acid sequence at least 80%, at least 90%, or 100% identical to the amino acid sequence of SEQ ID NO:63, and a CDR-H3 comprising an amino acid sequence at least 80%, at least 90%, or 100% identical to the amino acid sequence of SEQ ID NO:75. In some embodiments, an antibody, or binding portion thereof, comprises a CDR-L1, CDR-L2 and a CDR-L3 comprising the amino acid sequences of SEQ ID NOs:4, 19 and 29, respectively, and a CDR-H1, CDR-H2 and a CDR-H3 comprising the amino acid sequences of SEQ ID NOs:48, 63 and 75, respectively. In some embodiments, an antibody, or binding portion thereof, comprises a CDR-L1, CDR-L2 and a CDR-L3 comprising the amino acid sequences of SEQ ID NOs:4, 19 and 29, respectively, and a CDR-H1, CDR-H2 and a CDR-H3 comprising the amino acid sequences of SEQ ID NOs:49, 63 and 75, respectively. In some embodiments, an antibody, or binding portion thereof, comprises a CDR-L1, CDR-L2 and a CDR-L3 comprising the amino acid sequences of SEQ ID NOs:4, 19 and 29, respectively, and a CDR-H1, CDR-H2 and a CDR-H3 comprising the amino acid sequences of SEQ ID NOs:49, 63 and 74, respectively. In some embodiments, an antibody, or binding portion thereof, comprises a CDR-L1, CDR-L2 and a CDR-L3 comprising the amino acid sequences of SEQ ID NOs:4, 19 and 29, respectively, and a CDR-H1, CDR-H2 and a CDR-H3 comprising the amino acid sequences of SEQ ID NOs:48, 63 and 74, respectively.

In some embodiments an antibody, or binding portion thereof, comprises, or consists of, a CDR-L1 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:6 or 7, a CDR-L2 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:22, a CDR-L3 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:29 or 30, a CDR-H1 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:50 or 51, a CDR-H2 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:62, 63 or 64, and a CDR-H3 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:76.

In some embodiments an antibody, or binding portion thereof, comprises, or consists of, a CDR-L1 comprising an amino acid sequence at least 80%, at least 90%, or 100% identical to the amino acid sequence of SEQ ID NO:7, a CDR-L2 comprising an amino acid sequence at least 80%, at least 90%, or 100% identical to the amino acid sequence of SEQ ID NO:22, a CDR-L3 comprising an amino acid sequence at least 80%, at least 90%, or 100% identical to the amino acid sequence of SEQ ID NO:30, a CDR-H1 comprising an amino acid sequence at least 80%, at least 90%, or 100% identical to the amino acid sequence of SEQ ID NO:50, a CDR-H2 comprising an amino acid sequence at least 80%, at least 90%, or 100% identical to the amino acid sequence of SEQ ID NO:63, and a CDR-H3 comprising an amino acid sequence at least 80%, at least 90%, or 100% identical to the amino acid sequence of SEQ ID NO:76.

In some embodiments an antibody, or binding portion thereof, comprises a CDR-L1 comprising an amino acid sequence at least 80%, at least 90%, or 100% identical to the amino acid sequence of SEQ ID NO:6, a CDR-L2 comprising an amino acid sequence at least 80%, at least 90%, or 100% identical to the amino acid sequence of SEQ ID NO:22, a CDR-L3 comprising an amino acid sequence at least 80%, at least 90%, or 100% identical to the amino acid sequence of SEQ ID NO:29, a CDR-H1 comprising an amino acid sequence at least 80%, at least 90%, or 100% identical to the amino acid sequence of SEQ ID NO:50, a CDR-H2 comprising an amino acid sequence at least 80%, at least 90%, or 100% identical to the amino acid sequence of SEQ ID NO:63, and a CDR-H3 comprising an amino acid sequence at least 80%, at least 90%, or 100% identical to the amino acid sequence of SEQ ID NO:76. In some embodiments, an antibody, or binding portion thereof, comprises a CDR-L1, CDR-L2 and a CDR-L3 comprising the amino acid sequences of SEQ ID NOs:6, 22 and 29, respectively, and a CDR-H1, CDR-H2 and a CDR-H3 comprising the amino acid sequences of SEQ ID NOs:51, 63 and 76, respectively.

In some embodiments an antibody, or binding portion thereof, comprises, or consists of, a CDR-L1 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:8 or 9, a CDR-L2 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:23, a CDR-L3 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:31, a CDR-H1 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:52 or 53, a CDR-H2 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:65 or 66, and a CDR-H3 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:77.

In some embodiments an antibody, or binding portion thereof, comprises, or consists of, a CDR-L1 comprising an amino acid sequence at least 80%, at least 90%, or 100% identical to the amino acid sequence of SEQ ID NO:9, a CDR-L2 comprising an amino acid sequence at least 80%, at least 90%, or 100% identical to the amino acid sequence of SEQ ID NO:23, a CDR-L3 comprising an amino acid sequence at least 80%, at least 90%, or 100% identical to the amino acid sequence of SEQ ID NO:31, a CDR-H1 comprising an amino acid sequence at least 80%, at least 90%, or 100% identical to the amino acid sequence of SEQ ID NO:53, a CDR-H2 comprising an amino acid sequence at least 80%, at least 90%, or 100% identical to the amino acid sequence of SEQ ID NO:66, and a CDR-H3 comprising an amino acid sequence at least 80%, at least 90%, or 100% identical to the amino acid sequence of SEQ ID NO:77.

In some embodiments an antibody, or binding portion thereof, comprises a CDR-L1 comprising an amino acid sequence at least 80%, at least 90%, or 100% identical to the amino acid sequence of SEQ ID NO:8, a CDR-L2 comprising an amino acid sequence at least 80%, at least 90%, or 100% identical to the amino acid sequence of SEQ ID NO:23, a CDR-L3 comprising an amino acid sequence at least 80%, at least 90%, or 100% identical to the amino acid sequence of SEQ ID NO:31, a CDR-H1 comprising an amino acid sequence at least 80%, at least 90%, or 100% identical to the amino acid sequence of SEQ ID NO:53, a CDR-H2 comprising an amino acid sequence at least 80%, at least 90%, or 100% identical to the amino acid sequence of SEQ ID NO:65, and a CDR-H3 comprising an amino acid sequence at least 80%, at least 90%, or 100% identical to the amino acid sequence of SEQ ID NO:77. In some embodiments, an antibody, or binding portion thereof, comprises a CDR-L1, CDR-L2 and a CDR-L3 comprising the amino acid sequences of SEQ ID NOs:8, 23 and 31, respectively, and a CDR-H1, CDR-H2 and a CDR-H3 comprising the amino acid sequences of SEQ ID NOs:52, 65 and 77, respectively.

In some embodiments an antibody, or binding portion thereof, comprises, or consists of, a CDR-L1 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:10 or 11, a CDR-L2 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:24, a CDR-L3 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:29 or 30, a CDR-H1 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:50 or 54, a CDR-H2 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:62, 64 or 67, and a CDR-H3 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:78 or 79.

In some embodiments an antibody, or binding portion thereof, comprises, or consists of, a CDR-L1 comprising an amino acid sequence at least 80%, at least 90%, or 100% identical to the amino acid sequence of SEQ ID NO:11, a CDR-L2 comprising an amino acid sequence at least 80%, at least 90%, or 100% identical to the amino acid sequence of SEQ ID NO:24, a CDR-L3 comprising an amino acid sequence at least 80%, at least 90%, or 100% identical to the amino acid sequence of SEQ ID NO:30, a CDR-H1 comprising an amino acid sequence at least 80%, at least 90%, or 100% identical to the amino acid sequence of SEQ ID NO:50, a CDR-H2 comprising an amino acid sequence at least 80%, at least 90%, or 100% identical to the amino acid sequence of SEQ ID NO:62 or 64, and a CDR-H3 comprising an amino acid sequence at least 80%, at least 90%, or 100% identical to the amino acid sequence of SEQ ID NO:79.

In some embodiments an antibody, or binding portion thereof, comprises a CDR-L1 comprising an amino acid sequence at least 80%, at least 90%, or 100% identical to the amino acid sequence of SEQ ID NO:10, a CDR-L2 comprising an amino acid sequence at least 80%, at least 90%, or 100% identical to the amino acid sequence of SEQ ID NO:24, a CDR-L3 comprising an amino acid sequence at least 80%, at least 90%, or 100% identical to the amino acid sequence of SEQ ID NO:29, a CDR-H1 comprising an amino acid sequence at least 80%, at least 90%, or 100% identical to the amino acid sequence of SEQ ID NO:50, a CDR-H2 comprising an amino acid sequence at least 80%, at least 90%, or 100% identical to the amino acid sequence of SEQ ID NO:67, and a CDR-H3 comprising an amino acid sequence at least 80%, at least 90%, or 100% identical to the amino acid sequence of SEQ ID NO:79. In some embodiments, an antibody, or binding portion thereof, comprises a CDR-L1, CDR-L2 and a CDR-L3 comprising the amino acid sequences of SEQ ID NOs:10, 24 and 29, respectively, and a CDR-H1, CDR-H2 and a CDR-H3 comprising the amino acid sequences of SEQ ID NOs:54, 67 and 78, respectively.

In some embodiments an antibody, or binding portion thereof, comprises, or consists of, a CDR-L1 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:12 or 13, a CDR-L2 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:25, a CDR-L3 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:32, a CDR-H1 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:55 or 56, a CDR-H2 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:68 or 69, and a CDR-H3 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:80.

In some embodiments an antibody, or binding portion thereof, comprises, or consists of, a CDR-L1 comprising an amino acid sequence at least 80%, at least 90%, or 100% identical to the amino acid sequence of SEQ ID NO:13, a CDR-L2 comprising an amino acid sequence at least 80%, at least 90%, or 100% identical to the amino acid sequence of SEQ ID NO:25, a CDR-L3 comprising an amino acid sequence at least 80%, at least 90%, or 100% identical to the amino acid sequence of SEQ ID NO:32, a CDR-H1 comprising an amino acid sequence at least 80%, at least 90%, or 100% identical to the amino acid sequence of SEQ ID NO:56, a CDR-H2 comprising an amino acid sequence at least 80%, at least 90%, or 100% identical to the amino acid sequence of SEQ ID NO:69, and a CDR-H3 comprising an amino acid sequence at least 80%, at least 90%, or 100% identical to the amino acid sequence of SEQ ID NO:80.

In some embodiments an antibody, or binding portion thereof, comprises a CDR-L1 comprising an amino acid sequence at least 80%, at least 90%, or 100% identical to the amino acid sequence of SEQ ID NO:12, a CDR-L2 comprising an amino acid sequence at least 80%, at least 90%, or 100% identical to the amino acid sequence of SEQ ID NO:25, a CDR-L3 comprising an amino acid sequence at least 80%, at least 90%, or 100% identical to the amino acid sequence of SEQ ID NO:32, a CDR-H1 comprising an amino acid sequence at least 80%, at least 90%, or 100% identical to the amino acid sequence of SEQ ID NO:56, a CDR-H2 comprising an amino acid sequence at least 80%, at least 90%, or 100% identical to the amino acid sequence of SEQ ID NO:68, and a CDR-H3 comprising an amino acid sequence at least 80%, at least 90%, or 100% identical to the amino acid sequence of SEQ ID NO:80. In some embodiments, an antibody, or binding portion thereof, comprises a CDR-L1, CDR-L2 and a CDR-L3 comprising the amino acid sequences of SEQ ID NOs:12, 25 and 32, respectively, and a CDR-H1, CDR-H2 and a CDR-H3 comprising the amino acid sequences of SEQ ID NOs:55, 68 and 80, respectively.

In some embodiments an antibody, or binding portion thereof, comprises, or consists of, a CDR-L1 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:14 or 15, a CDR-L2 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:26, a CDR-L3 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:33, a CDR-H1 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:57, 58 or 59, a CDR-H2 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:70 or 71, and a CDR-H3 comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of SEQ ID NO:81

In some embodiments an antibody, or binding portion thereof, comprises, or consists of, a CDR-L1 comprising an amino acid sequence at least 80%, at least 90%, or 100% identical to the amino acid sequence of SEQ ID NO:15, a CDR-L2 comprising an amino acid sequence at least 80%, at least 90%, or 100% identical to the amino acid sequence of SEQ ID NO:26, a CDR-L3 comprising an amino acid sequence at least 80%, at least 90%, or 100% identical to the amino acid sequence of SEQ ID NO:33, a CDR-H1 comprising an amino acid sequence at least 80%, at least 90%, or 100% identical to the amino acid sequence of SEQ ID NO:59, a CDR-H2 comprising an amino acid sequence at least 80%, at least 90%, or 100% identical to the amino acid sequence of SEQ ID NO:70, and a CDR-H3 comprising an amino acid sequence at least 80%, at least 90%, or 100% identical to the amino acid sequence of SEQ ID NO:81. In some embodiments an antibody, or binding portion thereof, comprises, or consists of, a CDR-L1, a CDR-L2, a CDR-L3, a CDR-H1, a CDR-H2 and a CDR-H3 comprising an amino acid sequence at least 80%, at least 90%, or 100% identical to the amino acid sequence of SEQ ID NOs:15, 26, 33, 57, 70, and 81, respectively.

In some embodiments an antibody, or binding portion thereof, comprises a CDR-L1 comprising an amino acid sequence at least 80%, at least 90%, or 100% identical to the amino acid sequence of SEQ ID NO:14, a CDR-L2 comprising an amino acid sequence at least 80%, at least 90%, or 100% identical to the amino acid sequence of SEQ ID NO:26, a CDR-L3 comprising an amino acid sequence at least 80%, at least 90%, or 100% identical to the amino acid sequence of SEQ ID NO:33, a CDR-H1 comprising an amino acid sequence at least 80%, at least 90%, or 100% identical to the amino acid sequence of SEQ ID NO:59, a CDR-H2 comprising an amino acid sequence at least 80%, at least 90%, or 100% identical to the amino acid sequence of SEQ ID NO:71, and a CDR-H3 comprising an amino acid sequence at least 80%, at least 90%, or 100% identical to the amino acid sequence of SEQ ID NO:81. In some embodiments, an antibody, or binding portion thereof, comprises a CDR-L1, CDR-L2 and a CDR-L3 comprising the amino acid sequences of SEQ ID NOs:14, 26 and 33, respectively, and a CDR-H1, CDR-H2 and a CDR-H3 comprising the amino acid sequences of SEQ ID NOs:58, 71 and 81, respectively. In some embodiments, an antibody, or binding portion thereof, comprises a CDR-L1, CDR-L2 and a CDR-L3 comprising the amino acid sequences of SEQ ID NOs:14, 26 and 33, respectively, and a CDR-H1, CDR-H2 and a CDR-H3 comprising the amino acid sequences of SEQ ID NOs:57, 71 and 81, respectively.

In some embodiments a syndecan-1 antibody, or binding portion thereof, comprises a heavy chain variable region having at least 70%, 75%, 85%, 86%, 87%, 88%, 89%, 90%, 91%, 92%, 93%, 94%, 95%, 96%, 97%, 98%, or at least 99% identity to a sequence of Table 9. In some embodiments a syndecan-1 antibody, or binding portion thereof, comprises a heavy chain variable region sequence of Table 9.

TABLE 9
MOUSE VARIABLE HEAVY CHAIN SEQUENCES

| | Hybridoma | |
|---|---|---|
| | Clone/ | Mouse |
| SEQ ID | Antibody Name | Variable Heavy Chain Amino Acid Sequence |
| SEQ ID | F12P16F6 | QVQLQQSGAEVVKPGASVKLSCKASGYTFTSYYLYWV |
| NO: 82 | | KKGPGQGLDWIGEIYPRSGGTNINEKFLSKATLTADESS |
| | | STAYLQLSSLTSEDSAVYYCTRSLLYWGQGTTLIVSS |
| SEQ ID | F13P30A7 | EVQLVESGGGLVQPKGSLKLSCAASGFTFNTYAMNWV |
| NO: 83 | | RQAPGKGLEWIARIRSKSNNYATYYADSVKDRFTISRD |
| | | DSQSLLYLQMNNLKTEDTAIFYCVTDYGYVYFDAWGA |
| | | GTTVTVSS |
| SEQ ID | F13P18D8 | EVQLLESGGGLVQPEGSLKLSCAASGFAFNTYAMNWV |
| NO: 84 | | RQAPGKGLEWLARIRSKSNNYATYYADSVKDRFTISRD |
| | | DSQGMLYLQMNNLKTEDTAMYYCVTDYYYVYFDVW |
| | | GAGTTVTVSS |
| SEQ ID | F12P7G11 | QVQLQQSGAELARPGASVKLSCKASGYTFSSHWMQW |
| NO: 85 | | VKQRPGQGLEWIGAIYPGDGDTRFTQKFKGKATLTAD |
| | | KSSNTAYMQLSSLASEDSAVYYCARGIYYDRSRAMDY |
| | | WGQGTSVTVSS |
| SEQ ID | F13P14D3 | EVQLVESGGGLVQPKGSLKLSCATSGFTFNTYAMNWV |
| NO: 86 | | RQAPGKGLEWVARIRSKSNNYATYYVDSVKDRFTISRD |
| | | DSQSTVHLQMNNLKTEDTAIYYCVTDYGHVYFDVWG |
| | | AGTTVTVSS |
| SEQ ID | F11AP11E5 | QVQLQQSGAELVKPGASVKLSCKASGYTFTNYYMYW |
| NO: 87 | | VKQRPGQGLEWIGEINPGNGGTNFNEKFKNKATLTVD |
| | | KSSSTAYMQLSSLTSEDSAVYYCTTRFAYWGQGTLVIV |
| | | SA |
| SEQ ID | F12P18D4.a | EVQLQQSGPELVKPGASVKMSCKASGYTFADYYMKW |
| NO: 88 | | VKQSHGKSLEWIGDINPNSGDTFYNHKFKGKATLTVD |
| | | KSSSTAYMQLNSLTSEDSAVYYCARTYYDYWGQGTTL |
| | | TVSS |

In some embodiments a syndecan-1 antibody, or binding portion thereof, comprises a humanized heavy chain variable region having at least 70%, 75%, 85%, 86%, 87%, 88%, 89%, 90%, 91%, 92%, 93%, 94%, 95%, 96%, 97%, 98%, or at least 99% identity to a sequence of Table 10. In some embodiments a syndecan-1 antibody, or binding portion thereof, comprises a humanized heavy chain variable region sequence of Table 10.

TABLE 10
HUMANIZED HEAVY CHAINS

| | Heavy | Humanized Heavy Chain Variable Region Amino Acid |
|---|---|---|
| | Chain Name | Sequences 89-92 & Complete Humanized Heavy Chain |
| SEQ ID | | Sequence 93 |
| SEQ ID | P16F6 | QVQLVQSGAEVVKPGASVKLSCKASGYTFTSYYLYWVKKA |
| NO: 89 | abb/sdr-rep | PGQGLDWIGEIYPRSGGTNYAEKFQGRVTLTADTSTSTAYL |
| | | ELSSLTSEDTAVYYCTRSLLYWGQGTTLTVSS |
| SEQ ID | hF6 (cdr/ven- | QVQLVQSGAEVVKPGASVKLSCKASGYTFTSYYLYWVKKA |
| NO: 90 | rep) | PGQGLDWIGEIYPRSGGTNINEKFLSRVTLTADTSTSTAYLEL |
| | | SSLTSEDTAVYYCTRSLLYWGQGTTLTVSS |
| SEQ ID | P16F6 fra1- | QVQLQQSGAEVVKPGASVKLSCKASGYTFTSYYLYWVKKA |
| NO: 91 | rep | PGQGLDWIGEIYPRSGGTNINEKFLSRVTLTADTSTSTAYMD |
| | | LSSLTSEDTAVYYCTRSLLYWGQGTTLTVSS |
| SEQ ID | P16F6 fra2- | QVQLQQSGAEVVKPGASVKLSCKASGYTFTSYYLYWVKKA |
| NO: 92 | rep | PGQGLDWIGEIYPRSGGTNINEKFLSRVTITADESTSTVYMQ |
| | | LSSLTSEDSAVYYCTRSLLYWGQGTTLTVSS |
| SEQ ID | hF6 heavy | QVQLVQSGAEVVKPGASVKLSCKASGYTFTSYYLYWVKKA |
| NO: 93 | chain | PGQGLDWIGEIYPRSGGTNINEKFLSRVTLTADTSTSTAYLEL |
| | (cdr/ven-rep) | SSLTSEDTAVYYCTRSLLYWGQGTTLTVSSASTKGPSVFPLA |
| | (complete | PSSKSTSGGTAALGCLVKDYFPEPVTVSWNSGALTSGVHTF |
| | heavy chain | PAVLQSSGLYSLSSVVTVPSSSLGTQTYICNVNHKPSNTKVD |
| | sequence) | KKVEPKSCDKTHTCPPCPAPELLGGPSVFLFPPKPKDTLMISR |
| | | TPEVTCVVVDVSHEDPEVKFNWYVDGVEVHNAKTKPREEQ |
| | | YNSTYRVVSVLTVLHQDWLNGKEYKCKVSNKALPAPIEKTI |
| | | SKAKGQPREPQVYTLPPSREEMTKNQVSLTCLVKGFYPSDIA |
| | | VEWESNGQPENNYKTTPPVLDSDGSFFLYSKLTVDKSRWQ |
| | | QGNVFSCSVMHEALHNHYTQKSLSLSPGK |

In some embodiments an antibody, or binding portion thereof, comprises, or consists of, a heavy chain variable region comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to any one of the amino acid sequences of SEQ ID NOs:82 to 92, and/or a light chain variable region comprising an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to any one of the amino acid sequences of SEQ ID NOs:34 to 43. In some embodiments an antibody, or binding portion thereof, comprises a heavy chain variable region comprising an amino acid sequence at least 80%, at least 85%, at least 90%, or 100% identical to any one of the amino acid sequences of SEQ ID NOs:89-92, and a light chain variable region comprising an amino acid sequence at least 80%, at least 85%, at least 90%, or 100% identical to any one of the amino acid sequences of SEQ ID NOs:41 to 43. In some embodiments an antibody, or binding portion thereof, comprises a heavy chain variable region comprising an amino acid sequence at least 80%, at least 85%, at least 90%, or 100% identical to the amino acid sequences of SEQ ID NO:90, and a light chain variable region comprising an amino acid sequence at least 80%, at least 85%, at least 90%, or 100% identical to the amino acid sequences of SEQ ID NO:41.

In some embodiments an antibody, or binding portion thereof, comprises one or more CDRs selected from a light chain variable region of Tables 4 and 5. In some embodiments an antibody, or binding portion thereof, comprises one or more CDRs selected from a heavy chain variable region of Tables 9 and 10. In some embodiments an antibody, or binding portion thereof, comprises one or more CDRs selected from a light chain variable region of Tables 4 and 5 and one or more CDRs selected from a heavy chain variable region of Tables 9 and 10. In certain embodiments, an antibody, or binding portion thereof, comprises a CDR-L1, a CDR-L2, and a CDR-L3, each selected from any one of the light chain variable regions of Tables 4 and 5, and a CDR-H1, a CDR-H2, and a CDR-H3, each selected from any one of the heavy chain variable regions of Tables 9 and 10. An amino acid sequence of a CDR (e.g., a CDR-L1, CDR-L2, CDR-L3, CDR-H1, CDR-H2, and CDR-H3) can be identified within a heavy chain or light chain variable region disclosed herein by any suitable method described herein or known to those skilled in the art.

In some embodiments an antibody comprises a heavy chain comprising an amino acid sequence at least 80%, at least 85%, at least 90%, or 100% identical to the amino acid sequences of SEQ ID NO:93, and a light chain comprising an amino acid sequence at least 80%, at least 85%, at least 90%, or 100% identical to the amino acid sequences of SEQ ID NO:44.

In some embodiments an antibody, or binding portion thereof, comprises one or more suitable sequences selected from Tables 1-10 wherein the selected polypeptide sequence comprises 0 to 5, 1 to 5, 0 to 10, 1 to 10, 0 to 15, or 1 to 12 amino acid modifications, additions, deletions and/or substitutions. In some embodiments, an amino acid substitution is a conservative substitution where one amino acid is replaced with another amino acid of similar structure or having similar biochemical characteristics. Non-limiting examples of a conservative substitution include substituting a hydrophilic amino acid with another hydrophilic amino acid, substituting a hydrophobic amino acid with another hydrophobic amino acid, substituting an acidic amino acid with another acidic amino acid, substituting a basic amino acid with another basic amino acid, and substituting a neutral charged amino acid with another neutral charged amino acid. In some embodiments, an antibody, or binding portion thereof, comprises one or more amino acid analogues, non-native amino acids or amino acid derivatives.

In certain embodiments, an antibody, or binding portion thereof, comprises one or more framework regions (FR). Framework regions are often located between CDRs and/or flank CDR sequences of a heavy or light chain variable region of an antibody, or binding portion thereof. In mammals, a heavy chain variable region often comprises four framework regions and a light chain variable region often comprises four framework regions. Any suitable method can be used to identify one or more framework regions in an antibody, in a variable region of an antibody, or binding portion thereof. An antibody, or binding portion thereof, may comprise synthetic or naturally occurring framework regions which are unmodified or modified (e.g., optimized) as discussed below.

In some embodiments an antibody, or binding portion thereof, is chimeric, grafted and/or humanized. Chimeric, grafted and/or humanized antibodies often comprise modified or substituted constant regions and/or framework regions while maintaining binding specificity to syndecan-1, or a portion thereof. In some embodiments an antibody, or binding portion thereof, comprises constant regions, framework regions, or portions thereof, derived from a human antibody. In some embodiments an antibody, or binding portion thereof, comprises fully synthetic portions, one or more amino acids, or sequences of amino acids that are not found in native antibody molecules.

Naturally occurring framework regions, or portions thereof may be obtained from any suitable species. In certain embodiments the complementarity determining regions (CDRs) of the light and heavy chain variable regions of an antibody, or binding portion thereof, is grafted into framework regions from the same, or another, species. For example, one or more framework regions of an antibody, or binding portion thereof, may be derived from a rodent species (e.g., a mouse or rat) or a primate species (e.g., a human).

In certain embodiments, the CDRs of the light and/or heavy chain variable regions of an antibody, or binding portion thereof, are grafted to consensus human framework regions. To create consensus human framework regions, in certain embodiments, framework regions from several human heavy chain or light chain amino acid sequences are aligned to identify a consensus sequence. In certain embodiments, the heavy chain or light chain framework regions of an antibody, or binding portion thereof, are replaced with one or more framework regions, or portions thereof, from a different heavy chain or light chain variable region. In some embodiments an antibody, or binding portion thereof, comprises one or more human framework regions. In certain embodiments an antibody, or binding portion thereof, comprises at least 1, 2, 3, 4, 5, 6, 7, 8, 9 or 10 human framework regions. In some embodiments an antibody, or binding portion thereof, comprises one or more mouse framework regions. In certain embodiments an antibody, or binding portion thereof, comprises at least 1, 2, 3, 4, 5, 6, 7, 8, 9 or 10 mouse framework regions. In certain embodiments an antibody, or binding portion thereof, comprises one or more human framework regions and one or more mouse framework regions.

Methods of generating chimeric, humanized and/or optimized antibodies, or binding portions thereof, for example by modifying, substituting or deleting framework regions, or portions thereof, are known. Non-limiting examples of CDR grafting are described, e.g., in U.S. Pat. Nos. 6,180,370, 6,054,297, 5,693,762, 5,859,205, 5,693,761, 5,565,332, 5,585,089, and 5,530, 101, and in Jones et al., Nature, 321:522-525 (1986); Verhoeyen et al., Science, 239:1534-1536 (1988), and Winter, FEBS Letts., 430:92-94 (1998). Additional non-limiting examples of generating chimeric, grafted and/or humanized antibodies include U.S. Pat. Nos. 5,530,101; 5,707,622; 5,994,524; 6,245,894; Queen et al., (1988) PNAS 86:10029-10033; Riechmann et al., Nature (1988) 332:323-327; Antibody Engineering: Methods and Protocols, Vol. 248 of Methods in molecular biology, edited by Benny K. C. Lo, Springer Science & Business Media, (2004); and Antibody Engineering, Vol. 1, Roland E. Kontermann, Stefan Diibel, Edition 2, Publisher Springer Science & Business Media, (2010). In some embodiments an antibody, or binding portion thereof, is humanized by exchanging one or more framework regions, or portions thereof (e.g., one or more amino acids), with one or more framework regions, or portions thereof from a human antibody (e.g., see “Humanization of Antibodies” by Eduardo A. Padlan Publ. Landes BioScience 2002). In certain embodiments, an antibody, or binding portion thereof, is humanized or grafted by transferring one or more CDRs (e.g., 1, 2, 3, 4, 5 or all 6 CDRs) from a donor antibody (e.g., a mouse monoclonal antibody) to an acceptor antibody (e.g., a human antibody) while retaining the binding specificity of the donor antibody. In certain embodiments, the process of making a chimeric, grafted or humanized antibodies comprises making one or more amino acid substitutions, additions or deletions in a constant region or framework region of an antibody, or binding portion thereof. In certain embodiments, techniques such as “reshaping”, “hyperchimerization”, or “veneering/resurfacing” are used to produce a humanized antibody, or binding portion thereof. (e.g., see Vaswami et al., Annals of Allergy, Asthma, & Immunol. 81:105 (1998); Roguska et al., Prot. Engin., 9:895-904 (1996); and U.S. Pat. No. 6,072,035). In some aspects, an antibody, or binding portion thereof, is modified by a method discussed above, or by another suitable method, to reduce immunogenicity (e.g., see Gilliland et al., J. Immunol, 62(6):3663-71 (1999)).

In certain embodiments, an amino acid sequence of an antibody, or binding portion thereof, is modified to optimize binding affinity for a target (e.g., syndecan-1), species cross-reactivity, solubility and/or function (e.g., agonist activity, or lack thereof). In some embodiments a specific combination of CDRs disclosed herein is optimized for binding to syndecan-1, and/or to optimize a function or characteristic of an antibody, or binding portion thereof. For example, a characterized light chain variable region disclosed herein (e.g., a light chain variable region of any one of SEQ ID NOs:34-43) can be co-expressed, using a suitable expression system, with a library of heavy chain variable regions comprising a CDR-H1 and CDR-H2 of a characterized heavy chain variable region (e.g., a heavy chain variable region selected from Tables 6 or 7), where the CDR-H3 is replaced with a library of CDR-H3 sequences, which may include one or more CDR-H3 regions of Table 8, for example. The resulting light chain/heavy chain antibody can be screened for binding to syndecan-1 and/or for a specific function. Optimized antibodies can be identified and the amino acid sequence of the CDR-H3 can be identified by a suitable method. The above screening method can be used to identify antibodies, or binding portions thereof, comprising specific combinations of CDRs, or specific optimized CDR sequences (e.g., CDR sequences comprising amino acid substitutions, additions or deletions) that provide an antibody, or binding portion thereof, with improved binding specificity, binding affinity and/or function. Such methods of screening and optimizing an antibody, or binding portion thereof, are known (e.g., see Portolano et al., (1993) Journal of Immunology 150:880-887; and Clarkson et al., (1991) Nature 352:624-628). Such references teach methods of producing antibodies that bind a specific antigen by using known variable light chain, known variable heavy chains, or portion thereof (e.g., CDRs thereof) by screening a library of complementarity variable regions.

In certain embodiments, an antibody, or binding portion thereof, is modified to eliminate or add glycosylation sites in order to optimize affinity and/or function of an antibody, or binding portion thereof (e.g., see Co et al., Mol. Immunol, 30:1361-1367 (1993)). In some embodiments the number and/or type of glycosylation sites in an antibody, or binding portion thereof, are modified or altered. An N-linked glycosylation site is often characterized by the sequence Asn-X-Ser or Asn-X-Thr, where the amino acid residue designated as X are any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided in certain embodiments is a rearrangement of N-linked carbohydrate chains where one or more N-linked glycosylation sites (typically those that are naturally occurring) are eliminated and one or more new N-linked sites are created. In some embodiments an antibody, or binding portion thereof, is modified by deleting one or more cysteine residues or substituting one or more cysteine residues for another amino acid (e.g., serine) as compared to an unmodified antibody, or binding portion thereof. In certain embodiments cysteine variants are useful for optimizing expression, secretion, and/or solubility.

In certain embodiments an antibody, or binding portion thereof, is modified to include certain amino acid additions, substitutions, or deletions designed or intended, for example, to reduce susceptibility of an antibody, or binding portion thereof, to proteolysis, reduce susceptibility of an antibody, or binding portion thereof, to oxidation, increase serum half-life and/or confer or modify other physicochemical, pharmacokinetic or functional properties of an antibody, or binding portion thereof.

The antibody portion of a bi-specific binding agent described herein may comprise an antigen binding portion of an antibody (e.g., binding portion) that binds specifically to CD138. A binding portion of an antibody refers to the antigen binding portion of an antibody. In certain embodiments, a binding portion of an antibody comprises, at least, the minimal portion of an antibody that is sufficient to specifically bind to an antigen (e.g., syndecan-1, or a portion thereof). In certain embodiments, a binding portion of an antibody comprises one or more complementarity determining regions (CDRs) of an antibody that are necessary and sufficient to direct specific binding to CD138. In certain embodiments, a binding portion of an antibody comprises the heavy and light chain variable regions of an antibody. In certain embodiments a binding portion of antibody comprises or consists of a single polypeptide (e.g., single chain antibody). A single chain antibody may comprise one or more CDRs from a heavy and/or light chain of an antibody. In some embodiments a binding portion of antibody comprises or consists of two polypeptides (e.g., a heavy chain variable region and a light chain variable region). In some embodiments a binding portion of antibody comprises one or more structural portions (e.g., scaffolds, structural polypeptides, constant regions and/or framework regions). In some embodiments an antibody, or a binding portion of antibody is attached to a carrier or substrate (e.g., a polymer, a non-organic material, silicon, a bead, a particle or the like).

A bi-specific binding agent may comprise one binding portion of an antibody or multiple binding portions of an antibody. When a bi-specific binding agent comprises multiple binding portions of an antibody, each binding portion binds specifically to the same antigen (e.g., syndecan-1, or a portion thereof). In some embodiments a bi-specific binding agent comprises 1, 2, 3, 4, 5, 6, 7, 8, 9, or 10 or more binding portions of an antibody.

Non-limiting examples of a binding portion of an antibody include a single-chain antibody, Fab, Fab′, F(ab′)2, Fv fragment, single-chain Fv (scFv), scFv-Fc, (scFv)2-Fc, disulfide-linked Fvs (sdFv), VL (variable light chain), VH (variable heavy chain), diabody (Dab), triabody (trivalent), tetrabody (tetravalent), minibody ((scFV-CH3)2), IgGdeltaCH2, nanobodies, scFv-Igs, SVD-Igs, the like, and combinations thereof.

Nucleic acids, or portions thereof, that encode one or more polypeptides of a bi-specific binding agent, antibody, or binding portions thereof, may be cloned, subcloned, rearranged or modified for recombinant expression using a suitable cloning procedure and subsequently expressed using a suitable expression system by a method known to those skilled in the art (e.g., see Maniatis et al., Molecular Cloning: A Laboratory Manual, Cold Spring Harbor Laboratory, 1982; Antibody Engineering: Methods and Protocols, Vol. 248 of Methods in molecular biology, edited by Benny K. C. Lo, Springer Science & Business Media, 2004; Antibody Engineering, Vol. 1, Roland E. Kontermann, Stefan Diibel, Edition 2, Publisher Springer Science & Business Media, 2010; Antibody Phage Display: Methods and Protocols, Biomed Protocols, Vol. 178 of Methods in molecular biology, Editors Philippa M. O'Brien, Robert Aitken, Springer Science & Business Media, 2004).

In some embodiments an antibody, or binding portion thereof, binds specifically to a mammalian syndecan-1, or portion thereof. In certain embodiments, an antibody, or binding portion thereof, binds specifically to a mammalian syndecan-1, or portion thereof, with a binding affinity (KD) of 10⁻⁵ M or less, 10⁻⁶ M or less, 10⁻⁷ M or less, 10⁻⁸ M or less, 50 nM or less, 10 nM or less, or 1 nM or less. In certain embodiments, an antibody, or binding portion thereof, binds specifically to a mammalian syndecan-1, or portion thereof, with a binding affinity (KD) from about 10⁻⁵ to 10⁻¹⁵ M, 10⁻⁶ to 10⁻¹⁵ M, 10⁻⁷ to 10⁻¹⁵ M, 10⁻⁹ to 10⁻¹⁵ M, 10⁻⁹ to 10⁻¹⁴ M, 10⁻⁹ to 10⁻¹³ M, or 10⁻⁹ to about 10⁻¹² M. In some embodiments an antibody, or binding portion thereof, binds specifically to an extracellular domain or extracellular region of a mammalian syndecan-1, or a portion thereof. In certain aspects, an antibody, or binding portion thereof, binds specifically to a wild-type syndecan-1 produced by a cell of an unaltered (non-genetically modified) mammal found in nature. In certain aspects an antibody, or binding portion thereof, binds specifically to a naturally occurring syndecan-1 variant. In certain aspects an antibody, or binding portion thereof, binds specifically to a syndecan-1 comprising one or more amino acid substitutions, additions or deletions. In certain embodiments an antibody, or binding portion thereof, binds specifically to a syndecan-1 produced and/or expressed on the surface of a cell of a human, non-human primate, dog, cat, or rodent (e.g., a mouse or rat). In certain embodiments, an antibody, or binding portion thereof, binds specifically to one or more syndecan-1 polypeptides, or a portion thereof (e.g., an extracellular domain), comprising an amino acid sequence of any one of SEQ ID NOs: 1 and 126 to 130. In certain embodiments, an antibody, or binding portion thereof, binds specifically to one or more syndecan-1 polypeptides, or a portion thereof, having an amino acid sequence of any one of SEQ ID NOs: 1 and 126 to 130 with a binding affinity (KD) of 50 nM or less, 10 nM or less, or 1 nM or less. In certain embodiments, an antibody, or binding portion thereof, binds specifically to a human syndecan-1. In certain embodiments, an antibody, or binding portion thereof, binds specifically to an extracellular domain of human syndecan-1. In certain embodiments, an antibody, or binding portion thereof, binds specifically to a human syndecan-1, and/or an extracellular domain thereof.

In certain embodiments, an antibody, or binding portion thereof, binds specifically to a polypeptide sequence comprising or consisting of the amino acid sequence of AGEGPKEGEAVVLP (SEQ ID NO:94) or GPKEGEAVVLP (SEQ ID NO:95). In certain embodiments, an antibody, or binding portion thereof, binds specifically to a polypeptide sequence comprising or consisting of the amino acid sequence of AGEGPKEGEAVVLP (SEQ ID NO:94) or GPKEGEAVVLP (SEQ ID NO:95) with a binding affinity (KD) of 10⁻⁵ M or less, 10⁻⁶ M or less, 10⁻⁷ M or less, 10⁻⁸ M or less, 50 nM or less, 10 nM or less, or 1 nM or less. In certain embodiments, an antibody, or binding portion thereof, binds specifically to a polypeptide sequence comprising or consisting of the amino acid sequence of GX₁KEX₂EAX₃VLP (SEQ ID NO:96), wherein X₁, X₂ and X₃ are selected from any amino acid. In some embodiments X₁ is selected from proline, alanine, cysteine, glycine, serine, threonine, and valine, and/or X₂ is selected from proline, alanine, cysteine, glycine, serine, threonine, and valine, and/or X₃ is selected from proline, alanine, cysteine, glycine, serine, threonine, valine, methionine, leucine, isoleucine and phenylalanine. In certain embodiments, an antibody, or binding portion thereof, binds specifically to a polypeptide sequence comprising or consisting of the amino acid sequence of GX₁KEX₂EAX₃VLP (SEQ ID NO:96) with a binding affinity (KD) of 50 nM or less, 10 nM or less, or 1 nM or less, where X₁ is selected from proline, alanine, cysteine, glycine, serine, threonine, and valine, X₂ is selected from proline, alanine, cysteine, glycine, serine, threonine, and valine, and X₃ is selected from proline, alanine, cysteine, glycine, serine, threonine, valine, methionine, leucine, isoleucine and phenylalanine. In certain embodiments, X₁ is proline, X₂ is selected from alanine, glycine, or serine and X₃ is selected from alanine, glycine, and valine.

### FGFRs

Aberrantly activated FGFRs have been implicated in several human malignancies and overexpression of FGFR3 is sufficient to induce oncogenic transformation in several animal models. Several attempts to generate antibodies and antibody drug conjugates (ADCs) that target FGFRs have been made. However, these antibodies often recognize only one isoform of the FGFR or display a significant difference in binding affinity among the different FGFR isoforms.

Presented herein are bi-specific binding agents that comprise a Fynomer that binds one or more splice forms of FGFR3 with high affinity and specificity. In some embodiments, the bi-specific binding agents are capable of being internalized into a target cell upon binding.

### Fynomers

In some embodiments, a Fynomer is a small antigen-binding polypeptide (e.g., from 5-10 kDa, in some embodiments about 7 kDa) comprising a non-immunoglobulin scaffold derived from an SH3 domain of the human proto-oncogene tyrosine-protein kinase Fyn (p59-FYN, Slk, Syn, MGC45350, Gene ID 2534). Accordingly, in certain embodiments a Fynomer is a single chain polypeptide comprising an N-terminus (N-terminal amino acid) and a C-terminus (C-terminal amino acid) that can be expressed from a nucleic acid sequence using recombinant technology or can be chemically synthesized (e.g., by using a suitable solid phase chemistry). Fyn SH3-derived Fynomers are known in the art and have been described e.g. in Grabulovski et al. (2007) JBC, 282, p. 3196-3204; WO 2008/022759; Bertschinger et al. (2007) Protein Eng. Des. Sel. 20(2):57-68; and Gebauer and Skerra (2009) Curr. Opinion in Chemical Biology 13:245-255. A Fynomer can comprise a polypeptide of 50 to 80 amino acids in length. In some embodiments, a Fynomer comprises a polypeptide of 50 to 70, or 60 to 70 amino acids in length. In some embodiments, a Fynomer comprises a polypeptide of 60, 61, 62, 63, 64, 65, 66, 67, 68, 69 or 70 amino acids in length. Fynomers can be engineered to bind with high affinity and specificity to an antigen target of choice often through random mutation of two variable loops (RT-loop and src-loop), while the surrounding sequences of the Fynomer provide a structural scaffold that is substantially conserved among Fynomer sequences. It is believed that the amino acid sequence of these two variable loops, and sometimes amino acids immediately adjacent to the variable loops, substantially contribute to the specificity and binding affinity of a Fynomer to a selected antigen target. Although Fynomer sequences outside of the variable loops primarily provide for the structural scaffold, it is understood that certain amino acids within the scaffold region can be substituted without substantial loss of structure or binding specificity.

The amino acid sequences of the Fynomers presented herein bind specifically to an FGFR3, or one or more specific isoforms, or variants thereof. In some embodiments, a Fynomer described herein binds specifically to FGFR3, or one or more specific isoforms, or variants thereof. In some embodiments, a Fynomer described herein binds specifically to fibroblast growth factor receptor 3, isoforms 3b (FGFR3b). In some embodiments, a Fynomer described herein binds specifically to fibroblast growth factor receptor 3, isoform 3c (FGFR3c). In some embodiments, a Fynomer described herein binds specifically to both FGFR3b and FGFR3c. In some embodiments, an FGFR3 is a mammalian FGFR3. In some embodiments, an FGFR3 is a human, mouse, rat or monkey FGFR3 protein, or an isoform thereof. In some embodiments, an FGFR3 is a human FGFR3, or an isoform thereof. The amino acid sequence of human FGFR3b is shown in SEQ ID NO:97 and the amino acid sequence of human FGFR3c is shown in SEQ ID NO:98. In some embodiments, a Fynomer described herein binds specifically to human FGFR3b and/or human FGFR3c.

In some embodiment, a Fynomer (e.g., a Fynomer portion of a bi-specific binding agent disclosed herein) binds to an FGFR3, or isoform thereof, with a K_{D} of 1×10⁻⁷ M or less, a K_{D} of 1×10⁻⁸ M or less, a K_{D} of 1×10⁻⁹ M or less, or with a K_{D} of 1×10⁻¹⁰ M or less. In some embodiments, a Fynomer (e.g., a Fynomer portion of a bi-specific binding agent disclosed herein) binds to one or both isoforms FGFR3b and/or FGFR3c with a K_{D} of 1×10⁻⁷ M or less, a K_{D} of 1×10⁻⁸ M or less, a K_{D} of 1×10⁻⁹ M or less, or with a K_{D} of 1×10⁻¹⁰ M or less. In some embodiment, a Fynomer binds to both isoforms, FGFR3b and FGFR3c with a K_{D} of 10⁻⁷ to 10⁻¹² M, a K_{D} of 10⁻⁸ to 10⁻¹² M, or a K_{D} of 10⁻⁹ to 10⁻¹² M. In some embodiments, a Fynomer binds specifically to one or both isoforms FGFR3b and/or FGFR3c, and does not bind substantially to other related proteins such as FGFR1, FGFR2, or FGFR4. In some embodiments, a Fynomer that does not substantially bind to an FGFR, is a Fynomer that does not demonstrate any detectable specific binding, or binds to an FGFR with a K_{D} of greater than 5×10⁻⁶ M.

In some embodiments, a Fynomer portion of a bi-specific binding agent disclosed herein, binds to an FGFR3, or isoform thereof, in the presence of receptor bound ligand (e.g., FGF1). Accordingly, in some embodiments, a Fynomer portion of a bi-specific binding agent disclosed herein does not block, abrogate, or inhibit binding of an FGFR3 ligand (e.g., FGF1) to an FGFR3. In some embodiments, a Fynomer portion of a bi-specific binding agent disclosed herein does not block, abrogate, or inhibit binding of an FGFR3 ligand (e.g., FGF1) to an FGFR3. In some embodiments, a bi-specific binding agent disclosed herein does not block, abrogate, or inhibit binding of an FGFR3 ligand (e.g., FGF1) to an FGFR3. In some embodiments, a Fynomer portion of a bi-specific binding agent disclosed herein, and/or a bi-specific binding agent disclosed herein, binds to an FGFR3, or isoform thereof, in the presence of receptor bound ligand (e.g., FGF1).

In certain embodiments, a Fynomer comprises a polypeptide having an amino acid sequence at least 80%, at least 85%, at least 90%, at least 95% or 100% identical to the amino acid sequence GVTLFVALYDYEVYGPTPMLSFHKGEKFQIL(X₁)(X₂)(X₃)(X₄)GPYWEARSL(X₅)TGETG(X₆)IPSNYVAPVDSIQ (SEQ ID NO:99), where amino acids X₁ to X₆ are independently selected from any amino acid, and the Fynomer binds specifically to an FGFR3. In some embodiment, a Fynomer having a sequence at least 80%, at least 85%, at least 90%, at least 95% or 100% identical to the amino acid sequence of SEQ ID NO:99 binds specifically to an FGFR3 (e.g., human FGFR3). In some embodiment, a Fynomer having a sequence at least 80%, at least 85%, at least 90%, at least 95% or 100% identical to the amino acid sequence of SEQ ID NO:99 binds specifically to both FGFR3b and FGFR3c. The N-terminal RT loop and C-terminal src-loop of the Fynomer sequences provided herein are often underlined solely for purposes of illustration. In certain embodiments, a Fynomer comprises a polypeptide having an amino acid sequence at least 80%, at least 85%, at least 90%, at least 95% or 100% identical to the amino acid sequence of SEQ ID NO:99, where (i) amino acids X₁ to X₆ are independently selected from any amino acid, and (ii) the amino acid sequence EVYGPTPM (SEQ ID NO:100) at amino acid positions 12 to 19 of SEQ ID NO:99 and amino acids P and Y at amino acid positions 37 and 38 of SEQ ID NO:99 are conserved.

In certain embodiments X₁ is selected from N, R, H and K. In certain embodiments X₁ is N, R or K. In certain embodiments X₂ is selected from S, G, A, V, and P. In certain embodiments X₂ is selected from S, G, A, V, P, and any basic amino acid. In certain embodiments X₂ is S, G, K or R. In certain embodiments X₃ is S, G, A, V, or P. In certain embodiments X₃ is S or G. In certain embodiments X₄ is selected from any charged, basic or acidic amino acid. In certain embodiments X₄ is selected from S, G, A, V and P. In certain embodiments X₄ is E, Q, D, S or K. In certain embodiments X₅ is selected from S, G, A, V, P, S and T. In certain embodiments X₅ is T or A. In certain embodiments X₆ is selected from any hydrophobic amino acid. In certain embodiments X₆ is selected from any polar amino acid. In certain embodiments X₆ is selected from Q, N, H, S, T, Y, C, W, A, L, V and I. In certain embodiments X₆ is Y, W or L.

In certain embodiments X₁ is N, R or K; X₂ is S, G, K or R; X₃ is S or G; X₄ is E, Q, D, S or K; X₅ is T or A; and/or X₆ is Y, W or L.

In some embodiments, a Fynomer comprises an amino acid sequence at least 95% identical to the amino acid sequence of SEQ ID NO:99, where (i) amino acid positions X₁ to X₆ may be any amino acid sequence, (ii) the identity determination excludes amino acid positions X₁ to X₆, (iii) and the amino acid sequence EVYGPTPM (SEQ ID NO:100) in amino acid positions 12 to 19 of SEQ ID NO:99 and amino acids P and Y in amino acid positions 37 and 38 of SEQ ID NO:99 are conserved, and (iv) the Fynomer binds specifically to an FGFR3.

In some embodiments, a Fynomer comprises an amino acid sequence at least 95% identical to the amino acid sequence of SEQ ID NO:99, where (i) X₁ is N, R or K; X₂ is S, G, K or R; X₃ is S or G; X₄ is E, Q, D, S or K; X₅ is T or A; and X₆ is Y, W or L, (ii) the identity determination excludes amino acid positions X₁ to X₆, (iii) and the amino acid sequence EVYGPTPM (SEQ ID NO:100) in amino acid positions 12 to 19 of SEQ ID NO:99 and amino acids P and Y in amino acid positions 37 and 38 of SEQ ID NO:99 are conserved, and (iv) the Fynomer binds specifically to an FGFR3.

In certain embodiments, a Fynomer comprises a polypeptide having an amino acid sequence at least 80%, at least 85%, at least 90%, at least 95% or 100% identical to the amino acid sequence of GVTLFVALYDYEVYGPTPMLSFHKGEKFQILNSSEGPYWEARSLTTGETGLIPSNYVAPV DSIQ (SEQ ID NO:101; sometimes referred to herein as FF2L4C3), where the Fynomer binds specifically to an FGFR3. In certain embodiments, a Fynomer comprises a polypeptide having an amino acid sequence at least 80%, at least 85%, at least 90%, at least 95% or 100% identical to the amino acid sequence of SEQ ID NO:101, where the amino acid sequence EVYGPTPM (SEQ ID NO:100) at amino acid positions 12 to 19 of SEQ ID NO:101 and amino acids P and Y at amino acid positions 37 and 38 of SEQ ID NO:101 are conserved, and the Fynomer binds specifically to an FGFR3. In certain embodiments, a Fynomer comprises a polypeptide having an amino acid sequence at least 80%, at least 85%, at least 90%, at least 95% or 100% identical to the amino acid sequence of SEQ ID NO:101, where the amino acid sequence EVYGPTPM (SEQ ID NO:100) at amino acid positions 12 to 19 of SEQ ID NO:101, and the amino acid sequence NSSEGPY (SEQ ID NO:102) at amino acid positions 32 to 38 of SEQ ID NO:101 are conserved, and the Fynomer binds specifically to an FGFR3. In some embodiments, a Fynomer having an amino acid sequence at least 80%, at least 85%, at least 90%, or at least 95% identical to the amino acid sequence of SEQ ID NO:101, where the Fynomer binds specifically to an FGFR3 (e.g., FGFR3b and/or FGFR3c), is a Fynomer comprising 0 to 12, 0 to 8, 0 to 5, or 1 to 2 amino acid substitutions, additions and/or deletions that do not ablate or significantly reduce the ability of the Fynomer to specifically bind to an FGFR3. A significant reduction in specific binding is a reduction of binding affinity (i.e., K_{D}) in excess of 20% of the binding affinity of the Fynomer of SEQ ID NO:101 to human FGFR3b or FGFR3c. One of skill in the art can readily determine the binding affinity of a Fynomer to FGFR3b or FGFR3c using routine methods and such a determination would not require undue experimentation. Accordingly, one of skill in the art can readily determine how to make a Fynomer having an amino acid sequence at least 80%, at least 85%, at least 90%, or at least 95% identical to the amino acid sequence of SEQ ID NO:101, where the Fynomer binds specifically to an FGFR3. In certain embodiments, a Fynomer comprises or consists of the amino acid sequence of SEQ ID NO:101.

In certain embodiments, a Fynomer comprises a polypeptide having an amino acid sequence at least 80%, at least 85%, at least 90%, at least 95% or 100% identical to the amino acid sequence of GVTLFVALYDYEVYGPTPMLSFHKGEKFQILRGGQGPYWEARSLTTGETGLIPSNYVAP VDSIQ (SEQ ID NO:103); sometimes referred to herein as FF44L65G12) and the Fynomer binds specifically to an FGFR3. In certain embodiments, a Fynomer comprises a polypeptide having an amino acid sequence at least 80%, at least 85%, at least 90%, at least 95% or 100% identical to the amino acid sequence of SEQ ID NO:103, where the amino acid sequence EVYGPTPM (SEQ ID NO:100) at amino acid positions 12 to 19 of SEQ ID NO:103 and amino acids P and Y at amino acid positions 37 and 38 of SEQ ID NO:103 are conserved, and the Fynomer binds specifically to an FGFR3. In certain embodiments, a Fynomer comprises a polypeptide having an amino acid sequence at least 80%, at least 85%, at least 90%, at least 95% or 100% identical to the amino acid sequence of SEQ ID NO:103, where the amino acid sequence EVYGPTPM (SEQ ID NO:100) at amino acid positions 12 to 19 of SEQ ID NO:103, and the amino acid sequence RGGQGPY (SEQ ID NO:104) at amino acid positions 32 to 38 of SEQ ID NO:103 are conserved, and the Fynomer binds specifically to an FGFR3. In some embodiments, a Fynomer having an amino acid sequence at least 80%, at least 85%, at least 90%, or at least 95% identical to the amino acid sequence of SEQ ID NO:103, where the Fynomer binds specifically to an FGFR3 (e.g., FGFR3b and/or FGFR3c), is a Fynomer comprising 0 to 12, 0 to 8, 0 to 5, or 1 to 2 amino acid substitutions, additions and/or deletions that do not ablate or significantly reduce the ability of the Fynomer to specifically bind to an FGFR3. A significant reduction in specific binding is a reduction of binding affinity (i.e., K_{D}) in excess of 20% of the binding affinity of the Fynomer of SEQ ID NO:103 to human FGFR3b or FGFR3c. One of skill in the art can readily determine the binding affinity of a Fynomer to FGFR3b or FGFR3c using routine methods and such a determination would not require undue experimentation. Accordingly, one of skill in the art can readily determine how to make a Fynomer having an amino acid sequence at least 80%, at least 85%, at least 90%, or at least 95% identical to the amino acid sequence of SEQ ID NO:103, where the Fynomer binds specifically to an FGFR3. In certain embodiments, a Fynomer comprises or consists of the amino acid sequence of SEQ ID NO:103.

In certain embodiments, a Fynomer comprises a polypeptide having an amino acid sequence at least 80%, at least 85%, at least 90%, at least 95% or 100% identical to the amino acid sequence of GVTLFVALYDYEVYGPTPMLSFHKGEKFQILRGGDGPYWEARSLTTGETGLIPSNYVAP VDSIQ (SEQ ID NO:105; sometimes referred to herein as FF44L65G7) and the Fynomer binds specifically to an FGFR3. In certain embodiments, a Fynomer comprises a polypeptide having an amino acid sequence at least 80%, at least 85%, at least 90%, at least 95% or 100% identical to the amino acid sequence of SEQ ID NO:105, where the amino acid sequence EVYGPTPM (SEQ ID NO:100) at amino acid positions 12 to 19 of SEQ ID NO:105 and amino acids P and Y at amino acid positions 37 and 38 of SEQ ID NO:105 are conserved, and the Fynomer binds specifically to an FGFR3. In certain embodiments, a Fynomer comprises a polypeptide having an amino acid sequence at least 80%, at least 85%, at least 90%, at least 95% or 100% identical to the amino acid sequence of SEQ ID NO:105, where the amino acid sequence EVYGPTPM (SEQ ID NO:100) at amino acid positions 12 to 19 of SEQ ID NO:105, and amino acids RGGDGPY (SEQ ID NO:106) at amino acid positions 32 to 38 of SEQ ID NO:105 are conserved, and the Fynomer binds specifically to an FGFR3. In some embodiments, a Fynomer having an amino acid sequence at least 80%, at least 85%, at least 90%, or at least 95% identical to the amino acid sequence of SEQ ID NO:105, where the Fynomer binds specifically to an FGFR3 (e.g., FGFR3b and/or FGFR3c), is a Fynomer comprising 0 to 12, 0 to 8, 0 to 5, or 1 to 2 amino acid substitutions, additions and/or deletions that do not ablate or significantly reduce the ability of the Fynomer to specifically bind to an FGFR3. A significant reduction in specific binding is a reduction of binding affinity (i.e., K_{D}) in excess of 20% of the binding affinity of the Fynomer of SEQ ID NO:105 to human FGFR3b or FGFR3c. One of skill in the art can readily determine the binding affinity of a Fynomer to FGFR3b or FGFR3c using routine methods and such a determination would not require undue experimentation. Accordingly, one of skill in the art can readily determine how to make a Fynomer having an amino acid sequence at least 80%, at least 85%, at least 90%, or at least 95% identical to the amino acid sequence of SEQ ID NO:105, where the Fynomer binds specifically to an FGFR3. In certain embodiments, a Fynomer comprises or consists of the amino acid sequence of SEQ ID NO:105).

In certain embodiments, a Fynomer comprises a polypeptide having an amino acid sequence at least 80%, at least 85%, at least 90%, at least 95% or 100% identical to the amino acid sequence of GVTLFVALYDYEVYGPTPMLSFHKGEKFQILKGGSGPYWEARSLTTGETGLIPSNYVAP VDSIQ (SEQ ID NO:107; sometimes referred to herein as FF48L66G7 or “G7”) and the Fynomer binds specifically to an FGFR3. In certain embodiments, a Fynomer comprises a polypeptide having an amino acid sequence at least 80%, at least 85%, at least 90%, at least 95% or 100% identical to the amino acid sequence of SEQ ID NO:107, where the amino acid sequence EVYGPTPM (SEQ ID NO:100) at amino acid positions 12 to 19 of SEQ ID NO:107 and amino acids P and Y at amino acid positions 37 and 38 of SEQ ID NO:107 are conserved, and the Fynomer binds specifically to an FGFR3. In certain embodiments, a Fynomer comprises a polypeptide having an amino acid sequence at least 80%, at least 85%, at least 90%, at least 95% or 100% identical to the amino acid sequence of SEQ ID NO:107, where the amino acid sequence EVYGPTPM (SEQ ID NO:100) at amino acid positions 12 to 19 of SEQ ID NO:107, and amino acids KGGSGPY (SEQ ID NO:108) at amino acid positions 32 to 38 of SEQ ID NO:107 are conserved, and the Fynomer binds specifically to an FGFR3. In some embodiments, a Fynomer having an amino acid sequence at least 80%, at least 85%, at least 90%, or at least 95% identical to the amino acid sequence of SEQ ID NO:107, where the Fynomer binds specifically to an FGFR3 (e.g., FGFR3b and/or FGFR3c), is a Fynomer comprising 0 to 12, 0 to 8, 0 to 5, or 1 to 2 amino acid substitutions, additions and/or deletions that do not ablate or significantly reduce the ability of the Fynomer to specifically bind to an FGFR3. A significant reduction in specific binding is a reduction of binding affinity (i.e., K_{D}) in excess of 20% of the binding affinity of the Fynomer of SEQ ID NO:107 to human FGFR3b or FGFR3c. One of skill in the art can readily determine the binding affinity of a Fynomer to FGFR3b or FGFR3c using routine methods and such a determination would not require undue experimentation. Accordingly, one of skill in the art can readily determine how to make a Fynomer having an amino acid sequence at least 80%, at least 85%, at least 90%, or at least 95% identical to the amino acid sequence of SEQ ID NO:107, where the Fynomer binds specifically to an FGFR3. In certain embodiments, a Fynomer comprises or consists of the amino acid sequence of SEQ ID NO:107.

In certain embodiments, a Fynomer comprises a polypeptide having an amino acid sequence at least 80%, at least 85%, at least 90%, at least 95% or 100% identical to the amino acid sequence of GVTLFVALYDYEVYGPTPMLSFHKGEKFQILRKGKGPYWEARSLATGETGLIPSNYVAP VDSIQ (SEQ ID NO:109; sometimes referred to herein as FF43L65D5) and the Fynomer binds specifically to an FGFR3. In certain embodiments, a Fynomer comprises a polypeptide having an amino acid sequence at least 80%, at least 85%, at least 90%, at least 95% or 100% identical to the amino acid sequence of SEQ ID NO:109, where the amino acid sequence EVYGPTPM (SEQ ID NO:100) at amino acid positions 12 to 19 of SEQ ID NO:109 and amino acids P and Y at amino acid positions 37 and 38 of SEQ ID NO:109 are conserved, and the Fynomer binds specifically to an FGFR3. In certain embodiments, a Fynomer comprises a polypeptide having an amino acid sequence at least 80%, at least 85%, at least 90%, at least 95% or 100% identical to the amino acid sequence of SEQ ID NO:109, where the amino acid sequence EVYGPTPM (SEQ ID NO:100) at amino acid positions 12 to 19 of SEQ ID NO:109 and amino acids RKGKGPY (SEQ ID NO:110) at amino acid positions 32 to 38 of SEQ ID NO:109 are conserved, and the Fynomer binds specifically to an FGFR3. In some embodiments, a Fynomer having an amino acid sequence at least 80%, at least 85%, at least 90%, or at least 95% identical to the amino acid sequence of SEQ ID NO:109, where the Fynomer binds specifically to an FGFR3 (e.g., FGFR3b and/or FGFR3c), is a Fynomer comprising 0 to 12, 0 to 8, 0 to 5, or 1 to 2 amino acid substitutions, additions and/or deletions that do not ablate or significantly reduce the ability of the Fynomer to specifically bind to an FGFR3. A significant reduction in specific binding is a reduction of binding affinity (i.e., K_{D}) in excess of 20% of the binding affinity of the Fynomer of SEQ ID NO:109 to human FGFR3b or FGFR3c. One of skill in the art can readily determine the binding affinity of a Fynomer to FGFR3b or FGFR3c using routine methods and such a determination would not require undue experimentation. Accordingly, one of skill in the art can readily determine how to make a Fynomer having an amino acid sequence at least 80%, at least 85%, at least 90%, or at least 95% identical to the amino acid sequence of SEQ ID NO:109, where the Fynomer binds specifically to an FGFR3. In certain embodiments, a Fynomer comprises or consists of the amino acid sequence of SEQ ID NO:109.

In certain embodiments, a Fynomer comprises a polypeptide having an amino acid sequence at least 80%, at least 85%, at least 90%, at least 95% or 100% identical to the amino acid sequence of GVTLFVALYDYEVYGPTPMLSFHKGEKFQILRRGSGPYWEARSLTTGETGLIPSNYVAP VDSIQ (SEQ ID NO:111; sometimes referred to herein as FF44L65B7) and the Fynomer binds specifically to an FGFR3, or an isoform thereof (e.g., FGFR3b and/or FGFR3c). In certain embodiments, a Fynomer comprises a polypeptide having an amino acid sequence at least 80%, at least 85%, at least 90%, or at least 95% identical to the amino acid sequence of SEQ ID NO:111, where the amino acid sequence EVYGPTPM (SEQ ID NO:100) at amino acid positions 12 to 19 of SEQ ID NO:111 and amino acids P and Y at amino acid positions 37 and 38 of SEQ ID NO:111 are conserved, and the Fynomer binds specifically to an FGFR3 (e.g., FGFR3b and/or FGFR3c). In certain embodiments, a Fynomer comprises a polypeptide having an amino acid sequence at least 80%, at least 85%, at least 90%, or at least 95% identical to the amino acid sequence of SEQ ID NO:111, where the amino acid sequence EVYGPTPM (SEQ ID NO:100) at amino acid positions 12 to 19 of SEQ ID NO:111 and amino acids RRGSGPY (SEQ ID NO:112) at amino acid positions 32 to 38 of SEQ ID NO:111 are conserved, and the Fynomer binds specifically to an FGFR3 (e.g., FGFR3b and/or FGFR3c). In some embodiments, a Fynomer having an amino acid sequence at least 80%, at least 85%, at least 90%, or at least 95% identical to the amino acid sequence of SEQ ID NO:111, where the Fynomer binds specifically to an FGFR3 (e.g., FGFR3b and/or FGFR3c), is a Fynomer comprising 0 to 12, 0 to 8, 0 to 5, or 1 to 2 amino acid substitutions, additions and/or deletions that do not ablate or significantly reduce the ability of the Fynomer to specifically bind to an FGFR3. A significant reduction in specific binding is a reduction of binding affinity (i.e., K_{D}) in excess of 20% of the binding affinity of the Fynomer of SEQ ID NO:111 to human FGFR3b or FGFR3c. One of skill in the art can readily determine the binding affinity of a Fynomer to FGFR3b or FGFR3c using routine methods and such a determination would not require undue experimentation. Accordingly, one of skill in the art can readily determine how to make a Fynomer having an amino acid sequence at least 80%, at least 85%, at least 90%, or at least 95% identical to the amino acid sequence of SEQ ID NO:111, where the Fynomer binds specifically to an FGFR3. In certain embodiments, a Fynomer comprises or consists of the amino acid sequence of SEQ ID NO:111.

In certain embodiments, a Fynomer comprises a polypeptide having an amino acid sequence at least 80%, at least 85%, at least 90%, at least 95% or 100% identical to the amino acid sequence of GVTLFVALYDYEVMSTTALSFHKGEKFQILSQSPHGQYWEARSLTTGETG(X₇)IPSNYVA PVDSIQ (SEQ ID NO: 113), wherein the amino acid (X₇) is selected from any amino acid. In certain embodiments X₇ is selected from any hydrophobic amino acid. In certain embodiments X₇ is selected from any polar amino acid. In certain embodiments X₇ is selected from Q, N, H, S, T, Y, C, W, A, L, V and I. In certain embodiments X₇ is Y, W or L. In certain embodiments X₇ is W.

In certain embodiments, a Fynomer comprises a polypeptide having an amino acid sequence at least 80%, at least 85%, at least 90%, at least 95% or 100% identical to the amino acid sequence of SEQ ID NO:113 where (i) the amino acid at position X₇ may be any amino acid; (ii) the identity determination excludes the amino acid position X₇, (iii) the amino acid sequence EVMSTTA (SEQ ID NO: 114) at amino acid positions 12 to 18 of SEQ ID NO: 113 and SQSPH (SEQ ID NO: 115) at amino acid positions 31 to 35 of SEQ ID NO: 113 are conserved, (iv) the amino acids Q and Y at amino acid positions 37 and 38 of SEQ ID NO: 113 are conserved, and (v) the Fynomer binds specifically to an FGFR3 (e.g., FGFR3b, and/or FGFR3c).

In certain embodiments, a Fynomer comprises a polypeptide having an amino acid sequence at least 80%, at least 85%, at least 90%, at least 95% or 100% identical to the amino acid sequence of GVTLFVALYDYEVMSTTALSFHKGEKFQILSQSPHGQYWEARSLTTGETGWIPSNYVAP VDSIQ (SEQ ID NO: 116; sometimes referred to herein as FF40L54A5) and the Fynomer binds specifically to an FGFR3. In certain embodiments, a Fynomer comprises a polypeptide having an amino acid sequence at least 80%, at least 85%, at least 90%, at least 95% or 100% identical to the amino acid sequence of SEQ ID NO:116, where (i) the amino acid sequence EVMSTTA (SEQ ID NO: 114) at amino acid positions 12 to 18 of SEQ ID NO: 116 are conserved, (ii) the amino acid sequence SQSPH (SEQ ID NO:115) at amino acid positions 31 to 35 of SEQ ID NO:116 are conserved, (iii) the amino acids Q and Y at amino acid positions 37 and 38 of SEQ ID NO: 116 are conserved, and the Fynomer binds specifically to an FGFR3. In certain embodiments, a Fynomer comprises a polypeptide having an amino acid sequence at least 80%, at least 85%, at least 90%, at least 95% or 100% identical to the amino acid sequence of SEQ ID NO:116, where (i) the amino acid sequence EVMSTTA (SEQ ID NO:114) at amino acid positions 12 to 18 of SEQ ID NO:116 is conserved, (ii) the amino acid sequence SQSPHGQY (SEQ ID NO: 117) at amino acid positions 31 to 38 of SEQ ID NO: 116 are conserved, and the Fynomer binds specifically to an FGFR3. In some embodiments, a Fynomer having an amino acid sequence at least 80%, at least 85%, at least 90%, or at least 95% identical to the amino acid sequence of SEQ ID NO:116, where the Fynomer binds specifically to an FGFR3 (e.g., FGFR3b and/or FGFR3c), is a Fynomer comprising 0 to 12, 0 to 8, 0 to 5, or 1 to 2 amino acid substitutions, additions and/or deletions that do not ablate or significantly reduce the ability of the Fynomer to specifically bind to an FGFR3. A significant reduction in specific binding is a reduction of binding affinity (i.e., K_{D}) in excess of 20% of the binding affinity of the Fynomer of SEQ ID NO:116 to human FGFR3b or FGFR3c. One of skill in the art can readily determine the binding affinity of a Fynomer to FGFR3b or FGFR3c using routine methods and such a determination would not require undue experimentation. Accordingly, one of skill in the art can readily determine how to make a Fynomer having an amino acid sequence at least 80%, at least 85%, at least 90%, or at least 95% identical to the amino acid sequence of SEQ ID NO:116, where the Fynomer binds specifically to an FGFR3. In certain embodiments, a Fynomer comprises or consists of a polypeptide having an amino acid sequence of SEQ ID NO:116.

The Fynomer portion of a bi-specific binding agent can be attached to the antibody portion of a bi-specific binding agent at any suitable location using any suitable method. A Fynomer can be attached covalently or non-covalently to an antibody, or binding portion thereof. A Fynomer can be attached to the N-terminus (N-terminal amino acid) and/or C-terminus (C-terminal amino acid) of an antibody, or antigen binding portion thereof. In some embodiments, a Fynomer is attached to the N-terminus of a heavy chain and/or to the N-terminus of a light chain of an antibody, or antigen binding portion thereof. In some embodiments, a Fynomer is attached to the C-terminus of a heavy chain and/or to the C-terminus of a light chain of an antibody, or antigen binding portion thereof. In some embodiments, a Fynomer is attached to suitable location within a constant domain of an antibody, or antigen binding portion thereof.

In certain embodiments, a Fynomer is attached to an antibody, or within an antibody, by a peptide bond. In some embodiments, a bi-specific binding agent comprises a fusion protein comprising a Fynomer and a polypeptide of an antibody (e.g., a heavy chain, light chain, or single chain), where the Fynomer and the antibody polypeptide are joined by a peptide bond. Therefore, in some embodiments, a bi-specific binding agent is made using recombinant technology where a nucleic acid is configured to express a polypeptide comprising a Fynomer and an antibody, or portion thereof (e.g., a light chain or heavy chain) as a single polypeptide. In some embodiments, a bi-specific binding agent comprises a Fynomer and an antibody, or antigen binding portion thereof, where the C-terminal amino acid of the Fynomer is linked to an N-terminal amino acid of the antibody (e.g., an N-terminal amino acid of the heavy chain, or N-terminal amino acid of the light chain) by a peptide bond. In some embodiments, a bi-specific binding agent comprises a Fynomer and an antibody, or antigen binding portion thereof, where the N-terminal amino acid of the Fynomer is linked to a C-terminal amino acid of the antibody (e.g., a C-terminal amino acid of a heavy chain, or C-terminal amino acid of a light chain) by a peptide bond. In certain embodiments, a Fynomer peptide is integrated within a polypeptide of an antibody.

In some embodiments, a bi-specific binding agent comprises one or more Fynomers that bind specifically to an FGFR3. For example, a bi-specific binding agent may comprise an antibody having two heavy chains and two light chains and a Fynomer attached to the N-terminus of one or both heavy chains, a Fynomer attached to the N-terminus of one or both light chains, a Fynomer attached to the C-terminus of one or both heavy chains and/or a Fynomer attached to the C-terminus of one or both light chains of the antibody. Accordingly, in certain embodiments, a bi-specific binding agent comprises 1 to 12, 1 to 8 or 1 to 4 Fynomers. In some embodiments, a bi-specific binding agent comprise 1, 2, 3, 4, 5, 6, 7 or 8 Fynomers.

In some embodiments, a bi-specific binding agent comprises a linker between a Fynomer and an antibody. Non-limiting examples of a suitable linker include amino acids, peptides (e.g., 2 or more amino acids), an optionally substituted C1-C50 alkyl, optionally substituted C2-C50 alkenyl, alkynyl, acyl, acyloxy, alkoxy, aryloxy, cycloalkyl, cycloalkenyl, cycloalkoxy, aryl, aminocarbonyl, azido, carboxy, silanes, thiols, sulfoxide, sulfones, sulfonate ester, cyano, amide, amino, ester, phosphonic acid, polyethylene glycol (PEG), the like, derivatives thereof, polymers thereof and combinations thereof. Methods of attaching two or more molecules using a linker are known to those skilled in the art and such methods are sometimes referred to as “crosslinking”.

In some embodiments, a linker comprises a peptide comprising two or more amino acids, 2 to 100 amino acids, 5 to 100 amino acids, 2 to 50 amino acids, 5 to 50 amino acids, 2 to 25 amino acids, 5 to 25 amino acids, 2 to 20 amino acids, 5 to 20 amino acids, 2 to 10 amino acids or 5 to 10 amino acids. In some embodiments, a linker comprises a peptide comprising 2, 3, 4 or 5 amino acids. In some embodiments, a linker comprises a motif of (GGGGS)X (X=1, 2, 3, 4, 5, 6, 7, 8, 9 or 10) or of GGGGSGGGGSGGGGS.

The term “percent identical” or “percent identity” refers to sequence identity between two amino acid sequences. In some embodiments, identity is determined by comparing a position in each sequence which may be aligned for purposes of comparison. When an equivalent position in the compared sequences is occupied by the same amino acid, then the molecules are identical at that position. When the equivalent site is occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. In some embodiments an alignment program that permits gaps in the sequence is utilized to align the sequences. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term “binds specifically” refers to a bi-specific binding agent, Fynomer, antibody, or portion thereof, that binds to a target protein, peptide or epitope in preference to binding other molecules or other peptides as determined by, for example, a suitable in vitro assay (e.g., an ELISA, Immunoblot, Flow cytometry, and the like). A specific binding interaction discriminates over non-specific binding interactions by about 2-fold or more, often about 10-fold or more, and sometimes about 100-fold or more, 1000-fold or more, 10,000-fold or more, 100,000-fold or more, or 1,000,000-fold or more.

In some embodiments an antibody, or binding portion thereof, that binds specifically to syndecan-1, or a portion thereof, is an antibody, or binding portion thereof, that binds syndecan-1, or a portion thereof (e.g., an extracellular domain of syndecan-1), with a binding affinity constant (KD) equal to or less than 100 nM, equal to or less than 50 nM, equal to or less than 25 nM, equal to or less than 10 nM, equal to or less than 5 nM, equal to or less than 1 nM, equal to or less than 900 pM, equal to or less than 800 pM, equal to or less than 750 pM, equal to or less than 700 pM, equal to or less than 600 pM, equal to or less than 500 pM, equal to or less than 400 pM, equal to or less than 300 pM, equal to or less than 200 pM, or equal to or less than 100 pM. In some embodiments an antibody, or binding portion thereof, that binds specifically to syndecan-1, or a portion thereof, is an antibody, or binding portion thereof, that binds human syndecan-1, or a portion thereof (e.g., an extracellular domain of human syndecan-1), with a binding affinity constant (KD) equal to or less than 100 nM, equal to or less than 50 nM, equal to or less than 25 nM, equal to or less than 10 nM, equal to or less than 5 nM, equal to or less than 1 nM, equal to or less than 900 pM, equal to or less than 800 pM, equal to or less than 750 pM, equal to or less than 700 pM, equal to or less than 600 pM, equal to or less than 500 pM, equal to or less than 400 pM, equal to or less than 300 pM, equal to or less than 200 pM, or equal to or less than 100 pM. In some embodiments an antibody, or binding portion thereof, that binds specifically to syndecan-1, or a portion thereof, is an antibody, or binding portion thereof, that binds specifically to syndecan-1, or a portion thereof, derived from a non-human species (e.g., a non-human primate, or rodent; e.g., a mouse or rat), with a binding affinity constant (KD) equal to or less than 100 nM, equal to or less than 50 nM, equal to or less than 25 nM, equal to or less than 10 nM, equal to or less than 5 nM, equal to or less than 1 nM, equal to or less than 900 pM, equal to or less than 800 pM, equal to or less than 750 pM, equal to or less than 700 pM, equal to or less than 600 pM, equal to or less than 500 pM, equal to or less than 400 pM, equal to or less than 300 pM, equal to or less than 200 pM, or equal to or less than 100 pM. In certain embodiments, an antibody, or binding portion thereof, disclosed herein binds specifically to human syndecan-1, or a portion thereof, and binds specifically to syndecan-1, or a portion thereof, derived from a non-human primate. In certain embodiments, an antibody, or binding portion thereof, disclosed herein binds specifically to human syndecan-1, or a portion thereof, and binds specifically to syndecan-1, or a portion thereof, derived from a rodent (e.g., a mouse or rat). In certain embodiments, an antibody, or binding portion thereof, (i) binds specifically to a human syndecan-1, or portion thereof (e.g., an extracellular domain of human syndecan-1) with a binding affinity (KD) of 10 nM or less, or 1 nM or less, and (ii) binds specifically to a rat or mouse syndecan-1, or portion thereof (e.g., an extracellular domain of rat or mouse syndecan-1) with a binding affinity (KD) of 100 nM or less, 90 nM or less, 80 nM or less, 70 nM or less, 60 nM or less, 50 nM or less, 40 nM or less, 30 nM or less, 20 nM or less or 10 nM or less.

In certain embodiments, a bi-specific binding agent comprises an antibody, or binding portion thereof that competes for binding with an anti-syndecan-1 antibody described herein to syndecan-1, or to a polypeptide comprising the amino acid sequence of SEQ ID NO:94, 95 or 96. In certain embodiments, a bi-specific binding agent comprises an antibody portion that competes for binding with an anti-syndecan-1 antibody described herein to syndecan-1, where the anti-syndecan-1 antibody described herein comprises one or more CDRs shown in Tables 1-10, or one or more CDRs that are substantially similar to those shown in Tables 1-10. In certain embodiments, a bi-specific binding agent comprises an antibody portion that competes for binding with an anti-syndecan-1 antibody described herein to syndecan-1, where the anti-syndecan-1 antibody described herein comprises a CDR-L1 selected from Table 1, a CDR-L2 selected from Table 2, a CDR-L3 selected from Table 3, a CDR-H1 selected from Table 6, a CDR-H2 selected from Table 7, and a CDR-H3 selected from Table 8. In certain embodiments, a bi-specific binding agent comprises an antibody portion that competes for binding with an anti-syndecan-1 antibody described herein to syndecan-1, where the anti-syndecan-1 antibody described herein comprises a CDR-L1, a CDR-L2 a CDR-L3, a CDR-H1, a CDR-H2, and a CDR-H3 comprising the amino acid sequences of SEQ ID NOs:2, 17, 27, 47, 61 and 73, respectively. In certain embodiments, a bi-specific binding agent comprises an antibody, or antibody portion that binds to the same epitope of syndecan-1 as an anti-syndecan-1 antibody described herein. In certain embodiments, a bi-specific binding agent comprises an antibody, or antibody portion that binds specifically to the same epitope of syndecan-1 as an anti-syndecan-1 antibody described herein. In certain embodiments, a bi-specific binding agent comprises an antibody, or antibody portion that binds specifically to the amino acid sequence of SEQ ID NO:94, 95 or 96.

In certain embodiments, a bi-specific agent comprises an antibody portion having one or more CDR sequences that are distinct and/or different from an anti-syndecan-1 antibody described herein, where the bi-specific agent competes for binding to syndecan-1 with an anti-syndecan-1 antibody described herein.

Methods of identifying antibodies that compete for binding to an antigen are known. Any suitable method can be used to determine if a bi-specific agent, or antibody portion thereof competes for binding to syndecan-1 with an anti-syndecan-1 antibody described herein. For example, ELISA-based methods can be used where a syndecan-1 antigen, or portion thereof, is coated on a 96-well plate. A bi-specific agent is added and allowed to bind to the coated antigen. The plate is then washed and an anti-syndecan-1 antibody described herein is added to the plate and allowed to bind. The amount of binding of the anti-syndecan-1 antibody described herein is measured in the presence or absence of the bi-specific agent to determine if the bi-specific agent competes for binding with the anti-syndecan-1 antibody described herein. Other suitable methods known in the art can also be used.

In some embodiments a bi-specific binding agent comprises a label. As used herein, the terms “label” or “labeled” refers to incorporation of a detectable marker, e.g., by incorporation of a labeled amino acid or attachment to a polypeptide of biotin moieties that can be detected by labeled avidin (e.g., streptavidin containing a fluorescent marker or enzymatic activity that can be detected by optical or colorimetric methods). In certain embodiments, a label or marker can be attached to a bi-specific binding agent to generate a diagnostic agent. A bi-specific binding agent can be attached covalently or non-covalently to any suitable label or marker. Various methods of labeling polypeptides and glycoproteins are known to those skilled in the art and can be used. Non-limiting examples of labels for polypeptides include, but are not limited to fluorescent labels, enzymatic labels (e.g., horseradish peroxidase, β-galactosidase, luciferase, alkaline phosphatase), chemiluminescent labels, a metallic label, a chromophore, an electro-chemiluminescent label, a phosphorescent label, a quencher (e.g., a fluorophore quencher), a fluorescence resonance energy transfer (FRET) pair (e.g., donor and acceptor), a dye, an enzyme substrate, a small molecule, a mass tag, quantum dots, nanoparticles, biotinyl groups, predetermined polypeptide epitopes recognized by a secondary reporter (e.g., leucine zipper pair sequences, binding sites for secondary antibodies, metal binding domains, epitope tags), the like or combinations thereof.

In some embodiments a bi-specific binding agent comprises a suitable carrier. A bi-specific binding agent can be attached covalently or non-covalently to a suitable carrier. In some embodiments, a Fynomer portion of a bi-specific agent is attached to a carrier. In some embodiments an antibody portion of a bi-specific binding agent is attached to a carrier. Non-limiting examples of a carrier include agents or molecules that alter or extend the in vivo half-life of a bi-specific binding agent include polyethylene glycol, glycogen and/or carbohydrates (e.g., as introduced by glycosylation of a polypeptide), a dextran, a carrier or vehicle described in U.S. Pat. No. 6,660,843, the like or combinations thereof. In certain embodiments, a bi-specific agent, or portion thereof, is glycosylated. In some embodiments a label or carrier is bound to a bi-specific binding agent by use of a suitable linker.

In some embodiments a label, carrier, anti-neoplastic agent, toxin or linker is attached to a suitable thiol group of a bi-specific binding agent (e.g., a thiol group of a cysteine residue). In some embodiments a label, carrier, anti-neoplastic agent, toxin or linker is attached to a suitable reactive nitrogen of an amino group of a bi-specific binding agent. Any suitable amino acid residue of bi-specific binding agent can be substituted with an amino acid residue containing a thiol group (e.g., cysteine) or reactive nitrogen (e.g., lysine) for the purpose of attaching a label, carrier, anti-neoplastic agent, toxin or linker. Non-limiting examples of amino acids in an antibody portion of a bi-specific binding agent that can be substituted with a thiol containing amino acid residue or free amino group include A118, S119, S239, V282, T289, N361, and V422 of an IgG2 or an IgG₁ (corresponding to the EU numbering system), or a corresponding position in an IgG3 or IgG4. Accordingly, in some embodiments, a bi-specific binding agent described herein comprises an antibody comprising a human heavy chain constant region, wherein the heavy chain constant region of the IgG heavy chain of the antibody comprises an A118C (alanine at position 118 to cysteine), S119C (serine at position 119 to cysteine), S239C (serine at position 239 to cysteine), V282C (valine at position 282 to cysteine), T289C (threonine at position 289 to cysteine), N361C (asparagine at position 361 to cysteine), and/or a V422C (valine at position 422 to cysteine) substitution, where an anti-neoplastic agent or toxin is covalently attached to the thiol group of the indicated cysteine residue. In some embodiments, a bi-specific binding agent described herein comprises an antibody comprising a human heavy chain constant region, wherein the constant region of an IgG heavy chain of the antibody comprises an A118K (alanine at position 118 to lysine), S119K (serine at position 119 to lysine), S239K (serine at position 239 to lysine), V282K (valine at position 282 to lysine), T289K (threonine at position 289 to lysine), N361K (asparagine at position 361 to lysine), and/or a V422K (valine at position 422 to lysine) substitution, where an anti-neoplastic agent or toxin is covalently attached to the reactive nitrogen of the free amino group of the indicated lysine residue. Other non-limiting examples of attaching a label, carrier, anti-neoplastic agent, toxin and/or a linker to a bi-specific binding agent include reacting an amine with a succinimidyl ester (e.g., an N-hydroxysuccinimide (NHS) ester), an imidoester, a pentafluorophenyl (PFP) ester, a hydroxymethyl phosphine, an oxirane, an isothiocyanate, a sulfonyl halide, a haloacetyl derivative or any other carbonyl compound; reacting a carboxyl with a carbodiimide; reacting a sulfhydryl with a maleimide, a haloacetyl derivative, a pyridyldisulfide, or a vinyl sulfone; reacting an aldehyde with a hydrazine; reacting any non-selective group with diazirine or an aryl azide; reacting a hydroxyl with an isocyanate; reacting a hydroxylamine with a carbonyl compound; the like and combinations thereof.

### Anti-Neoplastic Agents, Toxins and Linking Groups

In certain embodiments, a bi-specific binding agent disclosed herein comprises an anti-neoplastic agent. In some embodiments, an antibody, or binding portion thereof comprises an anti-neoplastic agent. In some embodiments, a Fynomer comprises an anti-neoplastic agent. In some embodiments, a bi-specific binding agent, antibody or Fynomer comprises one or more (e.g., 1 to 20, 1 to 10 or 1 to 5) anti-neoplastic agents. In some embodiments, a bi-specific binding agent, antibody or Fynomer comprises 1, 2, 3, 4 or 5 anti-neoplastic agents.

Any suitable anti-neoplastic agent can be attached to a bi-specific binding agent, antibody or Fynomer disclosed herein. In some embodiments, an anti-neoplastic agent is an agent that is toxic to a neoplastic cell. In some embodiments, an anti-neoplastic agent comprises a toxic compound, a toxic molecule or toxic payload. Non-limiting examples of an anti-neoplastic agent include a dolastatin, an auristatin, a maytansine, a tubulysin, a calicheamicin, a pyrrolobenzodiazepine (PBD), a duocarmycin, a doxorubicin, a pseudomonas exotoxin-A (PE38), an irinotecan and analogs or derivatives thereof. In some embodiments, an anti-neoplastic agent comprises monomethyl auristatin E (MMAE) or monomethyl auristatin F (MMAF). In some embodiments, an anti-neoplastic agent comprises a pyrrolobenzodiazepine (PBD) toxin and/or a linking group.

In some embodiments, an anti-neoplastic agent is attached to an antibody portion or a Fynomer portion of a bi-specific binding agent. An anti-neoplastic agent may be attached covalently or non-covalently to a bi-specific binding agent, antibody or Fynomer. An anti-neoplastic agent may be attached directly to, or indirectly to (e.g., by means of a linker or linking group) to a bi-specific binding agent, antibody or Fynomer. For example, in some embodiments an anti-neoplastic agent is covalently attached to a bi-specific binding agent, antibody or Fynomer by a linker or a linking group.

An anti-neoplastic agent can be attached to a bi-specific binding agent at any suitable position of a bi-specific binding agent. In some embodiment, an anti-neoplastic agent is attached to a Fynomer portion of a bi-specific binding agent and/or to an antibody portion of a bi-specific binding agent. In some embodiments, an anti-neoplastic agent is attached to a constant region of an antibody portion (e.g., a constant region of an antibody) of a bi-specific binding agent. In some embodiments, an anti-neoplastic agent is directly or indirectly attached (e.g., by means of a linker or linking group) to suitable cysteine residue of a constant region of an antibody portion of a bi-specific binding agent.

In some embodiments, an anti-neoplastic agent comprises a pyrrolobenzodiazepine (PBD) toxin. In some embodiments, an anti-neoplastic agent comprises a linking group or a suitable linker. In some embodiments, an anti-neoplastic agent comprises a pyrrolobenzodiazepine (PBD) toxin and a linking group. In certain embodiments a pyrrolobenzodiazepine toxin is covalently linked to a linking group, and the linking group is covalently linked to a bi-specific binding agent described herein.

Non-limiting examples of PBD toxins and methods of making PBD toxins are described in the following patent application publications: US 2011/0256157, WO 2015/052322, US 2016/0106861, US 2007/0072846, US 2011/0201803, US 2010/0113425, US 2008/0167293, US 2014/0127239, US 2015/0158869, US 2015/0344482, US 2015/0111880, US 2015/0315196, US 2016/0015828, US 2014/0088089, US 2013/0035484, US 2011/0196148, US 2013/0028919, US 2013/0059800, US 2014/0274907, US 2014/0275522, US 2014/0234346, US 2013/0266595, US 2014/0302066, US 2014/0286970, US 2014/0294868, US 2016/0144052, US 2016/0031887, US 2014/0120118, US 2016/0250344, WO 2017/137553, WO 2017/137555 and WO 2017/186894, the entire contents of which are incorporated herein by reference in their entirety.

In some embodiments, a pyrrolobenzodiazepine toxin comprises the structure of chemical formula I:

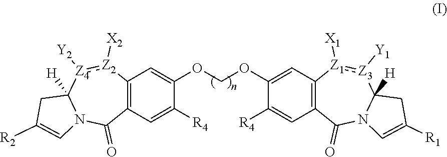

where Z₁ and Z₂ are both N; Z₃ and Z₄ are both C; the double-dash lines [symbol] represent a single bond or a double bond; n is 1 to 12; each of R₃ and R₄ are independently H, or a C₁₋₄ alkoxyl; and each of R₁ and R₂ are independently selected from the group consisting of H, C₁₋₅ alkyl, C₃₋₆ cycloalkyl, C₂₋₅ alkenyl, and a phenyl optionally substituted with R₅, where R₅ is selected from the group consisting of —NH₂, —NHR₆, and a piperazinyl substituted with R₇ having the structure

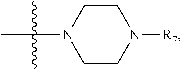

where R₆ comprises a linking group, and R₇ is null, or a C₁₋₅ alkyl; X₁ is null, a protecting group, or comprises a linking group; X₂ is null, a protecting group, or comprises a linking group; only one of X₁, X₂, R₁, and R₂ comprises a linking group; and each of Y₁ and Y₂ are independently either null, OH, or SO₃; provided that: (i) when X₁ comprises a linking group, Z₁ [symbol] Z₃ is N—C, (ii) when X₂ comprises a linking group, Z₂ [symbol] Z₄ is N—C, (iii) when X₁ comprises the protecting group, Z₁ [symbol] Z₃ is N—C, and (iv) when X₂ comprises the protecting group, Z₂ [symbol] Z₄ is N—C.
wherein null means the absence of the moiety or the presence of one or more hydrogens to complete a required valence.

In certain embodiments, a PBD toxin comprises only one linking group. For example, in chemical formula I, only one of X₁, X₂, R₁, and R₂ may comprise a linking group. For example, where X₁ comprises a linking group, X₂, R₁, and R₂ do not comprise a linking group.

In certain embodiments of the PBD toxin of chemical formula I, n is 1-12. In certain embodiments of the PBD toxin of chemical formula I, n is 1-10, 1-9, 1-7, 1-5, or 1-3. In certain embodiments of the PBD toxin of chemical formula I, n is 1, 2, 3, 4, 5, 6, 7, 8, 9, 10, 11 or 12. In some embodiments, n is 1, 3 or 5. In some embodiments, n is 3 or 5.

In certain embodiments of the PBD toxin of chemical formula I, R₃ and R₄ are independently C₁₋₄ alkoxyl. In certain embodiments of the PBD toxin of chemical formula I, R₃ and R₄ are independently selected from —O—CH₂CH₃ or —O—CH₃. In certain embodiments of the PBD toxin of chemical formula I, R₃ and R₄ are both —O—CH₃.

In certain embodiments of the PBD toxin of chemical formula I, R₁ and R₂ are independently selected from the group consisting of H, C₁₋₅ alkyl, C₃-C₆ cycloalkyl, and C₂₋₅ alkenyl. R₁ and R₂ can be the same or different. In some embodiments, R₁ and R₂ are independently selected from a C₁-C₃ alkyl and a C₂-C₃ alkenyl. In certain embodiments, R₁ and R₂ are independently selected from —CH₂CH₂CH₃ and —CH₃. In certain embodiments, both R₁ and R₂ are —CH₂CH₂CH₃ or —CH₃.

In certain embodiments of the PBD toxin of chemical formula I, R₁ and R₂ are independently selected from a C₃-C₆ cycloalkyl, and a phenyl optionally substituted with R₅, where R₅ is selected from the group consisting of —NH₂, —NHR₆, and a piperazinyl substituted with R₇ having the structure

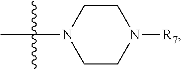

where R₆ comprises a linking group, and R₇ is null, or a C₁₋₅ alkyl. In certain embodiments, R₁ and R₂ are different and independently selected from a (i) a C₃-C₆ cycloalkyl, and (ii) a phenyl optionally substituted with R₅, where R₅ is selected from —NH₂, and —NHR₆, where R₆ comprises a linking group. In certain embodiments, R₁ and R₂ are different and independently selected from a (i) a C₃ cycloalkyl, and (ii) a phenyl substituted with —NH₂, or —NHR₆, where R₆ comprises a linking group. In certain embodiments, R₁ and R₂ are different and independently selected from a (i) a phenyl optionally substituted with R₅, where R₅ is selected from —NH₂, and —NHR₆, where R₆ comprises a linking group and (ii) a piperazinyl substituted with R₇ having the structure, where R₇ is null, or a C₁-C₂ alkyl. In certain embodiments, R₁ and R₂ are different and independently selected from a (i) a phenyl substituted with R₅, where R₅ is —NH₂, and —NHR₆, where R₆ comprises a linking group and (ii) a piperazinyl substituted with R₇ having the structure

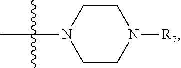

where R₇ is —CH₃. In certain embodiments, R₂ is phenyl substituted with 4-methylpiperazin-1-yl.

In certain embodiments of the PBD toxin of chemical formula I, X₁ is null, Y₁ is null, Z₁ [symbol] Z₃ is N=C, X₂ is null, Y₂ is null and Z₂ [symbol] Z₄ is N=C. In certain embodiments of the PBD toxin of chemical formula I, X₁ comprises the linking group, Y₁ is an OH, Z₂ [symbol] Z₄ is N=C, X₂ is null, and Y₂ is null. In certain embodiments of the PBD toxin of chemical formula I, X₁ comprises the linking group, Y₁ is a OH, Z₂ [symbol] Z₄ is N—C, X₂ is a protecting group, and Y₂ is OH.

In some embodiments, a PBD toxin comprises the structure of chemical formula VII shown below:

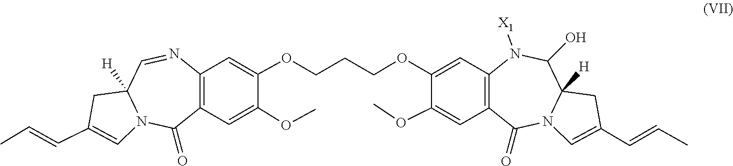

where X₁ comprises the linking group.

In some embodiments, a PBD toxin comprises the structure of chemical formula VIII shown below:

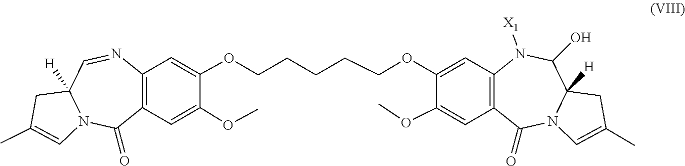

where X₁ comprises the linking group.

In some embodiments, a PBD toxin comprises the structure of chemical formula IX shown below:

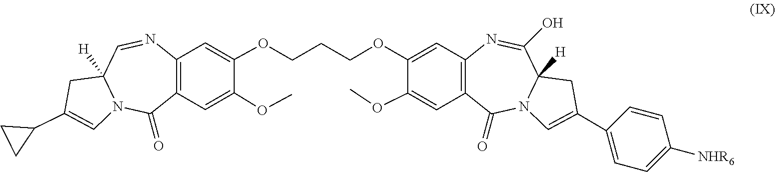

where R6 comprises the linking group.

In some embodiments, a PBD toxin comprises the structure of chemical formula X shown below:

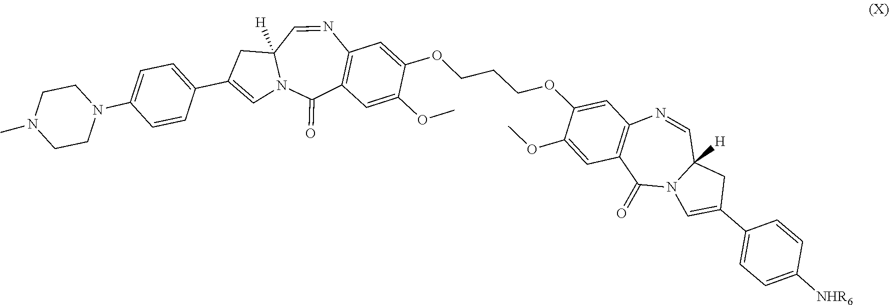

where R₆ comprises the linking group.

In some embodiments, a PBD toxin is attached (e.g., covalently linked) to a linking group by a suitable bond, moiety or group. In some embodiments, a PBD toxin is attached (e.g., covalently linked) to a linking group by a carbonyl linkage or an amide linkage. In some embodiments, a PBD toxin is attached (e.g., covalently linked) to a linking group by a carbamate group. In some embodiments, a PBD toxin is attached (e.g., covalently linked) to a linking group by an amide group. Non-limiting examples of attaching PBD toxin to a linking group are described in US 2017/0002096, US 2016/0331842, US 2015/0250896, US 2017/0080103, US 2016/0136300, US 2017/0152274, US 2015/0209444, US 2013/0274091, US 2017/0095570, US 2017/0157264, US 2015/0125474, US 2011/0256157, WO 2015/052322, US 2016/0106861, US 2007/0072846, US 2011/0201803, US 2010/0113425, US 2008/0167293, US 2014/0127239, US 2015/0158869, US 2015/0344482, US 2015/0111880, US 2015/0315196, US 2016/0015828, US 2014/0088089, US 2013/0035484, US 2011/0196148, US 2013/0028919, US 2013/0059800, US 2014/0274907, US 2014/0275522, US 2014/0234346, US 2013/0266595, US 2014/0302066, US 2014/0286970, US 2014/0294868, US 2016/0144052, US 2016/0031887, US 2014/0120118, US 2016/0250344, WO 2017/137553, WO 2017/137555 and WO 2017/186894, the entire contents of which are incorporated herein by reference in their entirety.

The term “null” as used herein means that an indicated moiety is absent from a structure, however, the indicated moiety may be replaced or occupied by one or more hydrogen atoms to complete a required valence. Further, in reference to any structure shown herein, one or more hydrogens may be present to complete a required valence of a carbon, nitrogen or oxygen atom shown in a structure. Accordingly, where not explicitly indicated, one or more hydrogen atoms may be present.

In some embodiments, an anti-neoplastic agent comprises a suitable linking group. In some embodiments a linking group facilitates a linkage between a bi-specific binding agent and a toxin (e.g., a PBD toxin). In some embodiments a linking group is cleavable. For example, in certain embodiments a linking group comprises an endopeptidase cleavage site which is recognized by an intracellular peptidase. An endopeptidase cleavage site provides a means of detaching and/or releasing an anti-neoplastic agent from a bi-specific binding agent after the bi-specific binding agent is internalized into a neoplastic cell. Non-limiting examples of linking groups and methods a making linking groups are described in W/2015/052322, US 2015/0158869, US 2015/0344482, US 2014/0127239, US 2017/0002096, US 2016/0331842, US 2015/0250896, US 2017/0080103, US 2016/0136300, US 2017/0152274, US 2015/0209444, US 2013/0274091, US 2017/0095570, US 2017/0157264 and US 2015/0125474, which are incorporated herein by reference in their entirety. In some embodiments, a linking group comprises a C1-C20 alkyl, a C1-C20 alkenyl, a C1-C20 alkoxyl, one or more amino acids or amino acid derivatives, a peptide comprising 1 to 20 amino acids, a phenyl group, a suitable polymer (e.g., polyethylene glycol), or a combination thereof.

In some embodiments, a linking group comprises the structure of chemical formula A:

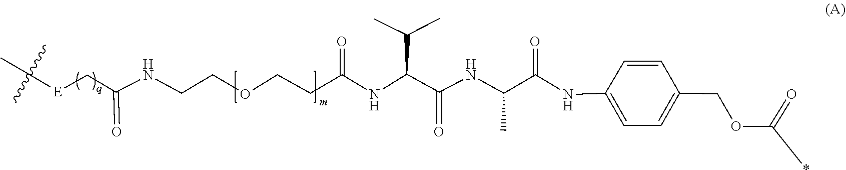

wherein the asterisk indicates the point of attachment of the linking group to a pyrrolobenzodiazepine toxin, the wavy line indicates the point of attachment of the linking group to a bi-specific binding agent, m is 0 to 20, q is 0 to 10 and E is a connecting group. In some embodiments of the linking group of chemical formula A, m is 1 to 20, 1 to 10, 1 to 8, 1 to 6, 1 to 4, 2 to 8 or 4 or 8. In some embodiments of the linking group of chemical formula A, m is selected from 1, 2, 3, 4, 5, 6, 7, 8, 9 or 10. In some embodiments of the linking group of chemical formula A, q is 1 to 10, 1 to 8, 1 to 6, or 1 to 4. In some embodiments of the linking group of chemical formula A, q is selected from 0, 1, 2, 3, 4, 5, 6, 7, 8, 9 or 10. In some embodiments of the linking group of chemical formula A, q is 0, 1 or 2. In some embodiments of the linking group of chemical formula A, m is 8 and q is 2.

In some embodiments, a linking group comprises the structure of chemical formula B:

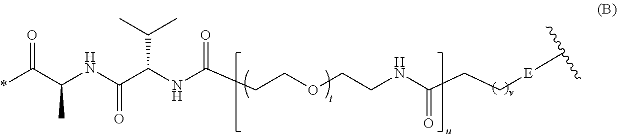

wherein the asterisk indicates the point of attachment of the linking group to a pyrrolobenzodiazepine toxin, the wavy line indicates the point of attachment of the linking group to a bi-specific binding agent, v is 0 to 10, and u is 0 or 1, wherein when u is 1, t is 1 to 10, and E is a connecting group. In some embodiments of the linking group of chemical formula B, v is 1 to 10, 1 to 8, 1 to 4, or 0 to 4. In some embodiments of the linking group of chemical formula B, v is selected from 0, 1, 2, 3, 4, 5, 6, 7 or 8. In some embodiments of the linking group of chemical formula B, when u is 1, t is 1 to 8, 1 to 5, 1 to 4, or 2 to 5. In some embodiments of the linking group of chemical formula B, when u is 1, t is selected from 0, 1, 2, 3, 4, 5, 6, 7, 8, 9 or 10. In some embodiments of the linking group of chemical formula B, t is 8, u is 1, and v is 2. In some embodiments of the linking group of chemical formula B, u is 0, and v is 4.

The connecting group E of chemical formulas A and B can comprise any suitable bond, linker or moiety non-limiting examples of which include a disulfide bond, a thioether bond, a thioester bond, an amide bond, an amine, a ketone, a carboxylate ether, a carbamate, an ester, a thioester, the like, or a combination thereof. In certain embodiments, E comprises a covalent linkage between the linking group and the bi-specific binding agent. In some embodiments, E comprises a covalent bond. In some embodiments, E comprises a reacted moiety that remains after a suitable conjugation reaction is conducted. A multitude of conjugation reactions are known in the art, any one of which can be used to covalently link a linking group disclosed herein to a bi-specific binding agent disclosed herein. Any suitable conjugation chemistry can be used to covalently attach a linking group to a bi-specific binding agent, either stochastically or site-specifically, non-limiting examples of which include a conjugation reaction described in Shan S. Wong (Published Jun. 18, 1991) Chemistry of Protein Conjugation and Cross-Linking, CRC Press; Greg T. Hermanson (Copyright 2013) Bioconjugate Techniques, Third Edition, Elsevier Inc.; and Thiol-X Chemistries in Polymer and Materials Science, RSC Polymer Chemistry Series No. 6 (2013) Edited by Andrew B. Lowe and Christopher N. Bowman, RCS Publishing, WO 2015/052322, US 2015/0158869, US 2015/0344482, US 2014/0127239, US 2017/0002096, US 2016/0331842, US 2015/0250896, US 2017/0080103, US 2016/0136300, US 2017/0152274, US 2015/0209444, US 2013/0274091, US 2017/0095570, US 2017/0157264 and US 2015/0125474, the entire contents of which are incorporated herein by reference in their entirety. Other non-limiting examples of conjugating an anti-neoplastic agent or linking group to a bi-specific binding agent include reacting an amine or amino group with an N-hydroxysuccinimide (NHS) ester, succinimidyl succinate, succinimidyl succinamide, succinimidyl propionate, succinimidyl carbonate, oxycarbonylimidazole, nitrophenyl carbonates, trichlorophenyl carbonate, tresylate, maleic anhydride, methylmaleic anhydride, an imidoester, a pentafluorophenyl (PFP) ester, a hydroxymethyl phosphine, an oxirane or any other carbonyl moiety; reacting a carboxyl moiety with a carbodiimide; reacting a sulfhydryl moiety with a maleimide, a haloacetyl, a pyridyldisulfide, orthopyridyldisulfide and/or a vinyl sulfone; reacting an aldehyde moiety with a hydrazine or hydrazide; reacting any non-selective group with diazirine and/or aryl azide; reacting a hydroxyl moiety with isocyanate; reacting a hydroxylamine moiety with a carbonyl moiety; the like and combinations thereof.

Accordingly, E is often defined by a chemistry used to conjugate a linking group to a bi-specific binding agent. In some embodiments, E comprises a suitable moiety configured to attach a linking group to a bi-specific binding agent. In some embodiments, a linking group is covalently linked to a bi-specific binding agent by means of a suitable sulfhydryl-sulfhydryl reaction, for example by use of a maleimide or pyridyldithiol reactive group that reacts with a reduced cysteine to form stable thioether bond. Additional non-limiting examples of reactive sulfhydryl reactive moieties include a haloacetyls, aziridines, acryloyls, arylating agents, vinylsulfones, a pyridyl disulfide, and TNB-thiol. In certain embodiments, a bi-specific binding agent is connected to E by a thioether bond formed between a cysteine thiol residue (e.g., a thiol) of the bi-specific binding agent and E. Accordingly, in certain embodiments, E comprises a disulfide bond or thioether bond. In some embodiments, for example where a maleimide reaction is used to covalently link a bi-specific binding agent to a linking group, E comprises the structure of chemical formula C:

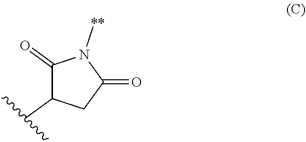

wherein the wavy line indicates the point of attachment to the binding agent and the double asterisk (**) indicates the point of attachment to the linking group. In certain embodiments the double asterisk of chemical formula C represents a thioether bond.

An anti-neoplastic agent, toxin, linking group or connecting group can be conjugated stochastically or site-specifically to any suitable amino acid of a bi-specific binding agent. In some embodiments, anti-neoplastic agent, toxin, linking group or connecting group is conjugated to one or more suitable cysteine residues of a bi-specific binding agent. In some embodiments, anti-neoplastic agent, toxin, linking group or connecting group is conjugated to one or more suitable lysine residues of a bi-specific binding agent. In certain embodiments, one or more amino acids of a bi-specific binding agent are substituted with an amino acid that is suitable for conjugation to anti-neoplastic agent, toxin, linking group or connecting group. Non-limiting examples of amino acids that can be substituted with a thiol containing amino acid residue or a lysine residue include A118, S119, S239, V282, T289, N361, and V422 of an IgG1 or IgG2 constant domain (corresponding to the EU numbering system), or a corresponding position in an IgG3 or IgG4 constant domain. Incorporation of a cysteine into a bi-specific binding agent or antibody by mutagenesis allows for direct conjugation of anti-neoplastic agent, toxin, linking group or connecting group to specific sites on the bi-specific binding agent or antibody, for example via a disulfide bond or thioether bond. For example, one or more amino acids of a bi-specific binding agent can be substituted with a cysteine, where the cysteine can be used for site-specific conjugation of an anti-neoplastic agent, toxin, linking group or connecting group using a suitable chemical reaction. Any suitable amino acid of a constant region of an antibody can be mutated to a cysteine or lysine for site-specific conjugation to an anti-neoplastic agent, toxin, linking group or connecting group.

In some embodiments, a linking group comprises a suitable enzyme cleavage site. In certain embodiments, an enzyme cleavage site comprises an enzyme recognition site of a mammalian protease. Accordingly, in some embodiments, a linking group, or portion thereof, is cleavable by a mammalian protease. A linking group may be cleaved by an enzyme present at or near a target site (e.g., at or near an FGFR3 or CD138 protein). An enzyme present at or near a target site may be intracellular, membrane bound, membrane associated or extracellular (e.g., secreted). For example, a linking group may be configured to be cleaved by a cell surface protease, a secreted protease, or an intracellular protease (e.g., a lysosomal protease). Non-limiting examples of enzyme cleavage sites include a protease recognition site of a lysosomal cysteine protease and/or a lysosomal aspartic protease. Non-limiting examples of lysosomal proteases include cathepsin B, C, H, I, J, K, L, M, N, O, P, S, T and X, and cathepsin D, E, F, G, and/or cathepsin A (carboxypeptidase A).

### Protecting Groups

In some embodiments, a PBD toxin comprises a suitable protecting group. Non-limiting examples of protecting groups and method of making protecting groups are described in the following patent application publications: US 2011/0256157, WO 2015/052322, US2011/0201803, US2008/0167293, US2014/0127239, US2015/0158869, US2015/0344482, US2015/0315196, US2015/0315196, US2014/0302066, US2006/0264622 and US2015/0133435, the entire contents of which are incorporated herein by reference in their entirety.

In some embodiments, a protecting group comprises the structure of chemical formula D below:

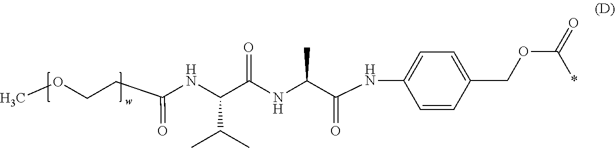

wherein the asterisk indicates a point of attachment to a pyrrolobenzodiazepine toxin; and w is 0 to 10. In some embodiments, w is 0 to 8, 0 to 6, 0 to 4, 1 to 10, 1 to 8, 1 to 5, or 1 to 4. In certain embodiments, w is selected from 0, 1, 2, 3, 4, 5, 6, 7, 8, 9 and 10. In some embodiments, w is 2.

In some embodiments, a protecting group is removable. In certain embodiments, a protecting group is cleavable using a suitable chemistry.

In some embodiments, an anti-neoplastic agent comprises a structure of chemical formula II:

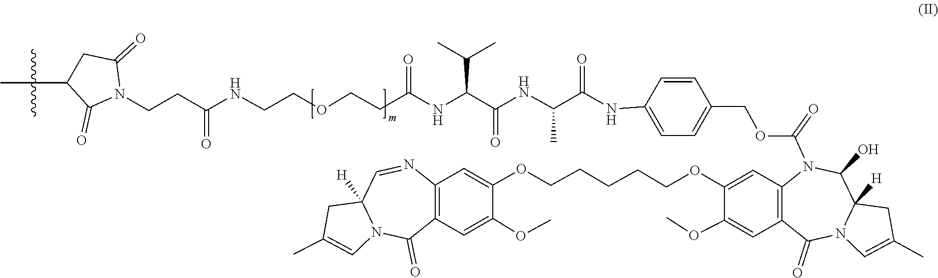

wherein m is 8 and the wavy line indicates the point of attachment to the binding agent.

In some embodiments, an anti-neoplastic agent comprises a structure of chemical formula III:

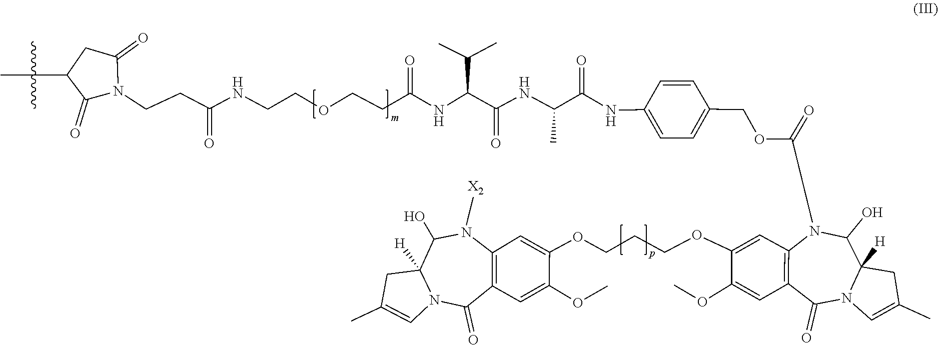

wherein m is 8, p is 1 or 3, X₂ is null, or is a protecting group and the wavy line indicates the point of attachment to the binding agent. In certain embodiments, an anti-neoplastic agent comprises a structure of chemical formula IV:

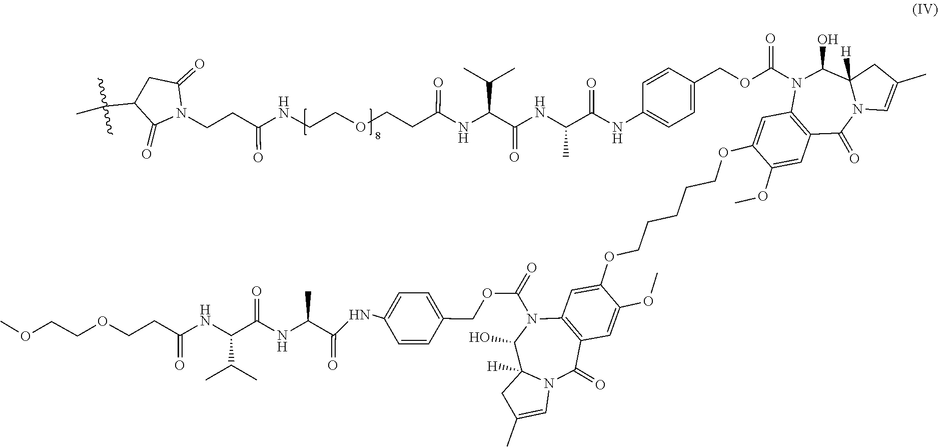

where the wavy line indicates the point of attachment to the binding agent.

In some embodiments, an anti-neoplastic agent comprises a structure of chemical formula V′:

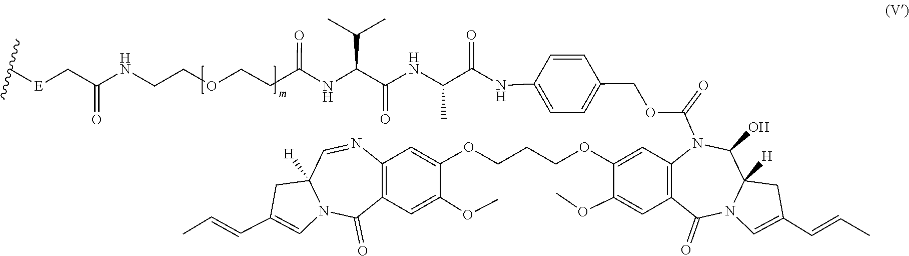

wherein m is 8, E is a suitable connecting group and the wavy line indicates the point of attachment to the binding agent. In some embodiments, E comprises a succinamide moiety of the structure C:

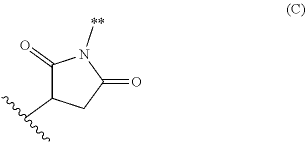

wherein the wavy line indicates the point of attachment to the
binding agent and the double asterisk indicates the point of attachment to the anti-neoplastic agent of chemical formula V. The anti-neoplastic agent of chemical formula V comprising the connecting group of structure C is sometimes referred to herein as chemical formula XI.

In some embodiments, an anti-neoplastic agent comprises a structure of chemical formula VI:

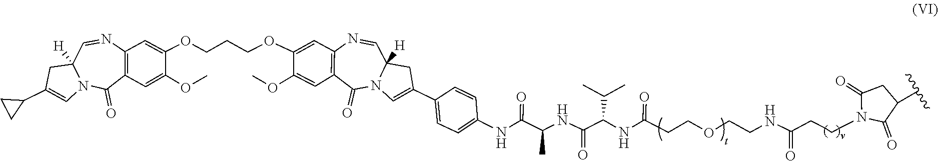

wherein t is 8, v is 1 and the wavy line indicates the point of attachment to the binding agent.

In some embodiments, an anti-neoplastic agent comprises a structure of chemical formula VII:

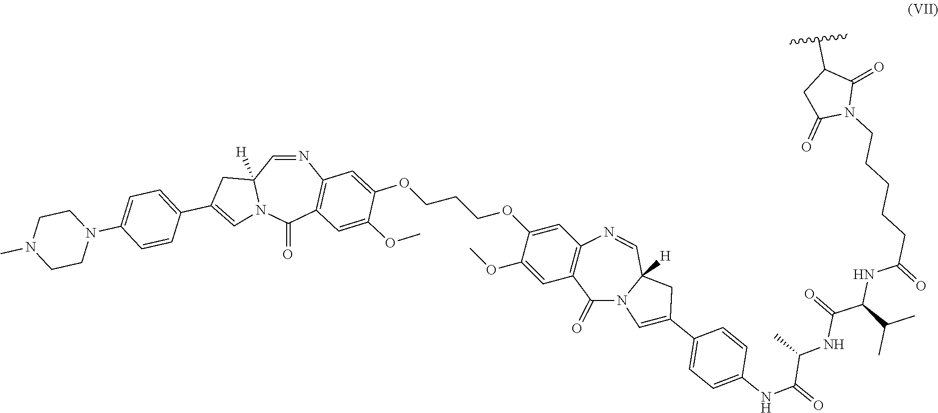 [SMILES: *C1CC(=O)N(CCCCCC(=O)N[C@H](C(=O)N[C@@H](C)C(=O)Nc2ccc(C3=CN4C(=O)c5cc(OC)c(OCCCOc6cc7c(cc6OC)C(=O)N6C=C(c8ccc(N9CCN(C)CC9)cc8)C[C@H]6C=N7)cc5N=C[C@@H]4C3)cc2)C(C)C)C1=O]

wherein the wavy line indicates the point of attachment to the binding agent.

In some embodiments, a bi-specific binding agent comprises an anti-neoplastic agent comprising a structure selected from any one of chemical formulas II, III, IV, V′, VI, VII and XI.

### Pharmaceutical Compositions

In some embodiments, a composition or pharmaceutical composition comprises a bi-specific binding agent described herein (e.g., a bi-specific binding agent comprising an anti-neoplastic agent). In some embodiments, a pharmaceutical composition comprises a bi-specific binding agent and a pharmaceutically acceptable excipient, diluent, additive or carrier.

A pharmaceutical composition can be formulated for a suitable route of administration. In some embodiments a pharmaceutical composition is formulated for subcutaneous (s.c.), intradermal, intramuscular, intraperitoneal and/or intravenous (i.v.) administration. In certain embodiments, a pharmaceutical composition can contain formulation materials for modifying, maintaining, or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates (e.g., phosphate buffered saline) or suitable organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); solvents (such as glycerin, propylene glycol or polyethylene glycol); diluents; excipients and/or pharmaceutical adjuvants. In particular, pharmaceutical compositions can comprise any suitable carrier, formulation, or ingredient, the like or combinations thereof as listed in “Remington: The Science And Practice Of Pharmacy” Mack Publishing Co., Easton, Pa., 19^{th} Edition, (1995) (hereafter, “Remington '95”), or “Remington: The Science And Practice Of Pharmacy”, Pharmaceutical Press, Easton, Pa., 22^{nd} Edition, (2013) (hereafter, “Remington 2013”), the contents of which are incorporated herein by reference in their entirety. The various materials listed herein, alone or in combination, can be incorporated into or used with the materials described in Remington '95 or Remington 2013. Any suitable techniques, carriers, and excipients can be used, including those understood in the art; e.g., as described in Remington '95 or Remington 2013.

In certain embodiments, a pharmaceutical composition comprises a suitable excipient, non-limiting example of which include anti-adherents (e.g., magnesium stearate), a binder, fillers, monosaccharides, disaccharides, other carbohydrates (e.g., glucose, mannose or dextrins), sugar alcohols (e.g., mannitol or sorbitol), coatings (e.g., cellulose, hydroxypropyl methylcellulose (HPMC), microcrystalline cellulose, synthetic polymers, shellac, gelatin, corn protein zein, enterics or other polysaccharides), starch (e.g., potato, maize or wheat starch), silica, colors, disintegrants, flavors, lubricants, preservatives, sorbents, sweeteners, vehicles, suspending agents, surfactants and/or wetting agents (such as pluronics, PEG, sorbitan esters, polysorbates such as polysorbate 20, polysorbate 80, triton, tromethamine, lecithin, cholesterol, tyloxapal), stability enhancing agents (such as sucrose or sorbitol), and tonicity enhancing agents (such as alkali metal halides, sodium or potassium chloride, mannitol, sorbitol), and/or any excipient disclosed in Remington '95 or Remington 2013. The term “binder” as used herein refers to a compound or ingredient that helps keeps a pharmaceutical mixture combined. Suitable binders for making pharmaceutical formulations and are often used in the preparation of pharmaceutical tablets, capsules and granules are known to those skilled in the art.

In some embodiments a pharmaceutical composition comprises a suitable pharmaceutically acceptable additive and/or carrier. Non-limiting examples of suitable additives include a suitable pH adjuster, a soothing agent, a buffer, a sulfur-containing reducing agent, an antioxidant and the like. Non-limiting examples of a sulfur-containing reducing agent includes those having a sulfhydryl group such as N-acetylcysteine, N-acetylhomocysteine, thioctic acid, thiodiglycol, thioethanolamine, thioglycerol, thiosorbitol, thioglycolic acid and a salt thereof, sodium thiosulfate, glutathione, and a C1-C7 thioalkanoic acid. Non-limiting examples of an antioxidant include erythorbic acid, dibutylhydroxytoluene, butylhydroxyanisole, alpha-tocopherol, tocopherol acetate, L-ascorbic acid and a salt thereof, L-ascorbyl palmitate, L-ascorbyl stearate, sodium bisulfite, sodium sulfite, triamyl gallate and propyl gallate, as well as chelating agents such as disodium ethylenediaminetetraacetate (EDTA), sodium pyrophosphate and sodium metaphosphate. Furthermore, diluents, additives and excipients may comprise other commonly used ingredients, for example, inorganic salts such as sodium chloride, potassium chloride, calcium chloride, sodium phosphate, potassium phosphate and sodium bicarbonate, as well as organic salts such as sodium citrate, potassium citrate and sodium acetate.

The pharmaceutical compositions used herein can be stable over an extended period of time, for example on the order of months or years. In some embodiments a pharmaceutical composition comprises one or more suitable preservatives. Non limiting examples of preservatives include benzalkonium chloride, benzoic acid, salicylic acid, thimerosal, phenethyl alcohol, methylparaben, propylparaben, chlorhexidine, sorbic acid, hydrogen peroxide, the like and/or combinations thereof. A preservative can comprise a quaternary ammonium compound, such as benzalkonium chloride, benzoxonium chloride, benzethonium chloride, cetrimide, sepazonium chloride, cetylpyridinium chloride, or domiphen bromide (BRADOSOL®). A preservative can comprise an alkyl-mercury salt of thiosalicylic acid, such as thimerosal, phenylmercuric nitrate, phenylmercuric acetate or phenylmercuric borate. A preservative can comprise a paraben, such as methylparaben or propylparaben. A preservative can comprise an alcohol, such as chlorobutanol, benzyl alcohol or phenyl ethyl alcohol. A preservative can comprise a biguanide derivative, such as chlorohexidine or polyhexamethylene biguanide. A preservative can comprise sodium perborate, imidazolidinyl urea, and/or sorbic acid. A preservative can comprise stabilized oxychloro complexes, such as known and commercially available under the trade name PURITE®. A preservative can comprise polyglycol-polyamine condensation resins, such as known and commercially available under the trade name POLYQUART® from Henkel KGaA. A preservative can comprise stabilized hydrogen peroxide. A preservative can be benzalkonium chloride. In some embodiments a pharmaceutical composition is free of preservatives.

In some embodiments a composition, pharmaceutical composition or bi-specific binding agent is substantially free of blood, or a blood product contaminant (e.g., blood cells, platelets, polypeptides, minerals, blood borne compounds or chemicals, and the like). In some embodiments a composition, pharmaceutical composition or bi-specific binding agent is substantially free of serum and serum contaminants (e.g., serum proteins, serum lipids, serum carbohydrates, serum antigens and the like). In some embodiments a composition, pharmaceutical composition or bi-specific binding agent is substantially free a pathogen (e.g., a virus, parasite or bacteria). In some embodiments a composition, pharmaceutical composition or bi-specific binding agent is substantially free of endotoxin. In some embodiments a composition, pharmaceutical composition or bi-specific binding agent is sterile. In certain embodiments, a composition or pharmaceutical composition comprises a bi-specific binding agent and a diluent (e.g., phosphate buffered saline (PBS)). In certain embodiments, a composition or pharmaceutical composition comprises a bi-specific binding agent and an excipient, (e.g., sodium citrate dehydrate, or polyoxyethylene-sorbitan-20 mono-oleate (polysorbate 80)).

The pharmaceutical compositions described herein may be configured for administration to a subject in any suitable form and/or amount according to the therapy in which they are employed. For example, a pharmaceutical composition configured for parenteral administration (e.g., by injection or infusion), may take the form of a suspension, solution or emulsion in an oily or aqueous vehicle and it may contain formulation agents, excipients, additives and/or diluents such as aqueous or non-aqueous solvents, co-solvents, suspending solutions, preservatives, stabilizing agents and or dispersing agents. In some embodiments a pharmaceutical composition suitable for parental administration may contain one or more excipients. In some embodiments a pharmaceutical composition is lyophilized to a dry powder form. In some embodiments a pharmaceutical composition is lyophilized to a dry powder form, which is suitable for reconstitution with a suitable pharmaceutical solvent (e.g., water, saline, an isotonic buffer solution (e.g., PBS), and the like). In certain embodiments, reconstituted forms of a lyophilized pharmaceutical composition are suitable for parental administration (e.g., intravenous administration) to a mammal.

In certain embodiments, a pharmaceutical composition is configured for oral administration and may be formulated as a tablet, microtablet, minitablets, micropellets, powders granules, capsules (e.g., capsules filled with microtablets, micropellets, powders or granules), emulsions or solutions. Pharmaceutical compositions configured for oral administration may comprise suitable coatings to delay or sustain release of the active ingredient (e.g., a bi-specific binding agent), non-limiting examples of which include enteric coatings such as fatty acids, waxes, shellac, plastics, methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, cellulose acetate trimellitate, sodium alginate, zein, plant fibers, the like and combinations thereof.

In some embodiments a pharmaceutical compositions described herein may be configured for topical administration and may include one or more of a binding and/or lubricating agent, polymeric glycols, gelatins, cocoa-butter or other suitable waxes or fats. In some embodiments a pharmaceutical composition described herein is incorporated into a topical formulation containing a topical carrier that is generally suited to topical drug administration and comprising any suitable material known to those skilled in the art. In certain embodiments, a topical formulation of a pharmaceutical composition is formulated for administration of a bi-specific binding agent from a topical patch.

In certain embodiments, an optimal pharmaceutical composition will be determined by one skilled in the art depending upon, for example, the intended route of administration, delivery format and desired dosage (see e.g., Remington '95 or Remington 2013, supra). In certain embodiments, such compositions may influence the physical state, stability, rate of in vivo release and rate of in vivo clearance of the antibody drug conjugates of the invention. A pharmaceutical composition can be manufactured by any suitable manner, including, e.g., by means of conventional mixing, dissolving, granulating, dragee-making, levigating, emulsifying, encapsulating, entrapping or tableting processes (e.g., see methods described in Remington '95 or Remington 2013).

In some embodiments, presented herein is a composition or pharmaceutical composition for use as a medicament for the treatment of a neoplasm in a subject, wherein the composition or pharmaceutical composition comprises a bi-specific binding agent described herein (e.g., a bi-specific binding agent comprising an anti-neoplastic agent). In some embodiments, presented herein is a composition or pharmaceutical composition comprising a bi-specific binding agent, or a bi-specific binding agent conjugated (e.g., covalently attached) to an anti-neoplastic agent or PBD toxin for use in the treatment of a neoplasm.

In some embodiments a composition, pharmaceutical composition or bi-specific binding agent (e.g., a bi-specific binding agent comprising an anti-neoplastic agent) described herein is used to treat a subject having or suspected of having a neoplasm. In some embodiments a composition, pharmaceutical composition or bi-specific binding agent (e.g., a bi-specific binding agent comprising an anti-neoplastic agent) described herein is administered to a subject having or suspected of having a neoplasm. In some embodiments, presented herein is a method of treating a subject having or suspected of having a neoplasm. In certain embodiments a method of treating a subject having or suspected of having a neoplasm comprises administering to the subject a therapeutically effective amount of a composition or bi-specific binding agent described herein. In certain embodiments a bi-specific binding agent, or pharmaceutical composition comprising a bi-specific binding agent, is administered to a subject, wherein the bi-specific binding agent binds specifically to an extracellular domain of human syndecan-1 and/or to an FGFR3 (e.g., an FGFR3, FGFR3b and/or FGFR3c).

In certain embodiments, a method of treating a subject comprises contacting a cell (e.g., one or more cells) of a subject with a therapeutically effective amount of a composition, pharmaceutical composition or bi-specific binding agent described herein. In certain embodiments, a method of treatment comprises contacting a cell (e.g., one or more cells) of a subject with a therapeutically effective amount of a bi-specific binding agent that specifically binding to an extracellular portion of human syndecan-1, or variant thereof, and/or to an FGFR3 (e.g., an FGFR3, FGFR3b and/or FGFR3c). The cell of a subject is often a cell that expresses an extracellular portion of syndecan-1. A cell of a subject may be found inside a subject (e.g., in vivo) or outside the subject (e.g., in vitro or ex vivo).

Non-limiting examples of a neoplasm that can be treated by a method described herein include a carcinoma, sarcoma, nervous system neoplasia (neoplasia of the nervous system), lymphoma, myeloma, leukemia, melanoma, mesothelioma, solid or soft tissue tumors, and secondary cancers (e.g., derived from a primary site)). Non-limiting examples of a carcinoma include respiratory system carcinomas, gastrointestinal system carcinomas, genitourinary system carcinomas, testicular carcinomas, prostatic carcinomas, endocrine system carcinomas, basal cell carcinoma of the skin, carcinoma of unknown primary, cholangiocarcinoma, ductal carcinoma in situ (DCIS), Merkel cell carcinoma, lung carcinoma, thymoma and thymic carcinoma, midline tract carcinoma, lung small cell carcinoma, thyroid carcinoma, liver hepatocellular carcinoma, squamous cell carcinoma, head and neck squamous carcinoma, breast carcinoma, epithelial carcinoma, adrenocortical carcinoma, ovarian surface epithelial carcinoma, and the like, further including carcinomas of the uterus, cervix, colon, pancreas, kidney, esophagus, stomach and ovary. Non-limiting examples of a sarcoma include Ewing sarcoma, lymphosarcoma, liposarcoma, osteosarcoma, breast sarcoma, soft tissue sarcoma, Kaposi sarcoma, rhabdomyosarcoma, uterine sarcoma, chondrosarcoma, leiomyosarcoma, fibrosarcoma and the like. Non-limiting examples of a nervous system neoplasia include glioma, glioblastoma, meningioma, neuroblastoma, retinoblastoma, astrocytoma, oligodendrocytoma and the like. Non-limiting examples of lymphomas, myelomas, and leukemia include acute and chronic lymphoblastic leukemia, myeloblastic leukemia, multiple myeloma, poorly differentiated acute leukemias (e.g., erythroblastic leukemia and acute megakaryoblastic leukemia), acute promyeloid leukemia (APML), acute myelogenous leukemia (AML), chronic myelogenous leukemia (CML), acute lymphoblastic leukemia (ALL), which includes B-lineage ALL and T-lineage ALL, chronic lymphocytic leukemia (CLL), prolymphocytic leukemia (PLL), hairy cell leukemia (HLL), Waldenstrom's macroglobulinemia (WM), non-Hodgkin lymphoma and variants, peripheral T-cell lymphomas, adult T-cell leukemia/lymphoma (ATL), cutaneous T-cell lymphoma (CTCL), large granular lymphocytic leukemia (LGF), Hodgkin's disease and Reed-Sternberg disease. Non-limiting examples of soft or solid tissue tumors include visceral tumors, seminomas, hepatomas, and other tumors of the breast, liver, lung, pancreas, uterus, ovary, testicle, head, neck, eye, brain, mouth, pharynx, vocal cord, ear, nose, esophagus, stomach, intestine, colon, adrenal gland, kidney, bone, bladder, urethra, carcinomas, lung, muscle, skin, feet, hands, and soft tissue. In some embodiments, a neoplasm that can be treated by a pharmaceutical composition or bi-specific binding agent disclosed herein is selected from a bladder cancer, breast cancer, colorectal cancer, cervical cancer, gastric cancer, liver cancer, hepatocellular cancer, hypopharynx cancer, lung cancer, adenocarcinoma, ovarian cancer and renal cancer. In some embodiments, a neoplasm that can be treated by a pharmaceutical composition or bi-specific binding agent disclosed herein is selected from a pancreatic cancer (e.g., a pancreatic adenocarcinoma, exocrine pancreatic cancer or pancreatic neuroendocrine cancer), a colorectal cancer (e.g., a colorectal adenocarcinoma), small intestinal malignancy, cholangiocarcinoma, non-small cell lung cancer (NSCLC), thyroid carcinoma, esophageal or esophagogastric junction (EGJ) cancer, gastric adenocarcinoma, liver hepatocellular carcinoma, head and neck squamous carcinoma, female genital tract malignancy, breast carcinoma, lung small cell carcinoma, ovarian surface epithelial carcinoma, retroperitoneal or peritoneal sarcoma, prostatic adenocarcinoma, neuroendocrine tumor, gastrointestinal stromal tumor, glioblastoma or non-epithelial ovarian cancer. In some embodiments, a neoplasm that can be treated by a pharmaceutical composition or bi-specific binding agent disclosed herein is a breast cancer, non-limiting examples of which include ductal carcinoma in situ (DCIS), invasive ductal carcinoma (IDC)(e.g., tubular carcinoma of the breast, medullary carcinoma of the breast, mucinous carcinoma of the breast, papillary carcinoma of the breast, and cribriform carcinoma of the breast), invasive lobular carcinoma (ILC), inflammatory breast cancer, lobular carcinoma in situ (LCIS), male breast cancer, molecular subtypes of breast cancer (e.g., Luminal B breast cancer or hormone-receptor positive breast cancer, Triple-negative breast cancer, HER2-enriched breast cancer, and normal-like breast cancer), Paget's disease of the nipple, phyllodes tumors of the breast, and metastatic breast cancer. In some embodiments a neoplasm that can be treated by a pharmaceutical composition or bi-specific binding agent disclosed herein is a triple negative breast cancer.

In some embodiments, the effectiveness of a treatment described herein can be determined or predicted, in part, by an amount of CD138 and/or FGFR3 (e.g., FGFR3, or an isoform thereof) that a neoplasm or neoplastic cell expresses. For example, without being limited to theory, a subject having a neoplasm or neoplastic cells expressing high levels of CD138 and/or FGFR3 may respond better to therapy with a bi-specific binding agent described herein than another subject having a neoplasm or neoplastic cells that express little or no CD138 or FGFR3. A neoplastic cell or cancer cell can be quickly assayed to determine an expression level of CD138 using a suitable anti-CD138 antibody and a suitable immunoassay (e.g., whole-cell ELISA, FACs, and the like). Likewise, a neoplastic cell or cancer cell can be quickly assayed for expression of an FGFR3 using a suitable anti-FGFR3 antibody and a suitable immunoassay. Accordingly, in some embodiments, a method of treating a subject having or suspected of having a neoplasm comprises administering a therapeutically effective amount of a bi-specific binding agent described herein, wherein the neoplasm, or neoplastic cells thereof, express a detectable level of CD138 and/or an FGFR3 (e.g., FGFR3, or an isoform thereof). In certain embodiments, a neoplasm that expresses detectable levels of CD138 and/or an FGFR3 is a neoplasm that is known or reported to express CD138 and/or an FGFR3. In certain embodiments, a neoplasm is suspected of expressing CD138 and/or an FGFR3 (e.g., by having a similar genotype or phenotype to another neoplastic cell type that is known to express CD138 and/or an FGFR3). In some embodiments, a neoplasm that expresses, or is suspected of expressing CD138 and/or an FGFR3 is a neoplasm that expresses an RNA transcript that encodes CD138 and/or an FGFR3, or a portion thereof. In some embodiments, a neoplasm that expresses, or is suspected of expressing CD138 and/or an FGFR3, is a neoplasm that expresses CD138 and/or an FGFR3 on its cell surface. Non-limiting examples of cancers with confirmed expression of CD138 and/or FGFR3 include adrenocortical carcinoma, bladder urothelial carcinoma, breast invasive carcinoma, cervical squamous cell carcinoma, endocervical adenocarcinoma, cholaniocarcinoma, colon adenocarcinoma, colorectal adenocarcinoma, lymphoid neoplasm diffuse large B-cell lymphoma, esophageal carcinoma, glioblastoma multiforme, glioma, head and neck squamous cell carcinoma, kidney chromophobe, pan-kidney cohort (KICH+KIRC+KIR), kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, stomach and esophageal carcinoma, testicular germ cell tumors, thyroid carcinoma, thymoma, uterine corpus endometrial carcinoma, uterine carcinosarcoma, and uveal melanoma.

In some embodiments, a method of treating a subject having or suspected of having a neoplasm comprises administering a therapeutically effective amount of a bi-specific binding agent described herein in combination with another anti-cancer therapy, non-limiting examples of which include a T-cell activating agent, an adjuvant, an anti-cancer vaccine, a radiation treatment, an immunotherapy (e.g., anti-HER2, or anti-CD20), a chemotherapy, and the like or combinations thereof. In some embodiments, a T-cell activating agent is an antibody that binds to CD3, OX40, GITR, CD137 (41BB), CD27, HVEM, LAG-3, TIM3, VISTA or BTLA.

Any suitable chemotherapeutic agent can be used for a method described herein. In some embodiments a chemotherapeutic agent comprises or consists of an alkylating agent, an anthracycline, cytoskeletal disruptors, epothilones (e.g., epothilone), histone deacetylase inhibitors (e.g., vorinostat, romidepsin), inhibitors of topoisomerase I (e.g., irinotecan, topotecan), inhibitors of topoisomerase II (e.g., etoposide, teniposide, tafluposidean), kinase inhibitors, peptide antibiotics (e.g., bleomycin, actinomycin), platinum-based agents (e.g., carboplatin, cisplatin, oxaliplatin), compounds targeting DNA repair enzyme poly-ADP ribose polymerase-1 (e.g., Parp inhibitors), retinoids (e.g., tretinoin, alitretinoin, bexarotene), vinca alkaloids and compounds (e.g., vinblastine, vincristine, vindesine, vinorelbine), anti-metabolites, plant extracts, plant alkaloids, nitrosourea, hormone, nucleoside or nucleotide analog and combinations thereof. Non-limiting examples of alkylating anti-neoplastic agents include Altretamine (hexamethylmelamine, HEXALEN®), Busulfan, Carmustine (BCNU), Chlorambucil, Cyclophosphamide, Dacarbazine (DTIC), Fotemustine, Ifosfamide, Lomustine (CCNU), Mechlorethamine, Melphalan, Procarbazine, semustine (MeCCNU), Streptozotocin, Temozolomide, Thiotepa (triethylenethio-phosphoramide), Carboplatin, Cisplatin, Oxaliplatin, monofunctional alkylators, nitrosoureas, temozolomide, the like or combinations thereof. Non-limiting examples of a DNA intercalating agent include acrolein, anthracycline, phosphoramide, Actinomycin D, bleomycin, idarubicin, daunorubicin, doxorubicin, elsamicin A, epirubicin, ethidium, m-AMSA, mitoxantrone, doxorubicin (Adriamycin, Doxil, Myocet, hydroxydaunorubicin, hydroxydaunomycin), Epirubicin, Idarubicin, Valrubicin, TAS-103, MLN944 (XR5944), Obatoclax, mechlorethamine, methotrexate, 6-mercaptopurine, thioguanine, 5-fluorouracil, cytosine arabinoside, 5-azacytidine (5-AZC) and 5-azacytidine related compounds, mithramycin, mitomycin C, hydroxyurea, carboplatin, oxiplatin, mitotane, a taxane, vinblastine, vincristine, dibromomannitol, gemcitabine, pemetrexed, the like or a combination thereof. Non-limiting examples of cytoskeletal disruptors (e.g., taxanes) include paclitaxel, taxol, and docetaxel. Non-limiting examples of kinase inhibitors include bortezomib, erlotinib, gefitinib, imatinib, vemurafenib, vismodegib, the like, analogs and compounds thereof. Non-limiting examples of nucleotide analogs include azacitidine, azathioprine, capecitabine, cytarabine, doxifluridine, fluorouracil, gemcitabine, hydroxyurea, mercaptopurine, methotrexate, tioguanine (formerly thioguanine), the like, analogs and compounds thereof. Non-limiting examples of PARP inhibitors are olaparib, rucaparib, niraparib, veliparib, talazoparib and the like, analogs and compounds thereof.

The term “subject” refers to a mammal. Any suitable mammal can be treated by a method or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, and pigs) and experimental animals (e.g., mouse, rat, rabbit, and guinea pig). In some embodiments a mammal is a human. A mammal can be any age or at any stage of development (e.g., an adult, teen, child, infant, or a mammal in utero). A mammal can be male or female. In some embodiments a subject in need is a subject who has or is suspected of having a neoplasm.

Any suitable method of administering a composition, pharmaceutical composition or bi-specific binding agent to a subject can be used. The exact formulation and route of administration for a composition for use according to the methods of the invention described herein can be chosen by the individual physician in view of the patient's condition. See, e.g., Fingl et al. 1975, in “The Pharmacological Basis of Therapeutics,” Ch. 1, p. 1; which is incorporated herein by reference in its entirety. Any suitable route of administration can be used for administration of a pharmaceutical composition or a bi-specific binding agent described herein. Non-limiting examples of routes of administration include topical or local (e.g., subcutaneously, transdermally or cutaneously, (e.g., on the skin or epidermis), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranial, intra-articular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments a composition herein is provided to a subject. A composition that is provided to a subject is sometimes provided to a subject for self-administration or for administration to a subject by another (e.g., a non-medical professional). For example a composition described herein can be provided with an instruction written by a medical practitioner that authorizes a patient to be provided a composition or treatment described herein (e.g., a prescription). In another example, a composition can be provided to a subject where the subject self-administers a composition orally, intravenously or by way of an inhaler, for example.

Alternately, one can administer compositions for use according to the methods of the invention in a local rather than systemic manner, for example, via direct application to the skin, mucous membrane or region of interest for treating, including using a depot or sustained release formulation.

In some embodiments a pharmaceutical composition comprising a bi-specific binding agent is administered alone (e.g., as a single active ingredient (AI or e.g., as a single active pharmaceutical ingredient (API)). In other embodiments, a pharmaceutical composition comprising a bi-specific binding agent is administered in combination with one or more additional AIs/APIs, for example, as two separate compositions or as a single composition where the one or more additional AIs/APIs are mixed or formulated together with the bi-specific binding agent in a pharmaceutical composition.

In certain embodiments, a bi-specific binding agent is delivered to a cell (e.g., a mammalian cell). A bi-specific binding agent can be delivered to a cell using any suitable method. In certain embodiments, delivering a bi-specific binding agent to a cell comprises contacting a mammalian cell, in vitro or in vivo, with a composition comprising a bi-specific binding agent under conditions that allow the bi-specific binding agent to bind to the cell.

A pharmaceutical composition can be manufactured by any suitable manner, including, e.g., by means of conventional mixing, dissolving, granulating, dragee-making, levigating, emulsifying, encapsulating, entrapping or tableting processes.

In some embodiments a method of treating a neoplasm in a subject comprising administering a therapeutically effective amount of a bi-specific binding agent, or a therapeutically effective amount of a pharmaceutical composition comprising a bi-specific binding agent, to the subject. A “therapeutically effective amount” means an amount sufficient to obtain an effective therapeutic outcome and/or an amount necessary and/or sufficient to prevent, terminate, block, inhibit, ameliorate, abrogate, slow, suppress, kill or reduce the growth, viability, metastasis, severity, onset, or a symptom of a neoplasm. In some embodiments, an effective therapeutic outcome can be determined by measuring and/or monitoring the number, size, viability, growth, mitosis, or metastasis of a neoplasm or neoplastic cells in a subject before and/or after treatment. Accordingly, in some embodiments, administering a therapeutically effective amount of a bi-specific binding agent, or a therapeutically effective amount of a pharmaceutical composition comprising a bi-specific binding agent, to a subject prevents, terminates, blocks, inhibits, ameliorates, abrogates, slows, suppresses, kills or reduces the growth, viability, metastasis, severity, onset, or a symptom of a neoplasm. In certain embodiments, administering a therapeutically effective amount of a bi-specific binding agent, or a therapeutically effective amount of a pharmaceutical composition comprising a bi-specific binding agent, to a subject induces death, necrosis, or apoptosis of some or all of the cancerous cells of a neoplasm. Determination of a therapeutically effective amount is well within the capability of those skilled in the art, especially in light of the detailed disclosure provided herein.

In some embodiments, an amount of a bi-specific binding agent in a composition is an amount that is at least a therapeutically effective amount and an amount low enough to minimize unwanted adverse reactions. The exact amount of a bi-specific binding agent will vary from subject to subject, depending on age, weight, and general condition of a subject, the severity of the condition being treated, and/or the amount of other therapeutic drugs administered. Thus, it is not always possible to specify an exact therapeutically effective amount of a bi-specific binding agent that can be administered to treat a neoplasm in a diverse group of subjects. As is well known, the specific dosage for a given patient under specific conditions and for a specific disease will routinely vary, but determination of the optimum amount in each case can readily be accomplished by simple routine procedures. Thus, a therapeutically effective amount of a bi-specific binding agent used to treat a neoplasm may be determined by one of ordinary skill in the art using routine experimentation.

In certain embodiments, an therapeutically effective amount of a bi-specific binding agent in a composition comprises a dose from about 0.01 mg/kg (e.g., per kg body weight of a subject) to 500 mg/kg, 0.1 mg/kg to 500 mg/kg, 0.1 mg/kg to 400 mg/kg, 0.01 mg/kg to 300 mg/kg, 0.1 mg/kg to 300 mg/kg, 0.1 mg/kg to 200 mg/kg, 0.1 mg/kg to 150 mg/kg, 0.1 mg/kg to 100 mg/kg, 0.1 mg/kg to 75 mg/kg, 0.1 mg/kg to 50 mg/kg, 0.1 mg/kg to 25 mg/kg, 0.1 mg/kg to 10 mg/kg, 0.1 mg/kg to 5 mg/kg or 0.1 mg/kg to 1 mg/kg. In some aspects the amount of a bi-specific binding agent can be about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg. In some embodiments a therapeutically effective amount of a bi-specific binding agent is between about 0.1 mg/kg to 500 mg/kg, or between about 1 mg/kg and about 300 mg/kg. Volumes suitable for various routes of administration are known in the art.

In some embodiments a bi-specific binding agent or a pharmaceutical composition comprising a bi-specific binding agent is administered at a suitable frequency and/or interval as needed to obtain an effective therapeutic outcome. In some embodiments, a pharmaceutical composition comprising a bi-specific binding agent is administered hourly, once a day, twice a day, three times a day, four times a day, five times a day, and/or at regular intervals, for example, every day, every other day, three times a week, weekly, every other week, once a month and/or simply at a frequency or interval as needed or recommended by a medical professional.

### Kits

In some embodiments, a pharmaceutical composition comprising an amount or dose of a bi-specific binding agent is provided in a kit, pack or dispensing device, which can contain one or more doses of a bi-specific binding agent. The kit or pack can for example comprise one or more suitable containers, vials, or blister packs and one or more suitable dispensing devices. In some embodiments, a kit or pack is accompanied by instructions for administration and/or a notice prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert.

In some embodiments a kit or pack comprises an amount of a bi-specific binding agent sufficient to treat a patient for 1 day to 1 year, 1 day to 180 days, 1 day to 120 days, 1 day to 90 days, 1 day to 60 days, 1 day to 30 days, or any day or number of days there between, 1-4 hours, 1-12 hours, or 1-24 hours.

A kit optionally includes a product label or packaging inserts including a description of the components or instructions for use in vitro, in vivo, or ex vivo, of the components therein. Exemplary instructions include instructions for a diagnostic method, treatment protocol or therapeutic regimen. In certain embodiments, a kit comprises packaging material, which refers to a physical structure housing components of the kit. The packaging material can maintain the components sterilely, and can be made of material commonly used for such purposes (e.g., paper, corrugated fiber, glass, plastic, foil, ampules, vials, tubes, etc.). Product labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, optical disk such as CD- or DVD-ROM/RAM, DVD, MP3, magnetic tape, or an electrical storage media such as RAM and ROM or hybrids of these such as magnetic/optical storage media, FLASH media or memory type cards. Product labels or inserts can include identifying information of one or more components therein, dose amounts, clinical pharmacology of the active ingredient(s) including mechanism of action, pharmacokinetics (PK) and pharmacodynamics (PD). Product labels or inserts can include information identifying manufacturer information, lot numbers, manufacturer location, date, information on an indicated condition, disorder, disease or symptom for which a kit component may be used. Product labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Kits of the invention therefore can additionally include labels or instructions for practicing any of the methods and uses of the invention described herein. Product labels or inserts can include information on potential adverse side effects and/or warnings.

In certain embodiments, a kit comprises one or more controls having a known amount of syndecan-1 and/or an FGFR3. In some embodiments, a kit comprises cells expressing syndecan-1 and/or an FGFR3. The cells in the kit can be maintained under appropriate storage conditions until the cells are ready to be used.

In some embodiments, a kit is a diagnostic kit comprising a bi-specific binding agent. A bi-specific binding agent comprised in a diagnostic kit can take any suitable form. In some embodiments, a diagnostic kit comprises a bi-specific binding agent and a detectable label. In certain embodiments, for example, a diagnostic kit comprises or consists of a stick test, including necessary reagents to perform the method of the invention and to produce, for example, a colorimetric result which can be compared against a color chart or standard curve. A diagnostic kit can also comprise components necessary for detecting a bi-specific binding agent that binds specifically to a cell, for example a secondary antibody.

### EXAMPLES

### Example 1—Generation of Anti-CD138 Antibodies

Monoclonal antibodies were generated against human CD138 that (i) bind with high affinity and specificity, (ii) display rapid internalization, and/or (iii) display cross-reactivity with cynomolgus monkey derived CD138. To generate antibodies, mice (Balb/C mice; female 6-8 weeks old) were immunized and boosted with a mix of CD138 peptides 1-3 (Fusion 12) or peptides 4-6 (Fusion 13), see Table 11.

These peptides were designed to be distal to glycosylation sites, and in regions that were poorly conserved between human and mouse, but strongly conserved between human and cynomolgus species. Mice were immunized with the indicated peptides which were conjugated to KLH carrier protein following an immunization schedule using Complete Freund's Adjuvant (CFA) for the primary injection and Incomplete Freund's Adjuvant for all the subsequent boosts. Antibody serum titers of the immunized mice were assessed for binding by human CD138 and CD138-Fc binding ELISA. Mice with high titers were selected for fusion. Hybridomas were produced by electrofusion of mouse B cells and SP2/O myeloma cells using an optimized method.

TABLE 11
CD138 peptides used for immunization.

| | | |
|---|---|---|
| Peptide 1 | AGEGPKEGEAVVLPEVEPG | SEQ ID NO: 118 |
| Peptide 2 | KEGEAVVLPEVEPGLTARE | SEQ ID NO: 119 |
| Peptide 3 | VVLPEVEPGLTAREQEATP | SEQ ID NO: 120 |
| Peptide 4 | PEPTGLEATTASTSTLP | SEQ ID NO: 121 |
| Peptide 5 | ETTQLPTTHQA | SEQ ID NO: 122 |
| Peptide 6 | ATTAQEPATSHPHRDMQPGHHETS | SEQ ID NO: 123 |

Unlike in traditional methods, cloning of hybridomas was performed simultaneously in a single step wherein fused cells were selected by HAT selection, and single cell colonies were transferred into 96 well plates containing HT media, grown under limited selection and supernatants screened for antigen binding. Positive hybridomas were expanded into larger volumes and the cells were frozen for storage.

### Primary Hybridoma FACS Screen on CD138 Positive Cells (H929)

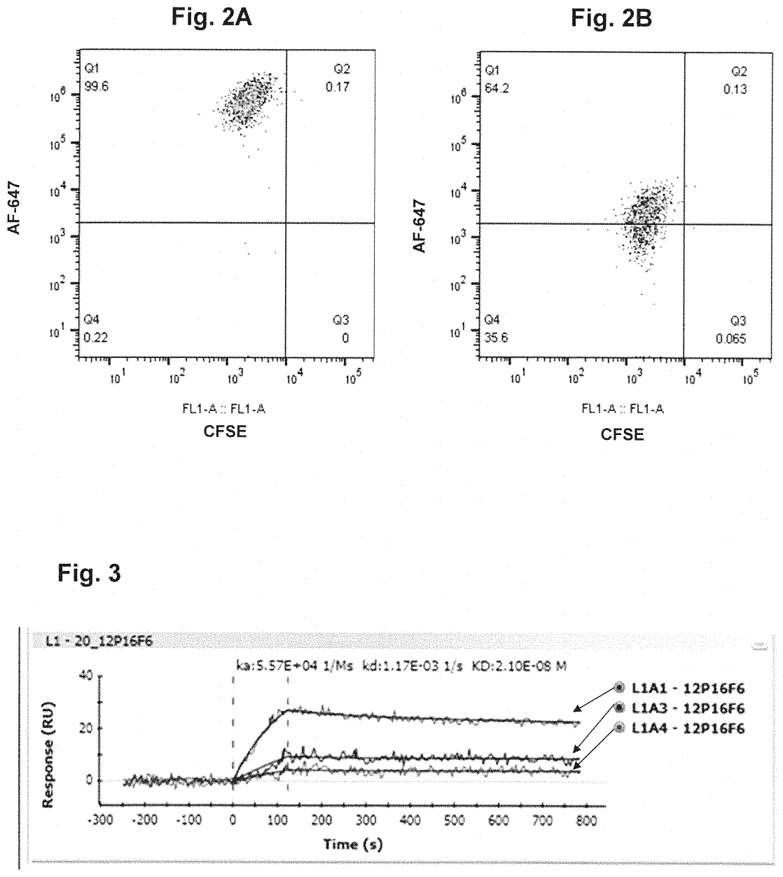

Hybridoma supernatants from Fusion 12 (plates 12-19) were screened by Fluorescence-activated cell sorting (FACS) using 96-well plates containing 20,000 H929 cells per well. H929 cells are a human B-lymphocyte cell line that expresses CD138 on its cell surface. Hybridoma supernatants were added to H929 cells for 1 hour at 4° C. The cells were washed followed by addition of an AlexaFluro 647 anti-mouse antibody and washed again to remove unbound antibody. The cells were analyzed by FACS to detect binding. FACS Data was analyzed by Flowjo software. If supernatant antibodies bound cells at a level ≥3 standard deviations above the average signal of the plate, they were selected for confirmation FACS and further characterization. A total of 5013 hybridomas from Fusion 12 underwent this primary FACS screen with 134 positive hits; a total hit rate of 2.7% in primary screening. A sample of the positive hybridoma clones from fusion 12 plate 16 is shown in Table 12 below. A FACS histogram of two representative positive hybridoma clones is shown in FIG. 2.

TABLE 12
Table from representative H929 FACS screen showing
representative positive hybridomas from fusion 12 plate 16.

| Hybridoma | | | | | | | Cells \| |
|---|---|---|---|---|---|---|---|
| Clone/ | | | | Q1 | | Q4 | Geom. |
| Antibody | | Well | | Geom. | | Geom. | Mean |
| Name | plate | ID | % Q1 | Mean | % Q4 | Mean | (FL4-A) |
| F12P16A4 | 16 | A4 | 58.3 | 3971 | 41.6 | 1139 | 2363 |
| F12P16B10 | 16 | B10 | 72 | 5051 | 27.8 | 1219 | 3409 |
| F12P16F6 | 16 | F6 | 99.6 | 7.07E+05 | 0.223 | 146 | 6.94E+05 |
| F12P16G3 | 16 | G3 | 64.1 | 4245 | 35.8 | 1136 | 2652 |
| F12P16H6 | 16 | H6 | 58 | 3733 | 41.7 | 1027 | 2182 |

* Geom. Mean indicates the average fluorescence intensity of cells within the indicated quadrant shown in FIG. 2;
Q1 indicates positive binding of antibody to H929 cells, while Q4 indicates unbound cells.

### Secondary FACS Screen of Fusion 12 Hybridomas

Hybridomas demonstrating positive binding in the primary FACS screening were selected and further characterized. For secondary screening, hybridoma supernatants were assayed by FACS for positive binding to H929 (CD138 expressing) and negative binding to ARH-77 (negative CD138 expression) cells. Negative cells were CSFE stained to help distinguish better positive and negative cell lines. The supernatants were then compared with their non-specific binding versus specific CD138 binding. The results of the representative secondary FACS screen are summarized in Table 13.

TABLE 13
FACS data summary for representative hybridoma clone
F12P16F6 (12P16F6) showing positive binding on
CD138-positive H292 cells and negative binding on
CD138-negative ARH77 cells.

| Hybridoma | | | | ARH77 |
|---|---|---|---|---|
| Clone/Antibody | H929 | H929 | % | Geom. |
| Name | Q1% | GeoMean | ARH77 (−) | Mean |
| F12P16F6 | 99.32% | 6.46E+05 | 21.85% | 1.12E+04 |

* Geom. Mean indicates the average fluorescence intensity of cells; Q1% indicates the percentage of H929 cells bound by antibody.

### Secondary ELISA Screen on Fusion 12 Hybridomas

CD138 and IgG1 isotype ELISAs were also conducted after the secondary screens to help confirm binding specificity and IgG type. CD138 binding ELISA was performed using recombinant CD138-Flag. Data indicated that all FACS-positive hybridomas also bound CD138 by ELISA. The IgG ELISA identified IgG positive antibodies. IgM antibodies were eliminated from further studies. A summary of the selection process to this point is shown in Table 14.

TABLE 14
Summary of primary and secondary screening results.

| | number of |
|---|---|
| | hybridoma |
| Screening Characterization | clones |
| Total Hybridomas Screened | 5013 |
| Primary Screen hits | 134 |
| No Expression | 39 |
| IgM positive | 23 |
| No Secondary Binding | 60 |
| IgG positive | 72 |
| Secondary FACS binders | 12 |

### Kinetic Binding of Representative Antibodies by SPR

Murine IgGs were purified from the hybridoma supernatants and the IgGs were subject to SPR (surface plasmon resonance) for kinetic binding measurements. Human or mouse CD138 His was immobilized on a GLM chip at 50 pg/mL on a BioRad Proteon. The antibodies were flowed over the bound chip at a rate of 30 μL/min at a concentration range of 167 nM to 10.4 nM to detect kinetic binding. KDs were measured using bivalent analyte fit. Kinetic results are shown in Table 15 and FIG. 3.

TABLE 15
SPR kinetic measurement for representative antibodies.

| Antibody | hCD138 ka | hCD138 kd | hCD138 | hCD138 | mCD138 |
|---|---|---|---|---|---|
| Name | (1/Ms) | (1/s) | K_{D} | K_{D} (nM) | K_{D} (nM) |
| mBT062 | 3.42 × 10⁵ | 6.66 × 10⁻⁴ | 1.95 × 10⁻⁹ | 2.0 | NB |
| F13P30A7 | 2.38 × 10⁵ | 1.27 × 10⁻³ | 5.32 × 10⁻⁹ | 5.3 | NA |
| F12P16F6 | 5.57 × 10⁴ | 1.17 × 10⁻³ | 2.10 × 10⁻⁸ | 21.0 | NB |
| F13P18D8 | 2.78 × 10⁵ | 5.23 × 10⁻⁴ | 1.88 × 10⁻⁹ | 1.9 | NA |
| F12P7G11 | 3.81 × 10⁶ | 3.30 × 10⁻² | 8.66 × 10⁻⁹ | 8.6 | NB |
| F13P14D3 | 1.07 × 10⁵ | 1.04 × 10⁻³ | 9.67 × 10⁻⁹ | 9.7 | NA |
| F11AP11E5 | 1.28 × 10⁵ | 1.17 × 10⁻² | 9.16 × 10⁻⁸ | 91.6 | NB |
| F12P18D4.a | 6.31 × 10⁵ | 1.8 × 10⁻² | 2.86 × 10⁻⁸ | 28.6 | 672 |

* NB = No binding; NA = Not Analyzed.

**Those marked NA were not analyzed by SPR but were shown to not cross-react with mouse CD138 via an ELISA binding assay (data not shown).

### Antibody Expression

Expression of two representative chimeric antibodies were assessed to determine the potential for scale-up production. Expi293 cells (250 mL) were transiently transfected with a vector directing the expression of 12P16F6 hIgG1 (also referred to as “chF6”) or 13P30A7 hIgG1 (also referred to as “chP30a7”). The chimeric antibody 12P16F6 hIgG1 includes the murine heavy chain variable region (SEQ ID NO:82) and light chain variable region (SEQ ID NO:34) of F12P16F6 and constant regions of human IgG1, kappa isotype. The chimeric antibody 13P30A7 hIgG1 includes the murine heavy chain variable region (SEQ ID NO:83) and light chain variable region (SEQ ID NO:35) of F13P30A7 and constant regions of human IgG1, kappa isotype. The results are summarized in Table 16 below. The expressed antibodies were also analyzed by SDS-PAGE and size exclusion chromatography (data not shown).

TABLE 16

| | Transfection | | | | Total | |
|---|---|---|---|---|---|---|
| | Volume | Purification | [Conc] | Volume | Yield | Endotoxin |
| Name | (mL) | Lot# | (mg/mL) | (mL) | (mg) | (EU/mg) |
| 12P16F6hlgG1 | 250 mL | AB150616-F6 | 2.21 | 3.2 | 7.07 | <4.5 |
| 13P30A7hlgG1 | 250 mL | AB150616-A7 | 1.02 | 1.05 | 1.07 | <9.8 |

### Cynomolgus Cross-Reactivity of Representative Antibodies

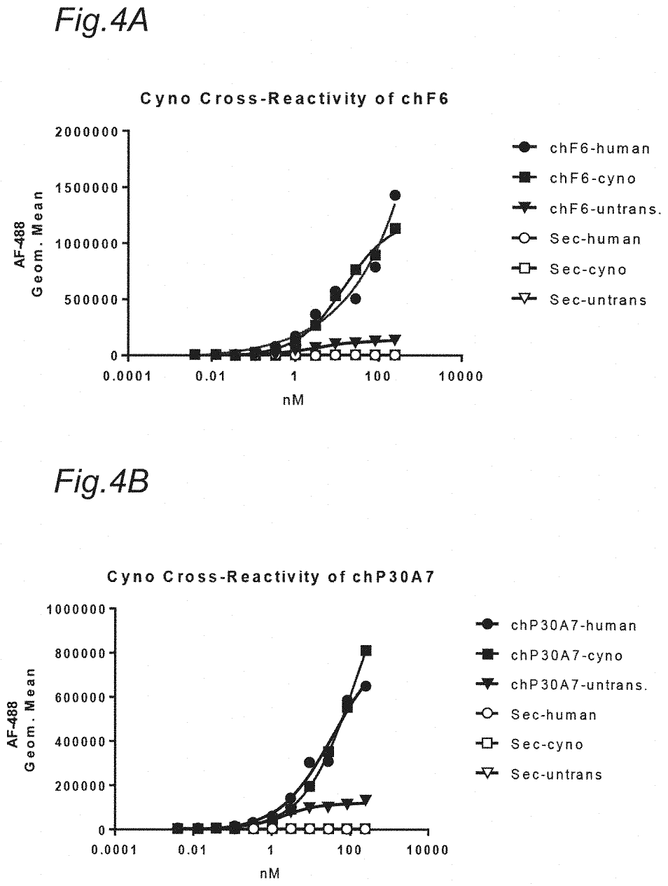

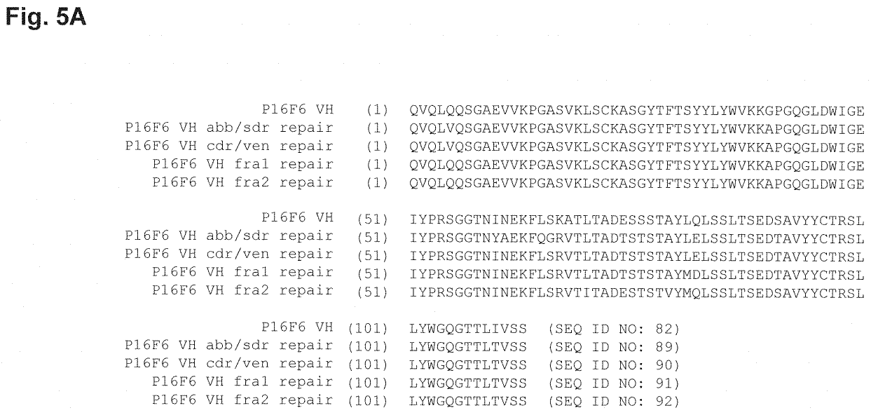

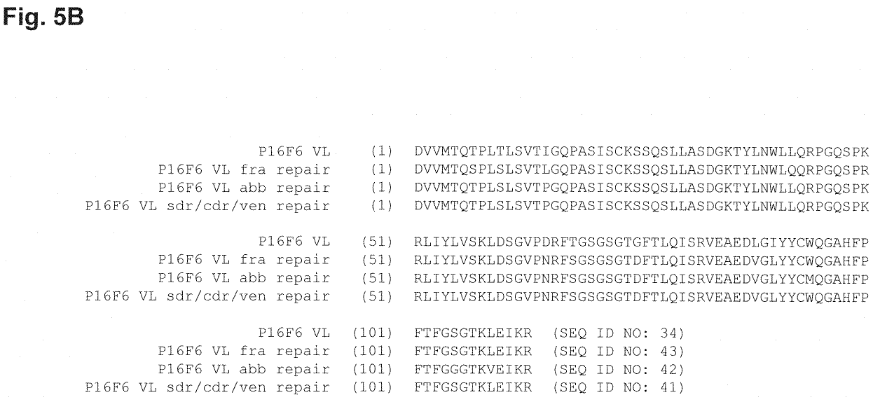

12P16F6 hIgG1 or 13P30A7 hIgG1 were tested for cross-reactivity to cynomolgus monkey CD138. Antibodies that cross-react with CD138 from cynomolgus monkey (cyno) have an advantage that they can be tested for toxicity in this strain of non-human primate prior to conducting efficacy trials. Briefly, a vector directing the cell-surface expression of human CD138 or cyno CD138 was transfected in Expi293 cells. Binding of 12P16F6 hIgG1 and 13P30A7 hIgG1 to transfected Expi293 cells was tested at 3-fold dilutions starting at 33.3 μg/mL. Secukinumab (Sec) was used as a negative control to ensure the transfected cells did not have any background binding. Representative antibodies 12P16F6 hIgG1 and 13P30A7 hIgG1 showed specific binding to both human and cyno CD138 (FIG. 4).

TABLE 17
Summary of data for representative CD138
hybridoma-derived antibodies.

| Antibody | SPR mCD138 KD | SPR hCD138 KD |
|---|---|---|
| Name | (nM) | (nM) |
| mBT062 | NB | 2 |
| IgG2a | NB | NB |
| F13P30A7 | NA | 5 |
| F12P16F6 | NB | 21 |
| F13P18D8 | NA | 2 |
| F12P7G11 | NB | 9 |
| F13P14D3 | NA | 10 |
| F11AP11E5 | NB | 92 |
| F12P18D4.a | 672 | 29 |

*NB = No detectable binding. NA = Not analyzed.

### Definitions of Certain Reagents and Materials Used in Example 1

Note that the name of a hybridoma clone here can refer to either the hybridoma cells or the antibody produced from the hybridoma cells, depending on the context in which the name is used. The name of a hybridoma clone often refers to the fusion (e.g., fusion #12 or #13, abbreviated F12 and F13 respectively), followed by the plate number preceded by the letter “P”, and the well number. For example, the hybridoma clone F12P16F6 (also referred to herein as 12P16F6 or P16F6), refers to an antibody obtained from a hybridoma derived from Fusion 12, plate 16, and well F6. mBT-062 is an IgG1, CD138 binding control antibody.

### Example 2—Humanization

A strategy was developed to design and create humanized versions of the murine anti-CD138 (anti-syndecan-1) antibodies described herein where the humanized version of the antibody retains the properties of the parental monoclonal antibody. Provided herein are examples of humanizing the chimeric monoclonal anti-CD138 antibody designated as chF6, which includes the human constant regions of a human IgG1/kappa isotype and the mouse variable regions of F12P16F6.

The humanized versions of chF6 generated herein were often benchmarked against the parent chF6 chimeric antibody. Other positive and negative controls were also used where appropriate.

Five humanization strategies were employed in parallel which resulted in the generation of three humanized F12P16F6 light chain sequences and four humanized F12P16F6 heavy chain sequences. In certain embodiments, the methods involve grafting of the murine complementarity determining regions (CDRs) onto human framework and constant regions. Each of the resulting three humanized light chains and four humanized heavy chains were expressed in combination with each other, and purified, which resulted in a total of twelve humanized anti-CD138 monoclonal antibodies. The humanized antibodies were analyzed for their expression/purification profiles, biophysical properties, binding to a CD138 peptide antigen, binding to native CD138, and specificity. Representative humanized antibodies were also evaluated for other biophysical properties.

### Methods

Expression and Purification of chP16F6

A vector directing the expression of the chimeric antibody chP16F6 was transfected in a volume of 250 ml into Expi293 cells using EXPIFECTAMINE™ 293 Transfection Kit. The supernatant was purified utilizing pH dependent, protein A purification. The chimeric antibodies were purified using HiTrap MabSelect SuRe 5 ml. After purification, the antibodies were buffer exchanged into 1× DPBS using Zeba spin columns. The recovery of chP16F6 was 7.1 mg at 2.21 mg/mL.

### Humanization of F12P16F6

Humanization of the heavy and light chain variable domains was performed using a method selected from (i) CDR grafting (designated as cdr) which was performed according to Jones et al. (1986) “Replacing the complementarity determining regions in a human antibody with those from a mouse” Nature 321:522-525 and Verhoeyen et al. (1988) “Reshaping human antibodies: grafting an anti-lysozyme activity” Science 239:1534-1536, where the CDRs as defined by Kabat et al. (1991) “Sequences of Proteins of Immunological Interest” 5th ed. US Department of Health and Human Services, Public Health Service, National Institutes of Health (NIH Publication No 91-3242), are grafted onto an appropriate human scaffold, while the critical framework residues are preserved; (ii) Grafting of abbreviated CDRs (designated as abb) which was performed according to Padlan et al. (1995) “Identification of specificity-determining residues in antibodies” FASEB J 9:133-139 were abbreviated CDRs, defined as residues 27D-34, 50-55, and 89-96 in the light chain, and 31-35B, 50-58, and 95-101 in the heavy chain, are grafted onto an appropriate human scaffold while the critical framework residues are preserved; (iii) SDR-transfer (designated as sdr) which was performed according to Padlan et al. (1995) “Identification of specificity determining residues in antibodies” FASEB J 9:133-139 where the residues that could be involved in antigen binding, are transplanted into an appropriate human sequence while the critical framework residues are preserved; (iv) The Frankenstein approach (designated as fra) which was perform according to Wu and Kabat (1992) “Possible use of similar framework region amino acid sequences between human and mouse immunoglobulins for humanizing mouse antibodies” Mol. Immunol. 29:1141-1146 where the CDRs are grafted onto a human scaffold made up of individual framework regions coming from appropriate human antibodies while the critical framework residues are preserved; and (v) Veneering (designated as ven) which was performed according to Padlan (1991) “A possible procedure for reducing the immunogenicity of antibody variable domains while preserving their ligand-binding properties” Mol. Immunol. 28:489-498 where the residues which are exposed in the nonhuman antibody, if the structure is known, or in a homologous molecule, if the structure is not known, are changed to the corresponding residues from an appropriate human antibody while the CDRs and the critical framework residues are preserved. In all of the methods described, ‘an appropriate human antibody’ is used to denote the closest human sequence (available in GenBank). The term “critical framework residue” is used to denote a residue that is deemed essential for the maintenance of three-dimensional structure (from the analysis of relevant high-resolution X-ray structures in the PDB). Sometimes a second “repaired” round of humanization was performed to improve the SEC profile of the antibody. Humanized antibodies produced in a second round are indicated by the designation rep or repair. The amino acid sequences of the resulting humanized heavy and light chain variable regions are shown in FIGS. 10A and 10B, respectively.

### Expression and Purification of Humanized P16F6 Repaired Constructs

Each of the four humanized heavy chains was paired with each of the 3 light chains to yield 12 different antibodies (Table 18). The 12 humanized P16F6 antibodies were expressed in Expi293 cells using EXPIFECTAMINE™ 293 Transfection Kit. All constructs were transfected in a volume of 125 ml except F6 cks-rep (more was needed for additional studies) and 375 ml of F6 f2ka-rep due to low protein expression. The supernatants were filtered through a 0.22 μm filter and treated with a protease inhibitor. Antibodies providing an expression level of >5 mg/L after buffer exchange and the ability to concentrate above ≥1 mg/mL were selected for further analysis.

TABLE 18

| | | Light Chain |
|---|---|---|
| | Heavy Chain Variable | Variable Region |
| Antibody | Region Name | Name |
| Name | (SEQ ID) | (SEQ ID) |
| F6 aka-rep | P16F6 abb/sdr-rep | P16F6 abb-rep |
| | (SEQ ID NO: 89) | (SEQ ID NO: 42) |
| F6 akf-rep | P16F6 abb/sdr-rep | P16F6 fra-rep |
| | (SEQ ID NO: 89) | (SEQ ID NO: 43) |
| F6 aks-rep | P16F6 abb/sdr-rep | P16F6 sdr/cdr/ven-rep |
| | (SEQ ID NO: 89) | (SEQ ID NO: 41) |
| F6 cka-rep | P16F6 cdr/ven-rep | P16F6 abb-rep |
| | (SEQ ID NO: 90) | (SEQ ID NO: 42) |
| F6 ckf-rep | P16F6 cdr/ven-rep | P16F6 fra-rep |
| | (SEQ ID NO: 90) | (SEQ ID NO: 43) |
| F6 cks-rep | P16F6 cdr/ven-rep | P16F6 sdr/cdr/ven-rep |
| (hF6) | (SEQ ID NO: 90) | (SEQ ID NO: 41) |
| F6 f1ka-rep | P16F6 fra1-rep | P16F6 abb-rep |
| | (SEQ ID NO: 91) | (SEQ ID NO: 42) |
| F6 f1kf-rep | P16F6 fra1-rep | P16F6 fra-rep |
| | (SEQ ID NO: 91) | (SEQ ID NO: 43) |
| F6 f1ks-rep | P16F6 fra1-rep | P16F6 sdr/cdr/ven-rep |
| | (SEQ ID NO: 91) | (SEQ ID NO: 41) |
| F6 f2ka-rep | P16F6 fra2-rep | P16F6 abb-rep |
| | (SEQ ID NO: 92) | (SEQ ID NO: 42) |
| F6 f2kf-rep | P16F6 fra2-rep | P16F6 fra-rep |
| | (SEQ ID NO: 92) | (SEQ ID NO: 43) |
| F6 f2ks-rep | P16F6 fra2-rep | P16F6 sdr/cdr/ven-rep |
| | (SEQ ID NO: 92) | (SEQ ID NO: 41) |

*F6 and P16F6 indicates that the humanized antibody chains were derived from F12P16F6.

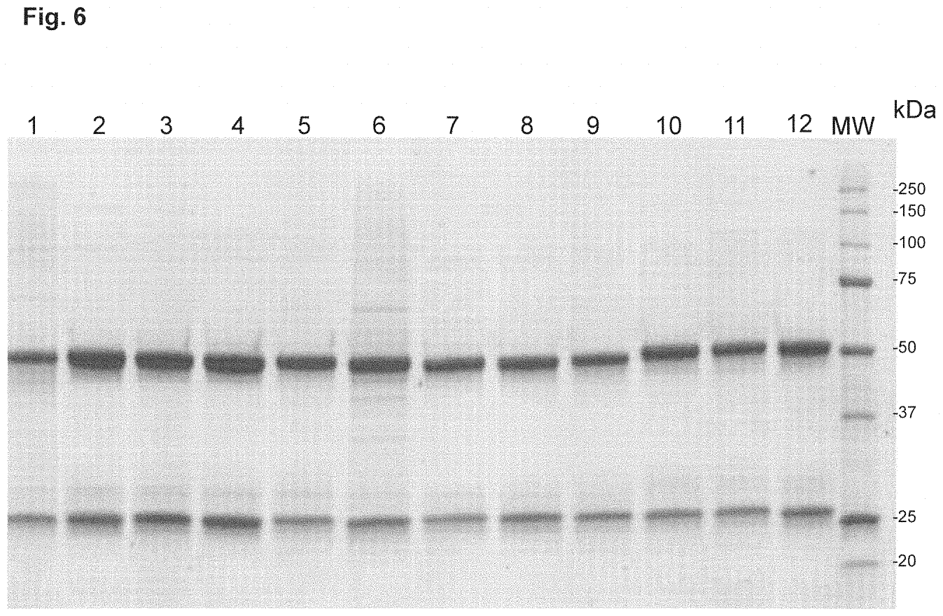

The antibodies were purified utilizing pH dependent, protein A purification (HiTrap MabSelect SuRe 5 mL). After purification, the antibodies were buffer exchanged into 1× DPBS using zeba spin columns. The recovery and relative stability as determined by size exclusion chromatography (SEC) analysis varied between the humanized antibodies (SEC profiles not shown). Table 19 summarizes the recovery, concentration and the percent monomer as determined by SEC. An SDS-PAGE analysis of eleven representative humanized antibodies is shown in FIG. 6. The nomenclature sometimes takes the form of hF6 xky-rep where h stands for humanized, F6 stands for F12P16F6-derived, x stands for the first letter of the first procedure used to generate the humanized heavy chain sequence (e.g., x can be a=abb, s=sdr, f=fra, or c=cdr), k stands for kappa light chain, and y stands for the first letter of the first procedure used to generate the humanized light chain sequence (a=abb, s=sdr, f=fra, c=cdr). The term “rep” ˜stands for “repaired” indicating that at least a second round of humanization was performed, often using a different method.

TABLE 19

| | Final | | | |
|---|---|---|---|---|
| Antibody | Recovery | Concentration | Recovery | SEC % |
| Name | (mg) | (mg/mL) | (mg/mL) | Monomer |
| F6 aka-rep | 1.2 | 1.44 | 9.6 | 75.2 |
| F6 akf-rep | 3.28 | 1.13 | 26.24 | 95.4 |
| F6 aks-rep | 4.89 | 1.63 | 39.12 | 95.2 |
| F6 cka-rep | 1.72 | 1.23 | 13.76 | 88.8 |
| F6 ckf-rep | 8.4 | 1.05 | 67.2 | 95.3 |
| F6 cks-rep | 15.25 | 1.22 | 68.8 | 99.3 |
| (hF6) | | | | |
| F6 f1ka-rep | 0.79 | 1.08 | 6.32 | 92.9 |
| F6 f1kf-rep | 2.86 | 1.1 | 22.88 | 94.3 |
| F6 f1ks-rep | 3.5 | 1.25 | 28 | 95.6 |
| F6 f2ka-rep | 0.37 | 1.06 | 2.96 | 79.1 |
| (Lot 1) | | | | |
| F6 f2ka-rep | 0.71 | 1.23 | 2.84 | 95 |
| (Lot 2) | | | | |
| F6 f2kf-rep | 1.8 | 1.8 | 14.4 | 95.1 |
| F6 f2ks-rep | 2.81 | 1.34 | 22.48 | 77.6 |

### CD138 Binding of Humanized P16F6 Repaired Constructs by FACS

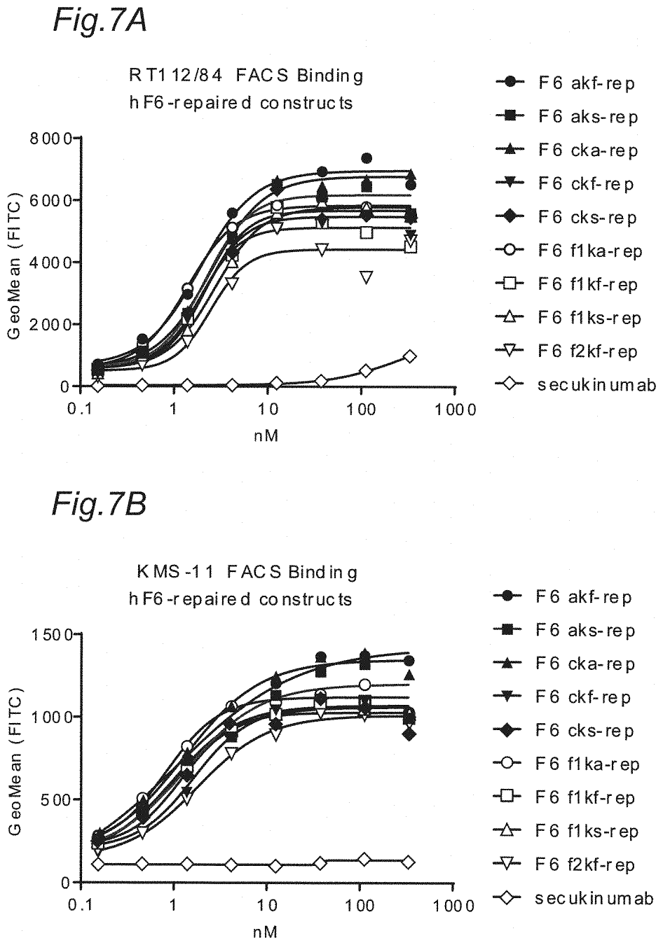

Analysis of cell-surface binding to human CD138 was performed on 9 representative humanized anti-CD138 antibodies by FACS (FIG. 7). Secukinumab was used as a negative control. Two cell lines expressing moderate levels of CD138 were used to test binding; multiple myeloma cell line KMS-11 and bladder cancer line RT112/84. In previous experiments, 12P16F6 hIgG1 showed an EC50 of approximately 9 nM in RT-122/84 cells and ˜3 nM in KMS-11 cells. EC50s were calculated using four parameter fit curves (Table 20). The constructs were also tested against ARH-77 cells, which are CD138 negative lymphoblasts. NB indicates no specific binding observed.

TABLE 20
Calculated EC50 values of binding to endogenous CD138

| Antibody Name | KMS-11 (nM) | RT-112 (nM) | ARH-77 (nM) |
|---|---|---|---|
| F6 akf-rep | 1.09 | 1.88 | NB |
| F6 aks-rep | 1.36 | 2.13 | NB |
| F6 cka-rep | 1.30 | 2.44 | NB |
| F6 ckf-rep | 1.64 | 2.09 | NB |
| F6 cks-rep | 1.2 | 2.2 | NB |
| (hF6) | | | |
| F6 f1ka-rep | 0.83 | 1.39 | NB |
| F6 f1kf-rep | 0.94 | 1.93 | NB |
| F6 f1ks-rep | 0.97 | 2.73 | NB |
| F6 f2kf-rep | 1.81 | 2.46 | NB |
| secukinumab | NB | NB | |

*NB = No detectable binding.

### CD138 Binding ELISA of Humanized P16F6 Repaired Constructs

A CD138 binding ELISA was performed with 9 representative humanized antibodies to determine binding to portion of the linear CD138 peptide used for immunization (FIG. 7). Plates were coated with the hCD138 peptide (AGEGPKEGEAVVLP; SEQ ID NO:94) and a negative control peptide (QAAVTSHPHGGMQPGLHETSA; SEQ ID NO:124), or a mouse CD138 peptide for which F12P16F6 does not bind. Coated plates were incubated with various dilutions of each of the 9 representative antibodies overnight and binding was detected with a goat anti-human IgG (H+L)-HRP. EC50s were determined using four parameter fit curves (Table 21). An ELISA was also performed to detect binding to plate-coated human CD138-Fc protein (Table 21). The analysis and results were similar.

TABLE 21

| | hCD138 peptid EC50 | hCD138-Fc EC50 |
|---|---|---|
| Antibody Name | (nM) | (nM) |
| 12P16F6 hIgG1 (chF6) | 0.599 | 0.4715 |
| F6 akf-rep | 1.54 | 0.8791 |
| F6 aks-rep | 1.285 | 0.9694 |
| F6 cka-rep | 1.654 | 0.8549 |
| F6 ckf-rep | 0.9812 | 0.5354 |
| F6 cks-rep (hF6) | 0.3299 | 0.1974 |
| F6 f1ka-rep | 0.7443 | 0.433 |
| F6 f1kf-rep | 0.4133 | 0.2247 |
| F6 f1ks-rep | 1.314 | 0.7616 |
| F6 f2kf-rep | 0.7257 | 0.4051 |

### Summary of Selected Study Results

Table 22 shows a summary of the analytic results for 13 representative humanized anti-CD138 monoclonal antibodies.

TABLE 22
Summary of biophysical characteristics of representative
humanized antibodies.

| | | | FACS | ELISA |
|---|---|---|---|---|
| Antibody | Recovery | SE % | binding | CD138 Fc |
| Name | (mg/L) | monomer | (KMS11) | binding |
| F6 aka-rep | 9.6 | 75.2 | — | — |
| F6 akf-rep | 26.24 | 95.4 | 1.09 | 0.8791 |
| F6 aks-rep | 39.12 | 95.2 | 1.36 | 0.9694 |
| F6 cka-rep | 13.76 | 88.8 | 1.30 | 0.8549 |
| F6 ckf-rep | 67.2 | 95.3 | 1.64 | 0.5354 |
| F6 cks-rep | 68.8 | 99.3 | 1.20 | 0.1974 |
| (hF6) | | | | |
| F6 f1ka-rep | 6.32 | 92.9 | 0.83 | 0.433 |
| F6 f1kf-rep | 22.88 | 94.3 | 0.94 | 0.2247 |
| F6 f1ks-rep | 28 | 95.6 | 0.97 | 0.7616 |
| F6 f2ka-rep | 2.96 | 79.1 | — | — |
| (lot 1) | | | | |
| F6 f2ka-rep | 2.84 | 95 | — | — |
| (lot 2) | | | | |
| F6 f2kf-rep | 14.4 | 95.1 | 1.81 | 0.4051 |
| F6 f2ks-rep | 22.48 | 77.6 | — | — |

### Example 3—Determination of Crystal Structure

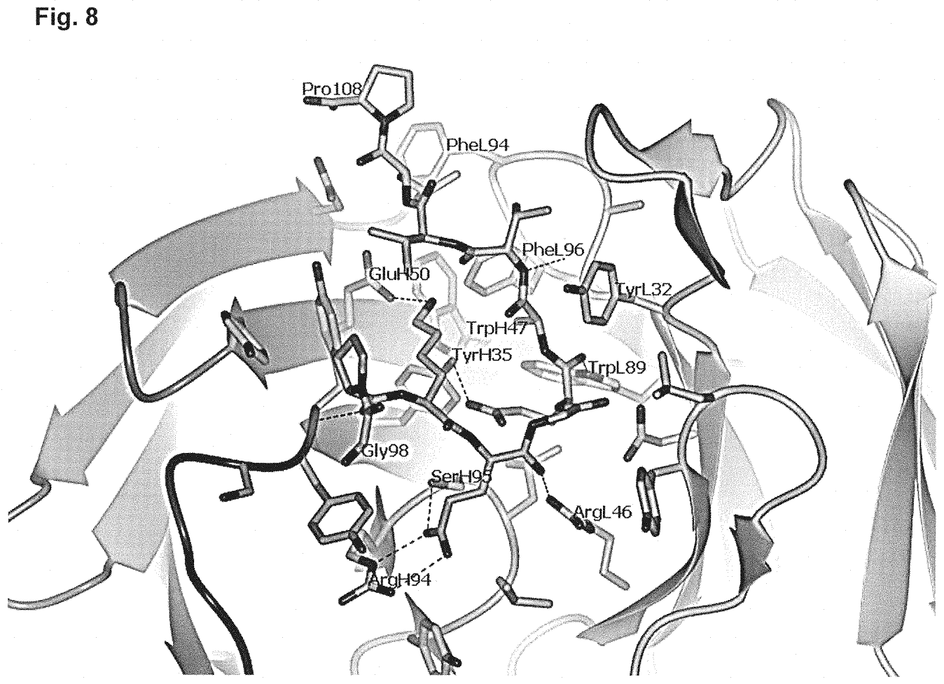

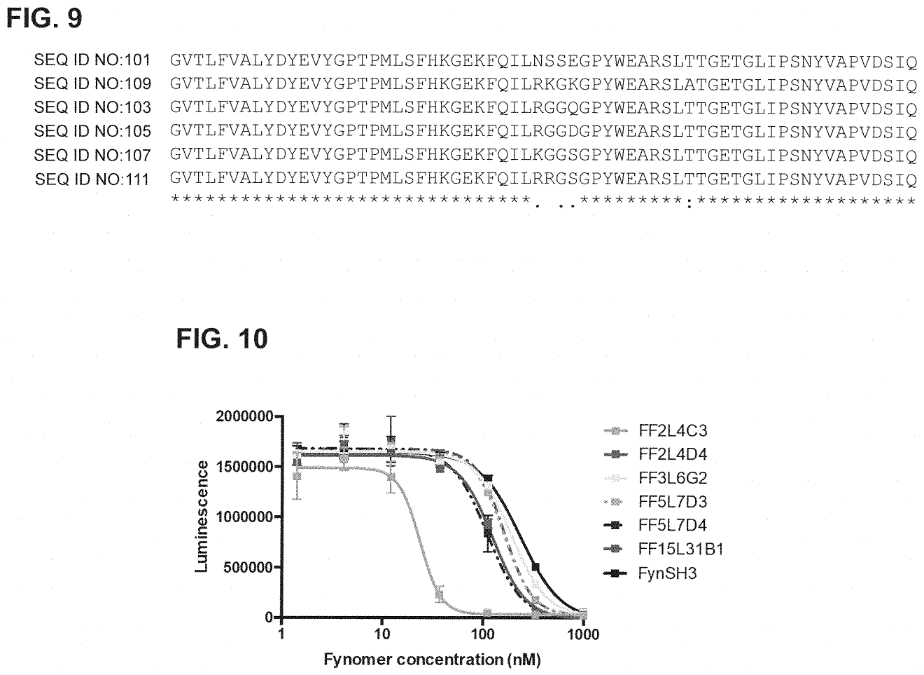

The X-ray crystal structure of a human syndecan-1 peptide in complex with a humanized anti-CD138 antibody Fab fragment was solved at 1.95 Å resolution. The structure included one copy each of syndecan-1 peptide and Fab per asymmetric unit (FIG. 8).

### Structure Description

The humanized antibody Fab comprises the humanized heavy chain variable region (SEQ ID NO:90) and the humanized light chain variable region (SEQ ID NO:41). The CDR canonical structures were analyzed in accordance with the PyIgClassify database. The heavy chain CDRs were classified as follows: H1-13-1 (CDR-length-cluster), H2-10-1 and H3-6-1. The light chain CDRs were classified as follows: L1-16-1, L2-8-1 and L3-9-cis7-1. Syndecan-1 peptide binds to a single Fab, and complex formation buries 540 Å2 of the solvent-accessible surface areas of syndecan-1 peptide and Fab (313.6 Å2 chains A and H; 226.4 Å2 chains A and L).

All visible syndecan-1 peptide residues from 98-108 participate in direct contacts with Fab (FIG. 8). The specific Fab residues involved in the interface are 31-33, 35, 47, 50, 52, 58, 94-96 and 101-102 from chain H and 27-28, 32, 34, 46, 49-50, 89-94 and 96 from chain L. This means that four residues from CDR H1 participate in the interface, along with three residues from CDR H2 and five residues from CDR H3. In addition, four residues from CDR L1, two residues from CDR L2 and seven residues from CDR L3 participate in the interface.

A 1 mL aliquot of Fab at 5.88 mg/mL (approx. 125 pM) was mixed with 250 pM syndecan-1 peptide (AGEGPKEGEAVVLP; SEQ ID NO:94) and incubated at 4° C. for two hours. The complex was fractionated on an S200 size exclusion column which had been pre-equilibrated with buffer containing 20 mM Tris pH 7.5 and 150 mM NaCl. Peak fractions were pooled and concentrated for crystallization. The final protein concentration as determined by Bradford assay was 3 mg/mL.

Approximately 400 crystallization conditions were screened by the hanging drop method of vapour diffusion in 96 well format using a mosquito robot (TTP Labtech). Crystal growth was observed at 20° C. in two conditions: 2.1 M DL-malic acid pH 7.0, and 60% Tacsimate pH 7.0. Crystallization was optimized further in 24 well format.

### Crystal Cooling and Data Collection

The crystal described was grown using the hanging drop method of vapour diffusion in a 24 well plate with a precipitant solution containing 1.7 M DL-Malic acid, pH 7.0. In house X-ray diffraction screening indicated that resolution could be improved by pre-soaking crystals in a solution containing 3.0 M DL-Malic acid, pH 7.0 for 24 hours. The crystal was cryo-cooled by capturing it in a loop directly from the soaking drop and plunging it into liquid nitrogen. A synchrotron data set was collected at ESRF beamline ID30A-1.

### Structure Solution and Refinement

Data processing in MOSFLM (CCP4) and AIMLESS (CCP4) indicated that the most likely space group was P212121 with unit cell dimensions a=60.6 Å, b=132.9 Å and c=51.2 Å, giving a total cell volume of 411706.34 Å. Calculation of the Matthews coefficient (2.14 Å3/Da and 42.7% solvent content) indicated that there was most probably one complete Fab-syndecan-1 complex per asymmetric unit. Models for use in molecular replacement (MR) were chosen by BLAST searching the sequences of each component (Fab heavy and light chains) against the PDB. Models with highest sequence identity were 3 sgo (Fab heavy chain) and 4ojf (Fab light chain). The large number of Fab crystal structures deposited in the PDB has revealed a wide variety in elbow angles present between variable and constant domains. This variation in elbow angles can cause the overall tertiary structure of two otherwise highly homologous Fab fragments to be significantly different, which in turn causes MR to fail. For this reason the hinge regions between the variable and constant domains of the heavy and light chains were removed to create four separate MR search ensembles (VH, VL, CH and CL). Amino acid residues were trimmed from the CDRs of the heavy and light variable domain models after visual inspection in COOT to prevent any potential clashes that might also cause MR to fail. All four of the input search ensembles (VH, VL, CH and CL) that were required to build a complete Fab were correctly located by MR using PHASER (McCoy et al., 2007) (CCP4). The MR output model was given 20 cycles of jelly body refinement using REFMAC5 (CCP4). The protein sequence was mutated to match that of Fab using CHAINSAW(CCP4). The model was improved iteratively through successive cycles of model building and refinement until all of the ordered regions of Fab visible in the electron density maps were complete. The heavy and light chain amino acids were renumbered in accordance with the Kabat antibody numbering convention. Electron density corresponding to the syndecan-1 peptide was clearly visible. Syndecan-1 amino acid residues were added by hand in COOT and the correct numbering was applied. Water molecules were added using the water placement option in COOT and the complete model was refined using REFMAC5 (CCP4). The final Fab model contained heavy chain residues 1-216 (chain H) and light chain residues 1-212 (chain L) with no breaks in either chain. The final syndecan-1 model contained residues 98-108 (chain A). The final model also contained 205 water molecules. Final Rwork=21.2%, Rfree=26.1%.

TABLE 23
Data Collection, Processing, and Refinement Statistics
Data collection and processing statistics

| Synchrotron, Beam line | ESRF, ID30A-1 | |
|---|---|---|
| Date and time of data collection | 28 Jan. 2017; | |
| | 04:13:17 | |
| Wavelength (Å) | 0.966 | |
| Detector type | Dectris Pilatus3 2M | |
| Transmission (%) | 100 | |
| Temperature (K.) | 100 | |
| Exposure time (s) | 0.1 | |
| Oscillation range per frame (°) | 0.2 | |
| Overall rotation (°) | 180 | |
| Resolution range (Å) | 44.76-1.95 | |
| Number of observed reflections | 197864 | |
| Number of unique reflections | 30915 | |
| Multiplicity (overall and last shell) | 6.4 | (6.1) |
| Completeness (%) (overall and last shell) | 99.9 | (99.9) |
| Rmerge (%) (overall and last shell) | 12.0 | (92.1) |
| Mean I/sigma (overall and last shell) | 12.1 | (1.1) |
| CC(½) (overall and last shell) | 0.996 | (0.592) |

| | |
|---|---|
| Space group | P212121 |
| Unit cell parameters (Å), (°) | 60.57 132.87 51.16 |
| 90.00 90.00 90.00 | |

Refinement statistics

| | |
|---|---|
| Refinement program | REFMAC5 |
| Resolution range (Å) | 66.43-1.95 |
| Number of reflections (working/test) | 29294/1561 |
| Rwork (%) | 21.2 |
| Rfree (%) | 26.1 |
| Protein residues modelled | 435 |
| Number of protein atoms modelled | 3324 |
| Number of water atoms modelled | 205 |
| RMSD Bond lengths (Å) | 0.007 |
| RMSD Bond angles (°) | 1.289 |
| Mean overall B value (Å2) | 28.9 |
| Ramachandran plot favoured (%) | 96.8 |
| Ramachandran plot allowed (%) | 3.2 |
| Ramachandran plot outlier region (%) | 0.0 |

### Example 4—FGFR3 Fynomer Production

The SH3 domain of the human Fyn kinase was successfully used as a scaffold to engineer Fynomers polypeptides that bind with high affinity and specificity to FGFR3 target proteins. A proprietary phage display library containing more than 8.5×10¹⁰ individual Fynomer clones was used for selections on different recombinant FGFR3 antigens. Different selection and screening strategies were used. In one strategy, recombinant FGFR3 isoform 3B and FGFR3 isoform 3C derived from human and/or monkey (e.g., Cynomolgus monkey, Macaca fascicularis) were used for selection of phage clones expressing a Fynomer that specifically binds to at least two FGFR3 splice isoforms (e.g., 3B and 3C). Several rounds of selection were conducted using different FGFR3 antigens and/or combinations of FGFR3 antigens. One goal of the Fynomer selection process was to isolate a Fynomer with (i) selective binding to both FGFR3b and FGFR3c, (ii) cross-reactivity to human and monkey, and possibly mouse, and (iii) the ability to internalize bound receptors. This representative example describes a process of selecting such a Fynomer.

Using recombinant human FGFR3b-Fc and FGFR3c-Fc as targets, we successfully selected and isolated several families of Fyn SH3-derived binding proteins that are capable of binding to both splice variants of human FGFR3 (FGFR3b and FGFR3c). We continued with the most promising candidate family for further studies.

Interestingly, a Fyn SH3-derived polypeptide referred to as FF2L4C3 (SEQ ID NO:101), carrying the RT-loop sequence “EVYGPTPM” (SEQ ID NO:100), was enriched during the selection process and showed very promising internalization properties among 29 tested anti-FGFR3 Fynomers (see FIG. 10). In more detail, 5 other sequence families were excluded from further analysis. The Fynomers belonging to the most promising sequence family showed the best affinities and internalization properties.

In order to obtain Fyn SH3-derived FGFR3 binders with higher affinities and improved internalization properties, FF2L4C3 (SEQ ID NO:101) was used as template for affinity maturation. The RT-loop sequence “EVYGPTP” (SEQ ID NO:131) was kept constant and was combined with a randomized n-src-loop repertoire (where a stretch of 4 to 6 randomized amino acid residues were introduced at the positions (X¹) to (X⁴) in SEQ ID NO:99). The process of affinity maturation library generation was essentially the same as described for cloning of the naïve library with a randomized n-src-loop (“library 0” as described in [25]).

After naïve and affinity maturation selections, enriched Fyn SH3-derived polypeptides were screened for binding to FGFR3 by lysate ELISA. DNAs encoding the Fyn SH3-derived binding proteins were cloned into the bacterial expression vector pQE12 (Qiagen) so that the resulting constructs carried a C-terminal myc-hexahistidine tag as described in Grabulovski et al. [26]. The polypeptides were expressed in the cytosol of E. coli bacteria in a 96-well format and 200 μL of cleared lysate per well was prepared as described in Bertschinger et al. [27]. Briefly, transformed bacterial colonies were picked from the agar plate and grown in a round bottom 96-well plate (Nunc, cat. no. 163320) in 200 μL 2×YT medium containing 100 pg/mL ampicillin and 0.1% (w/v) glucose. Protein expression was induced after growth for 3 h at 37° C. and rotational shaking at 200 r.p.m. by adding 1 mM IPTG (Applichem, Germany). Proteins were expressed overnight in a rotary shaker (200 r.p.m., 30° C.). Subsequently, the 96-well plate was centrifuged at 1800 g for 10 min and the supernatant was discarded. Bacterial pellets were lysed using BugBuster® plus Benzonase® (Millipore 70750-3) and lysates were subsequently cleared by centrifugation for 10 min at 1800× g. 60 μL lysate were mixed with 170 μL PBS and filtered through a 0.45 μm Multiscreen filter plate (Millipore MSHVN4510), in order to eliminate any residual bacterial debris.

Monoclonal bacterial lysates were used for ELISA. For the ELISA, Maxisorp plates were coated overnight with either 5 pg/mL huFGFR3b-Fc, 5 pg/mL huFGFR3c-Fc or 5 pg/mL poly IgG and blocked for at least 1 h with 2% MPBS. Cleared lysates containing soluble Fynomer with a C-terminal myc- and hexahistidine peptide tag were added in 2% MPBS containing murine monoclonal anti-myc tag antibody, clone 9E10 (Roche Applied Science 11 667 203 001) to the maxisorp plates. Bound Fynomer was detected via 9E10 by an anti-mouse IgG-horse radish peroxidase conjugate (Sigma-Aldrich A2554). The detection of peroxidase activity was done by adding BM blue POD substrate (Roche) and the reaction was stopped by adding 1 M H₂SO₄.

The DNA sequence of the specific binders was verified by DNA sequencing.

### Results

The amino acid sequences of representative ELISA positive Fyn SH3-derived polypeptides that bind specifically to FGFR3b and FGFR3c are presented in SEQ ID NOs: 101, 103, 105, 107, 109 and 111. The Fyn-SH3 derived polypeptides SEQ ID NOs: 103, 105, 107 and 109 are a selection of binders from a large pool of molecules that were obtained after affinity maturation of FF2L4C3 (SEQ ID NO:101), and are presented here because of their improved affinities and internalization properties (as shown herein).

More than 80 Fynomers derived from SEQ ID NO:101 were obtained and characterized, each demonstrating specific binding to FGFR3b and FGFR3c and demonstrating desirable biophysical properties, affinities and internalization properties. Fynomer having the amino acid sequences of SEQ ID NOs:103, 105, 107, 109, and 111 are representative of these binders.

### Example 5: Fyn SH3-Derived Polypeptides of the Invention Bind to Human FGFR3b and FGFR3c with High Affinities

This example shows the characterization of the preferred Fyn SH3-derived FGFR3-binding polypeptides by surface plasmon resonance and flow cytometry experiments.

### Methods

### a) Affinity Measurements by BIAcore

Affinities were measured using a BIAcore T200 instrument. One flow cell on a CM5 series S chip (GE Healthcare BR-1005-30) was coated with the anti-myc antibody 9E10 (Roche 11 667 203 001; coating density ranging between 6000 and 8000 RU) using the amine coupling kit (GE Healthcare BR100633).

The parental Fynomer FF2L4C3 (SEQ ID NO:101), at a concentration of 500 nM, and the Fynomers with SEQ ID NOs:103, 105, 107, 109 and 111, at a concentration of 100 nM, were captured on the 9E10 surface followed by injections of different concentrations of huFGFR3b-Fc, huFGFR3c-Fc or cynoFGFR3c-Fc (0 nM, 3.9 nM, 7.8 nM, 15.6 nM, 31.25 nM, 62.5 nM, 125 nM, 250 nM and 500 nM for the measurements of the parental Fynomer FF2L4C3, 0 nM, 0.046 nM, 0.14 nM, 0.41 nM, 1.2 nM, 3.7 nM, 11.1 nM, 33.3 nM and 100 nM for Fynomers with SEQ ID NOs: 103, 105, 107, 109 and 111). Sensograms were recorded and apparent kinetics constants were determined by curve fitting using the 1.1 Langmuir interaction model in the BIAevaluation 2.1 software.

### b) Affinity Measurements by Flow Cytometry

Binding of Fynomers to huFGFR3 on cells was analyzed by flow cytometry using KMS-11 cells (JCRB1179) as FGFR3-positive cells and N87 (ATCC, CRL-5822) as a FGFR3-negative control cell line. Both KMS-11 and N87 cells were maintained in RPMI1640 medium (Invitrogen 52400-25). All media were supplemented with 25 U/mL penicillin, 25 pg/mL streptomycin and 10% FCS. To harvest the semi-adherent KMS-11 cells from a T150 flask, the supernatant was removed into a 50 mL falcon tube, and the cells were washed with 10 ml PBS, which was also added to the falcon tube. 2 ml of Accutase (Sigma A6964) was added to the flask, and incubated for 10 min at 37° C. The Accutase was inactivated with the addition of 10 ml medium and added to the falcon tube, which was then centrifuged (250×g, 5 min) to pellet the cells. The cells were resuspended in FACS buffer (PBS+1% FCS+0.2% sodium azide) to a cell concentration of 1×10⁶ cells/mL and 100 μL was used per well (1×10⁵ cells/well) for the flow cytometry staining in a 96-well round bottomed plate (Nunc 163320). For adherent N87 cells the supernatant and wash were discarded, and only the Accutase-detached cells were collected and prepared.

Fynomers were co-incubated with the mouse anti-myc antibody (clone 9E10; Roche 11667149001) to allow cross linking of myc-tagged Fynomers prior to cell binding. Fynomers were diluted to 1 μM and co-incubated with 667 nM 9E10 anti-myc antibody (3:2 molar ratio) in FACS buffer, for approximately 10 minutes on ice.

This mixture was serially diluted 1 in 4 down to a Fynomer concentration of 0.06 nM (8 concentrations in total). Controls included the secondary antibody 9E10 only (no Fynomer; 667 nM 9E10), cells only (FACS buffer only) and an anti-FGFR3 antibody (R&D systems; cat. No. MAB766) at a concentration of 10 nM. Cells were centrifuged in the 96-well plate (250× g, 5 min) and were resuspended with the samples indicated above, before incubation on ice for 1 hr. The plate was centrifuged and washed (PBS+0.2% sodium azide), before centrifuging again. Then 50 μL of the secondary antibody anti-mIgG Alexa488 (Life Technologies A21202) was added to the cells at a concentration of 4 pg/mL, before incubating in the dark, on ice, for 45 min. The plate was centrifuged and washed twice with PBS+0.2% sodium azide, before resuspending in FACS buffer and FACS analysis (Millipore Guava easyCyte 8HT).

FACS data analysis was performed using Prism 6. The data was transformed (X=log X), and analyzed using a non-linear fit, log(agonist) vs. response—Variable slope (4 parameters).

### Results

### a) Affinity Measurements by BIAcore

The binding properties were analyzed by real-time interaction analysis on a BIAcore chip revealing the following dissociation constants (KD) for selected FGFR3-binding polypeptides:

TABLE 24
Apparent kinetics constants of the binding of FGFR3-binding Fynomers to
recombinant human FGFR3b, human FGFR3c and cynomolgus FGFR3c.

| | | | huFGFR3c | cyFGFR3c |
|---|---|---|---|---|
| | SEQ ID | huFGFR3b | KDₐₚₚ | KDₐₚₚ |
| Fynomer | NO. | KDₐₚₚ (pM) | (pM) | (pM) |
| FF2L4C3 | 101 | 4700 | 4600 | 5900 |
| FF44L65G12 | 103 | 690 | 685 | 110 |
| FF44L65G7 | 105 | 470 | 335 | 280 |
| FF48L66G7 (G7) | 107 | 260 | 190 | 210 |
| FF43L65D5 | 109 | 335 | 230 | 160 |
| FF44L65B7 | 111 | 100 | 250 | 260 |

The measured apparent affinities (Table 24) of the Fyn SH3-derived polypeptides (SEQ ID NOs:101, 103, 105, 107, 109 and 111) binding to FGFR3b and FGFR3c are surprisingly high considering the fact that sub-nanomolar values were obtained after only one round of affinity maturation. Moreover, these measurements confirmed the comparable binding properties of the Fyn SH3 derived polypeptides (SEQ ID NOs: 101, 103, 105, 107, 109 and 111) to both human isoforms of FGFR3 (FGFR3b and FGFR3c), and to cynomolgus FGFR3c (binding to cynomolgus FGFR3b was not tested).

### b) Affinity Measurements by Flow Cytometry

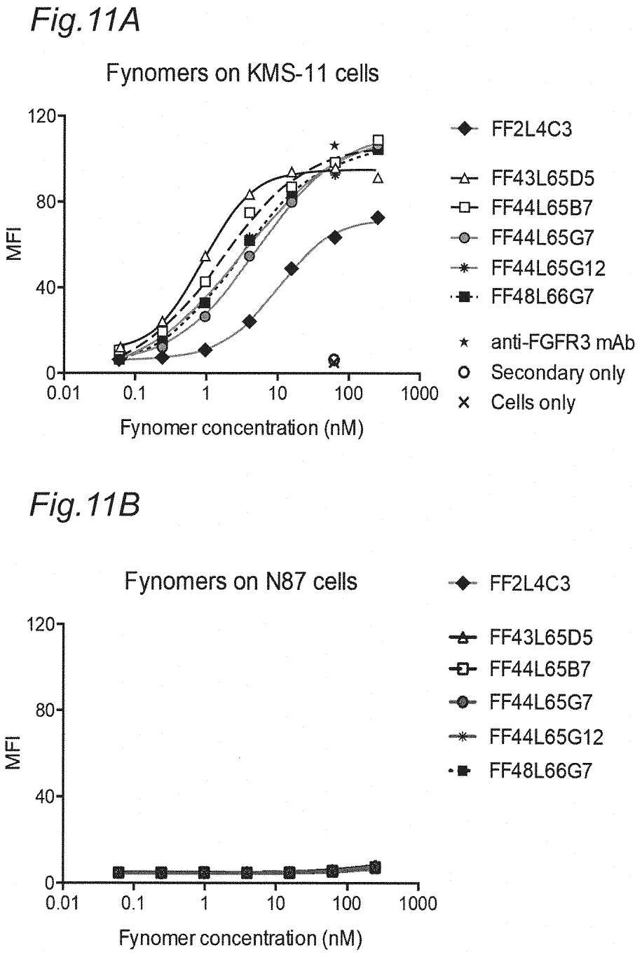

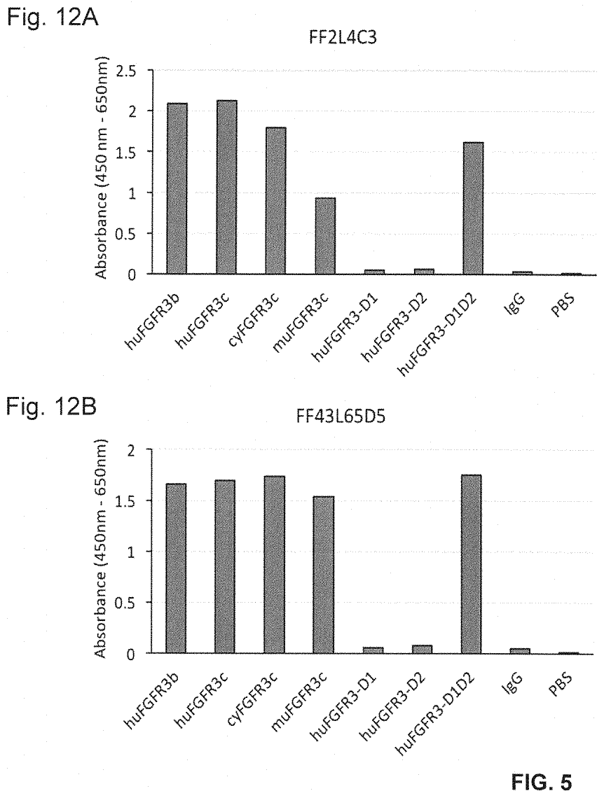

The binding properties were analyzed by flow cytometry using FGFR3-positive KMS-11 cells and FGFR3-negative N87 cells as negative control. The following EC50 values for selected FGFR3-binding polypeptides were measured as shown in Table 25 and FIG. 11.

TABLE 25
EC50 values determined on FGFR3-positive KMS-11 cells for Fyn
SH3-derived FGFR3-binding polypeptides.

| Fynomer | SEQ ID NO | EC50 (nM) |
|---|---|---|
| FF2L4C3 | 101 | 5.9 |
| FF44L65G12 | 103 | 2.2 |
| FF44L65G7 | 105 | 4.2 |
| FF48L66G7 (G7) | 107 | 2.3 |
| FF43L65D5 | 109 | 1.2 |
| FF44L65B7 | 111 | 0.6 |

EC50 values, in the low nanomolar range (Table 25), measured on a cell line expressing FGFR3 (FIG. 11A, KMS-11) confirmed the high apparent affinities measured by surface plasmon resonance (Table 24), and demonstrate binding to FGFR3 in the natural context of a cell surface. All the Fyn SH3-derived polypeptides (SEQ ID NOs: 101, 103, 105, 107, 109 and 111) binding to FGFR3 did not show unspecific binding on a cell line not expressing FGFR3 (FIG. 11B).

### Example 6: Fyn SH3-Derived Polypeptides of the Invention Specific to FGFR3 do not Interfere with Ligand Binding

It would be preferred if the Fyn SH3-derived polypeptides for binding both isoforms FGFR3b and FGFR3c to not interfere with ligand (e.g. FGF1) binding, as the ligand binding site is located in proximity to the splice site give rise to either FGFR3b or FGFR3c.

For the purpose of verifying the ability of the Fyn SH3-derived polypeptides to bind to FGFR3 in presence of one of its ligands, a BIAcore experiment was set up to measure the affinity of the Fynomers to FGFR3 in presence or absence of FGF1 (Fibroblast Growth Factor 1 is one of the major ligands of FGFR3).

In analogy to the method used for measuring the affinities (as described in Example 5), Fynomers at concentration of 100 nM (with the exception of FF2L4C3-SEQ ID NO:101 used at a concentration of 500 nM) were captured on the 9E10 surface followed by injections of different concentrations of huFGFR3c-Fc (0 nM, 11 nM, 33 nM, 100 nM) in presence or absence of 200 nM FGF1 (R&D systems 232-FA-025/CF). Sensograms were recorded and apparent kinetics constants were calculated using the BIAevaluation 2.1 software.

### Results

Independently of the presence or absence of 200 nM FGF1 in solution binding of the Fyn SH3-derived polypeptides to huFGFR3c was unchanged, showing that binding of the Fynomers to FGFR3 did not interfere with ligand binding.

TABLE 26
Shows the kinetics constants obtained in presence or absence
of 200 nM FGF1.

| | | | KDₐₚₚ (pM) to |
|---|---|---|---|
| | | | huFGFR3c-Fc |
| | | KDₐₚₚ (pM) to | (SEQ ID NO: 13) |
| | SEQ ID | huFGFR3c-Fc | in presence of |
| Fynomer | NO. | (SEQ ID NO: 13) | 200 nM FGF1 |
| FF2L4C3 | 101 | 4000 | 3800 |
| FF44L65G12 | 103 | 140 | 120 |
| FF44L65G7 | 105 | 320 | 230 |
| FF48L66G7 | 107 | 170 | 130 |
| (G7) | | | |
| FF43L65D5 | 109 | 60 | 70 |
| FF44L65B7 | 111 | 170 | 130 |

Even though, due to assay variability the values for KDₐₚₚ in absence of FGF1 are slightly different than the values obtained in the experiment shown in Example 2 (Table 24), this experiment shows that the Fyn SH3-derived polypeptides are able bind to FGFR3 even if the ligand (in this case FGF1) is bound to the ligand binding site. From this we conclude that the epitope bound by the Fyn SH3-derived polypeptides described here is located in a constant region of FGFR3.

### Example 7: Fyn SH3-Derived Polypeptides of the Invention Bind to the Domains D1-D2 of FGFR3

Specificity of Fyn-SH3 derived polypeptides binding to FGFR3 was tested by ELISA.

Different antigens were coated on the plate (Maxisorp plate; Nunc 439454): huFGFR3b-Fc, huFGFR3c-Fc, cyFGFR3c-Fc, muFGFR3c-His, huD1-Fc, huD2-Fc, huD1-D2-Fc.

The plate was coated with 100 μL antigen at 5 pg/mL (0.5 pg/well), and incubated at 4° C. overnight. The wells were washed 3× with PBS before being blocked with 200 μL 4% MPBS for 1 hr at RT. The wells were washed again, and 20 μL 10% MPBS containing 15 pg/mL 9E10 was added, before the addition of 80 μl Fynomer at 250 nM (200 nM final Fynomer concentration). The wells were incubated for 45 min at RT, before washing and the addition of 100 μL anti-mouse IgG-HRP (Sigma A2554) diluted 1:1000 in 2% MPBS. The wells were incubated for 30 min at RT, before washing 3× with 0.1% Tween-20 in PBS, and then 3× PBS. 100 μL BM POD Blue substrate (Roche 11 484 281 001) was added to each well followed by 50 μL 1M H₂SO₄ to stop the reaction. The absorbance 450 nm-650 nm was recorded using a Tecan M1000 instrument.

### Results

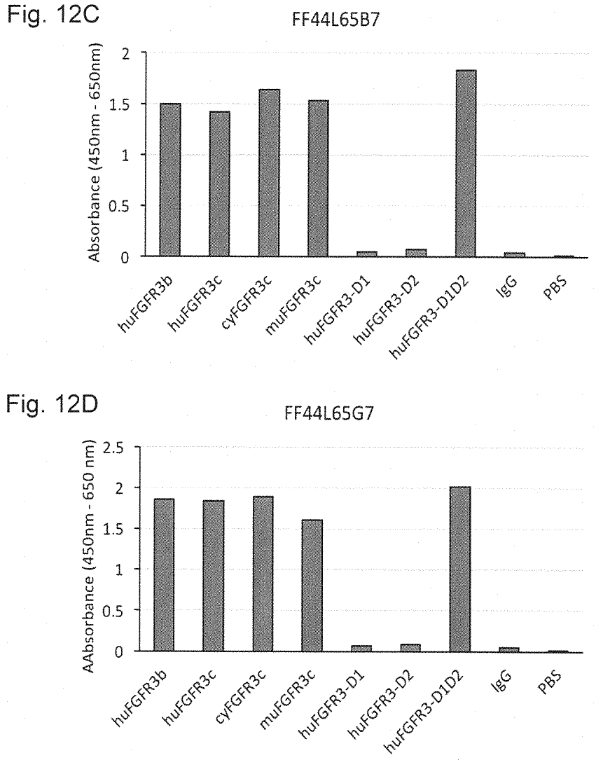

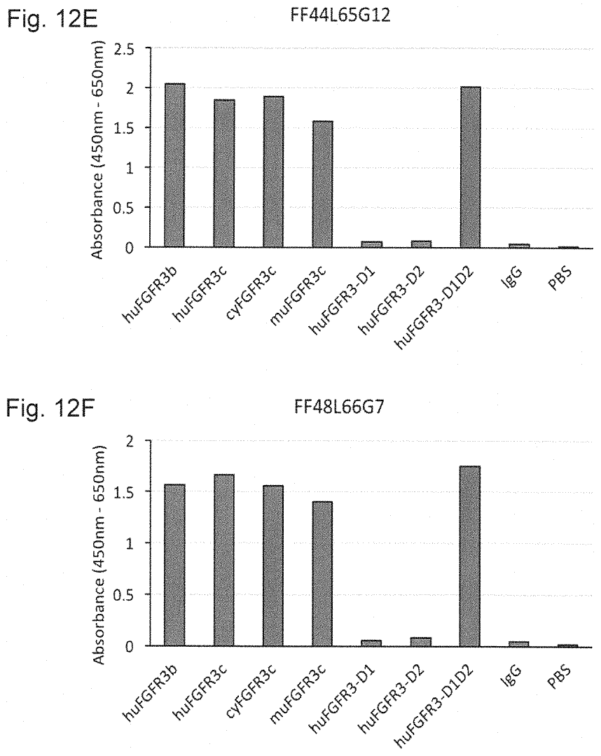

As shown in FIG. 12 A-F, the Fyn SH3-derived polypeptides all are cross-reactive to cynomolgus and murine FGFR3c. Interestingly all binders are specific for an epitope present only when the domains D1 and D2 are physically linked (see FIG. 12 A-F bar huFGFR3-D1D2), in fact no binding is observed if the single domains D1 or D2 (hFGFR3-D1 or huFGFR3-D2) are immobilized on the ELISA plate.

### Example 8: Fyn SH3-Derived Polypeptides of the Invention Cause Efficient Internalization of FGFR3

Internalization is a central feature of the Fyn SH3-derived polypeptides described here, and provides the opportunity to use these binders to deliver toxic payloads and/or fused proteins such as antibodies intracellularly.

In order to assess the ability of the Fyn SH3-derived polypeptides binding to FGFR3 to internalize upon binding to the target, an internalization assay based on the intracellular delivery of a cytotoxic agent was established.

The assay measures the cytotoxic effect of anti-FGFR3 Fynomers cross-linked with MMAF (Monomethyl auristatin F)-conjugated 9E10, on KMS-11 cells. MMAF is an antimitotic agent (blocks tubulin polymerization) and is active only upon internalization into the cells. Therefore, this assay indicates how well the Fynomers facilitate internalization of MMAF. 50 μL of KMS-11 cells at 2×10⁵ cells/mL were seeded into a 96-well flat bottomed plate (Corning Costar 3610), to give 10,000 cell per well. The cells were incubated for 4 hours to allow the cells to adhere (37° C., 5% CO₂). Fynomers and 9E10-MMAF were mixed at a 3:1 ratio. A 4× stock of Fynomer (4 pM) and a 4× stock of 9E10-MMAF (1.33 pM) were prepared in RPMI media (see section 5.4.1) and mixed 1:1 (40 μl+40 μL). This mixture was then serially diluted 1 in 3, to give a concentration range 1000 nM-50 pM. 50 μL of the sample was added to the 50 μL of cells (as seeded above), and incubated for 5 days (37° C., 5% CO2). Appropriate controls, the wild-type Fynomer FynSH3, MMAF-9E10 without Fynomer and also cells without addition of any reagents, were included. All samples were prepared in duplicate. After 5 days, 100 μL Cell titer glo (Promega G7573) was added to each well and incubated with gentle shaking for 10 min in the dark. As a read-out for cell viability, luminescence was measured using a Tecan M1000 instrument. Analysis was performed using Prism 6. The data was transformed (X=log X), and analyzed using a non-linear fit, log(inhibitor) vs. response—Variable slope (4 parameters).

### Results

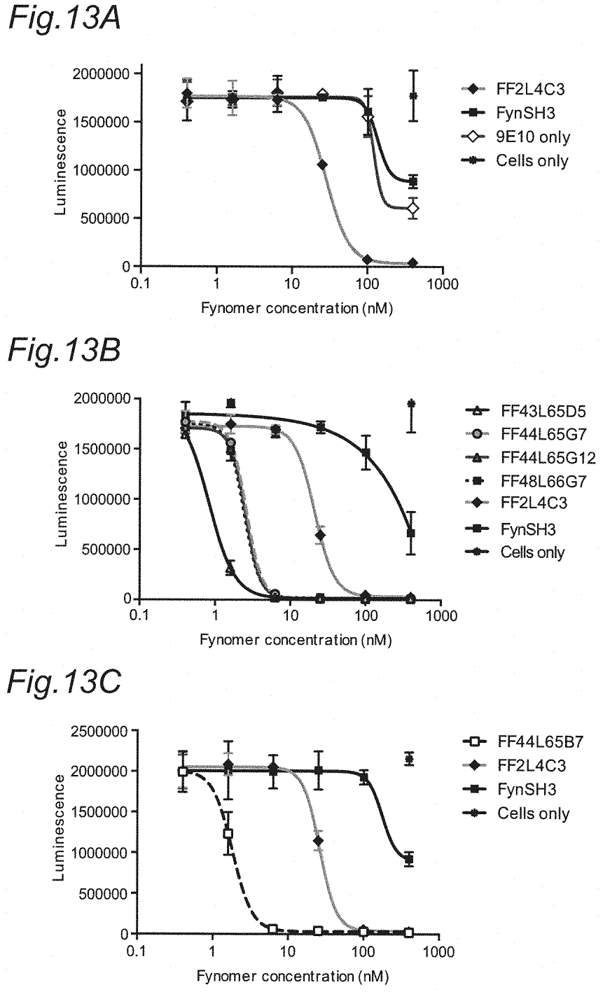

All Fyn SH3-derived FGFR3-binding polypeptides described here show increased cytotoxicity (e.g. internalization) compared to the cells treated with the MMAF-labeled secondary antibody only (9E10 in FIG. 13A) or the wild-type Fynomer FynSH3 in combination with MMAF-labeled 9E10 shown in all 3 experiments (FIG. 13 A-C indicated as FynSH3), that show cytotoxicity only at the highest concentration tested, probably due to the toxicity of MMAF itself. FIG. 13 shows the cytotoxicity profiles obtained in different experiments, and Table 27 shows the EC50 obtained for the different Fyn SH3-derived FGFR3-binding polypeptides.

TABLE 27
EC50 values determined in internalization assays using FGFR3+
KMS-11 cells for Fyn SH3-derived FGFR3-binding polypeptides.

| Fynomer | EC50 (nM) |
|---|---|
| FF2L4C3 | 28.5/21/26.4 |
| FF44L65G12 | 2.5 |
| FF44L65G7 | 2.6 |
| FF48L66G7 (G7) | 2.4 |
| FF43L65D5 | 0.8 |
| FF44L65B7 | 1.8 |

*Note:
for FF2L4C3 the 3 values obtained in the 3 experiments shown in FIG. 13 are indicated.

The data shown in FIG. 13 and Table 27 show that increased affinity also leads to more efficient internalization.

### Example 9: Alternative Fyn-SH3 Derived Polypeptide that Shows Excellent Binding and Internalization Properties and which are Derived of a Different Family

In addition to the family of sequences derived from SEQ ID NO:99, we identified an alternative Fynomer, FF40L54A5 (GVTLFVALYDYEVMSTTALSFHKGEKFQILSQSPHGQYWEARSLTTGETGWIPSNYVA PVDSIQ; SEQ ID NO:116), that surprisingly also shows excellent binding and internalization properties and shares manufacturability and cross-reactivity properties with the Fynomers derived from SEQ ID NO:99 (see Table 28). Fynomer FF40L54A5 was not expected to have excellent internalization properties as its sequence was derived from a Fynomer that showed only very poor internalization properties.

Table 28 summarizes the properties of Fynomer FF40L54A5.

TABLE 28
part 1

| | | | Affinities measured by BIAcore | | |
|---|---|---|---|---|---|
| | SEQ ID | Yield | huFGFR3b | huFGFR3c | cyFGFR3c |
| Fynomer | NO. | (mg/L) | KDₐₚₚ (pM) | KDₐₚₚ (pM) | KDₐₚₚ (pM) |
| FF40L54A5 | 116 | 5.4 | 170 | 170* | 160 |

TABLE 28
part 2

| | | | Affinities measured by | | |
|---|---|---|---|---|---|
| | | | BIAcore | | |
| | | | (competition with FGF1) | | |
| | | EC50 | | KDₐₚₚ | |
| | | (nM) for | | (pM) to | |
| | | binding | KDₐₚₚ | huFGFR3c- | EC50 |
| | | to | (pM) | Fc in | (nM) in |
| | | FGFR3⁺ | to | presence of | internal- |
| | SEQ ID | KMS-11 | huFGFR3c- | 200 nM | ization |
| Fynomer | NO. | cells | Fc | FGF1 | assay |
| FF40L54A5 | 116 | 6.6 | 210* | 230 | 4.7 |

*differences due to experimental variability

TABLE 28
part 3

| | | Specificity ELISA | | | | |
|---|---|---|---|---|---|---|
| | | | | Binding to | Binding to | Binding to |
| | | Cross- | Cross- | domain | domain | domains |
| | SEQ | reactivity to | reactivity to | D1 of | D2 of | D1-D2 of |
| Fynomer | ID NO. | cyFGFR3c | muFGFR3c | huFGFR3 | huFGFR3 | huFGFR3 |
| FF40L54A5 | 116 | +++ | +++ | − | − | +++ |

### Example 10—Fynomer-Antibody Bi-Specific Binding Agents (Fynomabs)

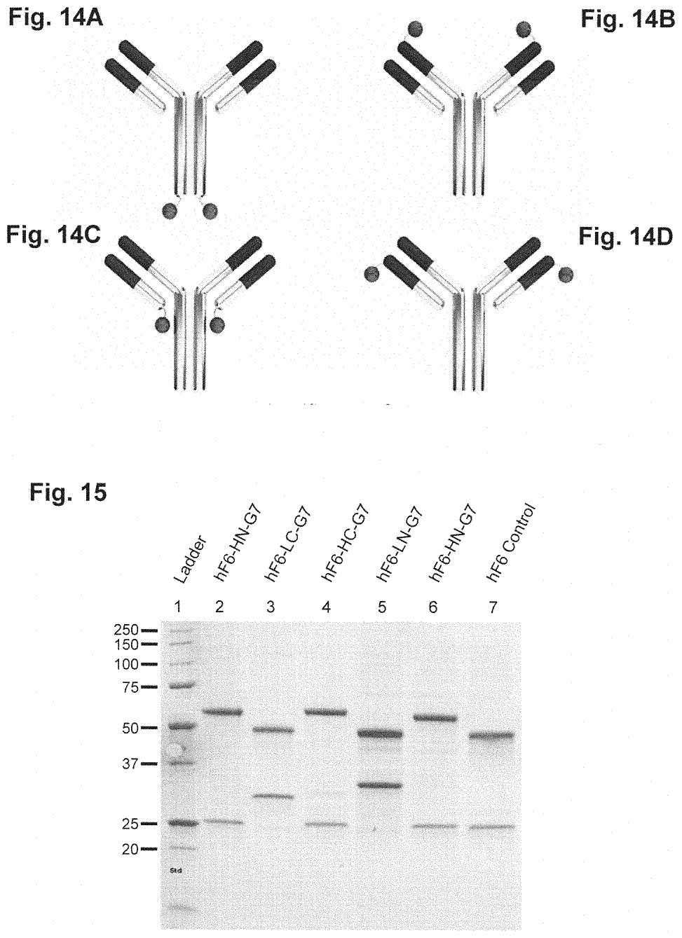

The following provides a non-limiting example of making a bi-specific binding agent that comprises one embodiment of an FGFR3 binding Fynomer and one embodiment of an anti-CD138 monoclonal antibody. Any embodiment of a Fynomer disclosed herein can be attached to any embodiment of an anti-CD138 antibody, or binding portion thereof that is disclosed herein to generate a bi-specific binding agent that binds specifically to an FGFR3 and to CD138. The FGFR3 binding Fynomer used in this example is designated as FF48L66G7 (“G7”, (SEQ ID NO:107)) which is attached to the humanized anti-CD138 monoclonal antibody designated as hF6. The hF6 antibody is a humanized IgG1/kappa antibody comprising the heavy chain variable region of SEQ ID NO:93 and the light chain variable region of SEQ ID NO:44. The G7 Fynomer binds specifically to both mouse and human FGFR3. The G7 Fynomer also binds specifically to both FGFR3b and FGFR3c. The G7 Fynomer was covalently attached by a linker GGGGSGGGGSGGGGS at four different positions on the hF6 monoclonal antibody using recombinant technology. The positions include the heavy chain or light chain N-terminus (indicated as HN and LN, respectively), and the heavy chain or light chain C-terminus (indicated as HC and LC, respectively)(e.g., see FIG. 14). Briefly, for expression of hF6 comprising G7 fused to the N-terminus of the heavy chain, a nucleic acid construct was generated comprising a coding region directing the expression of G7 located 5′ of, and in-frame with, a sequence encoding the linker and a coding region directing the expression of the hF6 heavy chain. The hF6-HN-G7 construct was then co-transfected into CHO cells with a nucleic acid construct directing the expression of the hF6 light chain. The resulting bi-specific binding agent comprising the full length hF6 antibody of IgG1/kappa isotype and the G7 Fynomer (hF6-HN-G7) was isolated and purified from the cell culture supernatant.

Similarly, for expression of hF6 comprising G7 fused to the C-terminus of the heavy chain, a nucleic acid construct was generated comprising a coding region directing the expression of the hF6 heavy chain located 5′ of, and in-frame with, a sequence encoding the linker and a coding region directing the expression of G7. For expression of hF6 comprising G7 fused to the N-terminus of the light chain, a nucleic acid construct was generated comprising a coding region directing the expression of G7 located 5′ of, and in-frame with, a sequence encoding the linker and a coding region directing the expression of the hF6 light chain. For expression of hF6 comprising G7 fused to the C-terminus of the light chain, a nucleic acid construct was generated comprising a coding region directing the expression of the hF6 light chain located 5′ of, and in-frame with, a sequence encoding the linker and a coding region directing the expression of G7. The indicated constructs were then co-transfected into CHO cells with a nucleic acid construct directing the expression of the corresponding heavy or light chain of hF6. The resulting bi-specific binding agents comprising the full length hF6 antibody of IgG1/kappa isotype and the G7 Fynomer (i.e., hF6-HC-G7, hF6-LC-G7, and hF6-LN-G7) were isolated and purified from the cell culture supernatants. Each of the resulting bi-specific binding agents were assayed for binding to CD138 and FGFR3 by ELISA, the results of which are shown in Table 29.

TABLE 29

| | mFGFR3c ELISA, | hCD138 ELISA, |
|---|---|---|
| Construct | EC50 (nM) | EC50 (nM) |
| hF6 (no Fynomer) | NB | 0.56 |
| hF6-LC-G7 | 0.94 | 0.98 |
| hF6-LN-G7 | 0.49 | 1.07 |
| hF6-HC-G7 | 0.91 | 0.55 |
| hF6-HN-G7 | 0.53 | 0.69 |

*NB = No binding.

In Table 28, the bi-specific construct designated hF6-LC-G7 indicates attachment of the G7 Fynomer to the C-terminal end of the light chain of the hF6 antibody (e.g., see embodiment of FIG. 14C), hF6-LN-G7 indicates attachment of the G7 Fynomer to the N-terminal end of the light chain of the hF6 antibody (e.g., see embodiment of FIG. 14D), hF6-HC-G7 indicates attachment of the G7 Fynomer to the C-terminal end of the heavy chain of the hF6 antibody (e.g., see embodiment of FIG. 14A), and hF6-HN-G7 indicates attachment of the G7 Fynomer to the N-terminal end of the heavy chain of the hF6 antibody (e.g., see embodiment of FIG. 14B). Table 28 show that the resulting bi-specific binding agents retained specific binding affinity to mouse FGFR3c and human CD138 as determined by ELISA.

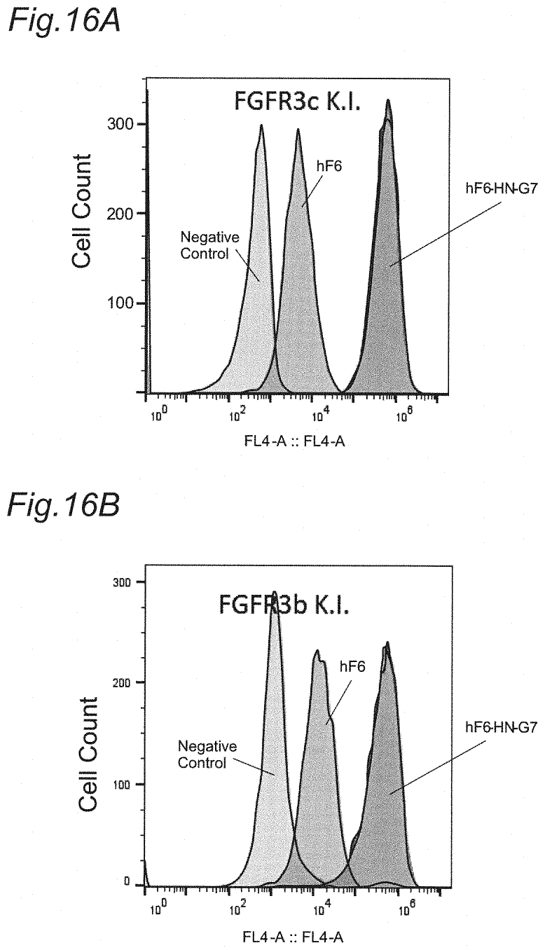

CHO cells (Chinese Hamster Ovary cells) were transfected to express the human FGFR3c (FIG. 16A) or human FGFR3b (FIG. 16B) on the cell surface. The representative bi-specific binding agent hF6-HN-G7 was then shown to specifically bind both human FGFR3b (FIG. 16B) and human FGFR3c (FIG. 16A) expressed on the cell surface of the transfected CHO cells by FACs analysis.

The representative binding data of Table 28 and FIG. 16 demonstrates that the bi-specific binding agents retain the binding specificity of the G7 Fynomer, as well as the binding specificity of the hF6 anti-CD138 antibody.

### Example 11—Fynomer/Antibody (Fynomab) Drug Conjugates

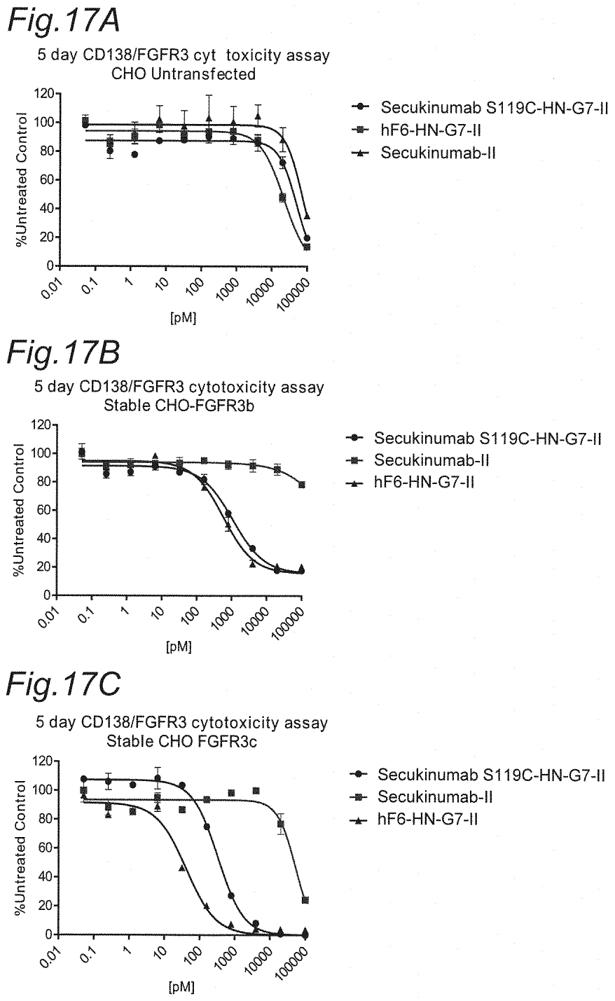

The representative bi-specific binding agent hF6-S1 19C-HN-G7 was site specifically conjugated to the PBD toxin of formula II to generate a representative drug conjugate of a bi-specific binding agent designated as hF6-S119C-HN-G7-II. hF6-S119C-HN-G7-II demonstrated specific cytotoxicity to CHO cells expressing human FGFR3b (FIG. 17B) and human FGFR3c (FIG. 17C) but did not demonstrate specific killing of untransfected CHO cells (FIG. 17A) at concentrations less than 10000 pM. The cytotoxicity observed at high concentrations of drug conjugated binding agents (i.e., >10000 pM) is most likely due to non-specific killing by the high concentration of toxin itself, independent of binding.

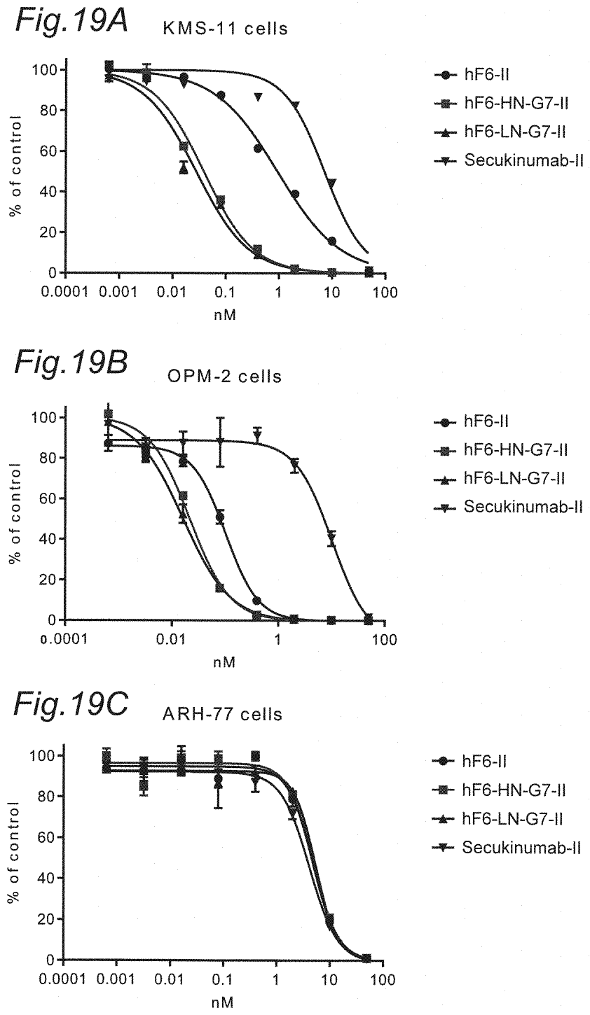

The representative bi-specific binding agent drug conjugates hF6-HN-G7-II and hF6-LN-G7-II also demonstrated specific cytotoxicity against the cell lines KMS-11 (a high CD138 expressing cell line), and OPM-2 (a medium CD138 expressing cell line), but not ARH-77 (a negative control cell line)(e.g., see FIG. 19).

The antibody portion (i.e., hF6) of the bi-specific binding agent hF6-HN-G7 was engineered to incorporate a cysteine amino acid substitution at one of five positions (i.e., S119, V282, T289, N361, and V422) in the hF6 antibody heavy chain constant region thereby providing the cysteine substituted antibodies designated as hF6-S119C, hF6-V282C, hF6-T289C, hF6-N361C and hF6-V422C. The corresponding bi-specific binding agents were designated as hF6-S119C-HN-G7, hF6-V282C-HN-G7, hF6-T289C-HN-G7, hF6-N361C-HN-G7 and hF6-V422C-HN-G7, respectively, wherein “G7” indicates the presence of the FF48L66G7 Fynomer that is attached to the N-terminus of the heavy chain of hF6. The engineered cysteine residues provided a conjugation point for site-specific conjugation (SSC) of an anti-neoplastic agent to each of the bi-specific binding agents. A representative sequence of the parent hF6-HN-G7 is shown below. The dotted underline indicates a signaling sequence, the single underline represents the Fynomer portion, the double underline represents an optional linker region which is followed by the heavy chain of the hF6 antibody. Each of the amino acids that were independently mutated to a cysteine (i.e., A118, S119, S239, V282, T289, N361 and V422) are indicated.

| SEQ ID NO: 125 | |
|---|---|
| | 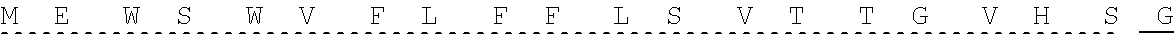 |
| 1 | ATGGAG TGGTCC TGGGTG TTTCTG TTCTTC CTCAGC GTGACC ACGGGA GTGCAT AGCGGC |
| | TACCTC ACCAGG ACCCAC AAAGAC AAGAAG GAGTCG CACTGG TGCCCT CACGTA TCGCCG |
| | V T L F V A L Y D Y E V Y G P T P M L S |
| 61 | GTGACC CTGTTT GTGGCC CTGTAC GACTAC GAGGTG TACGGC CCCACA CCCATG CTGTCC |
| | CACTGG GACAAA CACCGG GACATG CTGATG CTCCAC ATGCCG GGGTGT GGGTAC GACAGG |
| | F H K G E K F Q I L K G G S G P Y W E A |
| 121 | TTCCAC AAGGGC GAGAAG TTCCAG ATCCTG AAGGGC GGCTCC GGCCCC TACTGG GAGGCC |
| | AAGGTG TTCCCG CTCTTC AAGGTC TAGGAC TTCCCG CCGAGG CCGGGG ATGACC CTCCGG |
| | R S L T T G E T G L I P S N Y V A P V D |
| 181 | AGATCC CTGACC ACAGGC GAGACA GGCCTG ATCCCC TCCAAC TACGTG GCCCCC GTGGAC |
| | TCTAGG GACTGG TGTCCG CTCTGT CCGGAC TAGGGG AGGTTG ATGCAC CGGGGG CACCTG |
| | S I Q G G G G S G G G G S G G G G S Q V |
| 241 | TCCATT CAGGGC GGCGGA GGATCC GGCGGA GGAGGA AGTGGC GGAGGA GGAAGT CAAGTC |
| | AGGTAA GTCCCG CCGCCT CCTAGG CCGCCT CCTCCT TCACCG CCTCCT CCTTCA GTTCAG |
| | Q L V Q S G A E V V K P G A S V K L S C |
| 301 | CAACTG GTCCAA TCAGGT GCTGAG GTCGTG AAGCCA GGCGCG TCAGTT AAGCTC TCCTGC |
| | GTTGAC CAGGTT AGTCCA CGACTC CAGCAC TTCGGT CCGCGC AGTCAA TTCGAG AGGACG |
| | K A S G Y T F T S Y Y L Y W V K K A P G |
| 361 | AAGGCT TCCGGA TACACC TTCACA AGTTAT TATCTT TACTGG GTCAAA AAAGCA CCCGGT |
| | TTCCGA AGGCCT ATGTGG AAGTGT TCAATA ATAGAA ATGACC CAGTTT TTTCGT GGGCCA |
| | Q G L D W I G E I Y P R S G G T N I N E |
| 421 | CAGGGC CTCGAT TGGATC GGTGAA ATTTAC CCCCGC TCCGGA GGGACT AATATT AACGAA |
| | GTCCCG GAGCTA ACCTAG CCACTT TAAATG GGGGCG AGGCCT CCCTGA TTATAA TTGCTT |
| | K F L S R V T L T A D T S T S T A Y L E |
| 481 | AAGTTC CTGAGT CGAGTG ACACTT ACTGCT GATACT TCCACC TCCACC GCATAC CTCGAG |
| | TTCAAG GACTCA GCTCAC TGTGAA TGACGA CTATGA AGGTGG AGGTGG CGTATG GAGCTC |
| | L S S L T S E D T A V Y Y C T R S L L Y |
| 541 | TTGTCC TCCCTC ACATCC GAGGAT ACCGCC GTGTAC TACTGC ACAAGG AGCCTG TTGTAT |
| | AACAGG AGGGAG TGTAGG CTCCTA TGGCGG CACATG ATGACG TGTTCC TCGGAC AACATA |
| | 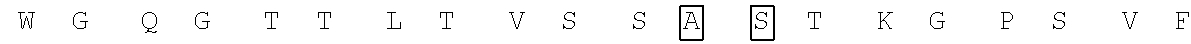 |
| 601 | TGGGGC CAAGGC ACTACA CTGACA GTGTCT TCAGCT TCCACC AAAGGT CCATCC GTGTTT |
| | ACCCCG GTTCCG TGATGT GACTGT CACAGA AGTCGA AGGTGG TTTCCA GGTAGG CACAAA |
| | P L A P S S K S T S G G T A A L G C L V |
| 661 | CCACTG GCACCC TCATCA AAAAGC ACTAGC GGCGGC ACCGCT GCTCTG GGGTGT CTGGTC |
| | GGTGAC CGTGGG AGTAGT TTTTCG TGATCG CCGCCG TGGCGA CGAGAC CCCACA GACCAG |
| | K D Y F P E P V T V S W N S G A L T S G |
| 721 | AAGGAC TATTTT CCTGAG CCTGTG ACAGTT AGCTGG AACAGC GGCGCC CTTACT AGCGGC |
| | TTCCTG ATAAAA GGACTC GGACAC TGTCAA TCGACC TTGTCG CCGCGG GAATGA TCGCCG |
| | V H T F P A V L Q S S G L Y S L S S V V |
| 781 | GTCCAC ACCTTT CCCGCC GTGCTT CAATCC TCTGGC CTCTAC TCCCTT TCAAGC GTGGTC |
| | CAGGTG TGGAAA GGGCGG CACGAA GTTAGG AGACCG GAGATG AGGGAA AGTTCG CACCAG |
| | T V P S S S L G T Q T Y I C N V N H K P |
| 841 | ACAGTC CCCAGC TCTTCA CTCGGT ACCCAG ACTTAT ATATGT AATGTT AATCAC AAGCCT |
| | TGTCAG GGGTCG AGAAGT GAGCCA TGGGTC TGAATA TATACA TTACAA TTAGTG TTCGGA |
| | S N T K V D K K V E P K S C D K T H T C |
| 901 | AGCAAC ACTAAG GTTGAT AAAAAG GTGGAG CCCAAA AGCTGT GACAAG ACGCAT ACATGC |
| | TCGTTG TGATTC CAACTA TTTTTC CACCTC GGGTTT TCGACA CTGTTC TGCGTA TGTACG |
| | 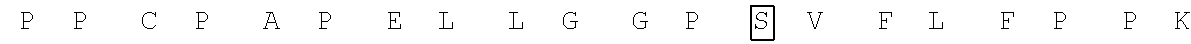 |
| 961 | CCTCCT TGCCCC GCCCCC GAGCTC CTGGGC GGCCCT TCCGTC TTTCTG TTCCCA CCCAAA |
| | GGAGGA ACGGGG CGGGGG CTCGAG GACCCG CCGGGA AGGCAG AAAGAC AAGGGT GGGTTT |
| | P K D T L M I S R T P E V T C V V V D V |
| 1021 | CCCAAA GATACC CTCATG ATCTCC AGAACG CCAGAA GTTACC TGCGTT GTTGTA GATGTG |
| | GGGTTT CTATGG GAGTAC TAGAGG TCTTGC GGTCTT CAATGG ACGCAA CAACAT CTACAC |
| | 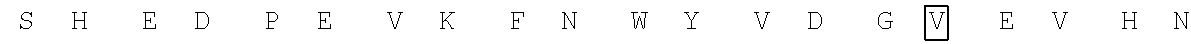 |
| 1081 | TCTCAC GAGGAC CCCGAA GTGAAG TTTAAC TGGTAT GTGGAT GGAGTG GAGGTT CATAAC |
| | AGAGTG CTCCTG GGGCTT CACTTC AAATTG ACCATA CACCTA CCTCAC CTCCAA GTATTG |
| | 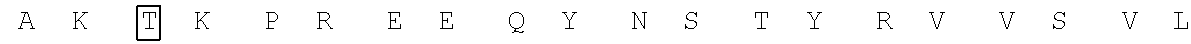 |
| 1141 | GCCAAG ACAAAA CCCCGC GAAGAG CAGTAC AATAGC ACATAT AGGGTC GTGAGC GTCCTC |
| | CGGTTC TGTTTT GGGGCG CTTCTC GTCATG TTATCG TGTATA TCCCAG CACTCG CAGGAG |
| | T V L H Q D W L N G K E Y K C K V S N K |
| 1201 | ACTGTC CTCCAC CAGGAC TGGTTG AACGGT AAGGAA TATAAA TGTAAA GTCTCC AACAAG |
| | TGACAG GAGGTG GTCCTG ACCAAC TTGCCA TTCCTT ATATTT ACATTT CAGAGG TTGTTC |
| | A L P A P I E K T I S K A K G Q P R E P |
| 1261 | GCTCTG CCCGCG CCAATT GAAAAA ACAATC TCAAAG GCAAAG GGCCAG CCACGG GAACCT |
| | CGAGAC GGGCGC GGTTAA CTTTTT TGTTAG AGTTTC CGTTTC CCGGTC GGTGCC CTTGGA |
| | 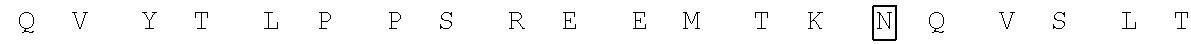 |
| 1321 | CAGGTC TACACA CTGCCA CCCAGC CGAGAG GAGATG ACTAAG AATCAG GTCTCT CTGACA |
| | GTCCAG ATGTGT GACGGT GGGTCG GCTCTC CTCTAC TGATTC TTAGTC CAGAGA GACTGT |
| | C L V K G F Y P S D I A V E W E S N G Q |
| 1381 | TGTCTG GTGAAG GGGTTT TATCCA TCTGAC ATTGCC GTTGAA TGGGAA TCAAAC GGGCAG |
| | ACAGAC CACTTC CCCAAA ATAGGT AGACTG TAACGG CAACTT ACCCTT AGTTTG CCCGTC |
| | P E N N Y K T T P P V L D S D G S F F L |
| 1441 | CCTGAA AATAAT TACAAG ACTACT CCTCCC GTACTG GACTCC GACGGG TCATTT TTCCTC |
| | GGACTT TTATTA ATGTTC TGATGA GGAGGG CATGAC CTGAGG CTGCCC AGTAAA AAGGAG |
| | 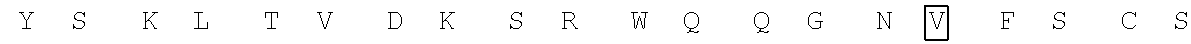 |
| 1501 | TACTCT AAACTT ACTGTC GATAAG TCAAGA TGGCAA CAGGGC AACGTC TTCAGT TGCAGC |
| | ATGAGA TTTGAA TGACAG CTATTC AGTTCT ACCGTT GTCCCG TTGCAG AAGTCA ACGTCG |
| | V M H E A L H N H Y T Q K S L S L S P G |
| 1561 | GTGATG CATGAA GCCCTC CATAAC CATTAT ACGCAG AAATCT CTCAGT CTGTCT CCCGGG |
| | CACTAC GTACTT CGGGAG GTATTG GTAATA TGCGTC TTTAGA GAGTCA GACAGA GGGCCC |
| | K * * |
| 1621 | AAGTAA TGA |
| | TTCATT ACT |

Amino acid sequences of the bi-specific binding agents with mutation in the heavy chain constant region of A118C, S119C, S239C, V282C, T289C, N361C and V422C, i.e. bi-specific binding agents hF6-A118C-HN-G7, hF6-S119C-HN-G7, hF6-S239C-HN-G7, hF6-V282C-HN-G7, hF6-T289C-HN-G7, hF6-N361C-HN-G7 and hF6-V422C-HN-G7 are provided as SEQ ID NOs:135-141.

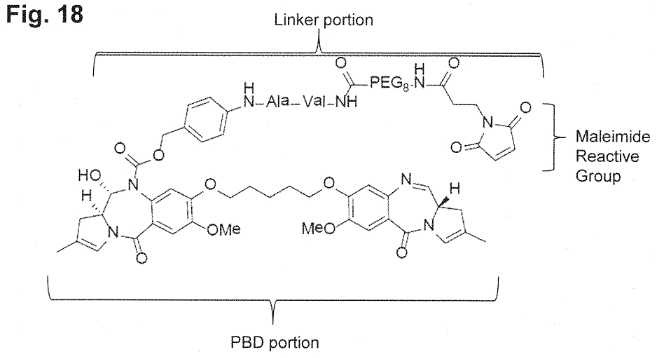

Each of the representative bi-specific binding agents were conjugated to the exemplary toxic pyrrolobenzodiazepine (PBD) of formula II (also see FIG. 18). Stochastically conjugated bi-specific binding agents were conjugated in-house using a modified DHAA reduction protocol. Briefly, antibody agents were diluted to 1.1 mg in dPBS and reduced with 2×-20× stabilized TCEP (bond breaker) for 1-3 hours at 25 to 37° C. with occasional swirling. Samples were then buffer exchanged into dPBS with 1 mM EDTA to remove TCEP and were oxidized using 2.5× DHAA for 2.5 hours at room temperature. Antibodies were buffer exchanged into dPBS to remove DHAA and incubated with toxin II at 8× for 1.5 hours at room temperature. Conjugated agents were then buffer exchanged into dPBS and the concentrations were determined by nanodrop. Site specific conjugations were achieved under less stringent reducing conditions. The resulting drug-conjugated bi-specific binding agents that were site-specifically conjugated were designated as hF6-S1 19C-HN-G7-II, hF6-V282C-HN-G7-II, hF6-T289C-HN-G7-II, hF6-N361C-HN-G7-II and hF6-V422C-HN-G7-II.

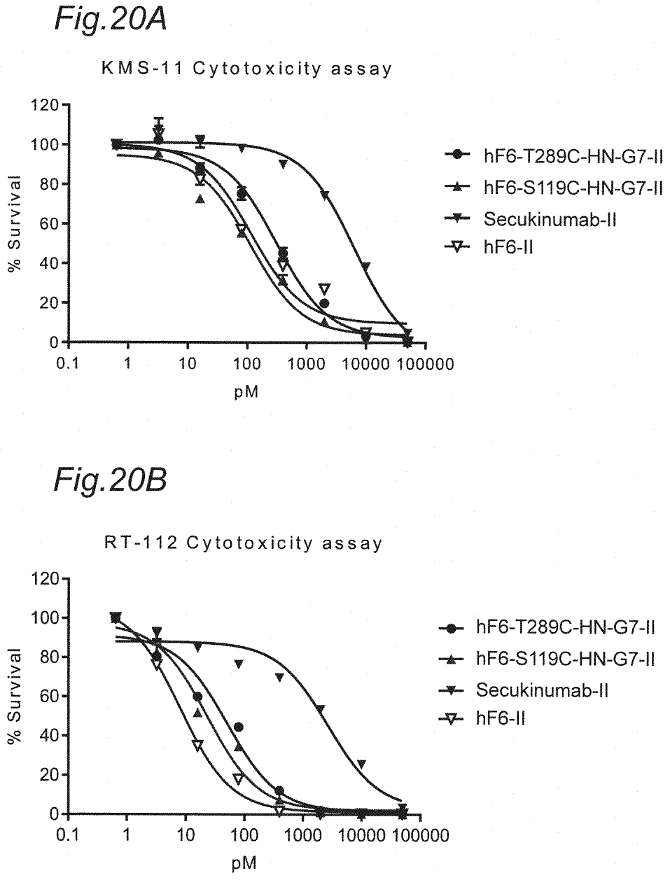

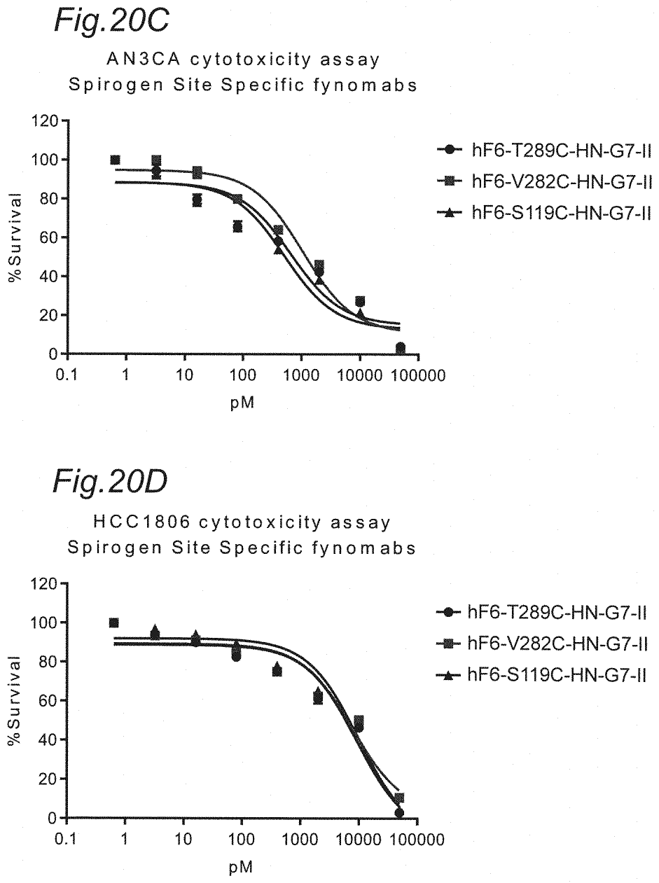

A 5-day cytotoxicity assay was carried out (N=3) using the ‘high CD138’ expressing cell line (KMS 11), ‘medium CD138’ cell line (RT112), and negative control cell line (ARH77). The results of the cytotoxicity assay are shown in the table below and in FIG. 20.

TABLE 30

| | KMS-11 | | RT112 | | ARH-77 | |
|---|---|---|---|---|---|---|
| | IC50 | % | IC50 | % | IC50 | % |
| | (pM) | max kill | (pM) | max kill | (pM) | max kill |
| hF6-S119C-HN-G7-II | 27.9 | 99.6 | 35.3 | 99.7 | 3301.7 | 98.1 |
| hF6-T289C- HN-G7-II | 83.8 | 99.6 | 52.6 | 99.7 | 4005.7 | 98.0 |
| hF6-V282C- HN-G7-II | 89.3 | 99.7 | 28.9 | 99.7 | 3552.7 | 98.3 |
| hF6-N361C- HN-G7-II | 28.4 | 98.4 | 75.0 | 99.7 | 5373.7 | 97.1 |
| hF6-V422C- HN-G7-II | 17.0 | 99.5 | 28.1 | 99.7 | 3876.7 | 97.9 |
| hF6-HN-G7-II | 17.9 | 99 | 7.1 | 99 | 1331.0 | 99 |
| Secukinumab-II | 38.4 | 99 | 393.9 | 99 | 839.0 | 99 |
| hF6-II | 114.1 | 99 | 19.6 | 99 | 1126.5 | 98 |

The representative drug-conjugated bi-specific binding agents designated as hF6-S119C-HN-G7-II, hF6-V282C-HN-G7-II, and hF6-T289C-HN-G7-II, were tested in an in vitro toxicity assay against KMS-11 cells (a high expressing CD138 cell line), RT-1 12 cells, AN3CA cells (a CD138/FGFR3 positive cell line), HCC1806 cells (a CD138/FGFR3 positive cell line) (e.g., see FIG. 20) and other cell types. The cell cytotoxicity results are summarized in the table below.

TABLE 31

| | CD138 | | | | | | | |
|---|---|---|---|---|---|---|---|---|
| | receptor | FGFR3 | | | | | | |
| | number | receptor | | | | | | |
| | on cell | number on | hF6-S119C-HN-G7-II | | hF6-T289C-HN-G7-II | | hF6-V282C-HN-G7-II | |
| | line | cell line | | % cell | | % cell | | % cell |
| Cell line | (qFACS) | (qFACS) | EC₅₀, pM | killing | EC₅₀, pM | killing | EC₅₀, pM | killing |
| KMS-11 | 122,818 | 37,455 | 113.2 | 99.6 | 311.7 | 99.4 | NT | NT |
| RT-112 | 589,939 | 4,264 | 23.41 | 99.7 | 50.82 | 99.5 | NT | NT |
| 8505C | 144,618 | 1,128 | 559.1 | 96.1 | 630.8 | 95.3 | NT | NT |
| OE19 | 418,816 | 6,410 | 554.4 | 70.3 | 696.4 | 69 | NT | NT |
| MHHES-1 | 789 | 11,410 | 1366.5 | 99.3 | 1394.5 | 99.2 | NT | NT |
| An3CA | 216,058 | 1,406 | 936.5 | 97.3 | 1044.1 | 96.5 | 1054 | 97.6 |
| HCC1806 | 135,321 | 1,554 | 3810.3 | 95.9 | 3712.4 | 97.5 | 8630 | 90 |
| HEC59 | 67,033 | 961 | 3642.5 | 91.7 | 6104 | 89.8 | NT | NT |

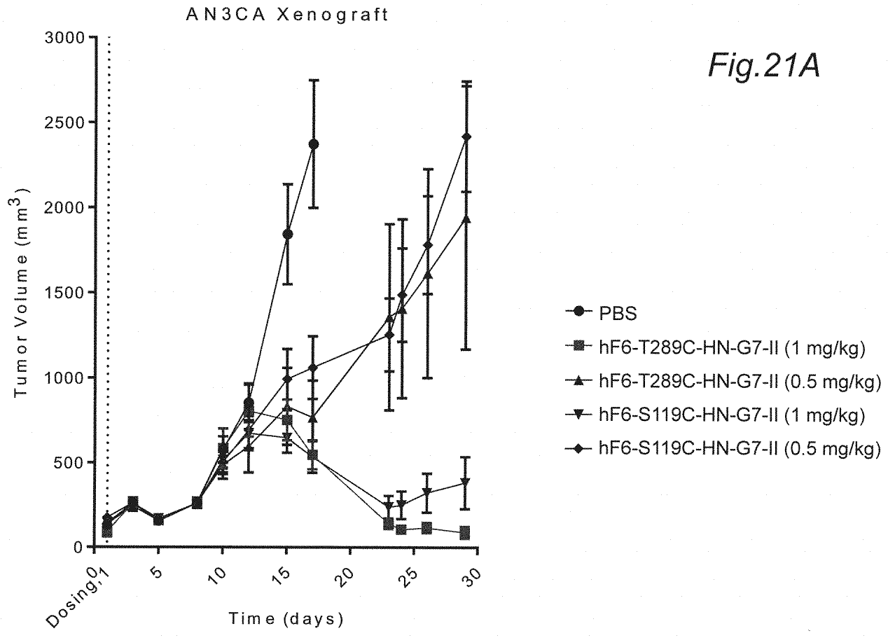

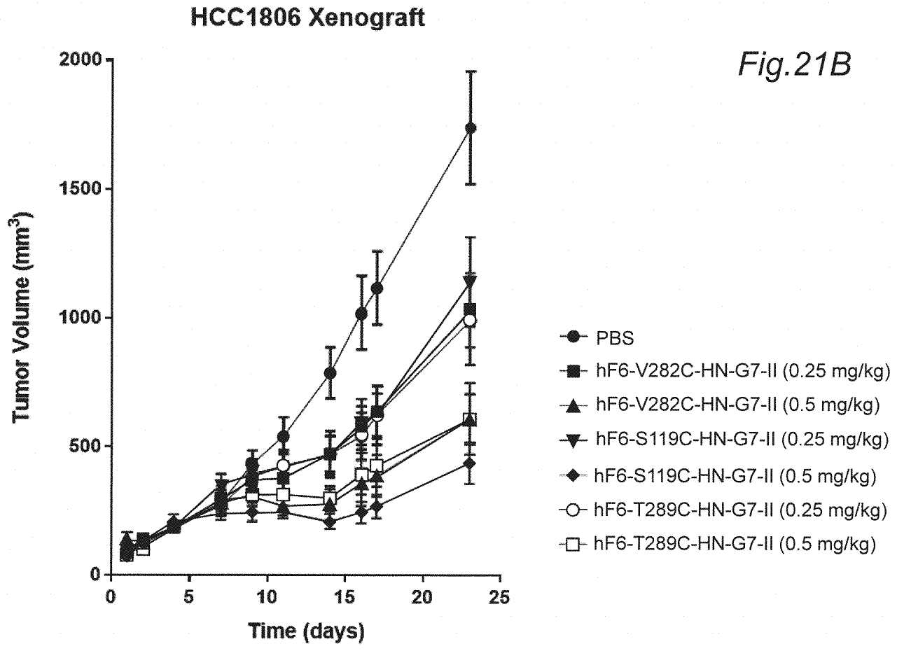

The representative drug-conjugated bi-specific binding agents designated as hF6-S119C-HN-G7-II, hF6-V282C-HN-G7-II, and hF6-T289C-HN-G7-II, were tested in an in vivo xenograft study at different doses (FIG. 21). Briefly, immunodeficient female mice (strain:Nu/Nu) were inoculated subcutaneously with 5×10⁶ An3CA or HCC1806 cells. Tumors were measured using digital calipers and the tumor volume was determined according to the formula 0.5236*L*W². Mice were randomized and dosed intravenously with the indicated bi-specific binding agent when tumors reached an average of 175 mm³ (AN3CA) or 195 mm³ (HCC1806). The results are summarized in Tables 32 (AN3CA) and 33 (ACC 1806) below.

TABLE 32
(AN3CA xenograft summary table)

| Compound | Dose | % T/C | Adjusted p-value |
|---|---|---|---|
| hF6-S119C-HN-G7-II | 1 mg/kg | 22.5 | 0.0001 |
| hF6-S119C-HN-G7-II | 0.5 mg/kg | 44.6 | 0.0006 |
| hF6-T289C-HN-G7-II | 1 mg/kg | 23.1 | 0.0001 |
| hF6-T289C-HN-G7-II | 0.5 mg/kg | 32.4 | 0.0001 |

TABLE 33
(HCC1806 xenograft summary table)

| Compound | Dose | % T/C | Adjusted p-value |
|---|---|---|---|
| hF6-S119C-HN-G7-II | 1 mg/kg | 5.1 | 0.0001 |
| hF6-S119C-HN-G7-II | 0.5 mg/kg | 8.3 | 0.0001 |
| hF6-T289C-HN-G7-II | 1 mg/kg | 3.9 | 0.0001 |
| hF6-T289C-HN-G7-II | 0.5 mg/kg | 10.6 | 0.0001 |

### Example 12—Certain Representative Sequences

Human syndecan-1 (syndecan-1)-UniProtKB-P18827
SEQ ID NO: 1
MRRAALWLWLCALALSLQPALPQIVATNLPPEDQDGSGDDSDNFSGSGAGAL
QDITLSQQTPSTWKDTQLLTAIPTSPEPTGLEATAASTSTLPAGEGPKEGEAVVLPEVEPG
LTAREQEATPRPRETTQLPTTHLASTTTATTAQEPATSHPHRDMQPGHHETSTPAGPSQA
DLHTPHTEDGGPSATERAAEDGASSQLPAAEGSGEQDFTFETSGENTAVVAVEPDRRNQ
SPVDQGATGASQGLLDRKEVLGGVIAGGLVGLIFAVCLVGFMLYRMKKKDEGSYSLEE
PKQANGGAYQKPTKQEEFYA
Mouse syndecan-1 (syndecan-1)-UniProtKB-P18828
SEQ ID NO: 126
MRRAALWLWLCALALRLQPALPQIVAVNVPPEDQDGSGDDSDNFSGSGTGAL
PDTLSRQTPSTWKDVWLLTATPTAPEPTSSNTETAFTSVLPAGEKPEEGEPVLHVEAEPG
FTARDKEKEVTTRPRETVQLPITQRASTVRVTTAQAAVTSHPHGGMQPGLHETSAPTAP
GQPDHQPPRVEGGGTSVIKEVVEDGTANQLPAGEGSGEQDFTFETSGENTAVAAVEPGL
RNQPPVDEGATGASQSLLDRKEVLGGVIAGGLVGLIFAVCLVAFMLYRMKKKDEGSYS
LEEPKQANGGAYQKPTKQEEFYA
Rat syndecan-1 (syndecan-1)-UniProtKB-P26260
SEQ ID NO: 127
MRRAALWLWLCALALRLQPALPQIVTANVPPEDQDGSGDDSDNFSGSGTGALP
DMTLSRQTPSTWKDVWLLTATPTAPEPTSRDTEATLTSILPAGEKPEEGEPVAHVEAEPD
FTARDKEKEATTRPRETTQLPVTQQASTAARATTAQASVTSHPHGDVQPGLHETLAPTA
PGQPDHQPPSVEDGGTSVIKEVVEDETTNQLPAGEGSGEQDFTFETSGENTAVAGVEPD
LRNQSPVDEGATGASQGLLDRKEVLGGVIAGGLVGLIFAVCLVAFMLYRMKKKDEGSY
SLEEPKQANGGAYQKPTKQEEFYA
Macaca mulatta (Rhesus macaque) syndecan-1-UniProtKB-A0A1D5RIX8
SEQ ID NO: 128
MGATAYIPNSNSLSALLRGLELPHQTELLRVRALPTLLCPCALCRAPGCVQIVA
TNLPPEDQDGSGDDSDNFSGSGAGALQDITLSQQTPSTWKDTWLLTATPMSPEPTGLEA
TAASTSTLPAGEGPKEGEAVVLLEVEPDLTAREQEATPQPTETTQLPTTHQAPTARATTA
QEPATSHPHRDMQPGHHETSAPAGPGQADLHTPRTEDGGPSATERAAEDGASSQLPAA
EGSGEQDFTFETSGENTAIVAVEPDHRNQSPVDPGATGASQGLLDRKEVLGGIIAGGLVG
LIFAVCLVGFMLYRMKKKDEGSYSLEEPKQANGGAYQKPTKQEEFYA
Canis lupus familiaris (Dog) (Canis familiaris) syndecan-1-UniProtKB-E2RT70
SEQ ID NO: 129
MRRAALWLWLCALALRLQPALPQIVATNVPPEDQDGSGDDSDNFSGSGAGAL
QDITLSQQTPSTWKDMALLTAMPTAQEPTGADDIDSSTSILLTREGPEGGEAVLVAEAEP
GFTDREKETAHPPSETTPHPTTHRASTARATTAQGPATLHPHRDAQPDHHQISVLAEPSQ
LDPHTPRVEDGGPSATERAAEDGVSTQLPAGEGSGEQDFTFDVSGENTAGTAVEPDQRN
QPPVDRGATGASQGLLDRKEVLGGVIAGGLVGLIFAVCLVGFMLYRMKKKDEGSYSLE
EPKQANGGAYQKPSKQEEFYA
Macaca fascicularis (Cynomolgus Monkey) Syndecan-1
SEQ ID NO: 130
MRRAALWLWLCALALSLQPAMPQIVATNLPPEDQDGSGDDSDNFSGSGAGAL
QDITLSQQTPSTWKDTWLVRATPMSPEPTGLEATAASTSTIQAGEGPKEGEAVVLLEVEP
DLTAREQEATPQPTETTQLPTTHQAPTARATTAQEPATSHPHRDMQPGHHETSAPAGPG
QADLHTPRTEDGGPSATERAAEDGASSQLPAAEGSGEQDFTFETSGENTAIVAVEPDHR
NQSPVDPGATGASQGLLDRKEVLGGIIAGGLVGLIFAVCLVGFMLYRMKKKDEGSYSLE
EPKQANGGAYQKPTKQEEFYA
Human, FGFR3, isoform b
(SEQ ID NO: 97)
ISESLGTEQRVVGRAAEVPGPEPGQQEQLVFGSGDAVELSCPPPGGGPMGPTV
WVKDGTGLVPSERVLVGPQRLQVLNASHEDSGAYSCRQRLTQRVLCHFSVRVTDAPSS
GDDEDGEDEAEDTGVDTGAPYWTRPERMDKKLLAVPAANTVRFRCPAAGNPTPSISWL
KNGREFRGEHRIGGIKLRHQQWSLVMESVVPSDRGNYTCVVENKFGSIRQTYTLDVLER
SPHRPILQAGLPANQTAVLGSDVEFHCKVYSDAQPHIQWLKHVEVNGSKVGPDGTPYV
TVLKSWISESVEADVRLRLANVSERDGGEYLCRATNFIGVAEKAFWLSVHGPRAAEEEL
VEADEAGSVYAG
Human, FGFR3, isoform c
(SEQ ID NO: 98)
ISESLGTEQRVVGRAAEVPGPEPGQQEQLVFGSGDAVELSCPPPGGGPMGPTV
WVKDGTGLVPSERVLVGPQRLQVLNASHEDSGAYSCRQRLTQRVLCHFSVRVTDAPSS
GDDEDGEDEAEDTGVDTGAPYWTRPERMDKKLLAVPAANTVRFRCPAAGNPTPSISWL
KNGREFRGEHRIGGIKLRHQQWSLVMESVVPSDRGNYTCVVENKFGSIRQTYTLDVLER
SPHRPILQAGLPANQTAVLGSDVEFHCKVYSDAQPHIQWLKHVEVNGSKVGPDGTPYV
TVLKTAGANTTDKELEVLSLHNVTFEDAGEYTCLAGNSIGFSHHSAWLVVLPAEEELVE
ADEAGSVYAG

### Example 13—Certain Embodiments

A1. A bi-specific binding agent comprising (a) an antibody, or antigen binding portion thereof, that binds specifically to syndecan-1 (CD138); and (b) a Fynomer that binds specifically to a fibroblast growth factor receptor 3 (FGFR3).

A1.1. The bi-specific binding agent of embodiment A1, wherein the syndecan-1 is a mammalian syndecan-1.

A1.2. The bi-specific binding agent of embodiment A1 or A1.1, wherein the syndecan-1 is selected from a human syndecan-1, a mouse syndecan-1 and a monkey syndecan-1.

A1.3. The bi-specific binding agent of any one of embodiments A1 to A1.2, wherein the bi-specific agent and/or the antibody, or antigen binding portion thereof, binds specifically to human syndecan-1 and mouse syndecan-1.

A1.4. The bi-specific binding agent of any one of embodiments A1 to A1.3, wherein the bi-specific agent and/or the antibody, or antigen binding portion thereof, binds specifically to human syndecan-1 and monkey syndecan-1.

A1.5. The bi-specific binding agent of any one of embodiments A1 to A1.4, wherein the bi-specific agent and/or the antibody, or antigen binding portion thereof, binds specifically to human syndecan-1, mouse syndecan-1 and monkey syndecan-1.

A1.6. The bi-specific binding agent of embodiment A1.2, A1.4 or A1.5, wherein the monkey syndecan-1 comprises a syndecan-1 expressed in, obtained from or isolated from a monkey of the genus Macaca.

A1.7. The bi-specific binding agent of embodiment A1.6, wherein the monkey syndecan-1 is a syndecan-1 expressed in, obtained from or isolated from a monkey of the species Macaca fascicularis (Cynomolgus Monkey).

A1.8. The bi-specific binding agent of embodiment A1.6, wherein the monkey syndecan-1 comprises or consists of the amino acid sequence of SEQ ID NO:128 or 130.

A2. The bi-specific binding agent of any one of embodiments A1.2 to A1.8, wherein the bi-specific agent and/or the antibody, or antigen binding portion thereof, binds specifically to the human syndecan-1, the monkey syndecan-1 and/or the mouse syndecan-1 with a KD of 50 nM or lower.

A3. The bi-specific binding agent of any one of embodiments A1 to A2, wherein the antibody, or antigen binding portion thereof, is covalently attached to the Fynomer.

A4. The bi-specific binding agent of any one of embodiments A1 to A3, wherein the antibody, or antigen binding portion thereof, binds specifically to an extracellular region of the syndecan-1.

A5. The bi-specific binding agent of any one of embodiments A1 to A4, wherein the antibody, or antigen binding portion thereof, binds specifically to a polypeptide comprising the amino acid sequence of AGEGPKEGEAVVLP (SEQ ID NO:94).

A5.1. The bi-specific binding agent of any one of embodiments A1 to A5, wherein the antibody, or antigen binding portion thereof, competes for binding with another binding agent that binds specifically to a polypeptide comprising or consisting of the amino acid sequence of AGEGPKEGEAVVLP (SEQ ID NO:94).

A5.2. The bi-specific binding agent of any one of embodiments A1 to A5.1, wherein the antibody, or antigen binding portion thereof, competes for binding to syndecan-1 with a second binding agent comprising or consisting of a CDR-L1 selected from Table 1, a CDR-L2 selected from Table 2, a CDR-L3 selected from Table 3, a CDR-H1 selected from Table 6, a CDR-H2 selected from Table 7, and a CDR-H3 selected from Table 8.

A5.3. The bi-specific binding agent of embodiment A5.2, wherein the second binding agent comprises a CDR-L1 having an amino acid sequence at least 85%, at least 90%, at least 95% or 100% identical to the amino acid sequence of SEQ ID NO:3, a CDR-L2 having an amino acid sequence at least 85%, at least 90%, at least 95% or 100% identical to the amino acid sequence of SEQ ID NO:18, a CDR-L3 having an amino acid sequence at least 85%, at least 90%, at least 95% or 100% identical to the amino acid sequence of SEQ ID NO:28, a CDR-H1 having an amino acid sequence at least 85%, at least 90%, at least 95% or 100% identical to the amino acid sequence of SEQ ID NO:47, a CDR-H2 having an amino acid sequence at least 85%, at least 90%, at least 95% or 100% identical to the amino acid sequence of SEQ ID NO:60, and a CDR-H3 having an amino acid sequence at least 85%, at least 90%, at least 95% or 100% identical to the amino acid sequence of SEQ ID NO:73.

A5.4. The bi-specific binding agent of embodiment A5.2, wherein the second binding agent comprises a CDR-L1 having an amino acid sequence at least 85%, at least 90%, at least 95% or 100% identical to the amino acid sequence of SEQ ID NO:2, a CDR-L2 having an amino acid sequence at least 85%, at least 90%, at least 95% or 100% identical to the amino acid sequence of SEQ ID NO:17, a CDR-L3 having an amino acid sequence at least 85%, at least 90%, at least 95% or 100% identical to the amino acid sequence of SEQ ID NO:27, a CDR-H1 having an amino acid sequence at least 85%, at least 90%, at least 95% or 100% identical to the amino acid sequence of SEQ ID NO:47, a CDR-H2 having an amino acid sequence at least 85%, at least 90%, at least 95% or 100% identical to the amino acid sequence of SEQ ID NO:61, and a CDR-H3 having an amino acid sequence at least 85%, at least 90%, at least 95% or 100% identical to the amino acid sequence of SEQ ID NO:73.

A5.5. The bi-specific binding agent of embodiment A5.4, wherein the second binding agent comprises a CDR-L2 having an amino acid sequence at least 85%, at least 90%, at least 95% or 100% identical to the amino acid sequence of SEQ ID NO:16, a CDR-H1 having an amino acid sequence at least 85%, at least 90%, at least 95% or 100% identical to the amino acid sequence of SEQ ID NO:45 or 46, and a CDR-H3 having an amino acid sequence at least 85%, at least 90%, at least 95% or 100% identical to the amino acid sequence of SEQ ID NO:72.

A5.6. The bi-specific binding agent of embodiment A5.2, wherein the second binding agent comprises a humanized light chain variable region and a humanized heavy chain variable region.

A5.7. The bi-specific binding agent of embodiment A5.6, wherein the second binding agent comprises a humanized light chain variable region having an amino acid sequence at least 85%, at least 90%, at least 95% or 100% identical to the amino acid sequence of SEQ ID NO:41 and a humanized heavy chain variable region having an amino acid sequence at least 85%, at least 90%, at least 95% or 100% identical to the amino acid sequence of SEQ ID NO:90.

A5.8. The bi-specific binding agent of embodiment A5.6, wherein the second binding agent comprises a humanized light chain having an amino acid sequence at least 85%, at least 90%, at least 95% or 100% identical to the amino acid sequence of SEQ ID NO:44 and a humanized heavy chain having an amino acid sequence at least 85%, at least 90%, at least 95% or 100% identical to the amino acid sequence of SEQ ID NO:93.

A6. The bi-specific binding agent of any one of embodiments A1 to A5.1, wherein the antibody, or antigen binding portion thereof, comprises the following light chain complementarity determining regions (CDR): (i) a CDR-L1 (light chain CDR1) comprising an amino acid sequence having 90% identity to an amino acid sequence selected from SEQ ID NOs:2-15; (ii) a CDR-L2 (light chain CDR2) comprising an amino acid sequence having at least 85%, or at least 90% identity to an amino acid sequence selected from SEQ ID NOs:16-26, and (iii) a CDR-L3 (light chain CDR3) comprising an amino acid sequence having at least 85%, or at least 90% identity to an amino acid sequence selected from SEQ ID NOs:27-33.

A6.1. The bi-specific binding agent of any one of embodiments A1 to A5.1, wherein the antibody, or antigen binding portion thereof, comprises the following light chain complementarity determining regions (CDR): (i) a CDR-L1 (light chain CDR1) comprising or consisting of an amino acid sequence selected from SEQ ID NOs:2-15; (ii) a CDR-L2 (light chain CDR2) comprising or consisting or an amino acid sequence selected from SEQ ID NOs:16-26, and (iii) a CDR-L3 (light chain CDR3) comprising or consisting of an amino acid sequence selected from SEQ ID NOs:27-33.

A7. The bi-specific binding agent of any one of embodiments A1 to A5.1, and A6 to A6.1, wherein the antibody, or antigen binding portion thereof, comprises the following heavy chain complementarity determining regions (CDR): (i) a CDR-H1 (heavy chain CDR1) comprising or consisting of an amino acid sequence having at least 85%, or at least 90% identity to an amino acid sequence selected from SEQ ID NOs:45-59; (ii) a CDR-H2 (heavy chain CDR2) comprising or consisting of an amino acid sequence having at least 85%, or at least 90% identity to an amino acid sequence selected from SEQ ID NOs:60-71, (iii) a CDR-H3 (heavy chain CDR3) comprising or consisting of an amino acid sequence having at least 85%, or at least 90% identity to an amino acid sequence selected from SEQ ID NOs:72-81.

A7.1. The bi-specific binding agent of any one of embodiments A1 to A5.1, and A6 to A6.1, wherein the antibody, or antigen binding portion thereof, comprises the following heavy chain complementarity determining regions (CDR): (i) a CDR-H1 (heavy chain CDR1) comprising or consisting of an amino acid sequence selected from SEQ ID NOs:45-59; (ii) a CDR-H2 (heavy chain CDR2) comprising or consisting of an amino acid sequence selected from SEQ ID NOs:60-71, (iii) a CDR-H3 (heavy chain CDR3) comprising or consisting of an amino acid sequence selected from SEQ ID NOs:72-81.

A8. The bi-specific binding agent of any one of embodiments A6 to A7.1, wherein the CDR-L3 is selected from SEQ ID NO:27 or SEQ ID NO:29; the CDR-L2 is selected from SEQ ID NO:17 or SEQ ID NO:20; and the CDR-L1 is selected from SEQ ID NO:2 or SEQ ID NO:4.

A9. The bi-specific binding agent of any one of embodiments A6 to A8, wherein the CDR-H3 is selected from SEQ ID NO:73 or SEQ ID NO:75; the CDR-H2 is selected from SEQ ID NO:61 or SEQ ID NO:63; and the CDR-H1 is selected from SEQ ID NO:47 or SEQ ID NO:50.

A10. The bi-specific binding agent of any one of embodiments A1 to A9, wherein the CDR-L1 comprises or consists of the amino acid sequence of SEQ ID NO:2, the CDR-L2 comprises or consists of the amino acid sequence of SEQ ID NO:17, the CDR-L3 comprises or consists of the amino acid sequence of SEQ ID NO:27, the CDR-H1 comprises the amino acid sequence of SEQ ID NO:47, the CDR-H2 comprises or consists of the amino acid sequence of SEQ ID NO:61, and the CDR-H3 comprises or consists of the amino acid sequence of SEQ ID NO:73.

A10.1. The bi-specific binding agent of embodiment A10, wherein the antibody, or antigen binding portion thereof, comprises a light chain variable region comprising or consisting of an amino acid sequence at least 85%, at least 90%, at least 95% or 100% identical to the amino acid sequence of SEQ ID NO:41 and a heavy chain variable region comprising or consisting of an amino acid sequence at least 85%, at least 90%, at least 95% or 100% identical to the amino acid sequence of SEQ ID NO:90.

A10.2. The bi-specific binding agent of embodiment A10.1, wherein the antibody, or antigen binding portion thereof, comprises a light chain comprising or consisting of an amino acid sequence at least 85%, at least 90%, at least 95% or 100% identical to the amino acid sequence of SEQ ID NO:44 and a heavy chain comprising or consisting of an amino acid sequence at least 85%, at least 90%, at least 95% or 100% identical to the amino acid sequence of SEQ ID NO:93.

A11. The bi-specific binding agent of any one of embodiments A1 to A9, wherein the CDR-L1 comprises or consists of the amino acid sequence of SEQ ID NO:4, the CDR-L2 comprises or consists of the amino acid sequence of SEQ ID NO:20, the CDR-L3 comprises or consists of the amino acid sequence of SEQ ID NO:29, the CDR-H1 comprises or consists of the amino acid sequence of SEQ ID NO:50, the CDR-H2 comprises or consists of the amino acid sequence of SEQ ID NO:63, and the CDR-H3 comprises or consists of the amino acid sequence of SEQ ID NO:75.

A12. The bi-specific binding agent of any one of embodiments A1 to A11, wherein the antibody, comprises a constant region of an IgG, IgD, IgE, IgA or IgM.

A13. The bi-specific binding agent of any one of embodiments A1 to A12, wherein the antibody, or antigen binding portion thereof, is a chimeric antibody and/or humanized antibody.

A13.1. The bi-specific binding agent of embodiments A13, wherein the chimeric antibody comprises a human constant domain of an IgG1.

A13.2. The bi-specific binding agent of embodiments A13 or A13.1, wherein the humanized antibody comprises one or more human framework regions, or 1, 2, 3, 4, 5 or more framework regions comprising or consisting an amino acid sequence having at least 85%, at least 90% identity, at least 95% identity, or at least 100% identity to a corresponding human framework region.

A14. The bi-specific binding agent of any one of embodiments A1 to A13.2, wherein the antibody, or antigen binding portion thereof binds specifically to an extracellular domain of a human syndecan-1, an extracellular domain of a monkey syndecan-1 and/or an extracellular domain of a mouse syndecan-1.

A15. The bi-specific binding agent of any one of embodiments A1 to A13, wherein the antibody, or antigen binding portion thereof binds specifically to the extracellular domain of the human syndecan-1, the monkey syndecan-1 and/or the mouse syndecan-1 with a binding affinity (KD) of 50 nM or less.

A16. The bi-specific binding agent of any one of embodiments A1 to A15, wherein the FGFR3 is a human fibroblast growth factor receptor 3 (FGFR3).

A17. The bi-specific binding agent of any one of embodiments A1 to A16, wherein the human FGFR3 comprises an FGFR3 isoform 3b or FGFR3 isoform 3c.

A18. The bi-specific binding agent of embodiment A16 or A17, wherein the Fynomer binds specifically to a human FGFR3 isoform 3b and an FGFR3 isoform 3c.

A19. The bi-specific binding agent of any one of embodiments A1 to A18, wherein the Fynomer comprises an RT-loop comprising or consisting of the amino acid sequence EVMSTTA (SEQ ID NO:114) and a SRC loop comprising or consisting of the amino acid sequence SQSPH (SEQ ID NO: 115).

A20. The bi-specific binding agent of any one of embodiments A1 to A19, wherein the Fynomer comprises or consists of a polypeptide having an amino acid sequence at least 85%, at least 90%, or at least 95% identical to the amino acid sequence GVTLFVALYDYEVMSTTALSFHKGEKFQILSQSPHGQYWEARSLTTGETG(X₇)IPSNYVA PVDSIQ (SEQ ID NO: 113), wherein the amino acid at position (X₇) is any amino acid.

A20.1. The bi-specific binding agent of any one of embodiments A1 to A19, wherein the Fynomer comprises or consists of a polypeptide having the amino acid sequence of GVTLFVALYDYEVMSTTALSFHKGEKFQILSQSPHGQYWEARSLTTGETG(X₇)IPSNYVA PVDSIQ (SEQ ID NO: 113), wherein the amino acid at position (X₇) is any amino acid.

A21. The bi-specific binding agent of embodiment A20 or A20.1, wherein (X₇) is selected from N, R, W and K.

A22. The bi-specific binding agent of any one of embodiments A1 to A18, wherein the Fynomer comprises an RT-loop comprising or consisting of the amino acid sequence EVYGPTPM (SEQ ID NO:100).

A23. The bi-specific binding agent of any one of embodiments A1 to A18, or A22, wherein the Fynomer comprises or consists of a polypeptide having an amino acid sequence at least 85%, at least 90%, at least 95% or 100% identical to the amino acid sequence GVTLFVALYDYEVYGPTPMLSFHKGEKFQIL(X₁)(X₂)(X₃)(X₄)GPYWEARSL(X₅)TGETG(X₆)IPSNYVAPVDSIQ (SEQ ID NO:99), wherein the amino acids (X₁), (X₂), (X₃), (X₄), (X₅) and (X₆) are selected from any amino acid.

A23.1. The bi-specific binding agent of A23, wherein the Fynomer comprises or consists of the amino acid sequence GVTLFVALYDYEVYGPTPMLSFHKGEKFQIL(X₁)(X₂)(X₃)(X₄)GPYWEARSL(X₅)TGETG(X₆)IPSNYVAPVDSIQ (SEQ ID NO:99), wherein the amino acids (X₁), (X₂), (X₃), (X₄), (X₅) and (X₆) are selected from any amino acid.

A24. The bi-specific binding agent of embodiment A23 or A23.1, wherein

(X1) is N, R, or K;

(X2) is S, G, K or R;

(X3) is S or G;

(X4) is E, Q, D, S or K;

(X5) is T or A; and

(X6) is Y, W or L.

A25. The bi-specific binding agent of any one of embodiments A1 to A18, wherein the Fynomer comprises a polypeptide comprising an amino acid sequence that is at least 85% identical, at least 90% identical, at least 95% identical or 100% identical to an amino acid sequence selected from:

(SEQ ID NO: 101; FF2L4C3)
GVTLFVALYDYEVYGPTPMLSFHKGEKFQILNSSEGPYWEARSLTTGETG
LIPSNYVAPVDSIQ;
(SEQ ID NO: 103; FF44L65G12)
GVTLFVALYDYEVYGPTPMLSFHKGEKFQILRGGQGPYWEARSLTTGETG
LIPSNYVAPVDSIQ;
(SEQ ID NO: 105; FF44L65G7)
GVTLFVALYDYEVYGPTPMLSFHKGEKFQILRGGDGPYWEARSLTTGETG
LIPSNYVAPVDSIQ;
(SEQ ID NO: 107; FF48L66G7; “G7”)
GVTLFVALYDYEVYGPTPMLSFHKGEKFQILKGGSGPYWEARSLTTGETG
LIPSNYVAPVDSIQ;
(SEQ ID NO: 109; FF43L65D5)
GVTLFVALYDYEVYGPTPMLSFHKGEKFQILRKGKGPYWEARSLATGETG
LIPSNYVAPVDSIQ;
(SEQ ID NO: 111; FF44L65B7)
GVTLFVALYDYEVYGPTPMLSFHKGEKFQILRRGSGPYWEARSLTTGETG
LIPSNYVAPVDSIQ;
and
(SEQ ID NO: 116; FF40L54A5)
GVTLFVALYDYEVMSTTALSFHKGEKFQILSQSPHGQYWEARSLTTGETG
WIPSNYVAPVDSIQ.

A25.1. The bi-specific binding agent of any one of embodiments A1 to A18, wherein the Fynomer comprises a polypeptide comprising an amino acid sequence that is at least 85% identical, at least 90% identical, at least 95% identical or 100% identical to the amino acid sequence of SEQ ID NO:107, where the amino acid sequence EVYGPTPM (SEQ ID NO:100) at amino acid positions 12 to 19 of SEQ ID NO:107 and amino acids P and Y at amino acid positions 37 and 38 of SEQ ID NO:107 are conserved, and the Fynomer binds specifically to an FGFR3.

A25.2. The bi-specific binding agent of any one of embodiments A1 to A18, wherein the Fynomer comprises a polypeptide comprising an amino acid sequence that is at least 85% identical, at least 90% identical, at least 95% identical or 100% identical to the amino acid sequence of SEQ ID NO:107, where the amino acid sequence EVYGPTPM (SEQ ID NO:100) at amino acid positions 12 to 19 of SEQ ID NO:107 and amino acids at amino acid positions 32 to 38 of SEQ ID NO:107 are conserved, and the Fynomer binds specifically to an FGFR3.

A25.3. The bi-specific binding agent of any one of embodiments A1 to A18, wherein the Fynomer comprises or consists of a polypeptide comprising or consisting of the amino acid sequence of SEQ ID NO:107.

A25.4. The bi-specific binding agent of any one of embodiments A1 to A25.3, wherein the Fynomer specifically binds to both human FGFR3 isoform b and FGFR3 isoform c.

A26. The bi-specific binding agent of any one of embodiments A1 to A25.4, wherein the Fynomer binds specifically to an extracellular region of the FGFR3, FGFR3b or FGFR3c.

A27. The bi-specific binding agent of any one of embodiments A1 to A26, wherein the Fynomer binds specifically to the FGFR3, FGFR3b, FGFR3c, or a portion thereof, with a binding affinity (KD) of from about 10⁻⁵ M to about 10⁻¹⁵ M.

A28. The bi-specific binding agent of any one of embodiments A1 to A27, wherein the Fynomer binds specifically to the FGFR3, FGFR3b, FGFR3c, or a portion thereof, with a binding affinity (KD) of 10⁻⁸ M or less.

A29. The bi-specific binding agent of any one of embodiments A1 to A28, wherein the Fynomer is glycosylated.

A30. The bi-specific binding agent of any one of embodiments A1 to A29, wherein the Fynomer is covalently attached to the antibody, or antigen binding portion thereof, by a linker.

A30.1 The bi-specific binding agent of embodiment A30, wherein the linker comprises or consists of a peptide bond.

A31. The bi-specific binding agent of embodiment A30 or A30.1, wherein the linker comprises or consists of a peptide comprising or consisting of one or more amino acids, 5 to 100 amino acids, 5 to 50 amino acids, 5 to 25 amino acids, 5 to 20 amino acids, or 5 to 10 amino acids.

A32. The bi-specific binding agent of embodiment A30, A30.1, or A31, wherein the linker comprises or consists of an optionally substituted C1-C50 alkyl, an optionally substituted C2-C50 alkenyl, an optionally substituted C2-C50 alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxy carbonyl, alkoxy carbonylacyl, aminocarbonyl, aminocarboyloxy, azido, phenyl, cycloalkylacyl, alkylthio, arylthio, oxysulfonyl, carboxy, thio, sulfoxide, sulfone, sulfonate esters, thiocyano, amide, amino, ester, halogenated alkyl, or a combination thereof.

A33. The bi-specific binding agent of any one of embodiments A1 to A29, wherein the antibody, or antigen binding portion thereof is non-covalently attached to the Fynomer by means of a binding pair.

A34. The bi-specific binding agent of any one of embodiments A1 to A33, wherein the Fynomer is attached to an N-terminal end of a heavy chain or light chain of the antibody, or antigen binding portion thereof.

A35.1. The bi-specific binding agent of any one of embodiments A1 to A33, wherein the Fynomer is covalently attached to an N-terminal end of a heavy chain of the antibody by a peptide bond.

A35.2. The bi-specific binding agent of embodiments A35.1, wherein a C-terminal amino acid of the Fynomer is covalently attached to an N-terminal amino acid of a heavy chain of the antibody by a peptide bond.

A35.3. The bi-specific binding agent of embodiments A35.1, wherein a C-terminal amino acid of the Fynomer is covalently attached to an N-terminal amino acid of a light chain of the antibody by a peptide bond.

A35.4. The bi-specific binding agent of any one of embodiments A1 to A33, wherein the Fynomer is attached to a carboxy-terminal end of a heavy chain or light chain of the antibody, or antigen binding portion thereof.

A35.5. The bi-specific binding agent of embodiments A35.4, wherein an N-terminal amino acid of the Fynomer is covalently attached to a carboxy-terminal amino acid of a heavy chain of the antibody by a peptide bond.

A35.6. The bi-specific binding agent of embodiments A35.4, wherein an N-terminal amino acid of the Fynomer is covalently attached to a carboxy-terminal amino acid of a light chain of the antibody by a peptide bond.

A35.7. The bi-specific binding agent of any one of embodiments A1 to A29, comprising (i) the Fynomer and heavy chain amino acid sequence of SEQ ID NO:125 and (ii) the light chain amino acid sequence of SEQ ID NO:44.

A35.8. The bi-specific binding agent of any one of embodiments A1 to A29, comprising or consisting of (i) the Fynomer and heavy chain amino acid sequence of SEQ ID NO:125, wherein the serine at amino acid position 213 of SEQ ID NO:125 is mutated to a cysteine, (ii) the light chain amino acid sequence of SEQ ID NO:44, and (iii) an anti-neoplastic agent covalently attached to the cysteine.

A36. The bi-specific binding agent of any one of embodiments A1 to A35.7, further comprising an anti-neoplastic agent.

A37. The bi-specific binding agent of embodiment A35.8 or A36, wherein the anti-neoplastic agent is selected from the group consisting of an auristatin, a dolastatin, a maytansine, a tubulysin, a calicheamicin, a pyrrolobenzodiazepine (PBD), a duocarmycin, a doxorubicin, a pseudomonas exotoxin-A (PE38), an irinotecan and a derivative of any one of the foregoing.

A38. The bi-specific binding agent of embodiment A35.8, A36 or A37, wherein the anti-neoplastic agent is covalently or non-covalently attached to the bi-specific binding agent.

A39. The bi-specific binding agent of embodiment A38, wherein the anti-neoplastic agent is attached to the antibody, or antigen binding portion thereof.

A40. The bi-specific binding agent of embodiment A38, wherein the anti-neoplastic agent is attached to the Fynomer.

A41. The bi-specific binding agent of any one of embodiments A35.8 to A40, wherein the anti-neoplastic agent is non-covalently attached to the bi-specific binding agent by means of a binding pair.

A42. The bi-specific binding agent of any one of embodiments A35.8 to A40, wherein the anti-neoplastic agent is covalently attached to the bi-specific binding agent by a linker.

A43. The bi-specific binding agent of any one of embodiments A35.8 to A42, wherein the anti-neoplastic agent comprises monomethyl auristatin E (MMAE) or monomethyl auristatin F (MMAF).

A44. The bi-specific binding agent of any one of embodiments A35.8 to A40, and A42, wherein the anti-neoplastic agent comprises or consists of a pyrrolobenzodiazepine toxin and a linking group; wherein the pyrrolobenzodiazepine toxin is covalently linked to the linking group and the linking group is covalently linked to the bi-specific binding agent.

A45. The bi-specific binding agent of embodiment A44, wherein the pyrrolobenzodiazepine toxin comprises or consists of the structure of chemical formula I:

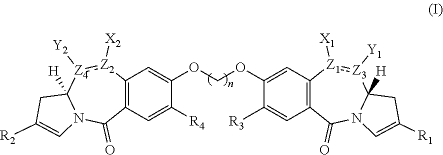

wherein
Z₁ and Z₂ are both N;
Z₃ and Z₄ are both C;

the double-dash lines [symbol] represent a single bond or a double bond;

n is 1 to 10;

each of R₃ and R₄ are independently H, or a C₁₋₄ alkoxyl; and
each of R₁ and R₂ are independently selected from the group consisting of H, C₁₋₅ alkyl, C₃₋₆ cycloalkyl, C₂₋₅ alkenyl, and a phenyl optionally substituted with R₅, wherein
   R₅ is selected from the group consisting of —NH₂, —NHR₆, and a piperazinyl substituted with R₇ having the structure

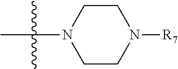

wherein R₆ comprises the linking group, and
R₇ is H, or a C₁₋₅ alkyl;
X₁ is null, a protecting group, or comprises the linking group;
X₂ is null, a protecting group, or comprises the linking group;

only one of X₁, X₂, R₁, and R₂ comprises the linking group; and

each of Y₁ and Y₂ are independently either null, OH, or SO₃H;
provided that:
(i) when X₁ comprises or consists of the linking group, Z₁ [symbol] Z₃ is N—C,
(ii) when X₂ comprises or consists of the linking group, Z₂ [symbol] Z₄ is N—C,
(iii) when X₁ is the protecting group, Z₁ [symbol] Z₃ is N—C, and
(iv) when X₂ is the protecting group, Z₂ [symbol] Z₄ is N—C,
   wherein null means that an indicated moiety is absent from the structure of chemical formula I or indicates the presence of one or more hydrogens, or where not explicitly specified, the one or more hydrogens may be present to complete a required valence.

A46. The bi-specific binding agent of embodiment A45, wherein n is 3, 4 or 5.

A47. The bi-specific binding agent of embodiment A45 or A46, wherein R₃ and R₄ are both —O—CH₃.

A48. The bi-specific binding agent of any one of embodiments A45 to A47, wherein R₁ and R₂ are both methyl.

A49. The bi-specific binding agent of any one of embodiments A45 to A47, wherein R₁ and R₂ are both —CH═CH—CH₃.

A50. The bi-specific binding agent of any one of embodiments A45 to A47, wherein R₂ is a cyclopropyl.

A51. The bi-specific binding agent of any one of embodiments A45 to A47, wherein R₂ is phenyl substituted with 4-methylpiperazin-1-yl.

A52. The bi-specific binding agent of embodiment A50 or A51, wherein R₁ is a phenyl optionally substituted with R₅, R₅ is —NHR₆ and R₆ comprises or consists of the linking group.

A53. The bi-specific binding agent of any one of embodiments A45 to A47 and A50 to A52, wherein X₁ is null, Y₁ is null, Z₁ [symbol] Z₃ is N=C, X₂ is null, Y₂ is null and Z₂ [symbol] Z₄ is N=C.

A54. The bi-specific binding agent of any one of embodiments A45 to A51, wherein X₁ comprises or consists of the linking group, Y₁ is OH, Z₂ [symbol] Z₄ is N=C, X₂ is null, and Y₂ is null.

A55. The bi-specific binding agent of any one of embodiments A45 to A51, wherein X₁ comprises or consists of the linking group, Y₁ is OH, Z₂ [symbol] Z₄ is N—C, X₂ is a protecting group, and Y₂ is OH.

A56. The bi-specific binding agent of any one of embodiments A44 to A55, wherein the linking group is attached to the pyrrolobenzodiazepine toxin by a carbamate group.

A57. The bi-specific binding agent of any one of embodiments A44 to A55, wherein the linking group is attached to the pyrrolobenzodiazepine toxin by an amide group.

A58. The bi-specific binding agent of any one of embodiments A44 to A57, wherein the linking group comprises or consists of the structure of chemical formula A:

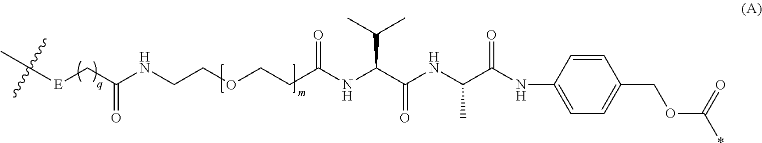

wherein
the asterisk indicates the point of attachment to the pyrrolobenzodiazepine toxin;
the wavy line indicates the point of attachment to the binding agent;
m is 1 to 20;
q is 0 to 10; and
E is a connecting group.

A59. The bi-specific binding agent of embodiment A58, wherein m is 4 or 8.

A60. The bi-specific binding agent of embodiment A58 or A59, wherein q is 0, 1 or 2.

A61. The bi-specific binding agent of embodiment A58, wherein m is 8 and q is 2.

A62. The bi-specific binding agent of any one of embodiments A44 to A57, wherein the linking group comprises or consists of the structure of chemical formula B:

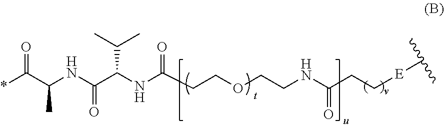

wherein
the asterisk indicates the point of attachment to the pyrrolobenzodiazepine toxin;
the wavy line indicates the point of attachment to the binding agent;
E is a connecting group;
v is 0 to 10; and
u is 0 or 1; wherein when u is 1, t is 1 to 10.

A63. The bi-specific binding agent of embodiment A62, wherein v is 1.

A64. The bi-specific binding agent of embodiment A62 or A63, wherein u is 1, and t is 8.

A65. The bi-specific binding agent of embodiment A62, wherein u is 0, and v is 4.

A66. The bi-specific binding agent of any one of embodiments A58 to A65, wherein the binding agent is connected to E by a thioether bond formed between a cysteine thiol residue of the binding agent and E.

A67. The bi-specific binding agent of any one of embodiments A58 to A66, wherein E comprises or consists of the structure of chemical formula C:

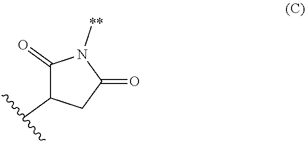

wherein the wavy line indicates the point of attachment to the binding agent and the double asterisk indicates the point of attachment to the linking group.

A68. The bi-specific binding agent of any one of embodiments A45 to A67, wherein the protecting group has the following structure (D):

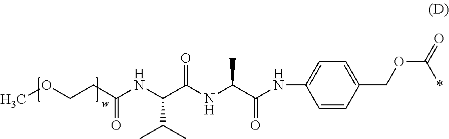

wherein the asterisk indicates the point of attachment to the pyrrolobenzodiazepine toxin; and

w is 1 to 5.

A69. The bi-specific binding agent of embodiment A68, wherein w is 2.

A70. The bi-specific binding agent of any one of embodiments A45 to A69, wherein the protecting group is a cleavable protecting group.

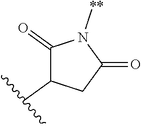

A71. The bi-specific binding agent of embodiment A44, wherein the anti-neoplastic agent comprises or consists of a structure selected from the group consisting of

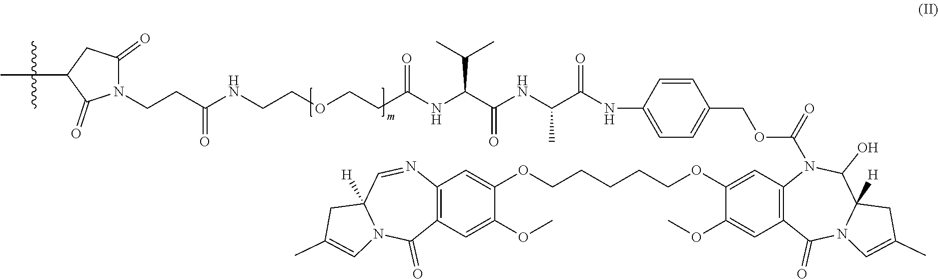

wherein m is 8, p is 3, and X₂ is a protecting group;

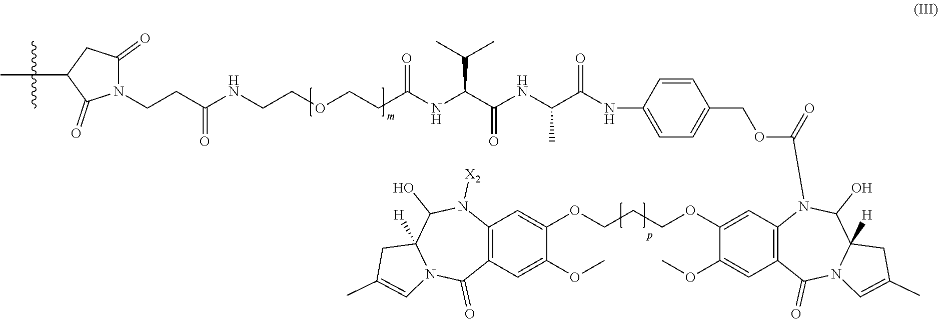

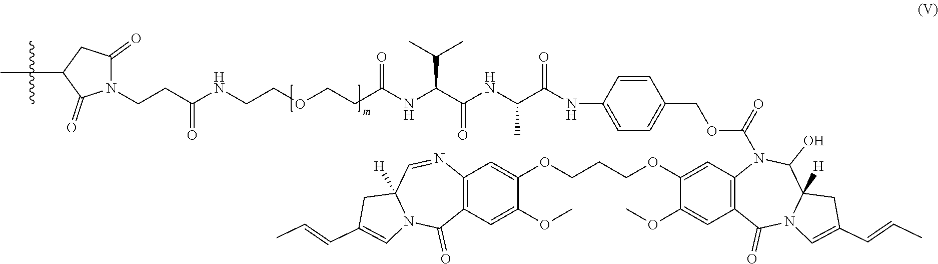

wherein m is 8;

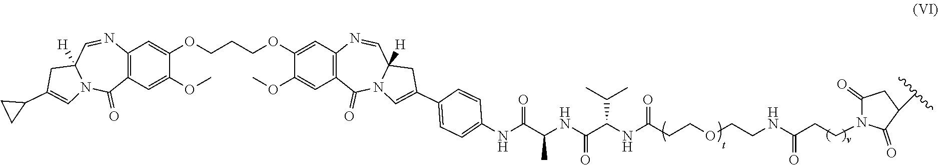

wherein t is 8, and v is 1; and

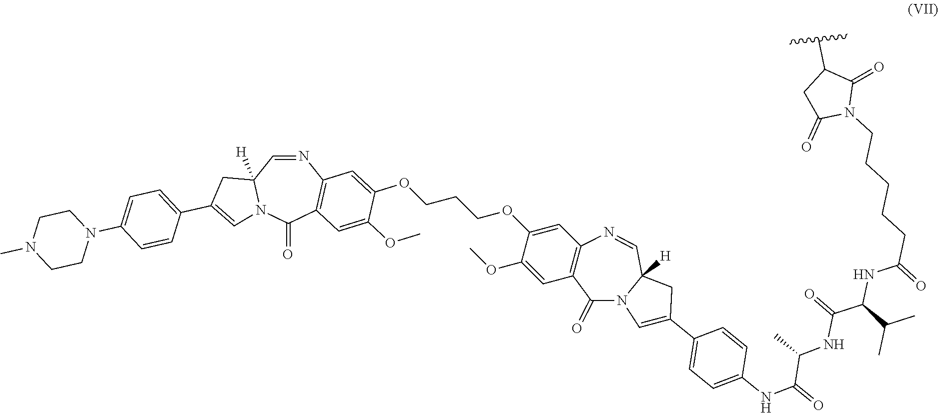 [SMILES: *C1CC(=O)N(CCCCCC(=O)N[C@H](C(=O)N[C@@H](C)C(=O)Nc2ccc(C3=CN4C(=O)c5cc(OC)c(OCCCOc6cc7c(cc6OC)C(=O)N6C=C(c8ccc(N9CCN(C)CC9)cc8)C[C@H]6C=N7)cc5N=C[C@@H]4C3)cc2)C(C)C)C1=O]

wherein the wavy line indicates the point of attachment to the binding agent.

A72. The bi-specific binding agent of embodiment A71, wherein the protecting group of X₂ has the following structure (D):

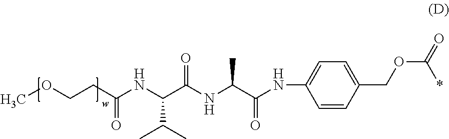

wherein the asterisk indicates the point of attachment to the anti-neoplastic agent or PBD toxin; and w is 1 to 5.

A73. The bi-specific binding agent of any one of embodiments A44 to A72, wherein at least one amino acid of the binding agent is mutated to a cysteine, and the cysteine is linked covalently to the linking group by a thiol ether bond.

A73.1. The bi-specific binding agent of embodiment A73, wherein the antibody comprises a human heavy chain constant region of an IgG₁ or IgG2, and the serine at position 119 of the heavy chain constant region of the antibody is mutated to a cysteine, and the cysteine is linked covalently to the linking group by a thiol ether bond.

A73.2. The bi-specific binding agent of any one of embodiments A44 to A72, wherein at least one amino acid of the binding agent is mutated to a lysine, and a free amino group of the lysine is linked covalently to the linking group.

A74. The bi-specific binding agent of embodiment A44 or A73.2, wherein the antineoplastic agent comprises or consists of the structure of chemical formula (II):

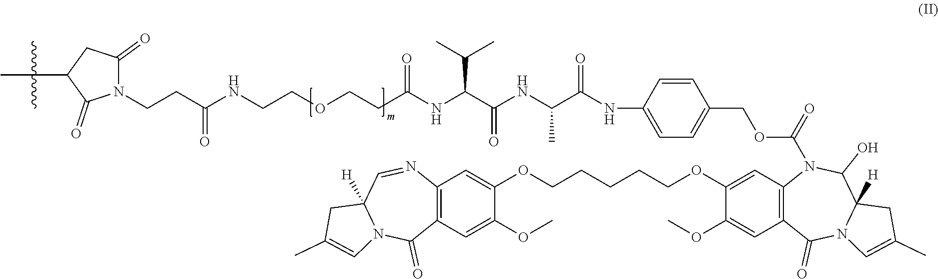

wherein m is 8, and the wavy line indicates the point of attachment to a thiol group of the binding agent.

A75. A pharmaceutical composition comprising the bi-specific binding agent of any one of embodiments A1 to A74 and a pharmaceutically acceptable excipient, diluent, additive or carrier.

A76. The pharmaceutical composition of embodiment A75, wherein the pharmaceutical composition is formulated as a sterile, lyophilized powder.

A77. The pharmaceutical composition of embodiment A75 or A76, wherein the pharmaceutical composition is formulated for intravenous administration to a mammal.

A77.1. The pharmaceutical composition of any one of embodiments A75 to A77 for use in the treatment of a neoplasm.

A77.2. The pharmaceutical composition of embodiment A77.1, wherein the neoplasm comprises a neoplastic cell or cancer cell that expresses syndecan-1.

A77.3. The pharmaceutical composition of any one of embodiments A77.1 to A77.2, wherein the neoplasm is selected from the group consisting of a carcinoma, sarcoma, nervous system neoplasia, lymphoma, myeloma, leukemia, melanoma, mesothelioma, solid or soft tissue tumors, and a secondary cancer.

A77.4. The pharmaceutical composition of any one of embodiments A77.1 to A77.3, wherein the neoplasm is selected from the group consisting of a bladder cancer, breast cancer, colorectal cancer, gastric cancer, pancreatic cancer, esophageal cancer, liver cancer, hepatocellular cancer, hypopharynx cancer, lung cancer, adenocarcinoma, ovarian cancer and renal cancer.

A77.5. The pharmaceutical composition of any one of embodiments A77.1 to A77.3, wherein the neoplasm is selected from the group consisting of a pancreatic adenocarcinoma, pancreatic neuroendocrine cancer, colorectal adenocarcinoma, small intestinal malignancy, cholangiocarcinoma, non-small cell lung cancer (NSCLC), thyroid carcinoma, esophageal or esophagogastric junction (EGJ) cancer, gastric adenocarcinoma, liver hepatocellular carcinoma, head and neck squamous carcinoma, female genital tract malignancy, breast carcinoma, triple negative breast cancer, lung small cell carcinoma, ovarian surface epithelial carcinoma, retroperitoneal or peritoneal sarcoma, prostatic adenocarcinoma, neuroendocrine tumor, gastrointestinal stromal tumor, glioblastoma and non-epithelial ovarian cancer.

A77.6. The pharmaceutical composition of any one of embodiments A77.1 to A77.3, wherein the neoplasm is selected from the group consisting of multiple myeloma, ovarian carcinoma, cervical cancer, endometrial cancer, thyroid cancer, testicular cancer, kidney carcinoma, gall bladder carcinoma, transitional cell bladder carcinoma, gastric cancer, prostate cancer, prostate adenocarcinoma, breast cancer, lung cancer, colon carcinoma, Hodgkin's and non-Hodgkin's lymphoma and multiple myeloma, chronic lymphocytic leukemia (CLL), acute lymphoblastic leukemia (ALL), T-cell acute lymphoblastic leukemia (T-ALL), B-cell malignancies, B-cell acute lymphoblastic leukemia (B-ALL), acute myeloblastic leukemia (AML), a solid tissue sarcoma, colon carcinoma, non-small cell lung carcinoma, squamous cell lung carcinoma, colorectal carcinoma, hepato-carcinoma, pancreatic cancer, brain cancer (e.g., neuroblastoma or meningioma), skin cancer (e.g., melanoma, basal cell carcinoma, or squamous cell carcinoma), and head and neck carcinoma.

A78. A method of treating a subject having, or suspected of having, a neoplasm comprising:
a) providing a subject having, or suspected of having, a neoplasm; and
b) administering to the subject a therapeutically effective amount of the bi-specific binding agent of any one of embodiments A1 to A74, or the pharmaceutical composition of any one of embodiments A75 to A77.

A79. The method of embodiment A78, wherein after the administering, the bi-specific binding agent blocks, inhibits, ameliorates, abrogates, or suppresses growth, viability or metastasis of the cancer.

A80. The method of any embodiment A78 to A79, wherein after the administering, the bi-specific binding agent induces death, necrosis or apoptosis of some or all of the cancer.

A81. The method of any one of embodiments A78 to A80, wherein the neoplasm comprises or consists of a carcinoma, sarcoma, nervous system neoplasia, lymphoma, myeloma, leukemia, melanoma, mesothelioma, solid or soft tissue tumors, or secondary cancers.

A82. The method of embodiment A81, wherein the neoplasm comprises or consists of a bladder cancer, breast cancer, colorectal cancer, gastric cancer, pancreatic cancer, esophageal cancer, liver cancer, hepatocellular cancer, hypopharynx cancer, lung cancer, adenocarcinoma, ovarian cancer or renal cancer.

A83. The method of embodiment A81 or A82, wherein the neoplasm comprises or consists of a pancreatic adenocarcinoma, pancreatic neuroendocrine cancer, colorectal adenocarcinoma, small intestinal malignancy, cholangiocarcinoma, non-small cell lung cancer (NSCLC), thyroid carcinoma, esophageal or esophagogastric junction (EGJ) cancer, gastric adenocarcinoma, liver hepatocellular carcinoma, head and neck squamous carcinoma, female genital tract malignancy, breast carcinoma, triple negative breast cancer, lung small cell carcinoma, ovarian surface epithelial carcinoma, retroperitoneal or peritoneal sarcoma, prostatic adenocarcinoma, neuroendocrine tumor, gastrointestinal stromal tumor, glioblastoma or non-epithelial ovarian cancer.

A84. The method of embodiment A81, wherein the neoplasm is selected from the group consisting of multiple myeloma, ovarian carcinoma, cervical cancer, endometrial cancer, thyroid cancer, testicular cancer, kidney carcinoma, gall bladder carcinoma, transitional cell bladder carcinoma, gastric cancer, prostate cancer, prostate adenocarcinoma, breast cancer, lung cancer, colon carcinoma, Hodgkin's and non-Hodgkin's lymphoma and multiple myeloma, chronic lymphocytic leukemia (CLL), acute lymphoblastic leukemia (ALL), T-cell acute lymphoblastic leukemia (T-ALL), B-cell malignancies, B-cell acute lymphoblastic leukemia (B-ALL), acute myeloblastic leukemia (AML), a solid tissue sarcoma, colon carcinoma, non-small cell lung carcinoma, squamous cell lung carcinoma, colorectal carcinoma, hepato-carcinoma, pancreatic cancer, brain cancer (e.g., neuroblastoma or meningioma), skin cancer (e.g., melanoma, basal cell carcinoma, or squamous cell carcinoma), and head and neck carcinoma.

A85. The method of any one of embodiments A78 to A84, further comprising administering a chemotherapeutic agent to the subject.

A86. The method of any one of embodiments A78 to A85, wherein the subject is a human.

A87. The bi-specific binding agent of any one of embodiments A1 to A74, or the pharmaceutical composition of any one of embodiments A75 to A77, for use in treating a subject having or suspected of having a neoplasm.

A88. The method or use of any one of embodiments A78 to A87, wherein the neoplasm comprises a neoplastic cell or cancer cell that expresses syndecan-1 or an FGFR3.

A89. The method or use of any one of embodiments A78 to A87, wherein the FGFR3 is a human FGFR3.

A90. The bi-specific binding agent of any one of embodiments A1 to A89, wherein the bi-specific agent is internalized into a cell upon binding to the syndecan-1 that is expressed on the surface of the cell and/or upon binding to an FGFR3 expressed on the surface of the cell.

A91. The bi-specific binding agent of any one of embodiments A1 to A90, comprising (i) the Fynomer and heavy chain amino acid sequence of SEQ ID NO:125 and (ii) the light chain amino acid sequence of SEQ ID NO:44.

A92. The bi-specific binding agent of any one of embodiments A1 to A90, comprising (i) the Fynomer and heavy chain amino acid sequence of SEQ ID NO:125, wherein the serine at amino acid position 213 of SEQ ID NO:125 is mutated to a cysteine and (ii) the light chain amino acid sequence of SEQ ID NO:44, wherein the pyrrolobenzodiazepine toxin is covalently attached to the thiol group of the cysteine.

B0. A binding agent comprising (a) an antibody, or antigen binding portion thereof, that binds specifically to syndecan-1 (CD138); and (b) an anti-neoplastic agent.

B1. The binding agent of embodiment B0, wherein the antibody, or antigen binding portion thereof, is covalently or non-covalently attached to the anti-neoplastic agent.

B2. The binding agent of embodiment B0 or B1, wherein the antibody, or antigen binding portion thereof, binds specifically to an extracellular region of the syndecan-1.

B3. The binding agent of any one of embodiments B0 to B2, wherein the syndecan-1 is a mammalian syndecan-1.

B4. The binding agent of any one of embodiments B0 to B3, wherein the antibody, or antigen binding portion thereof, binds specifically to a polypeptide comprising or consisting of the amino acid sequence of AGEGPKEGEAVVLP (SEQ ID NO:94).

B5. The binding agent of any one of embodiments B0 to B4, wherein the antibody, or antigen binding portion thereof, comprises the following light chain complementarity determining regions (CDR): (i) a CDR-L1 (light chain CDR1) comprising or consisting of an amino acid sequence having at least 85% identity or at least 90% identity to an amino acid sequence selected from SEQ ID NOs:2-15; (ii) a CDR-L2 (light chain CDR2) comprising or consisting of an amino acid sequence having at least 85% identity or at least 90% identity to an amino acid sequence selected from SEQ ID NOs:16-26, and (iii) a CDR-L3 (light chain CDR3) comprising or consisting of an amino acid sequence having at least 85% identity or at least 90% identity to an amino acid sequence selected from SEQ ID NOs:27-33.

B6. The binding agent of embodiment B5, wherein the CDR-L1 is selected from SEQ ID NO:2 and SEQ ID NO:4; the CDR-L2 is selected from SEQ ID NO:17 and SEQ ID NO:20; and the CDR-L3 is selected from SEQ ID NO:27 and SEQ ID NO:29.

B7. The binding agent of any one of embodiments B0 to B6, wherein the antibody, or antigen binding portion thereof, comprises the following heavy chain complementarity determining regions (CDR): (i) a CDR-H1 (heavy chain CDR1) comprising or consisting of an amino acid sequence having at least 85% identity or at least 90% identity to an amino acid sequence selected from SEQ ID NOs:45-59; (ii) a CDR-H2 (heavy chain CDR2) comprising or consisting of an amino acid sequence having at least 85% identity or at least 90% identity to an amino acid sequence selected from SEQ ID NOs:60-71, and (iii) a CDR-H3 (heavy chain CDR3) comprising or consisting of an amino acid sequence having at least 85% identity or at least 90% identity to an amino acid sequence selected from SEQ ID NOs:72-81.

B8. The binding agent of any one of embodiments B0 to B7, wherein the CDR-H3 is selected from SEQ ID NO:73 and SEQ ID NO:75; the CDR-H2 is selected from SEQ ID NO:61 and SEQ ID NO:63; and the CDR-H1 is selected from SEQ ID NO:47 and SEQ ID NO:50.

B9. The binding agent of any one of embodiments B0 to B8, wherein the CDR-L1 comprises or consists of the amino acid sequence of SEQ ID NO:2, the CDR-L2 comprises or consists of the amino acid sequence of SEQ ID NO:17, the CDR-L3 comprises or consists of the amino acid sequence of SEQ ID NO:27, the CDR-H1 comprises or consists of the amino acid sequence of SEQ ID NO:47, the CDR-H2 comprises or consists of the amino acid sequence of SEQ ID NO:61, and the CDR-H3 comprises or consists of the amino acid sequence of SEQ ID NO:73.

B10. The binding agent of any one of embodiments B0 to B8, wherein the CDR-L1 comprises or consists of the amino acid sequence of SEQ ID NO:4, the CDR-L2 comprises or consists of the amino acid sequence of SEQ ID NO:20, the CDR-L3 comprises or consists of the amino acid sequence of SEQ ID NO:29, the CDR-H1 comprises or consists of the amino acid sequence of SEQ ID NO:50, the CDR-H2 comprises or consists of the amino acid sequence of SEQ ID NO:63, and the CDR-H3 comprises or consists of the amino acid sequence of SEQ ID NO:75.

B11. The binding agent of any one of embodiments B0 to B10, wherein the antibody, comprises a constant region of an IgG, IgD, IgE, IgA or IgM.

B12. The binding agent of any one of embodiments B0 to B11, wherein the antibody, or antigen binding portion thereof, is humanized.

B13. The binding agent of any one of embodiments B0 to B12, wherein the antibody, or antigen binding portion thereof binds specifically to a human syndecan-1 with a binding affinity (KD) of 50 nM or less.

B14. The binding agent of any one of embodiments B0 to B13, wherein the anti-neoplastic agent is covalently attached to the antibody, or antigen binding portion thereof, by a linker.

B15. The binding agent of embodiment B14, wherein the linker comprises or consists of a peptide comprising one or more amino acids, 5 to 100 amino acids, 5 to 50 amino acids, 5 to 25 amino acids, 5 to 20 amino acids, or 5 to 10 amino acids.

B16. The binding agent of embodiment B14, wherein the linker comprises an optionally substituted C1-C50 alkyl, an optionally substituted C2-C50 alkenyl, an optionally substituted C2-C50 alkynyl, acyl, acyloxy, alkyloxycarbonyloxy, aryloxycarbonyloxy, cycloalkyl, cycloalkenyl, alkoxy, cycloalkoxy, aryl, heteroaryl, arylalkoxy carbonyl, alkoxy carbonylacyl, aminocarbonyl, aminocarboyloxy, azido, phenyl, cycloalkylacyl, alkylthio, arylthio, oxysulfonyl, carboxy, thio, sulfoxide, sulfone, sulfonate esters, thiocyano, amide, amino, ester, halogenated alkyl, or a combination thereof.

B17. The binding agent of any one of embodiments B0 to B16, wherein the antibody, or antigen binding portion thereof, is non-covalently attached to the anti-neoplastic agent by means of a binding pair.

B18. The binding agent of any one of embodiments B0 to B17, wherein the anti-neoplastic agent is attached to an amino-terminal end of a heavy chain or light chain of the antibody, or antigen binding portion thereof.

B19. The binding agent of any one of embodiments B0 to B18, wherein the anti-neoplastic agent is attached to a carboxy-terminal end of a heavy chain or light chain of the antibody, or antigen binding portion thereof.

B20. The binding agent of any one of embodiments B0 to B19, wherein the anti-neoplastic agent is selected from the group consisting of an auristatin, a dolastatin, a maytansine, a tubulysin, a calicheamicin, a duocarmycin, a doxorubicin, a pseudomonas exotoxin-A (PE38), an irinotecan and a derivative of any one of the foregoing.

B21. The binding agent of any one of embodiments B0 to B20, wherein the anti-neoplastic agent comprises a monomethyl auristatin E (MMAE) or a monomethyl auristatin F (MMAF).

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. The terms and expressions which have been employed are used as terms of description and not of limitation, and use of such terms and expressions do not exclude any equivalents of the features shown and described or segments thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

Thus, it should be understood that although the present technology has been specifically disclosed by representative embodiments and optional features, modification and variation of the concepts herein disclosed can be resorted to by those skilled in the art, and such modifications and variations are considered within the scope of this technology.

1. A bi-specific binding agent comprising
(a) an antibody, or antigen binding portion thereof, that binds specifically to syndecan-1 (CD138); and
(b) a Fynomer that binds specifically to a fibroblast growth factor receptor 3 (FGFR3), wherein the Fynomer comprises a polypeptide having an amino acid sequence of
(i)GVTLFVALYDYEVYGPTPMLSFHKGEKFQIL(X₁)(X₂)(X₃)(X₄)GPYWEARSL(X₅)TGETG(X₆)IPSNYVAPVDSIQ (SEQ ID NO:99) or
(ii)GVTLFVALYDYEVMSTTALSFHKGEKFQILSQSPHGQYWEARSLTTGETG(X₇)IPSNYVAPVDSIQ (SEQ ID NO:113), wherein the amino acids (X₁), (X₂), (X₃), (X₄), (X₅), (X₆) and (X₇) are selected from any amino acid.

2-5. (canceled)

6. The bi-specific binding agent of claim 1, wherein the antibody, or antigen binding portion thereof, comprises the following light chain and heavy chain complementarity determining regions (CDR):
(i) a CDR-L1 (light chain CDR1) comprising an amino acid sequence from SEQ ID NOs:2-15;
(ii) a CDR-L2 (light chain CDR2) comprising an amino acid sequence selected from SEQ ID NOs:16-26, and
(iii) a CDR-L3 (light chain CDR3) comprising an amino acid sequence selected from SEQ ID NOs:27-33,
(iv) a CDR-H1 (heavy chain CDR1) comprising an amino acid sequence selected from SEQ ID NOs:45-59:
(v) a CDR-H2 (heavy chain CDR2) comprising an amino acid sequence selected from SEQ ID NOs:60-71, and
(vi) a CDR-H3 (heavy chain CDR3) comprising an amino acid sequence selected from SEQ ID NOs:72-81.

7. (canceled)

8. The bi-specific binding agent of claim 6, wherein
(i) the CDR-L1 comprises an amino acid sequence of SEQ ID NO:3;
(ii) the CDR-L2 comprises an amino acid sequence of SEQ ID NO:18;
(iii) the CDR-L3 comprises an amino acid sequence of SEQ ID NO:28;
(iv) the CDR-H1 comprises an amino acid sequence of SEQ ID NO:46;
(v) the CDR-H2 comprises of an amino acid sequence of SEQ ID NO:60; and
(vi) the CDR-H3 comprises of an amino acid sequence of SEQ ID NO:72.

9. The bi-specific binding agent of claim 6, wherein
(i) the CDR-L1 comprises an amino acid sequence of SEQ ID NO:2;
(ii) the CDR-L2 comprises an amino acid sequence of SEQ ID NO:17;
(iii) the CDR-L3 comprises an amino acid sequence of SEQ ID NO:27;
(iv) the CDR-H1 comprises or consists of an amino acid sequence of SEQ ID NO:47;
(v) the CDR-H2 comprises an amino acid sequence of SEQ ID NO:61; and
(vi) the CDR-H3 comprises an amino acid sequence of SEQ ID NO:73.

10-12. (canceled)

13. The bi-specific binding agent of claim 6, wherein the antibody, or antigen binding portion thereof comprises a humanized light chain variable region comprising an amino acid sequence having at least 85% identity to an amino acid sequence selected from selected from the group consisting of SEQ ID NOs:41, 42, and 43, and/or a humanized heavy chain variable region comprising an amino acid sequence having at least 85% identity to an amino acid sequence selected from the group consisting of SEQ ID NOs:89, 90, 91 and 92.

14. The bi-specific binding agent of claim 13, wherein the antibody, or antigen binding portion thereof comprises a humanized light chain variable region comprising an amino acid sequence of SEQ ID NO:41 and a humanized heavy chain variable region comprising an amino acid sequence of SEQ ID NO:90.

15. The bi-specific binding agent of claim 14, wherein the antibody, or antigen binding portion thereof comprises a humanized light chain comprising an amino acid sequence of SEQ ID NO:44 and/or a humanized heavy chain comprising an amino acid sequence of SEQ ID NO:93.

16. (canceled)

17. The bi-specific binding agent of claim 6, wherein
(i) the CDR-L1 comprises an amino acid sequence of SEQ ID NO:4;
(ii) the CDR-L2 comprises an amino acid sequence of SEQ ID NO:20;
(iii) the CDR-L3 comprises an amino acid sequence of SEQ ID NO:29;
(iv) the CDR-H1 comprises an amino acid sequence of SEQ ID NO:50;
(v) the CDR-H2 comprises an amino acid sequence of SEQ ID NO:63; and
(vi) the CDR-H3 comprises an amino acid sequence of SEQ ID NO:75.

18-36. (canceled)

37. The bi-specific binding agent of claim 1, wherein the Fynomer binds specifically to the human FGFR3 isoform 3b and the FGFR3 isoform 3c.

38-41. (canceled)

42. The bi-specific binding agent of claim 1, wherein
(X₁) is N, R, or K;
(X₂) is S, G, K or R;
(X₃) is S or G;
(X₄) is E, Q, D, S or K;
(X₅) is T or A; and
(X₆) is Y, W or L.

43. The bi-specific binding agent of claim 42, wherein
(X₁) is R, or K;
(X₂) is G, K or R;
(X₃) is S;
(X₄) is Q, D, S or K;
(X₅) is T or A; and
(X₆) is W or L.

44. The bi-specific binding agent of claim 1, wherein the Fynomer comprises a polypeptide comprising an amino acid sequence that is at least 90% identical to an amino acid sequence selected from:
(SEQ ID NO: 101; FF2L4C3)
GVTLFVALYDYEVYGPTPMLSFHKGEKFQILNSSEGPYWEARSLTTGETG
LIPSNYVAPVDSIQ;
(SEQ ID NO: 103; FF44L65G12)
GVTLFVALYDYEVYGPTPMLSFHKGEKFQILRGGQGPYWEARSLTTGETG
LIPSNYVAPVDSIQ;
(SEQ ID NO: 105; FF44L65G7)
GVTLFVALYDYEVYGPTPMLSFHKGEKFQILRGGDGPYWEARSLTTGETG
LIPSNYVAPVDSIQ;
(SEQ ID NO: 107; FF48L66G7; “G7”)
GVTLFVALYDYEVYGPTPMLSFHKGEKFQILKGGSGPYWEARSLTTGETG
LIPSNYVAPVDSIQ;
(SEQ ID NO: 109; FF43L65D5)
GVTLFVALYDYEVYGPTPMLSFHKGEKFQILRKGKGPYWEARSLATGETG
LIPSNYVAPVDSIQ;
(SEQ ID NO: 111; FF44L65B7)
GVTLFVALYDYEVYGPTPMLSFHKGEKFQILRRGSGPYWEARSLTTGETG
LIPSNYVAPVDSIQ;
and
(SEQ ID NO: 116; FF40L54A5)
GVTLFVALYDYEVMSTTALSFHKGEKFQILSQSPHGQYWEARSLTTGETG
WIPSNYVAPVDSIQ.

45-48. (canceled)

49. The bi-specific binding agent of claim 1, wherein the Fynomer is covalently attached to the antibody, or antigen binding portion thereof, by a linker.

50-54. (canceled)

55. The bi-specific binding agent of claim 1, further comprising an anti-neoplastic agent.

56-61. (canceled)

62. The bi-specific binding agent of claim 55, wherein the anti-neoplastic agent comprises a pyrrolobenzodiazepine toxin.

63-93. (canceled)

94. The bi-specific binding agent of claim 1, wherein the antibody, or antigen binding portion thereof, competes for binding to syndecan-1 with a second binding agent comprises a CDR-L1 having an amino acid sequence selected from SEQ ID NOs:2-15, a CDR-L2 having an amino acid sequence selected from SEQ ID NOs:16-26, a CDR-L3 having an amino acid sequence selected from SEQ ID NOs:27-33, a CDR-H1 having an amino acid sequence selected from SEQ ID NOs:45-59, a CDR-H2 having an amino acid sequence selected from SEQ ID NOs:60-71, and a CDR-H3 having an amino acid sequence selected from SEQ ID NOs:72-81.

95-100. (canceled)

101. A pharmaceutical composition comprising the bi-specific binding agent of claim 1 and a pharmaceutically acceptable excipient, diluent, additive or carrier.

102-104. (canceled)

105. A method for treating a neoplasm in a subject, which comprises administering the binding agent of claim 1 to the subject in need thereof.

106. The method of claim 105, wherein the neoplasm is selected from a carcinoma, sarcoma, nervous system neoplasia, lymphoma, myeloma, leukemia, melanoma, mesothelioma, solid or soft tissue tumors, and a secondary cancer.

107-147. (canceled)
